(19) United States
(12) Patent Application Publication
(10) Pub. No.: US 2024/0167008 A1
(43) Pub. Date: May 23, 2024

(54) NOVEL CRISPR ENZYMES, METHODS, SYSTEMS AND USES THEREOF

(71) Applicant: Beam Therapeutics Inc., Cambridge, MA (US)

(72) Inventors: Bernd ZETSCHE, Cambridge, MA (US); Luis BARRERA, Cambridge, MA (US); David A. BORN, Cambridge, MA (US); Ming SUN, Cambridge, MA (US)

(73) Assignee: Beam Therapeutics Inc., Cambridge, MA (US)

(21) Appl. No.: 18/283,148

(22) PCT Filed: Mar. 23, 2022
(86) PCT No.: PCT/US2022/021523

§ 371 Date: Sep. 20, 2023

Related U.S. Application Data
(60) Provisional application No. 63/164,798, filed on Mar. 23, 2021.

(51) Int. Cl. C12N 9/22; A61K 31/7088; A61K 38/46; C12N 9/78; C12N 15/11; C12N 15/90

(52) U.S. Cl. CPC C12N 9/22; A61K 31/7088; A61K 38/465; C12N 9/78; C12N 15/11; C12N 15/907; C12Y 305/04004; C12Y 305/04005; C12N 2310/20

(57) ABSTRACT

The present invention provides novel systems, methods and compositions for making and using recombinantly engineered novel Cas9 enzymes optimized for human cells, for nucleic acid targeting and manipulation. The present invention is based on the discovery of novel Cas9 enzymes from Streptococcus constellatus, Sharpen spp. isolate RUG017, Veillonella parvula, Ezakiella peruensis, Lactobacillus fermentum strain AF15-40LB strain and Peptoniphilus sp. Marseille-P3761 bacteria that were codon-optimized and recombinantly produced for use in human cells. In some embodiments, novel Cas9 enzymes can be used for base editing. In some embodiments, the novel engineered Cas9 enzymes are used to treat human diseases.

### CROSS-REFERENCE TO RELATED APPLICATIONS

This application claims priority to U.S. Provisional Patent Application Ser. No. 63/164,798, filed on Mar. 23, 2021, which is incorporated by reference herein in its entirety for all purposes.

### BACKGROUND

Enzymes from the prokaryotic Clustered, Regularly Interspaced Short Palindromic Repeats (CRISPR) and CRISPR-associated protein (CRISPR-Cas) systems have been harnessed as reprogrammable and highly specific genome editing tools for use in eukaryotes. Besides genome editing and cleavage, CRISPR-Cas9 can be used to localize effector molecules to specific sites on the genome, allowing genetic and epigenetic regulation and transcriptional modulation through a variety of mechanisms.

However, diverse genomes and genomic targets require a variety of tools for effective genetic engineering, and there remains a need to expand the CRISPR toolbox through the discovery and engineering of novel Cas proteins that can recognize and target diverse sequences.

While CRISPR-Cas9 systems can be used to knock out a gene or modify the expression of a gene, certain kind of gene editing requires precise modifications to the target gene, such as editing a single base within the gene. Such precise modifications remain a challenge and requires a diverse gene editing toolkit to effectuate precise genomic modifications in a wide variety of target genes.

### SUMMARY OF THE INVENTION

The identification of novel Cas9 enzymes with specificity for unique protospacer adjacent motifs (PAM) allows for the expansion of the available tools for gene editing. The present invention provides, among other things, engineered, non-naturally occurring novel Cas9 enzymes isolated from Streptococcus constellatus, Sharpea spp. isolate RUG017, Veillonella parvula, Ezakiella peruensis, Lactobacillus fermentum strain AF15-40LB and Peptoniphilus sp. Marseille-P 3761 bacteria. The present invention is based, in part, on the surprising discovery that novel Cas9 enzymes discovered from different bacteria, which recognize specific PAM sequences can be engineered for expression in eukaryotic cells (e.g., human, plant, etc.). Accordingly, the described Cas9 enzymes and their variants are functional in eukaryotes. The examples provided herewith show use of engineered, non-naturally Cas9 enzymes in human cells with diverse PAM recognition sequences to target various genomic sites. For example, Cas9 engineered from Streptococcus constellatus, Ezakiella peruensis and Peptoniphilus sp. Marseille-P376/recognizes the consensus PAM sequence 5′-NGG-3′. The consensus PAM sequence recognized by Cas9 isolated from Sharpea spp. isolate RUG017 is 5′-NAGHC-3′. The consensus PAM sequence recognized by Cas9 isolated from Veillonella parvula was identified as 5′-NRHRRH-3′. The consensus PAM sequence recognized by Cas9 isolated from Lactobacillus fermentum strain AF15-40LB was identified as 5′-NNAAA-3′. (H=A, C or T; R=A or G).

In one aspect, an engineered, non-naturally occurring Cas9 protein modified from Streptococcus constellatus Cas9, Sharpea Cas9, Veillonella parvula Cas9, Ezakiella peruensis Cas9, Lactobacillus fermentum strain AF15-40LB Cas9 or Peptoniphilus sp. Marseille-P 3761 Cas9 is provided herein.

In some embodiments, the Streptococcus constellatus Cas9 protein has at least 80% sequence identity to

(SEQ ID NO: 1)
MGKPYSIGLDIGTNSVGWAVVTDDYKVPAKKMKVLGNTDKQSIKKNLLGA
LLFDSGETAEATRLKRTARRRYTRRKNRLRYLQEIFTGEMNKVDENFFQR
LDDSFLVDEDKRGEHHPIFGNIAAEVKYHDDFPTIYHLRRHLADTSKKAD
LRLVYLALAHMIKFRGHFLYEGDLKAENTDVQALFKDFVEEYDKTIEESH
LSEITVDALSILTEKVSKSSRLENLIAHYPTEKKNTLFGNLIALSLDLHP
NFKTNFQLSEDAKLQFSKDTYEEDLEGFLGEVGDEYADLFASAKNLYDAI
LLSGILTVDDNSTKAPLSASMVKRYEEHQKDLKKLKDFIKVNAPDQYNAI
FKDKNKKGYASYIESGVKQDEFYKYLKGILLKINGSGDFLDKIDREDFLR
KQRTFDNGSIPHQIHLQEMHAILRRQGEHYPFLKENQDKIEKILTFRIPY
YVGPLARKGSRFAWAEYKADEKITPWNFDDILDKEKSAEKFITRMTLNDL
YLPEEKVLPKHSPLYEAFTVYNELTKVKYVNEQGEAKFFDTNMKQEIFDH
VFKENRKVTKDKLLNYLNKEFEEFRIVNLTGLDKENKAFNSSLGTYHDLR
KILDKSFLDDKANEKTIEDIIQTLTLFEDREMIRQRLQKYSDIFTKAQLK
KLERRHYTGWGRLSYKLINGIRNKENKKTILDYLIDDGYANRNFMQLIND
DALSFKEEIARAQIIDDVDDIANVVHDLPGSPAIKKGILQSVKIVDELVK
VMGHNPANIIIEMARENQTTDKGRRNSQQRLKLLQDSLKNLDNPVNIKNV
ENQQLQNDRLFLYYIQNGKDMYTGETLDINNLSQYDIDHIIPQAFIKDNS
LDNRVLTRSDKNRGKSDDVPSIEVVHEMKSFWSKLLSVKLITQRKFDNLT
KAERGGLTEEDKAGFIKRQLVETRQITKHVAQILDERFNTEFDGNKRRIR
NVKIITLKSNLVSNFRKEFELYKVREINDYHHAHDAYLNAVVGNALLLKY
PQLEPEFVYGEYPKYNSYRSRKSATEKFLFYSNILRFFKKEDIQTNEDGE
IAWNKEKHIKILRKVLSYPQVNIVKKTEEQTGGFSKESILPKGESDKLIP
RKTKNSYWDPKKYGGFDSPVVAYSILVFADVEKGKSKKLRKVQDMVGITI
MEKKRFEKNPVDFLEQRGYRNVRLEKIIKLPKYSLFELENKRRRLLASAK
ELQKGNELVIPQRFTTLLYHSYRIEKDYEPEHREYVEKHKDEFKELLEYI
SVFSRKYVLADNNLTKIEMLFSKNKDAEVSSLAKSFISLLTFTAFGAPAA
FNFFGENIDRKRYTSVTECLNATLIHQSITGLYETRIDLSKLGED.

In some embodiments, the Sharpea Cas9 protein has at least 80% sequence identity to

(SEQ ID NO: 4)
MAKNKDIRYSIGLDIGTNSVGWAVMDEHYELLKKGNHHMWGSRLFDAAEPAATRRASRSIRR
RYNKRRERIRLLRDLLGDMVMEVDPTFFIRLLNVSELDEEDKQKNLGNDYKDNYNLFIEKDE
NDKTYYDKYPTIYHLRKELCENKEKADPRLIYLALHHIVKYRGNFLKEGQSFAKVYEDIEEK
LDNTLKKFMSLNDLDNLFVDNDINSMITVLSKIYQRSKKADDLLKIMNPTKEERAAYKEFTK
ALVGLKFNVSKMILAQEVKKDDKDIELDFSNVDYDSTVDGLQAELGEYIEFIEMLHSINSWV
ELQDILGNNSTISAAMVERYEEHKNDLRVLKKVIREELPDKYNEVFREDNPKLHNYLGYIKY
PKNTPVEEFYEYIKRLLAKVDTGEAREILERIDLEKFMLKQNSRINGSIPYQMQKDEMIQII
DNQSVYYPQLKENREKLISILEFRIPYYFGPLNTHSEFAWIKKFEDKQKERILPWNYDQIVD
IDATAEGFIERMQNTGTYFPDKPVMAKNSLTVSKFEVLNELNKIRINGKLIPVETKKELLSD
LFMKNKTITDKKLKDWLVTHQYYDTNEELKIEGYQKDLQFSTSLAPWIDFTKIFGEINASNY
QLIEKIIYDISIFEDKKILKRRLKKVYQLDDLLVDKILKLNYTGWSRLSEKLLTGIKSKNSK
ETILSILENSNMNLMEIINDESLGFKQIIEESNKKDIEGPFRYDEVKKLAGSPAIKRGIWQA
LLVVQEITKFMKHEPSHIYIEFAREEQEKVRTESRIAKLQKIYKDLNLQTKEDQLVYESLKK
EDAKKKIDTDALYLYYLQMGKSMYSGKPLDIDKLSTYHIDHILPRSLIKDDSLDNRVLVLPK
ENEWKLDSETVPFEIRNKMMGFWQKLHENGLMSNKKFFSLIRTDENEKDKKRFINRQLVETR
QIIKNVAVIINDHYTNTNVVTVRAELSHQFRERYKIYKNRDLNDLHHAHDAYIACILGQFIH
QNFGNMDVNMIYGQYKKNYKKDVQEHNNYGFILNSMNHIHENDDNSVIWDPSYIGKIKSCFC
YKDVYVTKKLEQNDAKLEDLTILPSDKNSENGVTKAKIPVNKYRKDVNKYGGFSGDAPIMLA
IEADKGKKHVRQVIAFPLRLKNYNDEERIKFIEKEKNLKNVKILTEVKKNQLILINHQYFFI
TGTNELVNATQLKLSAKNTKNLFNLVDANKHNKLESIDDANFNEVIQELICKLQEPIYSRYN
SIGKEFEDSYEKINAVTKQDKLYIIEYLIAIMSAKATQGYIKPELAREIGTNGKNKGRIKSF
TIDLNKTTFISTSVTGLESKKYKL.

In some embodiments, the Veillonella parvula Cas9 protein has at least 80% sequence identity to

(SEQ ID NO: 8)
MSIINFQRRGLMETQASNQLISSHLKGYPIKDYFVGLDIGTSSVGWAVINKAYELLKERSHK
MWGSRLFDEGESAVARRGERSMRRRLERRKLRLKLLEELFADAMAQVDPTFFMRLRESKYHY
EDKTTGHSSKHILFIDKNYNDQDYFKEYPTVYHLRSELMKSGTDDIRKLFLAVHHILKYRGN
FLYEGATFDSNASTLDDVIKQALENITENCFDCNSAISSIGQILMEAGKTKSDKAKAIEHLV
DTYIATDTVDTSSKTQKDQVKEDKKRLKAFANLVLGLNASLIDLFGSVEELEEDLKKLQITG
DTYDDKRDELAKAWSDEIYIIDDCKSVYDAIILLSIKEPGLTISESKVKAFNKHKDDLAILK
SLLKSDRSIYNTMFKVDEKGLHNYVHYIKQGRTEETSCNREDFYKYTKKIVEGLSDSKDKEY
ILSQIELQILLPLQRIKDNGVIPYQLHLEELKAILAKCGPKFPFLNEVADGFSVAEKLIKML
EFRIPYYVGPLNTHHNVDNGGFAWAVRKASGRVTPWNEDDKIDREKSAAAFIKNLTNKCTYL
LGEDVLPKSSLLYSEFMLLNELNNVRIDGKPLEKVVKEHLIEAVFKQDHKKMTKNRIEQFLK
DNGYISETHKHEITGLDGEIKNDLASYRDMVRILGDGEDRSMAEEIITDITIFGESKKMLRE
TLRKKFASCLDDEAIKKLTKLRYRDWGRLSQKLLNGIEGCDKAGDGTPETIIILMRNFSYNL
MELLGDKFSFMERIQEINAKLTEGQIVNPHDIIDDLALSPAVKRAVWQALRIVDEVAHIKKA
LPARIFVEVTRSNKNEKKKKDSRQKRLSDLYAAIKKDDVLLNGLNNEIFGELKSSLAKYDDA
ALRSKKLYLYYTQMGRCAYTGEIIELSLLNTDNYDIDHIYPRSLTKDDSFDNLVLCKRTANA
QKSDAYPISEEIQKTQKPFWTFLKQQGLISERKYERLTRITPLTADDLSGFIARQLVETNQS
VKAATTLLRRLYPGVDVVFVKAENVTDERHDNNFIKVRSLNHHHHAKDAYLNIVVGNVYHER
FTRNFRAFFKKNGANRTYNLAKMENYDVNCTNAKDGKAWDVKTSMDTVKKMMDSNDVRVTKR
LLEQTGALADATIYKATVAGKAKDGAYIGMKTKSSVFADVSKYGGMTKIKNAYSIIVQYTGK
KGEVIKEIVPLPIYLTNRNTTDQDLINYVASIIPQAKDISIIYGKLCINQLVKVNGFYYYLG
GKTNSKFCIDNAIQVIVSNEWIPYLKVLEKENNMRKDNKDLKANVVSTRALDNKHTIEVRIV
EEKNIEFFDYLVSKLKMPIYQKMKGNKAAELSEKGYGLFKKMSLEEQSIHLIELLNLLTNQK
TTFEVKPLGITASRSTVGSKISNQDEFKVINESITGLYSNEVTIV.

In some embodiments, the Ezakiella peruensis Cas9 protein has at least 80% sequence identity to

(SEQ ID NO: 14)
MTKVKDYYIGLDIGTSSVGWAVTDEAYNVLKENSKKMWGVRLEDDAKTAEERRGQRGARRRL
DRKKERLSLLQDFFAEEVAKVDPNFFLRLDNSDLYMEDKDQKLKSKYTLENDKDEKDKNFHK
KYPTIHHLLMDLIEDDSKKDIRLVYLACHYLLKNRGHFIFEGQKFDTKSSFENSLNELKVHL
NDEYGLDLEFDNENLINILTDPKLNKTAKKKELKSVIGDTKFLKAVSAIMIGSSQKLVDLFE
NPEDFDDSAIKSVDESTTSFDDKYSDYELALGDKIALVNILKEIYDSSILENLLKEADKSKD
GNKYISNAFVKKYNKHGQDLKEFKRLVRQYHKSAYEDIFRSEKVNDNYVSYTKSSISNNKRV
KANKFTDQEAFYKFAKKHLETIKYKINKVNGSKADLELIDGMLRDMEFKNEMPKIKSSDNGV
IPYQLKLMELNKILENQSKHHEFLNVSDEYGSVCDKIASIMEFRIPYYVGPLNPNSKYAWIK
KQKDSEITPWNFKDVVDLDSSREEFIDSLIGRCTYLKDEKVLPKASLLYNEYMVLNELNNLK
LNDLPITEEMKKKIFDQLFKTRKKVTLKAVANLLKKEFNINGEILLSGTDGDFKQGLNSYND
FKAIVGDKVDSDDYRDKIEEIIKLIVLYGDDKSYLQKKIKAGYGKYFTDSEIKKMAGLNYKD
WGRLSKKLLTGLEGANKITGERGSIIHEMREYNLNLMELMSASFTFTEEIQKLNPVDDRKLS
YEMVDELYLSPSVKRMLWQSLRIVDEIKNIMGTDSKKIFIEMARGKEEVKARKESRKNQLLK
FYKDGKKAFISEIGEERYSYLLSEIEGEEENKERWDNLYLYYTQLGRCMYSLEPIDISELSS
KNIYDQDHIYPKSKIYDDSIENRVLVKKDLNSKKGNSYPIPDEILNKNCYAYWKILYDKGLI
GQKKYTRLTRRTGFTDDELVQFISRQIVETRQATKETANLLKTICKNSEIVYSKAENASRER
QEFDIVKCRAVNDLHHMHDAYINIIVGNVYNTKFTKDPMNFVKKQEKARSYNLENMFKYDVK
RGGYTAWIADDEKGTVKNASIKRIRKELEGTNYRFTRMNYIESGALFNATLQRKNKGSRPLK
DKGPKSSIEKYGGYTNINKACFAVLDIKSKNKIERKLMPVEREIYAKQKNDKKLSDEIFSKY
LKDRFGIEDYRVVYPVVKMRTLLKIDGSYYFITGGSDKTLELRSALQLILPKKNEWAIKQID
KSSENDYLTIERIQDLTEELVYNTFDIIVNKFKTSVEKKSFLNLFQDDKIENIDFKFKSMDF
KEKCKTLLMLVKAIRASGVRQDLKSIDLKSDYGRLSSKINNIGNYQEFKIINQSITGLFENE
VDLLKL

In some embodiments, the Lactobacillus fermentum Cas9 protein has at least 80% sequence identity to

(SEQ ID NO: 84)
MKEYHIGLDIGTSSIGWAVTDSQFKLMRIKGKTAIGVRLFEEGKTAAERRTFRTTRRRLKRR
KWRLHYLDEIFAPHLQEVDENFLRRLKQSNIHPEDPAKNQAFIGKLLFPDLLKKNERGYPTL
IKMRDELPVEQRAHYPVINIYKLREAMINEDRQFDLREVYLAVHHIVKYRGHFLNNASVDKE
KVGRIDFDKSFNVLNEAYEELQNGEGSFTIEPSKVEKIGQLLLDTKMRKLDRQKAVAKLLEV
KVADKEETKRNKQIATAMSKLVLGYKADFATVAMANGNEWKIDLSSETSEDEIEKFREELSD
AQNDILTEITSLESQIMLNEIVPNGMSISESMMDRYWTHERQLAEVKEYLATQPASARKEFD
QVYNKYIGQAPKEKGFDLEKGLKKILSKKENWKEIDELLKAGDELPKQRTSANGVIPHQMHQ
QELDRIIEKQAKYYPWLATENPATGERDRHQAKYELDQLVSFRIPYYVGPLVTPEVQKATSG
AKFAWAKRKEDGEITPWNLWDKIDRAESAEAFIKRMTVKDTYLLNEDVLPANSLLYQKYNVL
NELNNVRVNGRRLSVGIKQDIYTELFKKKKTVKAGDVASLVMAKTRGVNKPSVEGLSDPKKF
NSNLATYLDLKSIVGDKVDDNRYQMDLENIIEWRSVFEDGEIFADKLTEVEWLTDEQRSALV
KKRYKGWGRLSKKLLTGIVDENGQRIIDLMWNTDQNFMQIVNQPVFKEQIDQLNQKAITNDG
MTLRERVESVLDDAYTSPQNKKAIWQVVRVVEDIVKAVGNAPKSISIEFARNEGNKGEITRS
RRTQLQKLFEDQAHELVKDTSLTEELEKAPDLSDRYYFYFTQGGKDMYTGDPINFDEISTKY
DIDHILPQSFVKDDSLDNRVLVSRAENNKKSDRVPAKLYAAKMKPYWNQLLKQGLITQRKFE
NLTMDVDQTIKYRSLGFVKRQLVETRQVIKLTANILGSMYQEAGTDIIETRAGLTKQLREEF
DLPKVREVNDYHHAVDAYLTTFAGQYLNRRYPKLRSFFVYGEYMKFKHGSDLKLRNFNFFHE
LMEGDKSQGKVVDQQTGELITTRDEVADYFDWVINLKVMLISNETYEETGKYFDASHESSSL
YLKNQNKKSKLVVPLKNKLQPEYYGAYTGITQGYMVILKLLDKKGGFGVYRIPRYAADILNK
CHDEVAYRNKIAEIISSDPRAPKSFEVVVPRVLKGTELVDGEEKFILSSYRYKVNATQLILP
VSDIKLIQDNFKALKKLNVEMQTKKLIEIYDNILRQVDKYYKLYDINKFRAKLHDGRSKFVE
LDDFGQDASKEKVIIKILRGLHFGSDLQNLKEIGFGTTPLGQFQVSEAGIRLSNTAFIIFKS
PTGLFNRKLYLKNL.

In some embodiments, the Peptoniphilus sp. Marseille-P3761 Cas9 protein has at least 80% sequence identity to

(SEQ ID NO: 86)
MEKKTNYTIGLDIGTDSVGWAVVKDDLELVKKRMKVLGNTETNYIKKNLWGSLLFESGQTAK
DRRLKRVARRRYERRRNRLTELQKIFAPAIDEVDENFFERLNESFLVPEDKAFSKNPIFGTL
GEDKTYYKTYPTIYHLRQHLADSEEKADVRLIYLALAHMIKYRGHFLIEGKLDTEHIAINEN
LEQFFESYNALFSEEPIELRKEELIAIENILREKNSRTVKEKRITSFLKDIGRANKQSPMMA
FITLIVGKKAKFKAAFNLEEEISLNLTDDSYDENLEILLNTIGSDFADLEDHAQRVYNAVEL
AGILSGDVKNTHAKLSAQMVAMYERHKEQLKEYKSFIKANLPDQYDMTFVAPKDAQKKDLKG
YAGYIDGNMSQDSFYKFVKDQLKEVPGSEKFLDSIEKEDELRKQRSFYNGVIPNQVHLAEME
AILDRQENYYPWLKENREKIISLLTFRIPYYVGPLADGQSEFAWLERKSDEKIKPWNFSDVV
DLDRSAEKFIEQLIGRDTYLPDEYVLPKKSLIYQKYMVENELTKIAYLDERQKRMNLSSVEK
KEIFETLFKKRSKVTEKQLVKFFENYLQIDNPTIFGIEDAFNADYSTYVELAKVPGMKSMMD
DPDNEDLMEEIVKILTVFEDRKMRRKQLEKYKERLSPEQIKELAKKHYTGWGRLSKKLLVGI
RDKETQKTILDYLVEDDNHSGGRQHLNRNLMQLINDDRLSFKKTIAELQMIDPSADLYAQVQ
EIAGSPAIKKGILLGLKIVDEIIRVMGEKPENIVIEMARENQTTARGKALSKRREAKIKEGL
AALGSSLLKENLPGNADLSQRKIYLYYTQNGKDIYLDEPLDFDRLSQYDEDHIIPQSFTVDN
SLDNLVLTNSSQNRGNKKDDVPSLEVVNRQLAYWRSLKDAGLMTQRKFDNLTKAMRGGLTDK
DRERFIQRQLVETRQITKNVAKLLDMRLNDKKDEAGNKIRETNIVLLKSAMASEFRKMERLY
KVRELNDYHHAHDAYLNAAIAINLLALYPYMADDEVYGEFRYKKKPQAEKATYEKLRQWNLI
KRFGEKQLFTPDHEDCWNKERDIKTIKKVMGYRQVNVVKKAEERTGMLFKETINGKTNKGSR
IPIKKDLDPSKYGGYIEEKMAYYAVISYEDKKKKPGKTIVGISIMDKKEFEYDSISYLGKLG
FSNPVVQIILKNYSLIAYPDGRRRYITGATKTTKGKVELQKANQIAMEQDLVNFIYHLKNYD
EISHPESYAFVQSHTDYFDRLEDSIEHYTRRFLDAETNINRLRRIYEEEKKKDPVDIEALVA
SFIELLKLTSAGAPADFIFMGEAISRRRYNSMTGLEDGQVIYQSLTGLYETRMRFED.

In some embodiments, the Cas9 protein comprises an amino acid sequence that is at least 85%, at least 90%, at least 92%, at least 95%, at least 96%, at least 97%, at least 98%, or at least 99% identical to SEQ ID NOs: 1, 4, 8, 14, 84 or 86.

In some embodiments, the Cas9 protein further comprises a nuclear localization sequence (NLS) and/or a FLAG, HIS or HA tag.

In some embodiments, the Streptococcus constellatus Cas9 has an amino acid sequence at least 80% identical to

(SEQ ID NO: 2)
MPKKKRKVGGKPYSIGLDIGTNSVGWAVVTDDYKVPAKKMKVLGNTDKQSIKKNLLGALLFD
SGETAEATRLKRTARRRYTRRKNRLRYLQEIFTGEMNKVDENFFQRLDDSELVDEDKRGEHH
PIFGNIAAEVKYHDDFPTIYHLRRHLADTSKKADLRLVYLALAHMIKERGHFLYEGDLKAEN
TDVQALFKDFVEEYDKTIEESHLSEITVDALSILTEKVSKSSRLENLIAHYPTEKKNTLFGN
LIALSLDLHPNFKTNFQLSEDAKLQFSKDTYEEDLEGELGEVGDEYADLFASAKNLYDAILL
SGILTVDDNSTKAPLSASMVKRYEEHQKDLKKLKDFIKVNAPDQYNAIFKDKNKKGYASYIE
SGVKQDEFYKYLKGILLKINGSGDELDKIDREDELRKQRTEDNGSIPHQIHLQEMHAILRRQ
GEHYPFLKENQDKIEKILTFRIPYYVGPLARKGSRFAWAEYKADEKITPWNEDDILDKEKSA
EKFITRMTLNDLYLPEEKVLPKHSPLYEAFTVYNELTKVKYVNEQGEAKFFDTNMKQEIFDH
VFKENRKVTKDKLLNYLNKEFEEFRIVNLTGLDKENKAFNSSLGTYHDLRKILDKSELDDKA
NEKTIEDIIQTLTLFEDREMIRQRLQKYSDIFTKAQLKKLERRHYTGWGRLSYKLINGIRNK
ENKKTILDYLIDDGYANRNEMQLINDDALSFKEEIARAQIIDDVDDIANVVHDLPGSPAIKK
GILQSVKIVDELVKVMGHNPANIIIEMARENQTTDKGRRNSQQRLKLLQDSLKNLDNPVNIK
NVENQQLQNDRLFLYYIQNGKDMYTGETLDINNLSQYDIDHIIPQAFIKDNSLDNRVLTRSD
KNRGKSDDVPSIEVVHEMKSFWSKLLSVKLITQRKFDNLTKAERGGLTEEDKAGFIKRQLVE
TRQITKHVAQILDERENTEFDGNKRRIRNVKIITLKSNLVSNFRKEFELYKVREINDYHHAH
DAYLNAVVGNALLLKYPQLEPEFVYGEYPKYNSYRSRKSATEKFLFYSNILRFFKKEDIQTN
EDGEIAWNKEKHIKILRKVLSYPQVNIVKKTEEQTGGFSKESILPKGESDKLIPRKTKNSYW
DPKKYGGFDSPVVAYSILVFADVEKGKSKKLRKVQDMVGITIMEKKRFEKNPVDFLEQRGYR
NVRLEKIIKLPKYSLFELENKRRRLLASAKELQKGNELVIPQRFTTLLYHSYRIEKDYEPEH
REYVEKHKDEFKELLEYISVFSRKYVLADNNLTKIEMLFSKNKDAEVSSLAKSFISLLTFTA
FGAPAAFNFFGENIDRKRYTSVTECLNATLIHQSITGLYETRIDLSKLGEDGKRPAATKKAG
QAKKKKGSYPYDVPDYAYPYDVPDYAYPYDVPDYA.

In some embodiments, the Sharpea Cas9 has an amino acid sequence at least 80% identical to

(SEQ ID NO: 5)
MPKKKRKVGAKNKDIRYSIGLDIGTNSVGWAVMDEHYELLKKGNHHMWGSRLFDAAEPAATR
RASRSIRRRYNKRRERIRLLRDLLGDMVMEVDPTFFIRLLNVSFLDEEDKQKNLGNDYKDNY
NLFIEKDENDKTYYDKYPTIYHLRKELCENKEKADPRLIYLALHHIVKYRGNFLKEGQSFAK
VYEDIEEKLDNTLKKEMSLNDLDNLFVDNDINSMITVLSKIYQRSKKADDLLKIMNPTKEER
AAYKEFTKALVGLKFNVSKMILAQEVKKDDKDIELDFSNVDYDSTVDGLQAELGEYIEFIEM
LHSINSWVELQDILGNNSTISAAMVERYEEHKNDLRVLKKVIREELPDKYNEVFREDNPKLH
NYLGYIKYPKNTPVEEFYEYIKRLLAKVDTGEAREILERIDLEKEMLKQNSRINGSIPYQMQ
KDEMIQIIDNQSVYYPQLKENREKLISILEFRIPYYFGPLNTHSEFAWIKKFEDKQKERILP
WNYDQIVDIDATAEGFIERMQNTGTYFPDKPVMAKNSLTVSKFEVLNELNKIRINGKLIPVE
TKKELLSDLFMKNKTITDKKLKDWLVTHQYYDTNEELKIEGYQKDLQFSTSLAPWIDFTKIF
GEINASNYQLIEKIIYDISIFEDKKILKRRLKKVYQLDDLLVDKILKLNYTGWSRLSEKLLT
GIKSKNSKETILSILENSNMNLMEIINDESLGFKQIIEESNKKDIEGPFRYDEVKKLAGSPA
IKRGIWQALLVVQEITKFMKHEPSHIYIEFAREEQEKVRTESRIAKLQKIYKDLNLQTKEDQ
LVYESLKKEDAKKKIDTDALYLYYLQMGKSMYSGKPLDIDKLSTYHIDHILPRSLIKDDSLD
NRVLVLPKENEWKLDSETVPFEIRNKMMGFWQKLHENGLMSNKKFFSLIRTDFNEKDKKRFI
NRQLVETRQIIKNVAVIINDHYTNTNVVTVRAELSHQFRERYKIYKNRDLNDLHHAHDAYIA
CILGQFIHQNFGNMDVNMIYGQYKKNYKKDVQEHNNYGFILNSMNHIHENDDNSVIWDPSYI
GKIKSCFCYKDVYVTKKLEQNDAKLEDLTILPSDKNSENGVTKAKIPVNKYRKDVNKYGGFS
GDAPIMLAIEADKGKKHVRQVIAFPLRLKNYNDEERIKFIEKEKNLKNVKILTEVKKNQLIL
INHQYFFITGTNELVNATQLKLSAKNTKNLFNLVDANKHNKLESIDDANENEVIQELICKLQ
EPIYSRYNSIGKEFEDSYEKINAVTKQDKLYIIEYLIAIMSAKATQGYIKPELAREIGTNGK
NKGRIKSFTIDLNKTTFISTSVTGLESKKYKLGKRPAATKKAGQAKKKKGSYPYDVPDYAYP
YDVPDYAYPYDVPDYA.

In some embodiments, the Veillonella parvula Cas9 has an amino acid sequence at least 80% identical to

(SEQ ID NO: 9)
MPKKKRKVGSIINFQRRGLMETQASNQLISSHLKGYPIKDYFVGLDIGTSSVGWAVINKAYE
LLKFRSHKMWGSRLFDEGESAVARRGFRSMRRRLERRKLRLKLLEELFADAMAQVDPTFFMR
LRESKYHYEDKTTGHSSKHILFIDKNYNDQDYFKEYPTVYHLRSELMKSGTDDIRKLFLAVH
HILKYRGNFLYEGATFDSNASTLDDVIKQALENITFNCEDCNSAISSIGQILMEAGKTKSDK
AKAIEHLVDTYIATDTVDTSSKTQKDQVKEDKKRLKAFANLVLGLNASLIDLFGSVEELEED
LKKLQITGDTYDDKRDELAKAWSDEIYIIDDCKSVYDAIILLSIKEPGLTISESKVKAFNKH
KDDLAILKSLLKSDRSIYNTMFKVDEKGLHNYVHYIKQGRTEETSCNREDFYKYTKKIVEGL
SDSKDKEYILSQIELQILLPLQRIKDNGVIPYQLHLEELKAILAKCGPKFPELNEVADGFSV
AEKLIKMLEFRIPYYVGPLNTHHNVDNGGFAWAVRKASGRVTPWNEDDKIDREKSAAAFIKN
LTNKCTYLLGEDVLPKSSLLYSEFMLLNELNNVRIDGKPLEKVVKEHLIEAVFKQDHKKMTK
NRIEQFLKDNGYISETHKHEITGLDGEIKNDLASYRDMVRILGDGEDRSMAEEIITDITIFG
ESKKMLRETLRKKFASCLDDEAIKKLTKLRYRDWGRLSQKLLNGIEGCDKAGDGTPETIIIL
MRNFSYNLMELLGDKESFMERIQEINAKLTEGQIVNPHDIIDDLALSPAVKRAVWQALRIVD
EVAHIKKALPARIFVEVTRSNKNEKKKKDSRQKRLSDLYAAIKKDDVLLNGLNNEIFGELKS
SLAKYDDAALRSKKLYLYYTQMGRCAYTGEIIELSLLNTDNYDIDHIYPRSLTKDDSFDNLV
LCKRTANAQKSDAYPISEEIQKTQKPFWTFLKQQGLISERKYERLTRITPLTADDLSGFIAR
QLVETNQSVKAATTLLRRLYPGVDVVFVKAENVTDERHDNNFIKVRSLNHHHHAKDAYLNIV
VGNVYHERFTRNFRAFFKKNGANRTYNLAKMENYDVNCTNAKDGKAWDVKTSMDTVKKMMDS
NDVRVTKRLLEQTGALADATIYKATVAGKAKDGAYIGMKTKSSVFADVSKYGGMTKIKNAYS
IIVQYTGKKGEVIKEIVPLPIYLINRNTTDQDLINYVASIIPQAKDISIIYGKLCINQLVKV
NGFYYYLGGKTNSKFCIDNAIQVIVSNEWIPYLKVLEKENNMRKDNKDLKANVVSTRALDNK
HTIEVRIVEEKNIEFFDYLVSKLKMPIYQKMKGNKAAELSEKGYGLFKKMSLEEQSIHLIEL
LNLLTNQKTTFEVKPLGITASRSTVGSKISNQDEFKVINESITGLYSNEVTIVGKRPAATKK
AGQAKKKKGSYPYDVPDYAYPYDVPDYAYPYDVPDYA.

In some embodiments, the Ezakiella peruensis Cas9 has an amino acid sequence at least 80% identical to

(SEQ ID NO: 15)
MPKKKRKVGTKVKDYYIGLDIGTSSVGWAVTDEAYNVLKENSKKMWGVRLEDDAKTAEERRG
QRGARRRLDRKKERLSLLQDFFAEEVAKVDPNFFLRLDNSDLYMEDKDQKLKSKYTLENDKD
FKDKNFHKKYPTIHHLLMDLIEDDSKKDIRLVYLACHYLLKNRGHFIFEGQKFDTKSSFENS
LNELKVHLNDEYGLDLEFDNENLINILTDPKLNKTAKKKELKSVIGDTKFLKAVSAIMIGSS
QKLVDLFENPEDFDDSAIKSVDFSTTSFDDKYSDYELALGDKIALVNILKEIYDSSILENLL
KEADKSKDGNKYISNAFVKKYNKHGQDLKEFKRLVRQYHKSAYFDIFRSEKVNDNYVSYTKS
SISNNKRVKANKFTDQEAFYKFAKKHLETIKYKINKVNGSKADLELIDGMLRDMEFKNEMPK
IKSSDNGVIPYQLKLMELNKILENQSKHHEFLNVSDEYGSVCDKIASIMEFRIPYYVGPLNP
NSKYAWIKKQKDSEITPWNFKDVVDLDSSREEFIDSLIGRCTYLKDEKVLPKASLLYNEYMV
LNELNNLKLNDLPITEEMKKKIFDQLFKTRKKVTLKAVANLLKKEFNINGEILLSGTDGDFK
QGLNSYNDFKAIVGDKVDSDDYRDKIEEIIKLIVLYGDDKSYLQKKIKAGYGKYFTDSEIKK
MAGLNYKDWGRLSKKLLTGLEGANKITGERGSIIHFMREYNLNLMELMSASFTFTEEIQKLN
PVDDRKLSYEMVDELYLSPSVKRMLWQSLRIVDEIKNIMGTDSKKIFIEMARGKEEVKARKE
SRKNQLLKFYKDGKKAFISEIGEERYSYLLSEIEGEEENKFRWDNLYLYYTQLGRCMYSLEP
IDISELSSKNIYDQDHIYPKSKIYDDSIENRVLVKKDLNSKKGNSYPIPDEILNKNCYAYWK
ILYDKGLIGQKKYTRLTRRTGFTDDELVQFISRQIVETRQATKETANLLKTICKNSEIVYSK
AENASRFRQEFDIVKCRAVNDLHHMHDAYINIIVGNVYNTKFTKDPMNFVKKQEKARSYNLE
NMFKYDVKRGGYTAWIADDEKGTVKNASIKRIRKELEGTNYRFTRMNYIESGALENATLQRK
NKGSRPLKDKGPKSSIEKYGGYTNINKACFAVLDIKSKNKIERKLMPVEREIYAKQKNDKKL
SDEIFSKYLKDRFGIEDYRVVYPVVKMRTLLKIDGSYYFITGGSDKTLELRSALQLILPKKN
EWAIKQIDKSSENDYLTIERIQDLTEELVYNTEDIIVNKFKTSVFKKSFLNLFQDDKIENID
FKFKSMDFKEKCKTLLMLVKAIRASGVRQDLKSIDLKSDYGRLSSKINNIGNYQEFKIINQS
ITGLFENEVDLLKLGKRPAATKKAGQAKKKKGSYPYDVPDYAYPYDVPDYAYPYDVPDYA.

In some embodiments, the Lactobacillus fermentum strain AF15-40LB Cas9 has an amino acid sequence at least 80% identical to

(SEQ ID NO: 85)
MPKKKRKVGKEYHIGLDIGTSSIGWAVTDSQFKLMRIKGKTAIGVRLFEEGKTAAERRTFRT
TRRRLKRRKWRLHYLDEIFAPHLQEVDENFLRRLKQSNIHPEDPAKNQAFIGKLLFPDLLKK
NERGYPTLIKMRDELPVEQRAHYPVTNIYKLREAMINEDRQFDLREVYLAVHHIVKYRGHEL
NNASVDKFKVGRIDFDKSENVLNEAYEELQNGEGSFTIEPSKVEKIGQLLLDTKMRKLDRQK
AVAKLLEVKVADKEETKRNKQIATAMSKLVLGYKADFATVAMANGNEWKIDLSSETSEDEIE
KFREELSDAQNDILTEITSLESQIMLNEIVPNGMSISESMMDRYWTHERQLAEVKEYLATQP
ASARKEFDQVYNKYIGQAPKEKGFDLEKGLKKILSKKENWKEIDELLKAGDELPKQRTSANG
VIPHQMHQQELDRIIEKQAKYYPWLATENPATGERDRHQAKYELDQLVSFRIPYYVGPLVTP
EVQKATSGAKFAWAKRKEDGEITPWNLWDKIDRAESAEAFIKRMTVKDTYLLNEDVLPANSL
LYQKYNVLNELNNVRVNGRRLSVGIKQDIYTELFKKKKTVKAGDVASLVMAKTRGVNKPSVE
GLSDPKKFNSNLATYLDLKSIVGDKVDDNRYQMDLENIIEWRSVFEDGEIFADKLTEVEWLT
DEQRSALVKKRYKGWGRLSKKLLTGIVDENGQRIIDLMWNTDQNFMQIVNQPVFKEQIDQLN
QKAITNDGMTLRERVESVLDDAYTSPQNKKAIWQVVRVVEDIVKAVGNAPKSISIEFARNEG
NKGEITRSRRTQLQKLFEDQAHELVKDTSLTEELEKAPDLSDRYYFYFTQGGKDMYTGDPIN
FDEISTKYDIDHILPQSFVKDDSLDNRVLVSRAENNKKSDRVPAKLYAAKMKPYWNQLLKQG
LITQRKFENLTMDVDQTIKYRSLGFVKRQLVETRQVIKLTANILGSMYQEAGTDIIETRAGL
TKQLREEFDLPKVREVNDYHHAVDAYLTTFAGQYLNRRYPKLRSFFVYGEYMKFKHGSDLKL
RNFNFFHELMEGDKSQGKVVDQQTGELITTRDEVADYFDWVINLKVMLISNETYEETGKYFD
ASHESSSLYLKNQNKKSKLVVPLKNKLQPEYYGAYTGITQGYMVILKLLDKKGGFGVYRIPR
YAADILNKCHDEVAYRNKIAEIISSDPRAPKSFEVVVPRVLKGTFLVDGEEKFILSSYRYKV
NATQLILPVSDIKLIQDNFKALKKLNVEMQTKKLIEIYDNILRQVDKYYKLYDINKFRAKLH
DGRSKFVELDDFGQDASKEKVIIKILRGLHFGSDLQNLKEIGFGTTPLGQFQVSEAGIRLSN
TAFIIFKSPTGLFNRKLYLKNLGKRPAATKKAGQAKKKKGSYPYDVPDYAYPYDVPDYAYPY
DVPDYA.

In some embodiments, the Peptoniphilus sp. Marseille-P3761 Cas9 has an amino acid sequence at least 80% identical to

(SEQ ID NO: 87)
MPKKKRKVGEKKTNYTIGLDIGTDSVGWAVVKDDLELVKKRMKVLGNTETNYIKKNLWGSLL
FESGQTAKDRRLKRVARRRYERRRNRLTELQKIFAPAIDEVDENFFFRLNESFLVPEDKAFS
KNPIFGTLGEDKTYYKTYPTIYHLRQHLADSEEKADVRLIYLALAHMIKYRGHFLIEGKLDT
EHIAINENLEQFFESYNALFSEEPIELRKEELIAIENILREKNSRTVKEKRITSFLKDIGRA
NKQSPMMAFITLIVGKKAKFKAAFNLEEEISLNLTDDSYDENLEILLNTIGSDFADLEDHAQ
RVYNAVELAGILSGDVKNTHAKLSAQMVAMYERHKEQLKEYKSFIKANLPDQYDMTFVAPKD
AQKKDLKGYAGYIDGNMSQDSFYKFVKDQLKEVPGSEKFLDSIEKEDFLRKQRSFYNGVIPN
QVHLAEMEAILDRQENYYPWLKENREKIISLLTFRIPYYVGPLADGQSEFAWLERKSDEKIK
PWNFSDVVDLDRSAEKFIEQLIGRDTYLPDEYVLPKKSLIYQKYMVENELTKIAYLDERQKR
MNLSSVEKKEIFETLFKKRSKVTEKQLVKFFENYLQIDNPTIFGIEDAFNADYSTYVELAKV
PGMKSMMDDPDNEDLMEEIVKILTVFEDRKMRRKQLEKYKERLSPEQIKELAKKHYTGWGRL
SKKLLVGIRDKETQKTILDYLVEDDNHSGGRQHLNRNLMQLINDDRLSFKKTIAELQMIDPS
ADLYAQVQEIAGSPAIKKGILLGLKIVDEIIRVMGEKPENIVIEMARENQTTARGKALSKRR
EAKIKEGLAALGSSLLKENLPGNADLSQRKIYLYYTQNGKDIYLDEPLDFDRLSQYDEDHII
PQSFTVDNSLDNLVLTNSSQNRGNKKDDVPSLEVVNRQLAYWRSLKDAGLMTQRKFDNLTKA
MRGGLTDKDRERFIQRQLVETRQITKNVAKLLDMRLNDKKDEAGNKIRETNIVLLKSAMASE
FRKMFRLYKVRELNDYHHAHDAYLNAAIAINLLALYPYMADDEVYGEFRYKKKPQAEKATYE
KLRQWNLIKRFGEKQLFTPDHEDCWNKERDIKTIKKVMGYRQVNVVKKAEERTGMLFKETIN
GKTNKGSRIPIKKDLDPSKYGGYIEEKMAYYAVISYEDKKKKPGKTIVGISIMDKKEFEYDS
ISYLGKLGFSNPVVQIILKNYSLIAYPDGRRRYITGATKTTKGKVELQKANQIAMEQDLVNF
IYHLKNYDEISHPESYAFVQSHTDYFDRLEDSIEHYTRRELDAETNINRLRRIYEEEKKKDP
VDIEALVASFIELLKLTSAGAPADFIEMGEAISRRRYNSMTGLEDGQVIYQSLTGLYETRMR
FEDGKRPAATKKAGQAKKKKGSYPYDVPDYAYPYDVPDYAYPYDVPDYA.

In some embodiments, the amino acid sequence of the Cas9 protein comprises at least one, at least two, at least three, at least four, at least five, at least six, at least seven, at least eight, at least nine, or at least 10 mutations in SEQ ID NOs: 1, 4, 8, 14, 84 or 86.

In some embodiments, the mutation is an amino acid substitution.

In some embodiments, the Cas9 protein has nickase activity.

In some embodiments, provided herein is a Cas9 protein wherein the Cas9 protein comprises a nickase mutation at an amino acid positions corresponds to one or more amino acids 10, 12, 17, 762, 840, 854, 863, 982, 983, 984, 986, 987 of wild type SpCas9.

In some embodiments, the at least one mutation results in an inactive Cas9 (dCas9).

In some embodiments, the Cas9 protein comprises at least one amino acid mutation in PAM Interacting, HNH and/or RuvC domain.

In some embodiments, provided herein is a Cas9 protein, wherein the mutation at an amino acid position corresponds to amino acid 14 in the RuvC domain of SirCas9.

In some embodiments, provided herein is a Cas9 protein, wherein the mutation at an amino acid position corresponds to amino acid 12 in the RuvC domain of EpeCas9.

In some embodiments, provided herein is a Cas9 protein, wherein the mutation at an amino acid position corresponds to amino acid 9 in the RuvC domain of LfeCas9.

In some embodiments, provided herein is a Cas9 protein, wherein the mutation at an amino acid position corresponds to amino acid 12 in the RuvC domain of PmaCas9.

In some embodiments, the Cas9 protein further comprises a nuclear localization sequence (NLS) and/or a FLAG, HIS or HA tag.

In one aspect, provided herein is an engineered, non-naturally occurring Cas9 fusion protein comprising a Cas9 protein having at least 80% identity to SEQ ID NOs: 1, 4, 8, 14, 84 or 86 and wherein the Cas9 protein is fused to a histone demethylase, a transcriptional activator, or to a deaminase.

In some embodiments, provided herein is an engineered, non-naturally occurring Cas9 fusion protein further comprising a nuclear localization sequence (NLS) and/or a FLAG, HIS or HA tag.

In some embodiments, provided herein is an engineered, non-naturally occurring Cas9 fusion protein having at least 80% identity to SEQ ID NOs: 2, 5, 9, 15, 85, 87, 95 or 96.

In some embodiments, the Cas9 protein is fused to a cytosine deaminase or to an adenosine deaminase.

In some embodiments, the Cas9 protein is fused to an adenosine deaminase and has an amino acid sequence at least 80% identical to

(a)
(SEQ ID NO: 20)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGGKPYSIGLAIGTNSVGWAVVTDDYKVPAKKMKVLGNTDKQSIK
KNLLGALLFDSGETAEATRLKRTARRRYTRRKNRLRYLQEIFTGEMNKVDENFFQRLDDSEL
VDEDKRGEHHPIFGNIAAEVKYHDDFPTIYHLRRHLADTSKKADLRLVYLALAHMIKERGHF
LYEGDLKAENTDVQALFKDFVEEYDKTIEESHLSEITVDALSILTEKVSKSSRLENLIAHYP
TEKKNTLFGNLIALSLDLHPNFKTNFQLSEDAKLQFSKDTYEEDLEGELGEVGDEYADLFAS
AKNLYDAILLSGILTVDDNSTKAPLSASMVKRYEEHQKDLKKLKDFIKVNAPDQYNAIFKDK
NKKGYASYIESGVKQDEFYKYLKGILLKINGSGDFLDKIDREDFLRKQRTFDNGSIPHQIHL
QEMHAILRRQGEHYPFLKENQDKIEKILTFRIPYYVGPLARKGSRFAWAEYKADEKITPWNF
DDILDKEKSAEKFITRMTLNDLYLPEEKVLPKHSPLYEAFTVYNELTKVKYVNEQGEAKFFD
TNMKQEIFDHVFKENRKVTKDKLLNYLNKEFEEFRIVNLTGLDKENKAFNSSLGTYHDLRKI
LDKSFLDDKANEKTIEDIIQTLTLFEDREMIRQRLQKYSDIFTKAQLKKLERRHYTGWGRLS
YKLINGIRNKENKKTILDYLIDDGYANRNEMQLINDDALSFKEEIARAQIIDDVDDIANVVH
DLPGSPAIKKGILQSVKIVDELVKVMGHNPANIIIEMARENQTTDKGRRNSQQRLKLLQDSL
KNLDNPVNIKNVENQQLQNDRLFLYYIQNGKDMYTGETLDINNLSQYDIDHIIPQAFIKDNS
LDNRVLTRSDKNRGKSDDVPSIEVVHEMKSFWSKLLSVKLITQRKEDNLTKAERGGLTEEDK
AGFIKRQLVETRQITKHVAQILDERENTEFDGNKRRIRNVKIITLKSNLVSNFRKEFELYKV
REINDYHHAHDAYLNAVVGNALLLKYPQLEPEFVYGEYPKYNSYRSRKSATEKFLFYSNILR
FFKKEDIQTNEDGEIAWNKEKHIKILRKVLSYPQVNIVKKTEEQTGGFSKESILPKGESDKL
IPRKTKNSYWDPKKYGGFDSPVVAYSILVFADVEKGKSKKLRKVQDMVGITIMEKKRFEKNP
VDFLEQRGYRNVRLEKIIKLPKYSLFELENKRRRLLASAKELQKGNELVIPQRFTTLLYHSY
RIEKDYEPEHREYVEKHKDEFKELLEYISVFSRKYVLADNNLTKIEMLESKNKDAEVSSLAK
SFISLLTFTAFGAPAAFNFFGENIDRKRYTSVTECLNATLIHQSITGLYETRIDLSKLGEDG
KRPAATKKAGQAKKKKGSYPYDVPDYAYPYDVPDYAYPYDVPDYA;
(b)
(SEQ ID NO: 6)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVFNAQKKAQSSTDGSSGSETPG
TSESATPESSGPKKKRKVGAKNKDIRYSIGLAIGTNSVGWAVMDEHYELLKKGNHHMWGSRL
FDAAEPAATRRASRSIRRRYNKRRERIRLLRDLLGDMVMEVDPTFFIRLLNVSFLDEEDKQK
NLGNDYKDNYNLFIEKDFNDKTYYDKYPTIYHLRKELCENKEKADPRLIYLALHHIVKYRGN
FLKEGQSFAKVYEDIEEKLDNTLKKEMSLNDLDNLFVDNDINSMITVLSKIYQRSKKADDLL
KIMNPTKEERAAYKEFTKALVGLKFNVSKMILAQEVKKDDKDIELDESNVDYDSTVDGLQAE
LGEYIEFIEMLHSINSWVELQDILGNNSTISAAMVERYEEHKNDLRVLKKVIREELPDKYNE
VFREDNPKLHNYLGYIKYPKNTPVEEFYEYIKRLLAKVDTGEAREILERIDLEKFMLKQNSR
TNGSIPYQMQKDEMIQIIDNQSVYYPQLKENREKLISILEFRIPYYFGPLNTHSEFAWIKKE
EDKQKERILPWNYDQIVDIDATAEGFIERMQNTGTYFPDKPVMAKNSLTVSKFEVLNELNKI
RINGKLIPVETKKELLSDLFMKNKTITDKKLKDWLVTHQYYDTNEELKIEGYQKDLQFSTSL
APWIDFTKIFGEINASNYQLIEKIIYDISIFEDKKILKRRLKKVYQLDDLLVDKILKLNYTG
WSRLSEKLLTGIKSKNSKETILSILENSNMNLMEIINDESLGFKQIIEESNKKDIEGPFRYD
EVKKLAGSPAIKRGIWQALLVVQEITKFMKHEPSHIYIEFAREEQEKVRTESRIAKLQKIYK
DLNLQTKEDQLVYESLKKEDAKKKIDTDALYLYYLQMGKSMYSGKPLDIDKLSTYHIDHILP
RSLIKDDSLDNRVLVLPKENEWKLDSETVPFEIRNKMMGFWQKLHENGLMSNKKFFSLIRTD
FNEKDKKRFINRQLVETRQIIKNVAVIINDHYTNTNVVTVRAELSHQFRERYKIYKNRDLND
LHHAHDAYIACILGQFIHQNFGNMDVNMIYGQYKKNYKKDVQEHNNYGFILNSMNHIHENDD
NSVIWDPSYIGKIKSCFCYKDVYVTKKLEQNDAKLFDLTILPSDKNSENGVTKAKIPVNKYR
KDVNKYGGFSGDAPIMLAIEADKGKKHVRQVIAFPLRLKNYNDEERIKFIEKEKNLKNVKIL
TEVKKNQLILINHQYFFITGTNELVNATQLKLSAKNTKNLFNLVDANKHNKLESIDDANENE
VIQELICKLQEPIYSRYNSIGKEFEDSYEKINAVTKQDKLYIIEYLIAIMSAKATQGYIKPE
LAREIGTNGKNKGRIKSFTIDLNKTTFISTSVTGLFSKKYKLGKRPAATKKAGQAKKKKGSY
PYDVPDYAYPYDVPDYAYPYDVPDYA;
(c)
(SEQ ID NO: 10)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVFNAQKKAQSSTDGSSGSETPG
TSESATPESSGPKKKRKVGSIINFQRRGLMETQASNQLISSHLKGYPIKDYFVGLAIGTSSV
GWAVTNKAYELLKFRSHKMWGSRLFDEGESAVARRGFRSMRRRLERRKLRLKLLEELFADAM
AQVDPTFFMRLRESKYHYEDKTTGHSSKHILFIDKNYNDQDYFKEYPTVYHLRSELMKSGTD
DIRKLFLAVHHILKYRGNFLYEGATFDSNASTLDDVIKQALENITENCFDCNSAISSIGQIL
MEAGKTKSDKAKAIEHLVDTYIATDTVDTSSKTQKDQVKEDKKRLKAFANLVLGLNASLIDL
FGSVEELEEDLKKLQITGDTYDDKRDELAKAWSDEIYIIDDCKSVYDAIILLSIKEPGLTIS
ESKVKAFNKHKDDLAILKSLLKSDRSIYNTMFKVDEKGLHNYVHYIKQGRTEETSCNREDFY
KYTKKIVEGLSDSKDKEYILSQIELQILLPLQRIKDNGVIPYQLHLEELKAILAKCGPKFPF
LNEVADGFSVAEKLIKMLEFRIPYYVGPLNTHHNVDNGGFAWAVRKASGRVTPWNEDDKIDR
EKSAAAFIKNLTNKCTYLLGEDVLPKSSLLYSEFMLLNELNNVRIDGKPLEKVVKEHLIEAV
FKQDHKKMTKNRIEQFLKDNGYISETHKHEITGLDGEIKNDLASYRDMVRILGDGFDRSMAE
EIITDITIFGESKKMLRETLRKKFASCLDDEAIKKLTKLRYRDWGRLSQKLLNGIEGCDKAG
DGTPETIIILMRNFSYNLMELLGDKESFMERIQEINAKLTEGQIVNPHDIIDDLALSPAVKR
AVWQALRIVDEVAHIKKALPARIFVEVTRSNKNEKKKKDSRQKRLSDLYAAIKKDDVLLNGL
NNEIFGELKSSLAKYDDAALRSKKLYLYYTQMGRCAYTGEIIELSLLNTDNYDIDHIYPRSL
TKDDSFDNLVLCKRTANAQKSDAYPISEEIQKTQKPFWTFLKQQGLISERKYERLTRITPLT
ADDLSGFIARQLVETNQSVKAATTLLRRLYPGVDVVFVKAENVTDERHDNNFIKVRSLNHHH
HAKDAYLNIVVGNVYHERFTRNFRAFFKKNGANRTYNLAKMENYDVNCTNAKDGKAWDVKTS
MDTVKKMMDSNDVRVTKRLLEQTGALADATIYKATVAGKAKDGAYIGMKTKSSVFADVSKYG
GMTKIKNAYSIIVQYTGKKGEVIKEIVPLPIYLTNRNTTDQDLINYVASIIPQAKDISIIYG
KLCINQLVKVNGFYYYLGGKTNSKFCIDNAIQVIVSNEWIPYLKVLEKENNMRKDNKDLKAN
VVSTRALDNKHTIEVRIVEEKNIEFFDYLVSKLKMPIYQKMKGNKAAELSEKGYGLFKKMSL
EEQSIHLIELLNLLTNQKTTFEVKPLGITASRSTVGSKISNQDEFKVINESITGLYSNEVTI
VGKRPAATKKAGQAKKKKGSYPYDVPDYAYPYDVPDYAYPYDVPDYA;
(d)
(SEQ ID NO: 11)
MPKKKRKVSIINFQRRGLMETQASNQLISSHLKGYPIKDYFVGLAIGTSSVGWAVINKAYEL
LKFRSHKMWGSRLFDEGESAVARRGFRSMRRRLERRKLRLKLLEELFADAMAQVDPTFEMRL
RESKYHYEDKTTGHSSKHILFIDKNYNDQDYFKEYPTVYHLRSELMKSGTDDIRKLFLAVHH
ILKYRGNFLYEGATFDSNASTLDDVIKQALENITENCFDCNSAISSIGQILMEAGKTKSDKA
KAIEHLVDTYIATDTVDTSSKTQKDQVKEDKKRLKAFANLVLGLNASLIDLFGSVEELEEDL
KKLQITGDTYDDKRDELAKAWSDEIYIIDDCKSVYDAIILLSIKEPGLTISESKVKAFNKHK
DDLAILKSLLKSDRSIYNTMFKVDEKGLHNYVHYIKQGRTEETSCNREDFYKYTKKIVEGLS
DSKDKEYILSQIELQILLPLQRIKDNGVIPYQLHLEELKAILAKCGPKFPFLNEVADGESVA
EKLIKMLEFRIPYYVGPLNTHHNVDNGGFAWAVRKASGRVTPWNEDDKIDREKSAAAFIKNL
TNKCTYLLGEDVLPKSSLLYSEFMLLNELNNVRIDGKPLEKVVKEHLIEAVFKQDHKKMTKN
RIEQFLKDNGYISETHKHEITGLDGEIKNDLASYRDMVRILGDGEDRSMAEEIITDITIFGE
SKKMLRETLRKKFASCLDDEAIKKLTKLRYRDWGRLSQKLLNGIEGCDKAGDGTPETIIILM
RNFSYNLMELLGDKFSFMERIQEINAKLTEGQIVNPHDIIDDLALSPAVKRAVWQALRIVDE
VAHIKKALPARIFVEVTRSNKNEKKKKDSRQKRLSDLYAAIKKDDVLLNGLNNEIFGELKSS
LAKYDDAALRSKKLYLYYTQMGRCAYTGEIIELSLLNTDNYDIDHIYPRSLTKDDSFDNLVL
CKRTANAQKSDAYPISEEIQKTQKPFWTFLKQQGLISERKYERLTRITPLTADDLSGFIARQ
LVETNQSVKAATTLLRRLYPGVDVVFVKAENVTDFRHDNNFIKVRSLNHHHHAKDAYLNIVV
GNVYHERFTRNFRAFFKKNGANRTYNLAKMFNYDVNCTNAKDGKAWDVKTSMDTVKKMMDSN
DVRVTKRLLEQTGALADATIYKATVAGKAKDGAYIGMKTKSSVFADVSKYGGMTKIKNAYSI
IVQYTGKKGEVIKEIVPLPIYLTNRNTTDQDLINYVASIIPQAKDISIIYGKLCINQLVKVN
GFYYYLGGKTNSKFCIDNAIQVIVSNEWIPYLKVLEKENNMRKDNKDLKANVVSTRALDNKH
TIEVRIVEEKNIEFFDYLVSKLKMPIYQKMKGNKAAELSEKGYGLFKKMSLEEQSIHLIELL
NLLTNQKTTFEVKPLGITASRSTVGSKISNQDEFKVINESITGLYSNEVTIVKRPAATKKAG
QAKKKKSGSETPGTSESATPESSGSEVEFSHEYWMRHALTLAKRARDEREVPVGAVLVLNNR
VIGEGWNRAIGLHDPTAHAEIMALRQGGLVMQNYRLYDATLYVTFEPCVMCAGAMIHSRIGR
VVFGVRNAKTGAAGSLMDVLHHPGMNHRVEITEGILADECAALLCRFFRMPRRVENAQKKAQ
SSTDPAAKRVKLDGSYPYDVPDYAYPYDVPDYAYPYDVPDYA;
(e)
(SEQ ID NO: 16)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGTKVKDYYIGLAIGTSSVGWAVTDEAYNVLKENSKKMWGVRLED
DAKTAEERRGQRGARRRLDRKKERLSLLQDFFAEEVAKVDPNFFLRLDNSDLYMEDKDQKLK
SKYTLFNDKDFKDKNFHKKYPTIHHLLMDLIEDDSKKDIRLVYLACHYLLKNRGHFIFEGQK
FDTKSSFENSLNELKVHLNDEYGLDLEFDNENLINILTDPKLNKTAKKKELKSVIGDTKFLK
AVSAIMIGSSQKLVDLFENPEDFDDSAIKSVDESTTSFDDKYSDYELALGDKIALVNILKEI
YDSSILENLLKEADKSKDGNKYISNAFVKKYNKHGQDLKEFKRLVRQYHKSAYFDIFRSEKV
NDNYVSYTKSSISNNKRVKANKFTDQEAFYKFAKKHLETIKYKINKVNGSKADLELIDGMLR
DMEFKNFMPKIKSSDNGVIPYQLKLMELNKILENQSKHHEFLNVSDEYGSVCDKIASIMEFR
IPYYVGPLNPNSKYAWIKKQKDSEITPWNFKDVVDLDSSREEFIDSLIGRCTYLKDEKVLPK
ASLLYNEYMVLNELNNLKLNDLPITEEMKKKIFDQLFKTRKKVTLKAVANLLKKEFNINGEI
LLSGTDGDFKQGLNSYNDFKAIVGDKVDSDDYRDKIEEIIKLIVLYGDDKSYLQKKIKAGYG
KYFTDSEIKKMAGLNYKDWGRLSKKLLTGLEGANKITGERGSIIHFMREYNLNLMELMSASF
TFTEEIQKLNPVDDRKLSYEMVDELYLSPSVKRMLWQSLRIVDEIKNIMGTDSKKIFIEMAR
GKEEVKARKESRKNQLLKFYKDGKKAFISEIGEERYSYLLSEIEGEEENKERWDNLYLYYTQ
LGRCMYSLEPIDISELSSKNIYDQDHIYPKSKIYDDSIENRVLVKKDLNSKKGNSYPIPDEI
LNKNCYAYWKILYDKGLIGQKKYTRLTRRTGFTDDELVQFISRQIVETRQATKETANLLKTI
CKNSEIVYSKAENASRFRQEFDIVKCRAVNDLHHMHDAYINIIVGNVYNTKFTKDPMNFVKK
QEKARSYNLENMEKYDVKRGGYTAWIADDEKGTVKNASIKRIRKELEGTNYRFTRMNYIESG
ALFNATLQRKNKGSRPLKDKGPKSSIEKYGGYTNINKACFAVLDIKSKNKIERKLMPVEREI
YAKQKNDKKLSDEIFSKYLKDREGIEDYRVVYPVVKMRTLLKIDGSYYFITGGSDKTLELRS
ALQLILPKKNEWAIKQIDKSSENDYLTIERIQDLTEELVYNTFDIIVNKFKTSVFKKSFLNL
FQDDKIENIDFKFKSMDFKEKCKTLLMLVKAIRASGVRQDLKSIDLKSDYGRLSSKINNIGN
YQEFKIINQSITGLFENEVDLLKLGKRPAATKKAGQAKKKKGSYPYDVPDYAYPYDVPDYAY
PYDVPDYA;
(f)
(SEQ ID NO: 17)
MPKKKRKVTKVKDYYIGLAIGTSSVGWAVTDEAYNVLKFNSKKMWGVRLFDDAKTAEERRGQ
RGARRRLDRKKERLSLLQDFFAEEVAKVDPNFFLRLDNSDLYMEDKDQKLKSKYTLENDKDF
KDKNFHKKYPTIHHLLMDLIEDDSKKDIRLVYLACHYLLKNRGHFIFEGQKEDTKSSFENSL
NELKVHLNDEYGLDLEFDNENLINILTDPKLNKTAKKKELKSVIGDTKFLKAVSAIMIGSSQ
KLVDLFENPEDFDDSAIKSVDESTTSFDDKYSDYELALGDKIALVNILKEIYDSSILENLLK
EADKSKDGNKYISNAFVKKYNKHGQDLKEFKRLVRQYHKSAYFDIFRSEKVNDNYVSYTKSS
ISNNKRVKANKFTDQEAFYKFAKKHLETIKYKINKVNGSKADLELIDGMLRDMEFKNEMPKI
KSSDNGVIPYQLKLMELNKILENQSKHHEFLNVSDEYGSVCDKIASIMEFRIPYYVGPLNPN
SKYAWIKKQKDSEITPWNFKDVVDLDSSREEFIDSLIGRCTYLKDEKVLPKASLLYNEYMVL
NELNNLKLNDLPITEEMKKKIFDQLFKTRKKVTLKAVANLLKKEFNINGEILLSGTDGDFKQ
GLNSYNDFKAIVGDKVDSDDYRDKIEEIIKLIVLYGDDKSYLQKKIKAGYGKYFTDSEIKKM
AGLNYKDWGRLSKKLLTGLEGANKITGERGSIIHEMREYNLNLMELMSASFTFTEEIQKLNP
VDDRKLSYEMVDELYLSPSVKRMLWQSLRIVDEIKNIMGTDSKKIFIEMARGKEEVKARKES
RKNQLLKFYKDGKKAFISEIGEERYSYLLSEIEGEEENKFRWDNLYLYYTQLGRCMYSLEPI
DISELSSKNIYDQDHIYPKSKIYDDSIENRVLVKKDLNSKKGNSYPIPDEILNKNCYAYWKI
LYDKGLIGQKKYTRLTRRTGFTDDELVQFISRQIVETRQATKETANLLKTICKNSEIVYSKA
ENASRFRQEFDIVKCRAVNDLHHMHDAYINIIVGNVYNTKFTKDPMNFVKKQEKARSYNLEN
MFKYDVKRGGYTAWIADDEKGTVKNASIKRIRKELEGTNYRFTRMNYIESGALENATLQRKN
KGSRPLKDKGPKSSIEKYGGYTNINKACFAVLDIKSKNKIERKLMPVEREIYAKQKNDKKLS
DEIFSKYLKDRFGIEDYRVVYPVVKMRTLLKIDGSYYFITGGSDKTLELRSALQLILPKKNE
WAIKQIDKSSENDYLTIERIQDLTEELVYNTFDIIVNKFKTSVFKKSFLNLFQDDKIENIDE
KFKSMDFKEKCKTLLMLVKAIRASGVRQDLKSIDLKSDYGRLSSKINNIGNYQEFKIINQSI
TGLFENEVDLLKLKRPAATKKAGQAKKKKSGSETPGTSESATPESSGSEVEFSHEYWMRHAL
TLAKRARDEREVPVGAVLVLNNRVIGEGWNRAIGLHDPTAHAEIMALRQGGLVMQNYRLYDA
TLYVTFEPCVMCAGAMIHSRIGRVVFGVRNAKTGAAGSLMDVLHHPGMNHRVEITEGILADE
CAALLCRFFRMPRRVENAQKKAQSSTDPAAKRVKLDGSYPYDVPDYAYPYDVPDYAYPYDVP
DYA;
(g)
(SEQ ID NO: 88)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVFNAQKKAQSSTDGSSGSETPG
TSESATPESSGPKKKRKVGKEYHIGLAIGTSSIGWAVTDSQFKLMRIKGKTAIGVRLFEEGK
TAAERRTFRTTRRRLKRRKWRLHYLDEIFAPHLQEVDENFLRRLKQSNIHPEDPAKNQAFIG
KLLFPDLLKKNERGYPTLIKMRDELPVEQRAHYPVTNIYKLREAMINEDRQFDLREVYLAVH
HIVKYRGHFLNNASVDKFKVGRIDEDKSENVLNEAYEELQNGEGSFTIEPSKVEKIGQLLLD
TKMRKLDRQKAVAKLLEVKVADKEETKRNKQIATAMSKLVLGYKADFATVAMANGNEWKIDL
SSETSEDEIEKFREELSDAQNDILTEITSLESQIMLNEIVPNGMSISESMMDRYWTHERQLA
EVKEYLATQPASARKEFDQVYNKYIGQAPKEKGFDLEKGLKKILSKKENWKEIDELLKAGDF
LPKQRTSANGVIPHQMHQQELDRIIEKQAKYYPWLATENPATGERDRHQAKYELDQLVSFRI
PYYVGPLVTPEVQKATSGAKFAWAKRKEDGEITPWNLWDKIDRAESAEAFIKRMTVKDTYLL
NEDVLPANSLLYQKYNVLNELNNVRVNGRRLSVGIKQDIYTELFKKKKTVKAGDVASLVMAK
TRGVNKPSVEGLSDPKKENSNLATYLDLKSIVGDKVDDNRYQMDLENIIEWRSVFEDGEIFA
DKLTEVEWLTDEQRSALVKKRYKGWGRLSKKLLTGIVDENGQRIIDLMWNTDQNFMQIVNQP
VFKEQIDQLNQKAITNDGMTLRERVESVLDDAYTSPQNKKAIWQVVRVVEDIVKAVGNAPKS
ISIEFARNEGNKGEITRSRRTQLQKLFEDQAHELVKDTSLTEELEKAPDLSDRYYFYFTQGG
KDMYTGDPINFDEISTKYDIDHILPQSFVKDDSLDNRVLVSRAENNKKSDRVPAKLYAAKMK
PYWNQLLKQGLITQRKFENLTMDVDQTIKYRSLGFVKRQLVETRQVIKLTANILGSMYQEAG
TDIIETRAGLTKQLREEFDLPKVREVNDYHHAVDAYLTTFAGQYLNRRYPKLRSFFVYGEYM
KFKHGSDLKLRNFNFFHELMEGDKSQGKVVDQQTGELITTRDEVADYFDWVINLKVMLISNE
TYEETGKYFDASHESSSLYLKNQNKKSKLVVPLKNKLQPEYYGAYTGITQGYMVILKLLDKK
GGFGVYRIPRYAADILNKCHDEVAYRNKIAEIISSDPRAPKSFEVVVPRVLKGTFLVDGEEK
FILSSYRYKVNATQLILPVSDIKLIQDNFKALKKLNVEMQTKKLIEIYDNILRQVDKYYKLY
DINKFRAKLHDGRSKFVELDDFGQDASKEKVIIKILRGLHFGSDLQNLKEIGFGTTPLGQFQ
VSEAGIRLSNTAFIIFKSPTGLFNRKLYLKNLGKRPAATKKAGQAKKKKGSYPYDVPDYAYP
YDVPDYAYPYDVPDYA;
(h)
(SEQ ID NO: 89)
MPKKKRKVGKEYHIGLAIGTSSIGWAVTDSQFKLMRIKGKTAIGVRLFEEGKTAAERRTERT
TRRRLKRRKWRLHYLDEIFAPHLQEVDENFLRRLKQSNIHPEDPAKNQAFIGKLLFPDLLKK
NERGYPTLIKMRDELPVEQRAHYPVINIYKLREAMINEDRQFDLREVYLAVHHIVKYRGHEL
NNASVDKFKVGRIDFDKSFNVLNEAYEELQNGEGSFTIEPSKVEKIGQLLLDTKMRKLDRQK
AVAKLLEVKVADKEETKRNKQIATAMSKLVLGYKADFATVAMANGNEWKIDLSSETSEDEIE
KFREELSDAQNDILTEITSLFSQIMLNEIVPNGMSISESMMDRYWTHERQLAEVKEYLATQP
ASARKEFDQVYNKYIGQAPKEKGFDLEKGLKKILSKKENWKEIDELLKAGDFLPKQRTSANG
VIPHQMHQQELDRIIEKQAKYYPWLATENPATGERDRHQAKYELDQLVSFRIPYYVGPLVTP
EVQKATSGAKFAWAKRKEDGEITPWNLWDKIDRAESAEAFIKRMTVKDTYLLNEDVLPANSL
LYQKYNVLNELNNVRVNGRRLSVGIKQDIYTELFKKKKTVKAGDVASLVMAKTRGVNKPSVE
GLSDPKKENSNLATYLDLKSIVGDKVDDNRYQMDLENIIEWRSVFEDGEIFADKLTEVEWLT
DEQRSALVKKRYKGWGRLSKKLLTGIVDENGQRIIDLMWNTDQNFMQIVNQPVFKEQIDQLN
QKAITNDGMTLRERVESVLDDAYTSPQNKKAIWQVVRVVEDIVKAVGNAPKSISIEFARNEG
NKGEITRSRRTQLQKLFEDQAHELVKDTSLTEELEKAPDLSDRYYFYFTQGGKDMYTGDPIN
FDEISTKYDIDHILPQSFVKDDSLDNRVLVSRAENNKKSDRVPAKLYAAKMKPYWNQLLKQG
LITQRKFENLTMDVDQTIKYRSLGFVKRQLVETRQVIKLTANILGSMYQEAGTDIIETRAGL
TKQLREEFDLPKVREVNDYHHAVDAYLTTFAGQYLNRRYPKLRSFFVYGEYMKFKHGSDLKL
RNENFFHELMEGDKSQGKVVDQQTGELITTRDEVADYFDWVINLKVMLISNETYEETGKYFD
ASHESSSLYLKNQNKKSKLVVPLKNKLQPEYYGAYTGITQGYMVILKLLDKKGGFGVYRIPR
YAADILNKCHDEVAYRNKIAEIISSDPRAPKSFEVVVPRVLKGTFLVDGEEKFILSSYRYKV
NATQLILPVSDIKLIQDNFKALKKLNVEMQTKKLIEIYDNILRQVDKYYKLYDINKFRAKLH
DGRSKFVELDDFGQDASKEKVIIKILRGLHFGSDLQNLKEIGFGTTPLGQFQVSEAGIRLSN
TAFIIFKSPTGLFNRKLYLKNLKRPAATKKAGQAKKKKSGSETPGTSESATPESSGSEVEFS
HEYWMRHALTLAKRARDEREVPVGAVLVLNNRVIGEGWNRAIGLHDPTAHAEIMALRQGGLV
MQNYRLYDATLYVTFEPCVMCAGAMIHSRIGRVVFGVRNAKTGAAGSLMDVLHHPGMNHRVE
ITEGILADECAALLCRFFRMPRRVENAQKKAQSSTDPAAKRVKLDGSYPYDVPDYAYPYDVP
DYAYPYDVPDYA;
(i)
(SEQ ID NO: 91)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGEKKTNYTIGLAIGTDSVGWAVVKDDLELVKKRMKVLGNTETNY
IKKNLWGSLLFESGQTAKDRRLKRVARRRYERRRNRLTELQKIFAPAIDEVDENFFERLNES
FLVPEDKAFSKNPIFGTLGEDKTYYKTYPTIYHLRQHLADSEEKADVRLIYLALAHMIKYRG
HFLIEGKLDTEHIAINENLEQFFESYNALFSEEPIELRKEELIAIENILREKNSRTVKEKRI
TSFLKDIGRANKQSPMMAFITLIVGKKAKFKAAFNLEEEISLNLTDDSYDENLEILLNTIGS
DFADLFDHAQRVYNAVELAGILSGDVKNTHAKLSAQMVAMYERHKEQLKEYKSFIKANLPDQ
YDMTFVAPKDAQKKDLKGYAGYIDGNMSQDSFYKFVKDQLKEVPGSEKFLDSIEKEDELRKQ
RSFYNGVIPNQVHLAEMEAILDRQENYYPWLKENREKIISLLTFRIPYYVGPLADGQSEFAW
LERKSDEKIKPWNESDVVDLDRSAEKFIEQLIGRDTYLPDEYVLPKKSLIYQKYMVENELTK
IAYLDERQKRMNLSSVEKKEIFETLFKKRSKVTEKQLVKFFENYLQIDNPTIFGIEDAFNAD
YSTYVELAKVPGMKSMMDDPDNEDLMEEIVKILTVFEDRKMRRKQLEKYKERLSPEQIKELA
KKHYTGWGRLSKKLLVGIRDKETQKTILDYLVEDDNHSGGRQHLNRNLMQLINDDRLSFKKT
IAELQMIDPSADLYAQVQEIAGSPAIKKGILLGLKIVDEIIRVMGEKPENIVIEMARENQTT
ARGKALSKRREAKIKEGLAALGSSLLKENLPGNADLSQRKIYLYYTQNGKDIYLDEPLDEDR
LSQYDEDHIIPQSFTVDNSLDNLVLTNSSQNRGNKKDDVPSLEVVNRQLAYWRSLKDAGLMT
QRKFDNLTKAMRGGLTDKDRERFIQRQLVETRQITKNVAKLLDMRLNDKKDEAGNKIRETNI
VLLKSAMASEFRKMFRLYKVRELNDYHHAHDAYLNAAIAINLLALYPYMADDEVYGEFRYKK
KPQAEKATYEKLRQWNLIKRFGEKQLFTPDHEDCWNKERDIKTIKKVMGYRQVNVVKKAEER
TGMLFKETINGKTNKGSRIPIKKDLDPSKYGGYIEEKMAYYAVISYEDKKKKPGKTIVGISI
MDKKEFEYDSISYLGKLGESNPVVQIILKNYSLIAYPDGRRRYITGATKTTKGKVELQKANQ
IAMEQDLVNFIYHLKNYDEISHPESYAFVQSHTDYFDRLEDSIEHYTRRFLDAETNINRLRR
IYEEEKKKDPVDIEALVASFIELLKLTSAGAPADFIFMGEAISRRRYNSMTGLEDGQVIYQS
LTGLYETRMRFEDGKRPAATKKAGQAKKKKGSYPYDVPDYAYPYDVPDYAYPYDVPDYA;
(j)
(SEQ ID NO: 92)
MPKKKRKVEKKTNYTIGLAIGTDSVGWAVVKDDLELVKKRMKVLGNTETNYIKKNLWGSLLF
ESGQTAKDRRLKRVARRRYERRRNRLTELQKIFAPAIDEVDENFFERLNESFLVPEDKAFSK
NPIFGTLGEDKTYYKTYPTIYHLRQHLADSEEKADVRLIYLALAHMIKYRGHFLIEGKLDTE
HIAINENLEQFFESYNALFSEEPIELRKEELIAIENILREKNSRTVKEKRITSFLKDIGRAN
KQSPMMAFITLIVGKKAKFKAAFNLEEEISLNLTDDSYDENLEILLNTIGSDFADLEDHAQR
VYNAVELAGILSGDVKNTHAKLSAQMVAMYERHKEQLKEYKSFIKANLPDQYDMTFVAPKDA
QKKDLKGYAGYIDGNMSQDSFYKFVKDQLKEVPGSEKFLDSIEKEDFLRKQRSFYNGVIPNQ
VHLAEMEAILDRQENYYPWLKENREKIISLLTFRIPYYVGPLADGQSEFAWLERKSDEKIKP
WNFSDVVDLDRSAEKFIEQLIGRDTYLPDEYVLPKKSLIYQKYMVFNELTKIAYLDERQKRM
NLSSVEKKEIFETLFKKRSKVTEKQLVKFFENYLQIDNPTIFGIEDAFNADYSTYVELAKVP
GMKSMMDDPDNEDLMEEIVKILTVFEDRKMRRKQLEKYKERLSPEQIKELAKKHYTGWGRLS
KKLLVGIRDKETQKTILDYLVEDDNHSGGRQHLNRNLMQLINDDRLSFKKTIAELQMIDPSA
DLYAQVQEIAGSPAIKKGILLGLKIVDEIIRVMGEKPENIVIEMARENQTTARGKALSKRRE
AKIKEGLAALGSSLLKENLPGNADLSQRKIYLYYTQNGKDIYLDEPLDFDRLSQYDEDHIIP
QSFTVDNSLDNLVLTNSSQNRGNKKDDVPSLEVVNRQLAYWRSLKDAGLMTQRKFDNLTKAM
RGGLTDKDRERFIQRQLVETRQITKNVAKLLDMRLNDKKDEAGNKIRETNIVLLKSAMASEF
RKMFRLYKVRELNDYHHAHDAYLNAAIAINLLALYPYMADDEVYGEFRYKKKPQAEKATYEK
LRQWNLIKRFGEKQLFTPDHEDCWNKERDIKTIKKVMGYRQVNVVKKAEERTGMLFKETING
KTNKGSRIPIKKDLDPSKYGGYIEEKMAYYAVISYEDKKKKPGKTIVGISIMDKKEFEYDSI
SYLGKLGFSNPVVQIILKNYSLIAYPDGRRRYITGATKTTKGKVELQKANQIAMEQDLVNFI
YHLKNYDEISHPESYAFVQSHTDYFDRLEDSIEHYTRRFLDAETNINRLRRIYEEEKKKDPV
DIEALVASFIELLKLTSAGAPADFIFMGEAISRRRYNSMTGLEDGQVIYQSLTGLYETRMRF
EDKRPAATKKAGQAKKKKSGSETPGTSESATPESSGSEVEFSHEYWMRHALTLAKRARDERE
VPVGAVLVLNNRVIGEGWNRAIGLHDPTAHAEIMALRQGGLVMQNYRLYDATLYVTFEPCVM
CAGAMIHSRIGRVVFGVRNAKTGAAGSLMDVLHHPGMNHRVEITEGILADECAALLCRFFRM
PRRVFNAQKKAQSSTDPAAKRVKLDGSYPYDVPDYAYPYDVPDYAYPYDVPDYA.

In some embodiments, the Cas9 protein is fused to a cytosine deaminase and has an amino acid sequence at least 80% identical to

(a)
(SEQ ID NO: 21)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWELSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGGKPYSIGLAIGTNSVGWAVVTDD
YKVPAKKMKVLGNTDKQSIKKNLLGALLFDSGETAEATRLKRTARRRYTRRKNRLRYLQEIF
TGEMNKVDENFFQRLDDSFLVDEDKRGEHHPIFGNIAAEVKYHDDFPTIYHLRRHLADTSKK
ADLRLVYLALAHMIKFRGHELYEGDLKAENTDVQALFKDEVEEYDKTIEESHLSEITVDALS
ILTEKVSKSSRLENLIAHYPTEKKNTLFGNLIALSLDLHPNFKTNFQLSEDAKLQFSKDTYE
EDLEGFLGEVGDEYADLFASAKNLYDAILLSGILTVDDNSTKAPLSASMVKRYEEHQKDLKK
LKDFIKVNAPDQYNAIFKDKNKKGYASYIESGVKQDEFYKYLKGILLKINGSGDFLDKIDRE
DFLRKQRTFDNGSIPHQIHLQEMHAILRRQGEHYPFLKENQDKIEKILTFRIPYYVGPLARK
GSRFAWAEYKADEKITPWNEDDILDKEKSAEKFITRMTLNDLYLPEEKVLPKHSPLYEAFTV
YNELTKVKYVNEQGEAKFFDTNMKQEIFDHVFKENRKVTKDKLLNYLNKEFEEFRIVNLTGL
DKENKAFNSSLGTYHDLRKILDKSFLDDKANEKTIEDIIQTLTLFEDREMIRQRLQKYSDIF
TKAQLKKLERRHYTGWGRLSYKLINGIRNKENKKTILDYLIDDGYANRNFMQLINDDALSFK
EEIARAQIIDDVDDIANVVHDLPGSPAIKKGILQSVKIVDELVKVMGHNPANIIIEMARENQ
TTDKGRRNSQQRLKLLQDSLKNLDNPVNIKNVENQQLQNDRLFLYYIQNGKDMYTGETLDIN
NLSQYDIDHIIPQAFIKDNSLDNRVLTRSDKNRGKSDDVPSIEVVHEMKSFWSKLLSVKLIT
QRKFDNLTKAERGGLTEEDKAGFIKRQLVETRQITKHVAQILDERENTEFDGNKRRIRNVKI
ITLKSNLVSNFRKEFELYKVREINDYHHAHDAYLNAVVGNALLLKYPQLEPEFVYGEYPKYN
SYRSRKSATEKFLFYSNILRFFKKEDIQTNEDGEIAWNKEKHIKILRKVLSYPQVNIVKKTE
EQTGGFSKESILPKGESDKLIPRKTKNSYWDPKKYGGFDSPVVAYSILVFADVEKGKSKKLR
KVQDMVGITIMEKKRFEKNPVDFLEQRGYRNVRLEKIIKLPKYSLFELENKRRRLLASAKEL
QKGNELVIPQRFTTLLYHSYRIEKDYEPEHREYVEKHKDEFKELLEYISVESRKYVLADNNL
TKIEMLFSKNKDAEVSSLAKSFISLLTFTAFGAPAAFNFFGENIDRKRYTSVTECLNATLIH
QSITGLYETRIDLSKLGEDGKRPAATKKAGQAKKKKGSSGGSGGSGGSTNLSDIIEKETGKQ
LVIQESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVHTA
YDESTDENVMLLTSDAPEYKPWALVIQDSNGENKIKMLYPYDVPDYAYPYDVPDYAYPYDVP
DYA;
(b)
(SEQ ID NO: 12)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGSIINFQRRGLMETQASNQLISSH
LKGYPIKDYFVGLAIGTSSVGWAVTNKAYELLKFRSHKMWGSRLFDEGESAVARRGERSMRR
RLERRKLRLKLLEELFADAMAQVDPTFFMRLRESKYHYEDKTTGHSSKHILFIDKNYNDQDY
FKEYPTVYHLRSELMKSGTDDIRKLFLAVHHILKYRGNFLYEGATFDSNASTLDDVIKQALE
NITFNCFDCNSAISSIGQILMEAGKTKSDKAKAIEHLVDTYIATDTVDTSSKTQKDQVKEDK
KRLKAFANLVLGLNASLIDLFGSVEELEEDLKKLQITGDTYDDKRDELAKAWSDEIYIIDDC
KSVYDAIILLSIKEPGLTISESKVKAFNKHKDDLAILKSLLKSDRSIYNTMFKVDEKGLHNY
VHYIKQGRTEETSCNREDFYKYTKKIVEGLSDSKDKEYILSQIELQILLPLQRIKDNGVIPY
QLHLEELKAILAKCGPKFPFLNEVADGESVAEKLIKMLEFRIPYYVGPLNTHHNVDNGGFAW
AVRKASGRVTPWNEDDKIDREKSAAAFIKNLTNKCTYLLGEDVLPKSSLLYSEFMLLNELNN
VRIDGKPLEKVVKEHLIEAVFKQDHKKMTKNRIEQFLKDNGYISETHKHEITGLDGEIKNDL
ASYRDMVRILGDGEDRSMAEEIITDITIFGESKKMLRETLRKKFASCLDDEAIKKLTKLRYR
DWGRLSQKLLNGIEGCDKAGDGTPETIIILMRNFSYNLMELLGDKFSFMERIQEINAKLTEG
QIVNPHDIIDDLALSPAVKRAVWQALRIVDEVAHIKKALPARIFVEVTRSNKNEKKKKDSRQ
KRLSDLYAAIKKDDVLLNGLNNEIFGELKSSLAKYDDAALRSKKLYLYYTQMGRCAYTGEII
ELSLLNTDNYDIDHIYPRSLTKDDSFDNLVLCKRTANAQKSDAYPISEEIQKTQKPFWTFLK
QQGLISERKYERLTRITPLTADDLSGFIARQLVETNQSVKAATTLLRRLYPGVDVVFVKAEN
VTDFRHDNNFIKVRSLNHHHHAKDAYLNIVVGNVYHERFTRNFRAFFKKNGANRTYNLAKMF
NYDVNCTNAKDGKAWDVKTSMDTVKKMMDSNDVRVTKRLLEQTGALADATIYKATVAGKAKD
GAYIGMKTKSSVFADVSKYGGMTKIKNAYSIIVQYTGKKGEVIKEIVPLPIYLTNRNTTDQD
LINYVASIIPQAKDISIIYGKLCINQLVKVNGFYYYLGGKTNSKFCIDNAIQVIVSNEWIPY
LKVLEKFNNMRKDNKDLKANVVSTRALDNKHTIEVRIVEEKNIEFFDYLVSKLKMPIYQKMK
GNKAAELSEKGYGLFKKMSLEEQSIHLIELLNLLTNQKTTFEVKPLGITASRSTVGSKISNQ
DEFKVINESITGLYSNEVTIVGKRPAATKKAGQAKKKKGSSGGSGGSGGSTNLSDIIEKETG
KQLVIQESILMLPEEVEEVIGNKPESDILVHTAYDESTDENVMLLTSDAPEYKPWALVIQDS
NGENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLYPYDVPDYAYPYDVPDYAY;
(c)
(SEQ ID NO: 18)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGTKVKDYYIGLAIGTSSVGWAVTD
EAYNVLKFNSKKMWGVRLFDDAKTAEERRGQRGARRRLDRKKERLSLLQDFFAEEVAKVDPN
FFLRLDNSDLYMEDKDQKLKSKYTLFNDKDFKDKNFHKKYPTIHHLLMDLIEDDSKKDIRLV
YLACHYLLKNRGHFIFEGQKEDTKSSFENSLNELKVHLNDEYGLDLEFDNENLINILTDPKL
NKTAKKKELKSVIGDTKFLKAVSAIMIGSSQKLVDLFENPEDFDDSAIKSVDESTTSEDDKY
SDYELALGDKIALVNILKEIYDSSILENLLKEADKSKDGNKYISNAFVKKYNKHGQDLKEFK
RLVRQYHKSAYFDIFRSEKVNDNYVSYTKSSISNNKRVKANKFTDQEAFYKFAKKHLETIKY
KINKVNGSKADLELIDGMLRDMEFKNFMPKIKSSDNGVIPYQLKLMELNKILENQSKHHEFL
NVSDEYGSVCDKIASIMEFRIPYYVGPLNPNSKYAWIKKQKDSEITPWNFKDVVDLDSSREE
FIDSLIGRCTYLKDEKVLPKASLLYNEYMVLNELNNLKLNDLPITEEMKKKIFDQLFKTRKK
VTLKAVANLLKKEFNINGEILLSGTDGDFKQGLNSYNDFKAIVGDKVDSDDYRDKIEEIIKL
IVLYGDDKSYLQKKIKAGYGKYFTDSEIKKMAGLNYKDWGRLSKKLLTGLEGANKITGERGS
IIHFMREYNLNLMELMSASFTFTEEIQKLNPVDDRKLSYEMVDELYLSPSVKRMLWQSLRIV
DEIKNIMGTDSKKIFIEMARGKEEVKARKESRKNQLLKFYKDGKKAFISEIGEERYSYLLSE
IEGEEENKFRWDNLYLYYTQLGRCMYSLEPIDISELSSKNIYDQDHIYPKSKIYDDSIENRV
LVKKDLNSKKGNSYPIPDEILNKNCYAYWKILYDKGLIGQKKYTRLTRRTGFTDDELVQFIS
RQIVETRQATKETANLLKTICKNSEIVYSKAENASRFRQEFDIVKCRAVNDLHHMHDAYINI
IVGNVYNTKFTKDPMNFVKKQEKARSYNLENMFKYDVKRGGYTAWIADDEKGTVKNASIKRI
RKELEGTNYRFTRMNYIESGALFNATLQRKNKGSRPLKDKGPKSSIEKYGGYTNINKACFAV
LDIKSKNKIERKLMPVEREIYAKQKNDKKLSDEIFSKYLKDRFGIEDYRVVYPVVKMRTLLK
IDGSYYFITGGSDKTLELRSALQLILPKKNEWAIKQIDKSSENDYLTIERIQDLTEELVYNT
FDIIVNKFKTSVFKKSFLNLFQDDKIENIDFKFKSMDFKEKCKTLLMLVKAIRASGVRQDLK
SIDLKSDYGRLSSKINNIGNYQEFKIINQSITGLFENEVDLLKLGKRPAATKKAGQAKKKKG
SSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQES
ILMLPEEVEEVIGNKPESDILVHTAYDESTDENVMLLTSDAPEYKPWALVIQDSNGENKIKM
LYPYDVPDYAYPYDVPDYAY;
(d)
(SEQ ID NO: 90)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGKEYHIGLAIGTSSIGWAVTDSQF
KLMRIKGKTAIGVRLFEEGKTAAERRTFRTTRRRLKRRKWRLHYLDEIFAPHLQEVDENFLR
RLKQSNIHPEDPAKNQAFIGKLLFPDLLKKNERGYPTLIKMRDELPVEQRAHYPVTNIYKLR
EAMINEDRQFDLREVYLAVHHIVKYRGHFLNNASVDKFKVGRIDEDKSENVLNEAYEELQNG
EGSFTIEPSKVEKIGQLLLDTKMRKLDRQKAVAKLLEVKVADKEETKRNKQIATAMSKLVLG
YKADFATVAMANGNEWKIDLSSETSEDEIEKFREELSDAQNDILTEITSLESQIMLNEIVPN
GMSISESMMDRYWTHERQLAEVKEYLATQPASARKEFDQVYNKYIGQAPKEKGFDLEKGLKK
ILSKKENWKEIDELLKAGDFLPKQRTSANGVIPHQMHQQELDRIIEKQAKYYPWLATENPAT
GERDRHQAKYELDQLVSFRIPYYVGPLVTPEVQKATSGAKFAWAKRKEDGEITPWNLWDKID
RAESAEAFIKRMTVKDTYLLNEDVLPANSLLYQKYNVLNELNNVRVNGRRLSVGIKQDIYTE
LFKKKKTVKAGDVASLVMAKTRGVNKPSVEGLSDPKKENSNLATYLDLKSIVGDKVDDNRYQ
MDLENIIEWRSVFEDGEIFADKLTEVEWLTDEQRSALVKKRYKGWGRLSKKLLTGIVDENGQ
RIIDLMWNTDQNFMQIVNQPVFKEQIDQLNQKAITNDGMTLRERVESVLDDAYTSPQNKKAI
WQVVRVVEDIVKAVGNAPKSISIEFARNEGNKGEITRSRRTQLQKLFEDQAHELVKDTSLTE
ELEKAPDLSDRYYFYFTQGGKDMYTGDPINFDEISTKYDIDHILPQSFVKDDSLDNRVLVSR
AENNKKSDRVPAKLYAAKMKPYWNQLLKQGLITQRKFENLTMDVDQTIKYRSLGFVKRQLVE
TRQVIKLTANILGSMYQEAGTDIIETRAGLTKQLREEFDLPKVREVNDYHHAVDAYLTTFAG
QYLNRRYPKLRSFFVYGEYMKFKHGSDLKLRNFNFFHELMEGDKSQGKVVDQQTGELITTRD
EVADYFDWVINLKVMLISNETYEETGKYFDASHESSSLYLKNQNKKSKLVVPLKNKLQPEYY
GAYTGITQGYMVILKLLDKKGGFGVYRIPRYAADILNKCHDEVAYRNKIAEIISSDPRAPKS
FEVVVPRVLKGTFLVDGEEKFILSSYRYKVNATQLILPVSDIKLIQDNFKALKKLNVEMQTK
KLIEIYDNILRQVDKYYKLYDINKFRAKLHDGRSKFVELDDFGQDASKEKVIIKILRGLHFG
SDLQNLKEIGFGTTPLGQFQVSEAGIRLSNTAFIIFKSPTGLFNRKLYLKNLGKRPAATKKA
GQAKKKKGSSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDIIEKETG
KQLVIQESILMLPEEVEEVIGNKPESDILVHTAYDESTDENVMLLTSDAPEYKPWALVIQDS
NGENKIKMLYPYDVPDYAYPYDVPDYAY;
(e)
(SEQ ID NO: 93)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWELSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGEKKTNYTIGLAIGTDSVGWAVVK
DDLELVKKRMKVLGNTETNYIKKNLWGSLLFESGQTAKDRRLKRVARRRYERRRNRLTELQK
IFAPAIDEVDENFFFRLNESFLVPEDKAFSKNPIFGTLGEDKTYYKTYPTIYHLRQHLADSE
EKADVRLIYLALAHMIKYRGHFLIEGKLDTEHIAINENLEQFFESYNALFSEEPIELRKEEL
IAIENILREKNSRTVKEKRITSFLKDIGRANKQSPMMAFITLIVGKKAKFKAAFNLEEEISL
NLTDDSYDENLEILLNTIGSDFADLEDHAQRVYNAVELAGILSGDVKNTHAKLSAQMVAMYE
RHKEQLKEYKSFIKANLPDQYDMTFVAPKDAQKKDLKGYAGYIDGNMSQDSFYKFVKDQLKE
VPGSEKFLDSIEKEDFLRKQRSFYNGVIPNQVHLAEMEAILDRQENYYPWLKENREKIISLL
TFRIPYYVGPLADGQSEFAWLERKSDEKIKPWNFSDVVDLDRSAEKFIEQLIGRDTYLPDEY
VLPKKSLIYQKYMVFNELTKIAYLDERQKRMNLSSVEKKEIFETLFKKRSKVTEKQLVKFFE
NYLQIDNPTIFGIEDAFNADYSTYVELAKVPGMKSMMDDPDNEDLMEEIVKILTVFEDRKMR
RKQLEKYKERLSPEQIKELAKKHYTGWGRLSKKLLVGIRDKETQKTILDYLVEDDNHSGGRQ
HLNRNLMQLINDDRLSFKKTIAELQMIDPSADLYAQVQEIAGSPAIKKGILLGLKIVDEIIR
VMGEKPENIVIEMARENQTTARGKALSKRREAKIKEGLAALGSSLLKENLPGNADLSQRKIY
LYYTQNGKDIYLDEPLDFDRLSQYDEDHIIPQSFTVDNSLDNLVLTNSSQNRGNKKDDVPSL
EVVNRQLAYWRSLKDAGLMTQRKFDNLTKAMRGGLTDKDRERFIQRQLVETRQITKNVAKLL
DMRLNDKKDEAGNKIRETNIVLLKSAMASEFRKMFRLYKVRELNDYHHAHDAYLNAAIAINL
LALYPYMADDEVYGEFRYKKKPQAEKATYEKLRQWNLIKRFGEKQLFTPDHEDCWNKERDIK
TIKKVMGYRQVNVVKKAEERTGMLFKETINGKTNKGSRIPIKKDLDPSKYGGYIEEKMAYYA
VISYEDKKKKPGKTIVGISIMDKKEFEYDSISYLGKLGFSNPVVQIILKNYSLIAYPDGRRR
YITGATKTTKGKVELQKANQIAMEQDLVNFIYHLKNYDEISHPESYAFVQSHTDYFDRLEDS
IEHYTRRFLDAETNINRLRRIYEEEKKKDPVDIEALVASFIELLKLTSAGAPADFIFMGEAI
SRRRYNSMTGLFDGQVIYQSLTGLYETRMRFEDGKRPAATKKAGQAKKKKGSSGGSGGSGGS
TNLSDIIEKETGKQLVIQESILMLPEEVEEVIGNKPESDILVHTAYDESTDENVMLLTSDAP
EYKPWALVIQDSNGENKIKMLSGGSGGSGGSTNLSDIIEKETGKQLVIQESILMLPEEVEEV
IGNKPESDILVHTAYDESTDENVMLLTSDAPEYKPWALVIQDSNGENKIKMLYPYDVPDYAY
PYDVPDYAY;
or
(f)
(SEQ ID NO: 94)
MPAAKRVKLDTNLSDIIEKETGKQLVIQESILMLPEEVEEVIGNKPESDILVHTAYDESTDE
NVMLLTSDAPEYKPWALVIQDSNGENKIKMLSGGSGGSGGSTNLSDIIEKETGKQLVIQESI
LMLPEEVEEVIGNKPESDILVHTAYDESTDENVMLLTSDAPEYKPWALVIQDSNGENKIKML
SGGSGGSGGSPKKKRKVEKKTNYTIGLAIGTDSVGWAVVKDDLELVKKRMKVLGNTETNYIK
KNLWGSLLFESGQTAKDRRLKRVARRRYERRRNRLTELQKIFAPAIDEVDENFFERLNESFL
VPEDKAFSKNPIFGTLGEDKTYYKTYPTIYHLRQHLADSEEKADVRLIYLALAHMIKYRGHF
LIEGKLDTEHIAINENLEQFFESYNALFSEEPIELRKEELIAIENILREKNSRTVKEKRITS
FLKDIGRANKQSPMMAFITLIVGKKAKFKAAFNLEEEISLNLTDDSYDENLEILLNTIGSDE
ADLFDHAQRVYNAVELAGILSGDVKNTHAKLSAQMVAMYERHKEQLKEYKSFIKANLPDQYD
MTFVAPKDAQKKDLKGYAGYIDGNMSQDSFYKFVKDQLKEVPGSEKFLDSIEKEDELRKQRS
FYNGVIPNQVHLAEMEAILDRQENYYPWLKENREKIISLLTFRIPYYVGPLADGQSEFAWLE
RKSDEKIKPWNFSDVVDLDRSAEKFIEQLIGRDTYLPDEYVLPKKSLIYQKYMVENELTKIA
YLDERQKRMNLSSVEKKEIFETLFKKRSKVTEKQLVKFFENYLQIDNPTIFGIEDAFNADYS
TYVELAKVPGMKSMMDDPDNEDLMEEIVKILTVFEDRKMRRKQLEKYKERLSPEQIKELAKK
HYTGWGRLSKKLLVGIRDKETQKTILDYLVEDDNHSGGRQHLNRNLMQLINDDRLSFKKTIA
ELQMIDPSADLYAQVQEIAGSPAIKKGILLGLKIVDEIIRVMGEKPENIVIEMARENQTTAR
GKALSKRREAKIKEGLAALGSSLLKENLPGNADLSQRKIYLYYTQNGKDIYLDEPLDEDRLS
QYDEDHIIPQSFTVDNSLDNLVLTNSSQNRGNKKDDVPSLEVVNRQLAYWRSLKDAGLMTQR
KFDNLTKAMRGGLTDKDRERFIQRQLVETRQITKNVAKLLDMRLNDKKDEAGNKIRETNIVL
LKSAMASEFRKMFRLYKVRELNDYHHAHDAYLNAAIAINLLALYPYMADDEVYGEFRYKKKP
QAEKATYEKLRQWNLIKRFGEKQLFTPDHEDCWNKERDIKTIKKVMGYRQVNVVKKAEERTG
MLFKETINGKTNKGSRIPIKKDLDPSKYGGYIEEKMAYYAVISYEDKKKKPGKTIVGISIMD
KKEFEYDSISYLGKLGESNPVVQIILKNYSLIAYPDGRRRYITGATKTTKGKVELQKANQIA
MEQDLVNFIYHLKNYDEISHPESYAFVQSHTDYFDRLEDSIEHYTRRELDAETNINRLRRIY
EEEKKKDPVDIEALVASFIELLKLTSAGAPADFIFMGEAISRRRYNSMTGLEDGQVIYQSLT
GLYETRMRFEDKRPAATKKAGQAKKKKGSSGGSSGGSSGSETPGTSESATPESSGGSSGGST
SEKGPSTGDPTLRRRIESWEFDVFYDPRELRKETCLLYEIKWGMSRKIWRSSGKNTTNHVEV
NFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILS
LPPCLKISRRWQNHLAFFRLHLQNCHYQTIPPHILLATGLIHPSVTWRYPYDVPDYAYPYDV
PDYAYPYDVPDYA.

In some embodiments, the Streptococcus constellatus Cas9 protein recognizes a PAM sequence comprising 5′-NGG-3′.

In some embodiments, the Streptococcus constellatus Cas9 protein recognizes a PAM sequence comprising 5′-NGC-3′.

In some embodiments, a Cas9 protein disclosed herein (e.g., SirCas9, VapCas9, EpeCas9, LfeCas9, or PmaCas9) recognizes a PAM sequence comprising 5′-NGC-3′.

In some embodiments, the Sharpea Cas9 protein recognizes a PAM sequence comprising 5′-NAGHC-3′ wherein H=A, C or T.

In some embodiments, the Veillonella parvula Cas9 protein recognizes a PAM sequence comprising 5′-NRHRRH-3′, wherein H is adenine, cytosine or thymine, and R is adenine or guanine.

In some embodiments, the Ezakiella peruensis Cas9 protein recognizes a PAM sequence comprising 5′-NGG-3′.

In some embodiments, the Lactobacillus fermentum strain AF15-40LB Cas9 protein recognizes a PAM sequence comprising 5′-NGG-3′.

In some embodiments, the Peptoniphilus sp. Marseille-P3761 Cas9 protein recognizes a PAM sequence comprising 5′-NNAAA-3′

In some embodiments, a nucleic acid encoding the Cas9 protein is provided.

In some embodiments, the nucleic acid is codon-optimized for expression in mammalian cells.

In some embodiments, the nucleic acid is codon-optimized for expression in human cells.

In some embodiments, a eukaryotic cell comprising the Cas9 protein is provided.

In some embodiments, the cell is a human cell. In some embodiments, the cell is a plant cell.

In one aspect, a method of cleaving a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In one aspect, a method of modifying a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the Cas9 protein is an inactive Cas9 (dCas9).

In some embodiments, the dCas9 is fused to a deaminase.

In some embodiments, the RNA guide comprises a crRNA and a tracrRNA.

In some embodiments, the RNA guide comprises a sgRNA.

In some embodiments, the sgRNA for use with Streptococcus constellatus Cas9 comprises a scaffold comprising a sequence having at least about 80% identity to

(SEQ ID NO: 3)
5′-
GUUUUAGAGCUGUGCUGUUUAAACAACACAGCAAGUUAAAAUAAGGCUU
UGUCCGUACUCAAGCUUGCAAAAGCGUGCACCGAUUCGGUGCU-3′.

In some embodiments, the sgRNA for use with Sharpea Cas9 comprises a scaffold comprising a sequence having at least about 80% identity to

(SEQ ID NO: 7)
5′-
GUUUUAGAGUUGUGUUAUUGAAAAAUAACACAACGAGUUAAAAUAAAGC
UUAUGCUUAAAUGCCAGCUUUGCUGGUGUCAUUUAGAUGACUUUACUAA
GGUUGCUUCGGCAACCUUUUU-3′.

In some embodiments, the sgRNA for use with Veillonella parvula Cas9 comprises a scaffold comprising a sequence having at least about 80% identity to

(SEQ ID NO: 13)
5′-
GUUUGAGAGUAGUGUGAAAACAUUACGAGUUCAAAUACAAAUUAAUUUA
CAAUGCCUUCGGGCUGCCCGACGUAGGGCACCUACUCUCAAUUCUUCGG
AAUUGAGUU-3′.

In some embodiments, the sgRNA for use with Ezakiella peruensis Cas9 comprises a scaffold comprising a sequence having at least about 80% identity to

(SEQ ID NO: 19)
5′-
GUUUGAGAGUUAUGUAAUUGAAAAAUUACAUGACGAGUUCAAAUAAAAA
UUUAUUCAAACCGCCUAUUUAUAGGCCGCAGAUGUUCUGCAUUAUGCUU
GCUAUUGCAAGCUU-3′.

In some embodiments, the sgRNA for use with Lactobacillus fermentum strain AF15-40LB Cas9 comprises a scaffold comprising a sequence having at least about 80% identity to

(SEQ ID NO: 95)
5′-
GUCUUGGAUGAGUGUGAAAACACUCAUAGUCAAGAUCAAACGAGUGGUU
UUCCACGAGUUAUUACUUUUGAGGUCUUAUAUGGCCCAUACAUAAAAAG
GAGUCGGAAUUUCCGGCUCCUUUUCUU-3′.

In some embodiments, the sgRNA for use with Peptoniphilus sp. Marseille-P3761 Cas9 comprises a scaffold comprising a sequence having at least about 80% identity to

(SEQ ID NO: 96)
5′-
GUUUUAGAGCCAUGUAGAAAUACAUUGCAAGUUAAAAUAAGGCUUUGUC
CGUAAUCAACUUGAAAAAGUGGCGCUGUUUCGGCGCUUU-3′.

In some embodiments, the crRNA comprises a guide sequence of between about 16 and 26 nucleotides long.

In some embodiments, the crRNA comprises a guide sequence between 18 and 24 nucleotides long.

In some embodiments, the break in the target nucleic acid is a single-stranded or double-stranded break.

In some embodiments, the break in the target nucleic acid is a single-stranded break.

In some embodiments, the Cas9 protein is a nuclease that cleaves both strands of the target nucleic acid sequence. In some embodiments, the Cas9 is a nickase that cleaves one strand of the target nucleic acid sequence.

In some embodiments, the target nucleic acid is 5′ to a protospacer adjacent motif (PAM) sequence.

In some embodiments, the Cas9 is operably linked to a promoter sequence for expression in a eukaryotic cell, and wherein the guide RNA is operably linked to a promoter sequence for expression in a eukaryotic cell.

In some embodiments, the eukaryotic cell is a human cell.

In some embodiments, the promoter sequence is a eukaryotic or viral promoter.

In one aspect, provided herein is an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NOs: 1, 4, 8, 14, 84 or 86 and wherein the Cas protein is capable of binding to the RNA guide and of causing a break in the target nucleic acid sequence complementary to the RNA guide.

In some embodiments, provided herein is an engineered, non-naturally occurring CRISPR-Cas system comprising a codon-optimized CRISPR-associated (Cas) protein having at least 80% sequence identity to SEQ ID NOs: 2, 5, 9, 15, 85, 87, 95 or 96, and wherein the Cas protein is capable of binding to the RNA guide and of causing a break in the target nucleic acid sequence complementary to the RNA guide.

In one aspect, provided herein is an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NOs: 1, 4, 8, 14, 84 or 86; wherein the Cas protein is fused to a deaminase, and wherein the Cas protein fusion is capable of binding to the RNA guide and of editing the target nucleic acid sequence complementary to the RNA guide.

In some embodiments, the engineered, non-naturally occurring CRISPR-Cas system comprises a codon-optimized CRISPR-associated (Cas) protein further comprising a nuclear localization sequence (NLS) and/or a FLAG, HIS or HA tag.

In some embodiments, the engineered, non-naturally occurring CRISPR-Cas system comprises a codon-optimized CRISPR-associated (Cas) protein having at least 80% sequence identity to SEQ ID NOs: 2, 5, 9, 15, 85, 87, 95 or 96, wherein the Cas protein is fused to a deaminase, and wherein the Cas protein fusion is capable of binding to the RNA guide and of editing the target nucleic acid sequence complementary to the RNA guide.

In one embodiment, the Cas9 protein is an inactive Cas9 (dCas9).

In one embodiment, the RNA guide comprises a crRNA and a tracrRNA.

In one embodiment, the RNA guide comprises an sgRNA.

In one embodiment, the Cas protein is operably linked to a promoter sequence for expression in a eukaryotic cell, and wherein the guide RNA is operably linked to a promoter sequence for expression in a eukaryotic cell.

In one embodiment, the eukaryotic cell is a human cell.

In one embodiment, the promoter sequence is a eukaryotic promoter sequence.

In one embodiment, a nucleic acid encoding the system described herein is provided.

In one embodiment, a vector comprising the system described herein is provided.

In one embodiment, the vector is a plasmid vector or a viral vector.

In one embodiment, the viral vector is an adeno associated virus (AAV) vector or a lentiviral vector.

In one embodiment, the viral vector is an AAV vector.

In one embodiment, more than one AAV vector is used for packaging the system.

In one embodiment, a method of treating a disorder or a disease in a subject in need thereof comprises administering to the subject the system described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the guide RNA is complementary to about 18-24 nucleotides.

In some embodiments, the guide RNA is complementary to 20 nucleotides.

In some embodiments, the base editor comprises a fusion protein.

In some embodiments, the base editor comprises an adenosine deaminase domain or a cytidine deaminase domain.

In some embodiments, provided herein is a method of editing a nucleobase of a polynucleotide, the method comprising contacting the polynucleotide with a base in complex with one or more guide RNAs, wherein the base editor comprises an adenosine deaminase domain, and wherein the one or more guide RNAs target the base editor to effect an A•T to G•C alteration in the polynucleotide.

In some embodiments, provided herein is a method of editing a nucleobase of a polynucleotide, the method comprising contacting the polynucleotide with a base editor in complex with one or more guide RNAs, wherein the base editor comprises a cytidine deaminase domain, and wherein the one or more guide RNAs target the base editor to effect a C•G to T•A alteration in the polynucleotide.

In some embodiments, the editing results in less than 50% indel formation in the target polynucleotide sequence.

In some embodiments, the editing generates a point mutation.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C. In another embodiment, the base editing activity is cytosine or cytidine deaminase activity, e.g., converting target C•G to T•A and adenosine or adenine deaminase activity, e.g., converting A•T to G•C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or an RNA recognition motif.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity. In some embodiments, the Cas is a high-accuracy Cas. In some embodiments, the Cas is a high-fidelity Cas. In some embodiments, the Cas is a SuperFi-Cas. In some embodiments, the high-accuracy, high-fidelity and SuperFi-Cas are as described in Bravo, J. et al. Structural basis for mismatch surveillance by CRISPR-Cas9 Nature, 603, March 2022.

crRNA: The term “CRISPR RNA” or “crRNA,” as used herein, refers to a RNA molecule including a guide sequence used by a CRISPR effector to target a specific nucleic acid sequence. Typically, crRNAs contains a sequence that mediates target recognition and a sequence that forms a duplex with a tracrRNA. In some embodiments, the crRNA: tracrRNA duplex binds to a CRISPR effector.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence, and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Linker: The term “linker” refers to any means, entity or moiety used to join two or more entities. In some embodiments, the linker is a covalent linker. In some embodiments, the linker is a non-covalent linker. Examples of covalent linkers include covalent bonds or a linker moiety covalently attached to one Or more of the proteins or domains to be linked. In some embodiments, the linker is a non-covalent bond, e.g., an organometallic bond through a metal center such as platinum atom. The joining can be permanent or reversible. For covalent linkages, various functionalities can be used, such as amide groups, including carbonic acid derivatives, ethers, esters, including, organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. The PAM is required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1A is a graph that shows a consensus PAM motif recognized by human codon-optimized Streptococcus constellatus Cas9. FIG. 1B is a graph that shows a consensus PAM motif recognized by human codon-optimized Sharpea spp. isolate RUG017 Cas9. FIG. 1C is a graph that shows a consensus PAM motif recognized by human codon-optimized Veillonella parvula Cas9. FIG. 1D is a graph that shows a consensus PAM motif recognized by human codon-optimized Ezakiella peruensis. FIG. 1E is a graph that shows a consensus PAM motif recognized by human codon-optimized Lactobacillus fermentum strain AF15-40LB. FIG. 1F is a graph that shows a consensus PAM motif recognized by human codon-optimized Peptoniphilus sp. Marseille-P3761.

FIG. 2A is a schematic that shows predicted RNA folding structure of sgRNA for human codon-optimized Streptococcus constellatus ScoCas9 using Geneious software. FIG. 2A depicts sgRNA comprising SEQ ID NO: 3. FIG. 2B is a schematic that shows predicted RNA folding structure of sgRNA for human codon-optimized Sharpea spp. isolate RUG017 SirCas9 using Geneious software. FIG. 2B depicts sgRNA comprising SEQ ID NO: 7. FIG. 2C is a schematic that shows predicted RNA folding structure of sgRNA for human codon-optimized Veillonella parvula VapCas9 using Geneious software. FIG. 2C depicts sgRNA comprising SEQ ID NO: 13. FIG. 2D is a schematic that shows predicted RNA folding structure of sgRNA for human codon-optimized Ezakiella peruensis EpeCas9 using Geneious software. FIG. 2D depicts sgRNA comprising SEQ ID NO: 19. FIG. 2E is a schematic that shows predicted RNA folding structure of sgRNA for human codon-optimized Lactobacillus fermentum strain AF15-40LB LfeCas9 using Geneious software. FIG. 2D depicts sgRNA comprising SEQ ID NO: 95. FIG. 2F is a schematic that shows predicted RNA folding structure of sgRNA for human codon-optimized Peptoniphilus sp. Marseille-P3761 PmaCas9 using Geneious software. FIG. 2D depicts sgRNA comprising SEQ ID NO: 96.

FIG. 3 is a graph that shows exemplary results of ex vivo cleavage activity of human codon-optimized ScoCas9 in HEK293T cells. The y-axis of the graph shows indel frequency obtained using various guide RNAs that targeted A-rich genomic test sites adjacent to a sequence corresponding to the PAM consensus motif (see FIG. 1A).

FIG. 4A is a schematic showing constructs of ScoCas9 D10A mutant fused at the N-terminal to an adenine base editor (ABE) or a cytosine base editor (CBE). FIG. 4B is a graph that shows results of indel frequency and adenine to guanine base (A-to-G) conversion percentage achieved with a base editor comprising an ABE fused to the N-terminus of a ScoCas9 D10A mutant. The A-to-G conversion percentage (y-axis) is plotted for various guide RNAs targeting A-rich genomic test sites (x-axis; Table 8) adjacent to a sequence corresponding to the PAM consensus motif (see FIG. 1A). FIG. 4C is a graph that shows results of indel frequency and cytosine to thymine base (C-to-T) conversion percentage achieved with a base editor comprising an ABE fused to the N-terminus of a ScoCas9 D10A mutant. The C-to-T conversion percentage (y-axis) is plotted for various guide RNAs targeting C-rich genomic test sites (x-axis; Table 8) adjacent to a sequence corresponding to the PAM consensus motif (see FIG. 1A).

FIG. 5A is a schematic showing constructs of WT SirCas9 and a SirCas9 D14A mutant fused at the N-terminus to an adenine base editor (ABE). FIG. 5B is a graph that shows results of the indel frequency and A-to-G conversion achieved with a base editor comprising an ABE fused to the N-terminus of a SirCas9 D14A mutant. The A-to-G conversion percentage (y-axis) is plotted for various guide RNAs targeting A-rich genomic test sites (x-axis; Table 9) adjacent to a sequence corresponding to the PAM consensus motif (see FIG. 1B).

FIG. 6A is a schematic of constructs showing WT VapCas9 and VapCas9 D38A mutant fused at the N-terminus to an adenine base editor (ABE) or a cytosine base editor (CBE). FIG. 6B is a graph that shows results of the indel frequency, A-to-G conversion achieved with a base editor comprising an ABE fused to the N-terminus of a VapCas9 D38A mutant and C-to-T conversion achieved with a base editor comprising a CBE fused to the N-terminus of a VapCas9 D38A. The A-to-G conversion percentage (y-axis) is plotted for various guide RNAs targeting A-rich genomic test sites (x-axis; Table 10) adjacent to a sequence corresponding to the PAM consensus motif (see FIG. 1C). The C-to-T conversion percentage (y-axis) is plotted for various guide RNAs targeting C-rich genomic test sites (x-axis; Table 10) adjacent to a sequence corresponding to the PAM consensus motif (see FIG. 1C).

FIG. 7A is a schematic of constructs showing ABE fused to the N-terminus of VapCas9 or to the C-terminus of VapCas9. FIG. 7B is a graph that shows a comparison of A-to-G conversion achieved with a base editor comprising an ABE fused to the N-terminus and an ABE fused to the C-terminus of VapCas9. The A-to-G conversion percentage (y-axis) is plotted for various guide RNAs targeting A-rich genomic test sites (x-axis; Table 11) adjacent to a sequence corresponding to the PAM consensus motif (see FIG. 1C)

FIG. 8A is a schematic of constructs showing WT EpeCas9 and EpeCas9 D38A mutant fused at the N-terminus to an ABE and a CBE. FIG. 8B is a graph that shows results of the indel frequency, A-to-G conversion achieved with a base editor comprising an ABE fused to the N-terminus of an EpeCas9 D38A mutant and C-to-T conversion achieved with a base editor comprising a CBE fused to the N-terminus of a EpeCas9 D38A. The A-to-G conversion percentage (y-axis) is plotted for various guide RNAs targeting A-rich genomic test sites (x-axis; Table 12) adjacent to a sequence corresponding to the PAM consensus motif (see FIG. 1D). The C-to-T conversion percentage (y-axis) is plotted for various guide

RNAs targeting C-rich genomic test sites (x-axis; Table 12) adjacent to a sequence corresponding to the PAM consensus motif (see FIG. 1D).

FIG. 9A is a schematic that shows WT LfeCas9 and LfeCas9 D9A mutant fused at the N-terminus to an ABE and a CBE. FIG. 9B is a graph that shows results of the indel frequency with LfeCas9. FIG. 9C is a graph that shows results of A-to-G conversion achieved with a base editor comprising an ABE fused to the N-terminus of an LfeCas9 D9A mutant. The A-to-G conversion percentage (y-axis) is plotted for various guide RNAs targeting A-rich genomic test sites (x-axis; Table 13) adjacent to a sequence corresponding to the PAM consensus motif (see FIG. 1E). FIG. 9D is a graph that shows results of C-to-T conversion achieved with a base editor comprising a CBE fused to the N-terminus of an LfeCas9 D9A mutant. The C-to-T conversion percentage (y-axis) is plotted for various guide RNAs targeting C-rich genomic test sites (x-axis; Table 13) adjacent to a sequence corresponding to the PAM consensus motif (see FIG. 1E).

FIG. 10A is a schematic that shows WT PmaCas9 and PmaCas9 D12A mutant fused at the N-terminus and C-terminus to an ABE and a CBE. FIG. 10B is a graph that shows results of A-to-G or C-to-T conversion achieved with a base editor comprising an ABE or a CBE fused to the N-terminus or C-terminus of an PmaCas9 D12A mutant. The A-to-G conversion percentage (y-axis) is plotted for various guide RNAs targeting A-rich genomic test sites (x-axis; Table 14) adjacent to a sequence corresponding to the PAM consensus motif (see FIG. 1F). The C-to-T conversion percentage (y-axis) is plotted for various guide RNAs targeting C-rich genomic test sites (x-axis; Table 14) adjacent to a sequence corresponding to the PAM consensus motif (see FIG. 1F).

FIG. 11A is a graph that shows exemplary results of indel frequency (y-axis; % indel frequency) measured by transfecting cells with two ScoCas9-NGC variants, ScoCas9-NGC-v1 and ScoCas9-NGC-v2 (x-axis). An untransfected cell control is also shown.

FIG. 11B is a graph that shows exemplary A-to-G conversion (y-axis; % A to G conversion) in HEK293T cells transfected with A-to-G base editors (ABE) comprising ScoCas9-NGC variants, ScoCas9-NGC-v1 and ScoCas9-NGC-v2 (x-axis) engineered to recognize an NGC PAM motif. The ScoCas9-NGG variant which does not recognize NGC showed no A-to-G conversion. A SpyCas9-NGC control vector showed A-to-G editing. An untransfected cell control is also shown.

### DETAILED DESCRIPTION

Clustered regularly interspaced short palindromic repeats (CRISPR) was first discovered as an adaptive immune system in bacteria and archaea, and then engineered to generate targeted DNA breaks in living cells and organisms. During the cellular DNA repair process, various DNA changes can be introduced. The diverse and expanding CRISPR toolbox allows programmable genome editing, epigenome editing and transcriptome regulation.

CRISPR-Cas systems comprise three main types (I, II, and III) based on their Cas gene organization, and the sequence and structure of component proteins. Each of the three CRISPR systems is characterized by a unique Cas gene: Cas3, a target-degrading nuclease/helicase in Type I; Cas9, an RNA-binding and target-degrading nuclease in type II; Cas10, a large protein for multiple functions in type III. The three CRISPR types also differ in their associated effector complexes. Type I Cas systems associate with Cascade effector complexes, type II effector complexes consist of a single Cas9 and one or more RNA molecules, and type III interference complexes are further divided into type III-A (Csm complex targeting DNA) and type III-B (Cmr complex targeting RNA). Cas proteins are important components of effector complexes in all CRISPR-Cas systems.

Current genome editing technologies have focused on Class II CRISPR-Cas systems, which contain single-protein effector nucleases for DNA cleavage, specifically, Cas9, a dual-RNA-guided nuclease which requires both CRISPR RNA (crRNA) and tracrRNA and contains both HNH and RuvC nuclease domains, and Cas12a, a single-RNA-guided nuclease which only requires crRNA and contains a single RuvC domain.

Various aspects of the invention are described in detail in the following sections. The use of sections is not meant to limit the invention. Each section can apply to any aspect of the invention. In this application, the use of “or” means “and/or” unless stated otherwise.

### Engineered, Non-Naturally Occurring Cas9 Protein

Described herein are engineered, non-naturally occurring Cas9 proteins modified from WT Cas9 obtained from Streptococcus constellatus (ScoCas9), Sharpea spp. isolate RUG017 (SirCas9), Veillonella parvula (VapCas9 or VpaCas9, used interchangeably herein), Ezakiella peruensis (EpeCas9), Lactobacillus fermentum (LfeCas9) and Peptoniphilus sp. Marseille-P 3761 (PmaCas9) bacteria.

In some embodiments, the engineered non-naturally occurring Cas9 protein described herein comprises an amino acid sequence at least 60% (e.g., 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more) identical to SEQ ID NO: 1, 4, 8, 14, 84 or 86. In some embodiments, the Cas9 protein has is 80% identical to SEQ ID NO: 1, 4, 8, 14, 84 or 86. In some embodiments, the amino acid sequence of the Cas9 protein is identical to SEQ ID NO: 1, 4, 8, 14, 84 or 86. Exemplary Cas9 amino acid sequences are provided in Table 1 below.

TABLE 1
Exemplary Cas9 Amino Acid Sequences

Wild Type Streptococcus constellatus Cas9
MGKPYSIGLDIGTNSVGWAVVTDDYKVPAKKMKVLGNTDKQSIKKNLLGALLFDSGETAEA
TRLKRTARRRYTRRKNRLRYLQEIFTGEMNKVDENFFQRLDDSFLVDEDKRGEHHPIFGNI
AAEVKYHDDFPTIYHLRRHLADTSKKADLRLVYLALAHMIKFRGHFLYEGDLKAENTDVQA
LFKDFVEEYDKTIEESHLSEITVDALSILTEKVSKSSRLENLIAHYPTEKKNTLFGNLIAL
SLDLHPNFKTNFQLSEDAKLQFSKDTYEEDLEGFLGEVGDEYADLFASAKNLYDAILLSGI
LTVDDNSTKAPLSASMVKRYEEHQKDLKKLKDFIKVNAPDQYNAIFKDKNKKGYASYIESG
VKQDEFYKYLKGILLKINGSGDFLDKIDREDFLRKQRTFDNGSIPHQIHLQEMHAILRRQG
EHYPFLKENQDKIEKILTFRIPYYVGPLARKGSRFAWAEYKADEKITPWNEDDILDKEKSA
EKFITRMTLNDLYLPEEKVLPKHSPLYEAFTVYNELTKVKYVNEQGEAKFFDINMKQEIFD
HVFKENRKVTKDKLLNYLNKEFEEFRIVNLTGLDKENKAFNSSLGTYHDLRKILDKSFLDD
KANEKTIEDIIQTLTLFEDREMIRQRLQKYSDIFTKAQLKKLERRHYTGWGRLSYKLINGI
RNKENKKTILDYLIDDGYANRNFMQLINDDALSFKEEIARAQIIDDVDDIANVVHDLPGSP
AIKKGILQSVKIVDELVKVMGHNPANIIIEMARENQTTDKGRRNSQQRLKLLQDSLKNLDN
PVNIKNVENQQLQNDRLFLYYIQNGKDMYTGETLDINNLSQYDIDHIIPQAFIKDNSLDNR
VLTRSDKNRGKSDDVPSIEVVHEMKSFWSKLLSVKLITQRKEDNLTKAERGGLTEEDKAGE
IKRQLVETRQITKHVAQILDERENTEFDGNKRRIRNVKIITLKSNLVSNFRKEFELYKVRE
INDYHHAHDAYLNAVVGNALLLKYPQLEPEFVYGEYPKYNSYRSRKSATEKFLFYSNILRF
FKKEDIQTNEDGEIAWNKEKHIKILRKVLSYPQVNIVKKTEEQTGGFSKESILPKGESDKL
IPRKTKNSYWDPKKYGGFDSPVVAYSILVFADVEKGKSKKLRKVQDMVGITIMEKKRFEKN
PVDFLEQRGYRNVRLEKIIKLPKYSLFELENKRRRLLASAKELQKGNELVIPQRFTTLLYH
SYRIEKDYEPEHREYVEKHKDEFKELLEYISVFSRKYVLADNNLTKIEMLESKNKDAEVSS
LAKSFISLLTFTAFGAPAAFNFFGENIDRKRYTSVTECLNATLIHQSITGLYETRIDLSKL
GED (SEQ ID NO: 1).
Streptococcus constellatus Cas9 with Nuclear Localization Signal (NLS)
and Linker
MPKKKRKVGGKPYSIGLDIGTNSVGWAVVTDDYKVPAKKMKVLGNTDKQSIKKNLLGALLF
DSGETAEATRLKRTARRRYTRRKNRLRYLQEIFTGEMNKVDENFFQRLDDSELVDEDKRGE
HHPIFGNIAAEVKYHDDFPTIYHLRRHLADTSKKADLRLVYLALAHMIKFRGHFLYEGDLK
AENTDVQALFKDFVEEYDKTIEESHLSEITVDALSILTEKVSKSSRLENLIAHYPTEKKNT
LFGNLIALSLDLHPNFKTNFQLSEDAKLQFSKDTYEEDLEGELGEVGDEYADLFASAKNLY
DAILLSGILTVDDNSTKAPLSASMVKRYEEHQKDLKKLKDFIKVNAPDQYNAIFKDKNKKG
YASYIESGVKQDEFYKYLKGILLKINGSGDFLDKIDREDFLRKQRTFDNGSIPHQIHLQEM
HAILRRQGEHYPFLKENQDKIEKILTFRIPYYVGPLARKGSRFAWAEYKADEKITPWNEDD
ILDKEKSAEKFITRMTLNDLYLPEEKVLPKHSPLYEAFTVYNELTKVKYVNEQGEAKFEDT
NMKQEIFDHVFKENRKVTKDKLLNYLNKEFEEFRIVNLTGLDKENKAFNSSLGTYHDLRKI
LDKSFLDDKANEKTIEDIIQTLTLFEDREMIRQRLQKYSDIFTKAQLKKLERRHYTGWGRL
SYKLINGIRNKENKKTILDYLIDDGYANRNFMQLINDDALSFKEEIARAQIIDDVDDIANV
VHDLPGSPAIKKGILQSVKIVDELVKVMGHNPANIIIEMARENQTTDKGRRNSQQRLKLLQ
DSLKNLDNPVNIKNVENQQLQNDRLFLYYIQNGKDMYTGETLDINNLSQYDIDHIIPQAFI
KDNSLDNRVLTRSDKNRGKSDDVPSIEVVHEMKSFWSKLLSVKLITQRKFDNLTKAERGGL
TEEDKAGFIKRQLVETRQITKHVAQILDERENTEFDGNKRRIRNVKIITLKSNLVSNERKE
FELYKVREINDYHHAHDAYLNAVVGNALLLKYPQLEPEFVYGEYPKYNSYRSRKSATEKEL
FYSNILRFFKKEDIQTNEDGEIAWNKEKHIKILRKVLSYPQVNIVKKTEEQTGGFSKESIL
PKGESDKLIPRKTKNSYWDPKKYGGFDSPVVAYSILVFADVEKGKSKKLRKVQDMVGITIM
EKKRFEKNPVDFLEQRGYRNVRLEKIIKLPKYSLFELENKRRRLLASAKELQKGNELVIPQ
RFTTLLYHSYRIEKDYEPEHREYVEKHKDEFKELLEYISVFSRKYVLADNNLTKIEMLESK
NKDAEVSSLAKSFISLLTFTAFGAPAAFNFFGENIDRKRYTSVTECLNATLIHQSITGLYE
TRIDLSKLGEDGKRPAATKKAGQAKKKKGSYPYDVPDYAYPYDVPDYAYPYDVPDYA
(SEQ ID NO: 2).
Wild Type Sharpea Cas9
MAKNKDIRYSIGLDIGTNSVGWAVMDEHYELLKKGNHHMWGSRLFDAAEPAATRRASRSIR
RRYNKRRERIRLLRDLLGDMVMEVDPTFFIRLLNVSELDEEDKQKNLGNDYKDNYNLFIEK
DENDKTYYDKYPTIYHLRKELCENKEKADPRLIYLALHHIVKYRGNFLKEGQSFAKVYEDI
EEKLDNTLKKEMSLNDLDNLFVDNDINSMITVLSKIYQRSKKADDLLKIMNPTKEERAAYK
EFTKALVGLKFNVSKMILAQEVKKDDKDIELDESNVDYDSTVDGLQAELGEYIEFIEMLHS
INSWVELQDILGNNSTISAAMVERYEEHKNDLRVLKKVIREELPDKYNEVFREDNPKLHNY
LGYIKYPKNTPVEEFYEYIKRLLAKVDTGEAREILERIDLEKFMLKQNSRINGSIPYQMQK
DEMIQIIDNQSVYYPQLKENREKLISILEFRIPYYFGPLNTHSEFAWIKKFEDKQKERILP
WNYDQIVDIDATAEGFIERMQNTGTYFPDKPVMAKNSLTVSKFEVLNELNKIRINGKLIPV
ETKKELLSDLFMKNKTITDKKLKDWLVTHQYYDTNEELKIEGYQKDLQFSTSLAPWIDFTK
IFGEINASNYQLIEKIIYDISIFEDKKILKRRLKKVYQLDDLLVDKILKLNYTGWSRLSEK
LLTGIKSKNSKETILSILENSNMNLMEIINDESLGFKQIIEESNKKDIEGPFRYDEVKKLA
GSPAIKRGIWQALLVVQEITKFMKHEPSHIYIEFAREEQEKVRTESRIAKLQKIYKDLNLQ
TKEDQLVYESLKKEDAKKKIDTDALYLYYLQMGKSMYSGKPLDIDKLSTYHIDHILPRSLI
KDDSLDNRVLVLPKENEWKLDSETVPFEIRNKMMGFWQKLHENGLMSNKKFFSLIRTDENE
KDKKRFINRQLVETRQIIKNVAVIINDHYTNTNVVTVRAELSHQFRERYKIYKNRDLNDLH
HAHDAYIACILGQFIHQNFGNMDVNMIYGQYKKNYKKDVQEHNNYGFILNSMNHIHENDDN
SVIWDPSYIGKIKSCFCYKDVYVTKKLEQNDAKLFDLTILPSDKNSENGVTKAKIPVNKYR
KDVNKYGGFSGDAPIMLAIEADKGKKHVRQVIAFPLRLKNYNDEERIKFIEKEKNLKNVKI
LTEVKKNQLILINHQYFFITGTNELVNATQLKLSAKNTKNLFNLVDANKHNKLESIDDANF
NEVIQELICKLQEPIYSRYNSIGKEFEDSYEKINAVTKQDKLYIIEYLIAIMSAKATQGYI
KPELAREIGTNGKNKGRIKSFTIDLNKTT FISTSVTGLESKKYKL (SEQ ID NO: 4).
Sharpea Cas9 with Nuclear Localization Signal (NLS) and Linker
MPKKKRKVGAKNKDIRYSIGLDIGTNSVGWAVMDEHYELLKKGNHHMWGSRLFDAAEPAAT
RRASRSIRRRYNKRRERIRLLRDLLGDMVMEVDPTFFIRLLNVSFLDEEDKQKNLGNDYKD
NYNLFIEKDFNDKTYYDKYPTIYHLRKELCENKEKADPRLIYLALHHIVKYRGNELKEGQS
FAKVYEDIEEKLDNTLKKEMSLNDLDNLFVDNDINSMITVLSKIYQRSKKADDLLKIMNPT
KEERAAYKEFTKALVGLKFNVSKMILAQEVKKDDKDIELDFSNVDYDSTVDGLQAELGEYI
EFIEMLHSINSWVELQDILGNNSTISAAMVERYEEHKNDLRVLKKVIREELPDKYNEVFRE
DNPKLHNYLGYIKYPKNTPVEEFYEYIKRLLAKVDTGEAREILERIDLEKEMLKQNSRING
SIPYQMQKDEMIQIIDNQSVYYPQLKENREKLISILEFRIPYYFGPLNTHSEFAWIKKFED
KQKERILPWNYDQIVDIDATAEGFIERMQNTGTYFPDKPVMAKNSLTVSKFEVLNELNKIR
INGKLIPVETKKELLSDLFMKNKTITDKKLKDWLVTHQYYDTNEELKIEGYQKDLQFSTSL
APWIDFTKIFGEINASNYQLIEKIIYDISIFEDKKILKRRLKKVYQLDDLLVDKILKLNYT
GWSRLSEKLLTGIKSKNSKETILSILENSNMNLMEIINDESLGFKQIIEESNKKDIEGPER
YDEVKKLAGSPAIKRGIWQALLVVQEITKEMKHEPSHIYIEFAREEQEKVRTESRIAKLQK
IYKDLNLQTKEDQLVYESLKKEDAKKKIDTDALYLYYLQMGKSMYSGKPLDIDKLSTYHID
HILPRSLIKDDSLDNRVLVLPKENEWKLDSETVPFEIRNKMMGFWQKLHENGLMSNKKFFS
LIRTDENEKDKKRFINRQLVETRQIIKNVAVIINDHYTNTNVVTVRAELSHQFRERYKIYK
NRDLNDLHHAHDAYIACILGQFIHQNFGNMDVNMIYGQYKKNYKKDVQEHNNYGFILNSMN
HIHENDDNSVIWDPSYIGKIKSCFCYKDVYVTKKLEQNDAKLFDLTILPSDKNSENGVTKA
KIPVNKYRKDVNKYGGFSGDAPIMLAIEADKGKKHVRQVIAFPLRLKNYNDEERIKFIEKE
KNLKNVKILTEVKKNQLILINHQYFFITGTNELVNATQLKLSAKNTKNLENLVDANKHNKL
ESIDDANFNEVIQELICKLQEPIYSRYNSIGKEFEDSYEKINAVTKQDKLYIIEYLIAIMS
AKATQGYIKPELAREIGTNGKNKGRIKSFTIDLNKTTFISTSVTGLFSKKYKLGKRPAATK
KAGQAKKKKGSYPYDVPDYAYPYDVPDYAYPYDVPDYA (SEQ ID NO: 5).
Wild Type Veillonella parvula Cas9
MSIINFQRRGLMETQASNQLISSHLKGYPIKDYFVGLDIGTSSVGWAVINKAYELLKFRSH
KMWGSRLFDEGESAVARRGFRSMRRRLERRKLRLKLLEELFADAMAQVDPTFFMRLRESKY
HYEDKTTGHSSKHILFIDKNYNDQDYFKEYPTVYHLRSELMKSGTDDIRKLFLAVHHILKY
RGNFLYEGATFDSNASTLDDVIKQALENITENCFDCNSAISSIGQILMEAGKTKSDKAKAI
EHLVDTYIATDTVDTSSKTQKDQVKEDKKRLKAFANLVLGLNASLIDLFGSVEELEEDLKK
LQITGDTYDDKRDELAKAWSDEIYIIDDCKSVYDAIILLSIKEPGLTISESKVKAFNKHKD
DLAILKSLLKSDRSIYNTMFKVDEKGLHNYVHYIKQGRTEETSCNREDFYKYTKKIVEGLS
DSKDKEYILSQIELQILLPLQRIKDNGVIPYQLHLEELKAILAKCGPKFPFLNEVADGESV
AEKLIKMLEFRIPYYVGPLNTHHNVDNGGFAWAVRKASGRVTPWNEDDKIDREKSAAAFIK
NLTNKCTYLLGEDVLPKSSLLYSEFMLLNELNNVRIDGKPLEKVVKEHLIEAVFKQDHKKM
TKNRIEQFLKDNGYISETHKHEITGLDGEIKNDLASYRDMVRILGDGFDRSMAEEIITDIT
IFGESKKMLRETLRKKFASCLDDEAIKKLTKLRYRDWGRLSQKLLNGIEGCDKAGDGTPET
IIILMRNFSYNLMELLGDKESFMERIQEINAKLTEGQIVNPHDIIDDLALSPAVKRAVWQA
LRIVDEVAHIKKALPARIFVEVTRSNKNEKKKKDSRQKRLSDLYAAIKKDDVLLNGLNNEI
FGELKSSLAKYDDAALRSKKLYLYYTQMGRCAYTGEIIELSLLNTDNYDIDHIYPRSLTKD
DSFDNLVLCKRTANAQKSDAYPISEEIQKTQKPFWTFLKQQGLISERKYERLTRITPLTAD
DLSGFIARQLVETNQSVKAATTLLRRLYPGVDVVFVKAENVTDERHDNNFIKVRSLNHHHH
AKDAYLNIVVGNVYHERFTRNFRAFFKKNGANRTYNLAKMENYDVNCTNAKDGKAWDVKTS
MDTVKKMMDSNDVRVTKRLLEQTGALADATIYKATVAGKAKDGAYIGMKTKSSVFADVSKY
GGMTKIKNAYSIIVQYTGKKGEVIKEIVPLPIYLTNRNTTDQDLINYVASIIPQAKDISII
YGKLCINQLVKVNGFYYYLGGKTNSKFCIDNAIQVIVSNEWIPYLKVLEKENNMRKDNKDL
KANVVSTRALDNKHTIEVRIVEEKNIEFFDYLVSKLKMPIYQKMKGNKAAELSEKGYGLFK
KMSLEEQSIHLIELLNLLTNQKTTFEVKPLGITASRSTVGSKISNQDEFKVINESITGLYS
NEVTIV (SEQ ID NO: 8).
Veillonella parvula Cas9 with Nuclear Localization Signal (NLS) and
Linker
MPKKKRKVGSIINFQRRGLMETQASNQLISSHLKGYPIKDYFVGLDIGTSSVGWAVTNKAY
ELLKFRSHKMWGSRLFDEGESAVARRGFRSMRRRLERRKLRLKLLEELFADAMAQVDPTFF
MRLRESKYHYEDKTTGHSSKHILFIDKNYNDQDYFKEYPTVYHLRSELMKSGTDDIRKLFL
AVHHILKYRGNFLYEGATFDSNASTLDDVIKQALENITENCEDCNSAISSIGQILMEAGKT
KSDKAKAIEHLVDTYIATDTVDTSSKTQKDQVKEDKKRLKAFANLVLGLNASLIDLFGSVE
ELEEDLKKLQITGDTYDDKRDELAKAWSDEIYIIDDCKSVYDAIILLSIKEPGLTISESKV
KAFNKHKDDLAILKSLLKSDRSIYNTMFKVDEKGLHNYVHYIKQGRTEETSCNREDFYKYT
KKIVEGLSDSKDKEYILSQIELQILLPLQRIKDNGVIPYQLHLEELKAILAKCGPKFPELN
EVADGFSVAEKLIKMLEFRIPYYVGPLNTHHNVDNGGFAWAVRKASGRVTPWNEDDKIDRE
KSAAAFIKNLTNKCTYLLGEDVLPKSSLLYSEFMLLNELNNVRIDGKPLEKVVKEHLIEAV
FKQDHKKMTKNRIEQFLKDNGYISETHKHEITGLDGEIKNDLASYRDMVRILGDGEDRSMA
EEIITDITIFGESKKMLRETLRKKFASCLDDEAIKKLTKLRYRDWGRLSQKLLNGIEGCDK
AGDGTPETIIILMRNFSYNLMELLGDKESFMERIQEINAKLTEGQIVNPHDIIDDLALSPA
VKRAVWQALRIVDEVAHIKKALPARIFVEVTRSNKNEKKKKDSRQKRLSDLYAAIKKDDVL
LNGLNNEIFGELKSSLAKYDDAALRSKKLYLYYTQMGRCAYTGEIIELSLLNTDNYDIDHI
YPRSLTKDDSFDNLVLCKRTANAQKSDAYPISEEIQKTQKPFWTFLKQQGLISERKYERLT
RITPLTADDLSGFIARQLVETNQSVKAATTLLRRLYPGVDVVFVKAENVTDERHDNNFIKV
RSLNHHHHAKDAYLNIVVGNVYHERFTRNFRAFFKKNGANRTYNLAKMFNYDVNCTNAKDG
KAWDVKTSMDTVKKMMDSNDVRVTKRLLEQTGALADATIYKATVAGKAKDGAYIGMKTKSS
VFADVSKYGGMTKIKNAYSIIVQYTGKKGEVIKEIVPLPIYLTNRNTTDQDLINYVASIIP
QAKDISIIYGKLCINQLVKVNGFYYYLGGKTNSKFCIDNAIQVIVSNEWIPYLKVLEKENN
MRKDNKDLKANVVSTRALDNKHTIEVRIVEEKNIEFFDYLVSKLKMPIYQKMKGNKAAELS
EKGYGLFKKMSLEEQSIHLIELLNLLTNQKTTFEVKPLGITASRSTVGSKISNQDEFKVIN
ESITGLYSNEVTIVGKRPAATKKAGQAKKKKGSYPYDVPDYAYPYDVPDYAYPYDVPDYA
(SEQ ID NO: 9).
Wild Type Ezakiella peruensis Cas9
MTKVKDYYIGLDIGTSSVGWAVTDEAYNVLKENSKKMWGVRLEDDAKTAEERRGQRGARRR
LDRKKERLSLLQDFFAEEVAKVDPNFFLRLDNSDLYMEDKDQKLKSKYTLENDKDFKDKNF
HKKYPTIHHLLMDLIEDDSKKDIRLVYLACHYLLKNRGHFIFEGQKFDTKSSFENSLNELK
VHLNDEYGLDLEFDNENLINILTDPKLNKTAKKKELKSVIGDTKELKAVSAIMIGSSQKLV
DLFENPEDEDDSAIKSVDESTTSFDDKYSDYELALGDKIALVNILKEIYDSSILENLLKEA
DKSKDGNKYISNAFVKKYNKHGQDLKEFKRLVRQYHKSAYFDIFRSEKVNDNYVSYTKSSI
SNNKRVKANKFTDQEAFYKFAKKHLETIKYKINKVNGSKADLELIDGMLRDMEFKNEMPKI
KSSDNGVIPYQLKLMELNKILENQSKHHEFLNVSDEYGSVCDKIASIMEFRIPYYVGPLNP
NSKYAWIKKQKDSEITPWNFKDVVDLDSSREEFIDSLIGRCTYLKDEKVLPKASLLYNEYM
VLNELNNLKLNDLPITEEMKKKIFDQLFKTRKKVTLKAVANLLKKEFNINGEILLSGTDGD
FKQGLNSYNDFKAIVGDKVDSDDYRDKIEEIIKLIVLYGDDKSYLQKKIKAGYGKYFTDSE
IKKMAGLNYKDWGRLSKKLLTGLEGANKITGERGSIIHFMREYNLNLMELMSASFTFTEEI
QKLNPVDDRKLSYEMVDELYLSPSVKRMLWQSLRIVDEIKNIMGTDSKKIFIEMARGKEEV
KARKESRKNQLLKFYKDGKKAFISEIGEERYSYLLSEIEGEEENKFRWDNLYLYYTQLGRC
MYSLEPIDISELSSKNIYDQDHIYPKSKIYDDSIENRVLVKKDLNSKKGNSYPIPDEILNK
NCYAYWKILYDKGLIGQKKYTRLTRRTGFTDDELVQFISRQIVETRQATKETANLLKTICK
NSEIVYSKAENASRFRQEFDIVKCRAVNDLHHMHDAYINIIVGNVYNTKFTKDPMNFVKKQ
EKARSYNLENMFKYDVKRGGYTAWIADDEKGTVKNASIKRIRKELEGTNYRFTRMNYIESG
ALFNATLQRKNKGSRPLKDKGPKSSIEKYGGYTNINKACFAVLDIKSKNKIERKLMPVERE
IYAKQKNDKKLSDEIFSKYLKDRFGIEDYRVVYPVVKMRTLLKIDGSYYFITGGSDKTLEL
RSALQLILPKKNEWAIKQIDKSSENDYLTIERIQDLTEELVYNTFDIIVNKFKTSVFKKSF
LNLFQDDKIENIDFKFKSMDFKEKCKTLLMLVKAIRASGVRQDLKSIDLKSDYGRLSSKTN
NIGNYQEFKIINQSITGLFENEVDLLKL (SEQ ID NO: 14).
Ezakiella peruensis Cas9 with Nuclear Localization Signal (NLS) and
Linker
MPKKKRKVGTKVKDYYIGLDIGTSSVGWAVTDEAYNVLKFNSKKMWGVRLFDDAKTAEERR
GQRGARRRLDRKKERLSLLQDFFAEEVAKVDPNFFLRLDNSDLYMEDKDQKLKSKYTLEND
KDFKDKNFHKKYPTIHHLLMDLIEDDSKKDIRLVYLACHYLLKNRGHFIFEGQKFDTKSSF
ENSLNELKVHLNDEYGLDLEFDNENLINILTDPKLNKTAKKKELKSVIGDTKFLKAVSAIM
IGSSQKLVDLFENPEDFDDSAIKSVDESTTSFDDKYSDYELALGDKIALVNILKEIYDSSI
LENLLKEADKSKDGNKYISNAFVKKYNKHGQDLKEFKRLVRQYHKSAYFDIFRSEKVNDNY
VSYTKSSISNNKRVKANKFTDQEAFYKFAKKHLETIKYKINKVNGSKADLELIDGMLRDME
FKNFMPKIKSSDNGVIPYQLKLMELNKILENQSKHHEFLNVSDEYGSVCDKIASIMEFRIP
YYVGPLNPNSKYAWIKKQKDSEITPWNFKDVVDLDSSREEFIDSLIGRCTYLKDEKVLPKA
SLLYNEYMVLNELNNLKLNDLPITEEMKKKIFDQLFKTRKKVTLKAVANLLKKEFNINGEI
LLSGTDGDFKQGLNSYNDFKAIVGDKVDSDDYRDKIEEIIKLIVLYGDDKSYLQKKIKAGY
GKYFTDSEIKKMAGLNYKDWGRLSKKLLTGLEGANKITGERGSIIHFMREYNLNLMELMSA
SFTFTEEIQKLNPVDDRKLSYEMVDELYLSPSVKRMLWQSLRIVDEIKNIMGTDSKKIFIE
MARGKEEVKARKESRKNQLLKFYKDGKKAFISEIGEERYSYLLSEIEGEEENKFRWDNLYL
YYTQLGRCMYSLEPIDISELSSKNIYDQDHIYPKSKIYDDSIENRVLVKKDLNSKKGNSYP
IPDEILNKNCYAYWKILYDKGLIGQKKYTRLTRRTGFTDDELVQFISRQIVETRQATKETA
NLLKTICKNSEIVYSKAENASRFRQEFDIVKCRAVNDLHHMHDAYINIIVGNVYNTKFTKD
PMNFVKKQEKARSYNLENMFKYDVKRGGYTAWIADDEKGTVKNASIKRIRKELEGTNYRFT
RMNYIESGALFNATLQRKNKGSRPLKDKGPKSSIEKYGGYTNINKACFAVLDIKSKNKIER
KLMPVEREIYAKQKNDKKLSDEIFSKYLKDREGIEDYRVVYPVVKMRTLLKIDGSYYFITG
GSDKTLELRSALQLILPKKNEWAIKQIDKSSENDYLTIERIQDLTEELVYNTFDIIVNKFK
TSVFKKSFLNLFQDDKIENIDFKEKSMDFKEKCKTLLMLVKAIRASGVRQDLKSIDLKSDY
GRLSSKINNIGNYQEFKIINQSITGLFENEVDLLKLGKRPAATKKAGQAKKKKGSYPYDVP
DYAYPYDVPDYAYPYDVPDYA (SEQ ID NO: 15).
Wild Type Lactobacillus fermentum strain AF15-40LB Cas9
MKEYHIGLDIGTSSIGWAVTDSQFKLMRIKGKTAIGVRLFEEGKTAAERRTFRTTRRRLKR
RKWRLHYLDEIFAPHLQEVDENFLRRLKQSNIHPEDPAKNQAFIGKLLFPDLLKKNERGYP
TLIKMRDELPVEQRAHYPVTNIYKLREAMINEDRQFDLREVYLAVHHIVKYRGHFLNNASV
DKFKVGRIDFDKSFNVLNEAYEELQNGEGSFTIEPSKVEKIGQLLLDTKMRKLDRQKAVAK
LLEVKVADKEETKRNKQIATAMSKLVLGYKADFATVAMANGNEWKIDLSSETSEDEIEKER
EELSDAQNDILTEITSLESQIMLNEIVPNGMSISESMMDRYWTHERQLAEVKEYLATQPAS
ARKEFDQVYNKYIGQAPKEKGFDLEKGLKKILSKKENWKEIDELLKAGDFLPKQRTSANGV
IPHQMHQQELDRIIEKQAKYYPWLATENPATGERDRHQAKYELDQLVSFRIPYYVGPLVTP
EVQKATSGAKFAWAKRKEDGEITPWNLWDKIDRAESAEAFIKRMTVKDTYLLNEDVLPANS
LLYQKYNVLNELNNVRVNGRRLSVGIKQDIYTELFKKKKTVKAGDVASLVMAKTRGVNKPS
VEGLSDPKKENSNLATYLDLKSIVGDKVDDNRYQMDLENIIEWRSVFEDGEIFADKLTEVE
WLTDEQRSALVKKRYKGWGRLSKKLLTGIVDENGQRIIDLMWNTDQNFMQIVNQPVFKEQI
DQLNQKAITNDGMTLRERVESVLDDAYTSPQNKKAIWQVVRVVEDIVKAVGNAPKSISIEF
ARNEGNKGEITRSRRTQLQKLFEDQAHELVKDTSLTEELEKAPDLSDRYYFYFTQGGKDMY
TGDPINFDEISTKYDIDHILPQSFVKDDSLDNRVLVSRAENNKKSDRVPAKLYAAKMKPYW
NQLLKQGLITQRKFENLTMDVDQTIKYRSLGFVKRQLVETRQVIKLTANILGSMYQEAGTD
IIETRAGLTKQLREEFDLPKVREVNDYHHAVDAYLTTFAGQYLNRRYPKLRSFFVYGEYMK
FKHGSDLKLRNFNFFHELMEGDKSQGKVVDQQTGELITTRDEVADYFDWVINLKVMLISNE
TYEETGKYFDASHESSSLYLKNQNKKSKLVVPLKNKLQPEYYGAYTGITQGYMVILKLLDK
KGGFGVYRIPRYAADILNKCHDEVAYRNKIAEIISSDPRAPKSFEVVVPRVLKGTFLVDGE
EKFILSSYRYKVNATQLILPVSDIKLIQDNFKALKKLNVEMQTKKLIEIYDNILRQVDKYY
KLYDINKFRAKLHDGRSKFVELDDFGQDASKEKVIIKILRGLHFGSDLQNLKEIGFGTTPL
GQFQVSEAGIRLSNTAFIIFKSPTGLENRKLYLKNL (SEQ ID NO: 84).
Lactobacillus fermentum strain AF15-40LB Cas9 with Nuclear Localization
Signal (NLS) and Linker
MPKKKRKVGKEYHIGLDIGTSSIGWAVTDSQFKLMRIKGKTAIGVRLFEEGKTAAERRTER
TTRRRLKRRKWRLHYLDEIFAPHLQEVDENFLRRLKQSNIHPEDPAKNQAFIGKLLFPDLL
KKNERGYPTLIKMRDELPVEQRAHYPVINIYKLREAMINEDRQFDLREVYLAVHHIVKYRG
HFLNNASVDKFKVGRIDFDKSFNVLNEAYEELQNGEGSFTIEPSKVEKIGQLLLDTKMRKL
DRQKAVAKLLEVKVADKEETKRNKQIATAMSKLVLGYKADFATVAMANGNEWKIDLSSETS
EDEIEKFREELSDAQNDILTEITSLESQIMLNEIVPNGMSISESMMDRYWTHERQLAEVKE
YLATQPASARKEFDQVYNKYIGQAPKEKGFDLEKGLKKILSKKENWKEIDELLKAGDELPK
QRTSANGVIPHQMHQQELDRIIEKQAKYYPWLATENPATGERDRHQAKYELDQLVSFRIPY
YVGPLVTPEVQKATSGAKFAWAKRKEDGEITPWNLWDKIDRAESAEAFIKRMTVKDTYLLN
EDVLPANSLLYQKYNVLNELNNVRVNGRRLSVGIKQDIYTELFKKKKTVKAGDVASLVMAK
TRGVNKPSVEGLSDPKKFNSNLATYLDLKSIVGDKVDDNRYQMDLENIIEWRSVFEDGEIF
ADKLTEVEWLTDEQRSALVKKRYKGWGRLSKKLLTGIVDENGQRIIDLMWNTDQNFMQIVN
QPVFKEQIDQLNQKAITNDGMTLRERVESVLDDAYTSPQNKKAIWQVVRVVEDIVKAVGNA
PKSISIEFARNEGNKGEITRSRRTQLQKLFEDQAHELVKDTSLTEELEKAPDLSDRYYFYF
TQGGKDMYTGDPINFDEISTKYDIDHILPQSFVKDDSLDNRVLVSRAENNKKSDRVPAKLY
AAKMKPYWNQLLKQGLITQRKFENLTMDVDQTIKYRSLGFVKRQLVETRQVIKLTANILGS
MYQEAGTDIIETRAGLTKQLREEFDLPKVREVNDYHHAVDAYLTTFAGQYLNRRYPKLRSF
FVYGEYMKFKHGSDLKLRNFNFFHELMEGDKSQGKVVDQQTGELITTRDEVADYEDWVINL
KVMLISNETYEETGKYFDASHESSSLYLKNQNKKSKLVVPLKNKLQPEYYGAYTGITQGYM
VILKLLDKKGGFGVYRIPRYAADILNKCHDEVAYRNKIAEIISSDPRAPKSFEVVVPRVLK
GTFLVDGEEKFILSSYRYKVNATQLILPVSDIKLIQDNFKALKKLNVEMQTKKLIEIYDNI
LRQVDKYYKLYDINKFRAKLHDGRSKEVELDDFGQDASKEKVIIKILRGLHFGSDLQNLKE
IGFGTTPLGQFQVSEAGIRLSNTAFIIFKSPTGLFNRKLYLKNL
GKRPAATKKAGQAKKKKGSYPYDVPDYAYPYDVPDYAYPYDVPDYA (SEQ ID NO: 85).
Wild Type Peptoniphilus sp. Marseille-P3761 Cas9
MEKKTNYTIGLDIGTDSVGWAVVKDDLELVKKRMKVLGNTETNYIKKNLWGSLLFESGQTA
KDRRLKRVARRRYERRRNRLTELQKIFAPAIDEVDENFFERLNESFLVPEDKAFSKNPIFG
TLGEDKTYYKTYPTIYHLRQHLADSEEKADVRLIYLALAHMIKYRGHFLIEGKLDTEHIAI
NENLEQFFESYNALFSEEPIELRKEELIAIENILREKNSRTVKEKRITSFLKDIGRANKQS
PMMAFITLIVGKKAKFKAAFNLEEEISLNLTDDSYDENLEILLNTIGSDFADLFDHAQRVY
NAVELAGILSGDVKNTHAKLSAQMVAMYERHKEQLKEYKSFIKANLPDQYDMTFVAPKDAQ
KKDLKGYAGYIDGNMSQDSFYKFVKDQLKEVPGSEKFLDSIEKEDFLRKQRSFYNGVIPNQ
VHLAEMEAILDRQENYYPWLKENREKIISLLTFRIPYYVGPLADGQSEFAWLERKSDEKIK
PWNFSDVVDLDRSAEKFIEQLIGRDTYLPDEYVLPKKSLIYQKYMVFNELTKIAYLDERQK
RMNLSSVEKKEIFETLFKKRSKVTEKQLVKFFENYLQIDNPTIFGIEDAFNADYSTYVELA
KVPGMKSMMDDPDNEDLMEEIVKILTVFEDRKMRRKQLEKYKERLSPEQIKELAKKHYTGW
GRLSKKLLVGIRDKETQKTILDYLVEDDNHSGGRQHLNRNLMQLINDDRLSFKKTIAELQM
IDPSADLYAQVQEIAGSPAIKKGILLGLKIVDEIIRVMGEKPENIVIEMARENQTTARGKA
LSKRREAKIKEGLAALGSSLLKENLPGNADLSQRKIYLYYTQNGKDIYLDEPLDEDRLSQY
DEDHIIPQSFTVDNSLDNLVLTNSSQNRGNKKDDVPSLEVVNRQLAYWRSLKDAGLMTQRK
FDNLTKAMRGGLTDKDRERFIQRQLVETRQITKNVAKLLDMRLNDKKDEAGNKIRETNIVL
LKSAMASEFRKMFRLYKVRELNDYHHAHDAYLNAAIAINLLALYPYMADDEVYGEFRYKKK
PQAEKATYEKLRQWNLIKRFGEKQLFTPDHEDCWNKERDIKTIKKVMGYRQVNVVKKAEER
TGMLFKETINGKINKGSRIPIKKDLDPSKYGGYIEEKMAYYAVISYEDKKKKPGKTIVGIS
IMDKKEFEYDSISYLGKLGFSNPVVQIILKNYSLIAYPDGRRRYITGATKTTKGKVELQKA
NQIAMEQDLVNFIYHLKNYDEISHPESYAFVQSHTDYFDRLFDSIEHYTRRFLDAETNINR
LRRIYEEEKKKDPVDIEALVASFIELLKLTSAGAPADFIFMGEAISRRRYNSMTGLEDGQV
IYQSLTGLYETRMRFED (SEQ ID NO: 86).
Peptoniphilus sp. Marseille-P3761 Cas9 with Nuclear Localization Signal
(NLS) and Linker
MPKKKRKVGEKKTNYTIGLDIGTDSVGWAVVKDDLELVKKRMKVLGNTETNYIKKNLWGSL
LFESGQTAKDRRLKRVARRRYERRRNRLTELQKIFAPAIDEVDENFFFRLNESFLVPEDKA
FSKNPIFGTLGEDKTYYKTYPTIYHLRQHLADSEEKADVRLIYLALAHMIKYRGHFLIEGK
LDTEHIAINENLEQFFESYNALFSEEPIELRKEELIAIENILREKNSRTVKEKRITSFLKD
IGRANKQSPMMAFITLIVGKKAKFKAAFNLEEEISLNLTDDSYDENLEILLNTIGSDFADL
FDHAQRVYNAVELAGILSGDVKNTHAKLSAQMVAMYERHKEQLKEYKSFIKANLPDQYDMT
FVAPKDAQKKDLKGYAGYIDGNMSQDSFYKFVKDQLKEVPGSEKFLDSIEKEDELRKQRSF
YNGVIPNQVHLAEMEAILDRQENYYPWLKENREKIISLLTFRIPYYVGPLADGQSEFAWLE
RKSDEKIKPWNFSDVVDLDRSAEKFIEQLIGRDTYLPDEYVLPKKSLIYQKYMVENELTKI
AYLDERQKRMNLSSVEKKEIFETLFKKRSKVTEKQLVKFFENYLQIDNPTIFGIEDAFNAD
YSTYVELAKVPGMKSMMDDPDNEDLMEEIVKILTVFEDRKMRRKQLEKYKERLSPEQIKEL
AKKHYTGWGRLSKKLLVGIRDKETQKTILDYLVEDDNHSGGRQHLNRNLMQLINDDRLSFK
KTIAELQMIDPSADLYAQVQEIAGSPAIKKGILLGLKIVDEIIRVMGEKPENIVIEMAREN
QTTARGKALSKRREAKIKEGLAALGSSLLKENLPGNADLSQRKIYLYYTQNGKDIYLDEPL
DFDRLSQYDEDHIIPQSFTVDNSLDNLVLTNSSQNRGNKKDDVPSLEVVNRQLAYWRSLKD
AGLMTQRKFDNLTKAMRGGLTDKDRERFIQRQLVETRQITKNVAKLLDMRLNDKKDEAGNK
IRETNIVLLKSAMASEFRKMFRLYKVRELNDYHHAHDAYLNAAIAINLLALYPYMADDEVY
GEFRYKKKPQAEKATYEKLRQWNLIKRFGEKQLFTPDHEDCWNKERDIKTIKKVMGYRQVN
VVKKAEERTGMLFKETINGKTNKGSRIPIKKDLDPSKYGGYIEEKMAYYAVISYEDKKKKP
GKTIVGISIMDKKEFEYDSISYLGKLGFSNPVVQIILKNYSLIAYPDGRRRYITGATKTTK
GKVELQKANQIAMEQDLVNFIYHLKNYDEISHPESYAFVQSHTDYFDRLEDSIEHYTRREL
DAETNINRLRRIYEEEKKKDPVDIEALVASFIELLKLTSAGAPADFIFMGEAISRRRYNSM
TGLFDGQVIYQSLTGLYETRMRFEDGKRPAATKKAGQAKKKKGSYPYDVPDYAYPYDVPDY
AYPYDVPDYA (SEQ ID NO: 87).
NLS (bold), can be substituted with different NLSs
Linker (underlined), can be removed or extended
3xHA tag (italics), can be substituted with different tags

In some embodiments, the Cas9 protein comprises one or more mutations in reference to SEQ ID NO: 1, 4, 8, 14, 84 or 86. For example, the amino acid sequence of the Cas9 protein comprises at least one, at least two, at least three, at least four, at least five, at least six, at least seven, at least eight, at least nine, at least 10 mutations in SEQ ID NO: 1, 4, 8, 14, 84 or 86. Various mutations are known in the art, and include for example, amino acid substitutions.

In some embodiments, two or more catalytic domains of Cas9 (RuvC1, RuvCII, RuvCIII) are mutated to produce an inactive, or “dead” Cas9 (dCas9) that lacks nucleic acid cleavage activity. In some embodiments, the one or more mutations are in the PAM Interacting, HNH, and or the RuvC domains. In some embodiments, Cas9 is mutated to reduce DNA cleavage activity to less than about 25%, 15%, 10%, 5%, 1%, 0.1%, 0.01% or lower with respect to its non-mutated form.

In some embodiments a nickase-mutant version of Cas9 is provided. In some embodiments, the nickase mutant has one or more amino acid substitutions in the RuvC and/or the HNH domains. Various nickase mutations are known with respect to SpCas9 (Streptococcus pyogenes) and include for example mutations at one or more of amino acid positions 10, 12, 17, 762, 840, 854, 863, 982, 983, 984, 986, 987 of wild type SpCas9. For example, an aspartic acid-to-alanine substitution that corresponds to D10A in SpCas9 results in the creation of a nickase. In some embodiments, the Cas9 described herein has one or more mutations that result in the creation of a nickase. In some embodiments, the Cas9 described herein has one or more mutations at an amino acid position that corresponds to one or more of amino acids 10, 12, 17, 762, 840, 854, 863, 982, 983, 984, 986, 987 of SpCas9.

In some embodiments, the mutation is an aspartic acid-to-alanine substitution (D10A) in the RuvC domain of ScoCas9. In some embodiments, the mutation is an aspartic acid-to-alanine substitution (D14A) in the RuvC domain of SirCas9. In some embodiments, the mutation is an aspartic acid-to-alanine substitution (D38A) in the RuvC domain of VapCas9 (e.g., corresponding to D10A in SpCas9). In some embodiments, the mutation is an aspartic acid-to-alanine substitution (D12A) in the RuvC domain of EpeCas9. In some embodiments, the mutation is an aspartic acid-to-alanine substitution (D9A) in the RuvC domain of LfeCas9. In some embodiments, the mutation is an aspartic acid-to-alanine substitution (D12A) in the RuvC domain of PmaCas9.

In some embodiments, the mutation is an aspartic acid-to-glycine substitution (D10G) in the RuvC domain of ScoCas9. In some embodiments, the mutation is an aspartic acid-to-glycine substitution (D14G) in the RuvC domain of SirCas9. In some embodiments, the mutation is an aspartic acid-to-glycine substitution (D38G) in the RuvC domain of VapCas9 (e.g., corresponding to D10G in SpCas9). In some embodiments, the mutation is an aspartic acid-to-glycine substitution (D12G) in the RuvC domain of EpeCas9. In some embodiments, the mutation is an aspartic acid-to-glycine substitution (D9A) in the RuvC domain of LfeCas9. In some embodiments, the mutation is an aspartic acid-to-glycine substitution (D12G) in the RuvC domain of PmaCas9.

In some embodiments, such one or more mutations described herein converts Cas9 to an inactive, or “dead” version of Cas9 (dCas9). Accordingly, in some embodiments, the Cas9 protein comprises one or more mutations that inhibits the ability of Cas9 to cleave both strands of a DNA duplex.

In some embodiments, when coexpressed with a guide RNA, dead Cas9 generates a DNA recognition complex that can specifically interfere with transcriptional elongation, RNA polymerase binding, or transcription factor binding. In some embodiments, dead Cas9 is used to specifically target effector proteins of various functions to specific nucleic acid target sites.

In some embodiments, a high-fidelity Cas9 variant comprises enhanced specificity, which minimizes off-target cleavage. In some embodiments, engineered variants, for example, ‘hyper-accurate Cas9’ (N692A, M694A, Q695A and/or H698A mutations corresponding to SpyCas9) and/or ‘high-fidelity Cas9’ (N467A, R661A, Q695A and/or Q926A mutations corresponding to SpyCas9) are used which comprise mutations mainly within the REC3 domain and achieve higher specificity and fidelity. High-fidelity variants reduce the capacity of Cas9 to stabilize mismatches and reduce off-target DNA cleavage. In some embodiments, the increase in specificity is accompanied by a loss in efficiency of on-target cleavage by about 100 fold. In some embodiments, a SuperFi-Cas9 is used, which is a high-fidelity variant that maintains on-target cleavage rates comparable to wild-type Cas9. In some embodiments, the SuperFi-Cas9 comprises mutations in the RuvC loop. In some embodiments, the mutations inhibit formation of a kinked conformation that facilitates subsequent cleavage of gRNA-TS duplex. In some embodiments, the Y1016, R1019, Y1010, Y1013, K1031, Q1027 and/or V1018 residues corresponding to SpyCas9 are mutated, for example, to aspartic acid. (Bravo, J. et al. Structural basis for mismatch surveillance by CRISPR-Cas9 Nature, 603, March 2022).

The engineered, non-naturally occurring Cas9 is has an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) identical to a Cas9 amino sequence at SEQ ID NOs. 2, 5, 9, 15, 85, 87, 95, or 96.

In some embodiments, the engineered non-naturally occurring Cas9 is encoded in a nucleic acid molecule codon-optimized for human cells (e.g., codon optimized for expression, stability, etc.).

Exemplary Cas9 sequences with Nuclear Localization Signal (NLS) and a linker is provided in Table 2 below.

TABLE 2
Exemplary Cas9 Sequence with NLS and Linker

Sequence of ScoCas9 with Nuclear Localization Signal (NLS) and Linker
MPKKKRKVGGKPYSIGLDIGTNSVGWAVVTDDYKVPAKKMKVLGNTDKQSIKKNLLGALLE
DSGETAEATRLKRTARRRYTRRKNRLRYLQEIFTGEMNKVDENFFQRLDDSFLVDEDKRGE
HHPIFGNIAAEVKYHDDFPTIYHLRRHLADTSKKADLRLVYLALAHMIKERGHFLYEGDLK
AENTDVQALFKDEVEEYDKTIEESHLSEITVDALSILTEKVSKSSRLENLIAHYPTEKKNT
LFGNLIALSLDLHPNFKTNFQLSEDAKLQFSKDTYEEDLEGFLGEVGDEYADLFASAKNLY
DAILLSGILTVDDNSTKAPLSASMVKRYEEHQKDLKKLKDFIKVNAPDQYNAIFKDKNKKG
YASYIESGVKQDEFYKYLKGILLKINGSGDFLDKIDREDFLRKQRTFDNGSIPHQIHLQEM
HAILRRQGEHYPFLKENQDKIEKILTFRIPYYVGPLARKGSRFAWAEYKADEKITPWNEDD
ILDKEKSAEKFITRMTLNDLYLPEEKVLPKHSPLYEAFTVYNELTKVKYVNEQGEAKFEDT
NMKQEIFDHVFKENRKVTKDKLLNYLNKEFEEFRIVNLTGLDKENKAFNSSLGTYHDLRKI
LDKSFLDDKANEKTIEDIIQTLTLFEDREMIRQRLQKYSDIFTKAQLKKLERRHYTGWGRL
SYKLINGIRNKENKKTILDYLIDDGYANRNFMQLINDDALSFKEEIARAQIIDDVDDIANV
VHDLPGSPAIKKGILQSVKIVDELVKVMGHNPANIIIEMARENQTTDKGRRNSQQRLKLLQ
DSLKNLDNPVNIKNVENQQLQNDRLFLYYIQNGKDMYTGETLDINNLSQYDIDHIIPQAFI
KDNSLDNRVLTRSDKNRGKSDDVPSIEVVHEMKSFWSKLLSVKLITQRKFDNLTKAERGGL
TEEDKAGFIKRQLVETRQITKHVAQILDERENTEFDGNKRRIRNVKIITLKSNLVSNERKE
FELYKVREINDYHHAHDAYLNAVVGNALLLKYPQLEPEFVYGEYPKYNSYRSRKSATEKEL
FYSNILRFFKKEDIQTNEDGEIAWNKEKHIKILRKVLSYPQVNIVKKTEEQTGGFSKESIL
PKGESDKLIPRKTKNSYWDPKKYGGFDSPVVAYSILVFADVEKGKSKKLRKVQDMVGITIM
EKKRFEKNPVDFLEQRGYRNVRLEKIIKLPKYSLFELENKRRRLLASAKELQKGNELVIPQ
RFTTLLYHSYRIEKDYEPEHREYVEKHKDEFKELLEYISVFSRKYVLADNNLTKIEMLESK
NKDAEVSSLAKSFISLLTFTAFGAPAAFNFFGENIDRKRYTSVTECLNATLIHQSITGLYE
TRIDLSKLGEDGKRPAATKKAGQAKKKKGSYPYDVPDYAYPYDVPDYAYPYDVPDYA
(SEQ ID NO: 2).
Sequence of Sharpea Cas9 with Nuclear Localization Signal (NLS) and Linker
MPKKKRKVGAKNKDIRYSIGLDIGTNSVGWAVMDEHYELLKKGNHHMWGSRLFDAAEPAAT
RRASRSIRRRYNKRRERIRLLRDLLGDMVMEVDPTFFIRLLNVSFLDEEDKQKNLGNDYKD
NYNLFIEKDENDKTYYDKYPTIYHLRKELCENKEKADPRLIYLALHHIVKYRGNELKEGQS
FAKVYEDIEEKLDNTLKKEMSLNDLDNLFVDNDINSMITVLSKIYQRSKKADDLLKIMNPT
KEERAAYKEFTKALVGLKFNVSKMILAQEVKKDDKDIELDFSNVDYDSTVDGLQAELGEYI
EFIEMLHSINSWVELQDILGNNSTISAAMVERYEEHKNDLRVLKKVIREELPDKYNEVFRE
DNPKLHNYLGYIKYPKNTPVEEFYEYIKRLLAKVDTGEAREILERIDLEKFMLKQNSRING
SIPYQMQKDEMIQIIDNQSVYYPQLKENREKLISILEFRIPYYFGPLNTHSEFAWIKKFED
KQKERILPWNYDQIVDIDATAEGFIERMQNTGTYFPDKPVMAKNSLTVSKFEVLNELNKIR
INGKLIPVETKKELLSDLFMKNKTITDKKLKDWLVTHQYYDTNEELKIEGYQKDLQFSTSL
APWIDFTKIFGEINASNYQLIEKIIYDISIFEDKKILKRRLKKVYQLDDLLVDKILKLNYT
GWSRLSEKLLTGIKSKNSKETILSILENSNMNLMEIINDESLGFKQIIEESNKKDIEGPER
YDEVKKLAGSPAIKRGIWQALLVVQEITKFMKHEPSHIYIEFAREEQEKVRTESRIAKLQK
IYKDLNLQTKEDQLVYESLKKEDAKKKIDTDALYLYYLQMGKSMYSGKPLDIDKLSTYHID
HILPRSLIKDDSLDNRVLVLPKENEWKLDSETVPFEIRNKMMGFWQKLHENGLMSNKKFFS
LIRTDENEKDKKRFINRQLVETRQIIKNVAVIINDHYTNTNVVTVRAELSHQFRERYKIYK
NRDLNDLHHAHDAYIACILGQFIHQNFGNMDVNMIYGQYKKNYKKDVQEHNNYGFILNSMN
HIHFNDDNSVIWDPSYIGKIKSCFCYKDVYVTKKLEQNDAKLEDLTILPSDKNSENGVTKA
KIPVNKYRKDVNKYGGFSGDAPIMLAIEADKGKKHVRQVIAFPLRLKNYNDEERIKFIEKE
KNLKNVKILTEVKKNQLILINHQYFFITGTNELVNATQLKLSAKNTKNLFNLVDANKHNKL
ESIDDANFNEVIQELICKLQEPIYSRYNSIGKEFEDSYEKINAVTKQDKLYIIEYLIAIMS
AKATQGYIKPELAREIGTNGKNKGRIKSFTIDLNKTTFISTSVTGLESKKYKLGKRPAATK
KAGQAKKKKGSYPYDVPDYAYPYDVPDYAYPYDVPDYA (SEQ ID NO: 5).
Sequence of Veillonella parvula Cas9 with Nuclear Localization Signal (NLS)
and Linker
MPKKKRKVGSIINFQRRGLMETQASNQLISSHLKGYPIKDYFVGLDIGTSSVGWAVINKAY
ELLKFRSHKMWGSRLFDEGESAVARRGFRSMRRRLERRKLRLKLLEELFADAMAQVDPTFF
MRLRESKYHYEDKTTGHSSKHILFIDKNYNDQDYFKEYPTVYHLRSELMKSGTDDIRKLEL
AVHHILKYRGNFLYEGATFDSNASTLDDVIKQALENITFNCFDCNSAISSIGQILMEAGKT
KSDKAKAIEHLVDTYIATDTVDTSSKTQKDQVKEDKKRLKAFANLVLGLNASLIDLEGSVE
ELEEDLKKLQITGDTYDDKRDELAKAWSDEIYIIDDCKSVYDAIILLSIKEPGLTISESKV
KAFNKHKDDLAILKSLLKSDRSIYNTMFKVDEKGLHNYVHYIKQGRTEETSCNREDFYKYT
KKIVEGLSDSKDKEYILSQIELQILLPLQRIKDNGVIPYQLHLEELKAILAKCGPKFPELN
EVADGFSVAEKLIKMLEFRIPYYVGPLNTHHNVDNGGFAWAVRKASGRVTPWNEDDKIDRE
KSAAAFIKNLTNKCTYLLGEDVLPKSSLLYSEFMLLNELNNVRIDGKPLEKVVKEHLIEAV
FKQDHKKMTKNRIEQFLKDNGYISETHKHEITGLDGEIKNDLASYRDMVRILGDGEDRSMA
EEIITDITIFGESKKMLRETLRKKFASCLDDEAIKKLTKLRYRDWGRLSQKLLNGIEGCDK
AGDGTPETIIILMRNFSYNLMELLGDKESFMERIQEINAKLTEGQIVNPHDIIDDLALSPA
VKRAVWQALRIVDEVAHIKKALPARIFVEVTRSNKNEKKKKDSRQKRLSDLYAAIKKDDVL
LNGLNNEIFGELKSSLAKYDDAALRSKKLYLYYTQMGRCAYTGEIIELSLLNTDNYDIDHI
YPRSLTKDDSFDNLVLCKRTANAQKSDAYPISEEIQKTQKPFWTFLKQQGLISERKYERLT
RITPLTADDLSGFIARQLVETNQSVKAATTLLRRLYPGVDVVFVKAENVTDERHDNNFIKV
RSLNHHHHAKDAYLNIVVGNVYHERFTRNFRAFFKKNGANRTYNLAKMENYDVNCTNAKDG
KAWDVKTSMDTVKKMMDSNDVRVTKRLLEQTGALADATIYKATVAGKAKDGAYIGMKTKSS
VFADVSKYGGMTKIKNAYSIIVQYTGKKGEVIKEIVPLPIYLTNRNTTDQDLINYVASIIP
QAKDISIIYGKLCINQLVKVNGFYYYLGGKTNSKFCIDNAIQVIVSNEWIPYLKVLEKENN
MRKDNKDLKANVVSTRALDNKHTIEVRIVEEKNIEFFDYLVSKLKMPIYQKMKGNKAAELS
EKGYGLFKKMSLEEQSIHLIELLNLLTNQKTTFEVKPLGITASRSTVGSKISNQDEFKVIN
ESITGLYSNEVTIVGKRPAATKKAGQAKKKKGSYPYDVPDYAYPYDVPDYAYPYDVPDYA
(SEQ ID NO: 9).
Sequence of Ezakiella peruensis Cas9 with Nuclear Localization Signal (NLS)
and Linker
MPKKKRKVGTKVKDYYIGLDIGTSSVGWAVTDEAYNVLKFNSKKMWGVRLEDDAKTAEERR
GQRGARRRLDRKKERLSLLQDFFAEEVAKVDPNFFLRLDNSDLYMEDKDQKLKSKYTLEND
KDFKDKNFHKKYPTIHHLLMDLIEDDSKKDIRLVYLACHYLLKNRGHFIFEGQKEDTKSSF
ENSLNELKVHLNDEYGLDLEFDNENLINILTDPKLNKTAKKKELKSVIGDTKFLKAVSAIM
IGSSQKLVDLFENPEDFDDSAIKSVDFSTTSFDDKYSDYELALGDKIALVNILKEIYDSSI
LENLLKEADKSKDGNKYISNAFVKKYNKHGQDLKEFKRLVRQYHKSAYFDIFRSEKVNDNY
VSYTKSSISNNKRVKANKFTDQEAFYKFAKKHLETIKYKINKVNGSKADLELIDGMLRDME
FKNFMPKIKSSDNGVIPYQLKLMELNKILENQSKHHEFLNVSDEYGSVCDKIASIMEFRIP
YYVGPLNPNSKYAWIKKQKDSEITPWNFKDVVDLDSSREEFIDSLIGRCTYLKDEKVLPKA
SLLYNEYMVLNELNNLKLNDLPITEEMKKKIFDQLFKTRKKVTLKAVANLLKKEFNINGEI
LLSGTDGDFKQGLNSYNDFKAIVGDKVDSDDYRDKIEEIIKLIVLYGDDKSYLQKKIKAGY
GKYFTDSEIKKMAGLNYKDWGRLSKKLLTGLEGANKITGERGSIIHFMREYNLNLMELMSA
SFTFTEEIQKLNPVDDRKLSYEMVDELYLSPSVKRMLWQSLRIVDEIKNIMGTDSKKIFIE
MARGKEEVKARKESRKNQLLKFYKDGKKAFISEIGEERYSYLLSEIEGEEENKFRWDNLYL
YYTQLGRCMYSLEPIDISELSSKNIYDQDHIYPKSKIYDDSIENRVLVKKDLNSKKGNSYP
IPDEILNKNCYAYWKILYDKGLIGQKKYTRLTRRTGFTDDELVQFISRQIVETRQATKETA
NLLKTICKNSEIVYSKAENASRFRQEFDIVKCRAVNDLHHMHDAYINIIVGNVYNTKFTKD
PMNFVKKQEKARSYNLENMFKYDVKRGGYTAWIADDEKGTVKNASIKRIRKELEGTNYRFT
RMNYIESGALFNATLQRKNKGSRPLKDKGPKSSIEKYGGYTNINKACFAVLDIKSKNKIER
KLMPVEREIYAKQKNDKKLSDEIFSKYLKDRFGIEDYRVVYPVVKMRTLLKIDGSYYFITG
GSDKTLELRSALQLILPKKNEWAIKQIDKSSENDYLTIERIQDLTEELVYNTFDIIVNKFK
TSVFKKSFLNLFQDDKIENIDFKFKSMDFKEKCKTLLMLVKAIRASGVRQDLKSIDLKSDY
GRLSSKTNNIGNYQEFKIINQSITGLFENEVDLLKLGKRPAATKKAGQAKKKKGSYPYDVP
DYAYPYDVPDYAYPYDVPDYA (SEQ ID NO: 15).
Sequence of Lactobacillus fermentum strain AF15-40LB Cas9 with Nuclear
Localization Signal (NLS) and Linker
MPKKKRKVGKEYHIGLDIGTSSIGWAVTDSQFKLMRIKGKTAIGVRLFEEGKTAAERRTER
TTRRRLKRRKWRLHYLDEIFAPHLQEVDENFLRRLKQSNIHPEDPAKNQAFIGKLLFPDLL
KKNERGYPTLIKMRDELPVEQRAHYPVTNIYKLREAMINEDRQFDLREVYLAVHHIVKYRG
HFLNNASVDKFKVGRIDFDKSFNVLNEAYEELQNGEGSFTIEPSKVEKIGQLLLDTKMRKL
DRQKAVAKLLEVKVADKEETKRNKQIATAMSKLVLGYKADFATVAMANGNEWKIDLSSETS
EDEIEKFREELSDAQNDILTEITSLESQIMLNEIVPNGMSISESMMDRYWTHERQLAEVKE
YLATQPASARKEFDQVYNKYIGQAPKEKGFDLEKGLKKILSKKENWKEIDELLKAGDELPK
QRTSANGVIPHQMHQQELDRIIEKQAKYYPWLATENPATGERDRHQAKYELDQLVSFRIPY
YVGPLVTPEVQKATSGAKFAWAKRKEDGEITPWNLWDKIDRAESAEAFIKRMTVKDTYLLN
EDVLPANSLLYQKYNVLNELNNVRVNGRRLSVGIKQDIYTELFKKKKTVKAGDVASLVMAK
TRGVNKPSVEGLSDPKKENSNLATYLDLKSIVGDKVDDNRYQMDLENIIEWRSVFEDGEIF
ADKLTEVEWLTDEQRSALVKKRYKGWGRLSKKLLTGIVDENGQRIIDLMWNTDQNEMQIVN
QPVFKEQIDQLNQKAITNDGMTLRERVESVLDDAYTSPQNKKAIWQVVRVVEDIVKAVGNA
PKSISIEFARNEGNKGEITRSRRTQLQKLFEDQAHELVKDTSLTEELEKAPDLSDRYYFYF
TQGGKDMYTGDPINFDEISTKYDIDHILPQSFVKDDSLDNRVLVSRAENNKKSDRVPAKLY
AAKMKPYWNQLLKQGLITQRKFENLTMDVDQTIKYRSLGFVKRQLVETRQVIKLTANILGS
MYQEAGTDIIETRAGLTKQLREEFDLPKVREVNDYHHAVDAYLTTFAGQYLNRRYPKLRSF
FVYGEYMKFKHGSDLKLRNFNFFHELMEGDKSQGKVVDQQTGELITTRDEVADYEDWVINL
KVMLISNETYEETGKYFDASHESSSLYLKNQNKKSKLVVPLKNKLQPEYYGAYTGITQGYM
VILKLLDKKGGFGVYRIPRYAADILNKCHDEVAYRNKIAEIISSDPRAPKSFEVVVPRVLK
GTFLVDGEEKFILSSYRYKVNATQLILPVSDIKLIQDNFKALKKLNVEMQTKKLIEIYDNI
LRQVDKYYKLYDINKFRAKLHDGRSKFVELDDEGQDASKEKVIIKILRGLHFGSDLQNLKE
IGFGTTPLGQFQVSEAGIRLSNTAFIIFKSPTGLFNRKLYLKNL
GKRPAATKKAGQAKKKKGSYPYDVPDYAYPYDVPDYAYPYDVPDYA (SEQ ID NO: 85).
Sequence of Peptoniphilus sp. Marseille-P3761 Cas9 with Nuclear Localization
Signal (NLS) and Linker
MPKKKRKVGEKKTNYTIGLDIGTDSVGWAVVKDDLELVKKRMKVLGNTETNYIKKNLWGSL
LFESGQTAKDRRLKRVARRRYERRRNRLTELQKIFAPAIDEVDENFFERLNESELVPEDKA
FSKNPIFGTLGEDKTYYKTYPTIYHLRQHLADSEEKADVRLIYLALAHMIKYRGHFLIEGK
LDTEHIAINENLEQFFESYNALFSEEPIELRKEELIAIENILREKNSRTVKEKRITSFLKD
IGRANKQSPMMAFITLIVGKKAKFKAAFNLEEEISLNLTDDSYDENLEILLNTIGSDFADL
FDHAQRVYNAVELAGILSGDVKNTHAKLSAQMVAMYERHKEQLKEYKSFIKANLPDQYDMT
FVAPKDAQKKDLKGYAGYIDGNMSQDSFYKFVKDQLKEVPGSEKFLDSIEKEDFLRKQRSF
YNGVIPNQVHLAEMEAILDRQENYYPWLKENREKIISLLTFRIPYYVGPLADGQSEFAWLE
RKSDEKIKPWNFSDVVDLDRSAEKFIEQLIGRDTYLPDEYVLPKKSLIYQKYMVENELTKI
AYLDERQKRMNLSSVEKKEIFETLFKKRSKVTEKQLVKFFENYLQIDNPTIFGIEDAFNAD
YSTYVELAKVPGMKSMMDDPDNEDLMEEIVKILTVFEDRKMRRKQLEKYKERLSPEQIKEL
AKKHYTGWGRLSKKLLVGIRDKETQKTILDYLVEDDNHSGGRQHLNRNLMQLINDDRLSFK
KTIAELQMIDPSADLYAQVQEIAGSPAIKKGILLGLKIVDEIIRVMGEKPENIVIEMAREN
QTTARGKALSKRREAKIKEGLAALGSSLLKENLPGNADLSQRKIYLYYTQNGKDIYLDEPL
DFDRLSQYDEDHIIPQSFTVDNSLDNLVLTNSSQNRGNKKDDVPSLEVVNRQLAYWRSLKD
AGLMTQRKFDNLTKAMRGGLTDKDRERFIQRQLVETRQITKNVAKLLDMRLNDKKDEAGNK
IRETNIVLLKSAMASEFRKMFRLYKVRELNDYHHAHDAYLNAAIAINLLALYPYMADDEVY
GEFRYKKKPQAEKATYEKLRQWNLIKRFGEKQLFTPDHEDCWNKERDIKTIKKVMGYRQVN
VVKKAEERTGMLFKETINGKTNKGSRIPIKKDLDPSKYGGYIEEKMAYYAVISYEDKKKKP
GKTIVGISIMDKKEFEYDSISYLGKLGFSNPVVQIILKNYSLIAYPDGRRRYITGATKTTK
GKVELQKANQIAMEQDLVNFIYHLKNYDEISHPESYAFVQSHTDYFDRLEDSIEHYTRRFL
DAETNINRLRRIYEEEKKKDPVDIEALVASFIELLKLTSAGAPADFIFMGEAISRRRYNSM
TGLFDGQVIYQSLTGLYETRMRFEDGKRPAATKKAGQAKKKKGSYPYDVPDYAYPYDVPDY
AYPYDVPDYA (SEQ ID NO: 87).
Sequence of ScoCas9 variant with Nuclear Localization Signal (NLS) and Linker
(ScoCas9-NGC-v1)
MPKKKRKVGMGKPYSIGLDIGTNSVGWAVVTDDYKVPAKKMKVLGNTDKQSIKKNLLGALL
FDSGETAEATRLKRTARRRYTRRKNRLRYLQEIFTGEMNKVDENFFQRLDDSELVDEDKRG
EHHPIFGNIAAEVKYHDDFPTIYHLRRHLADTSKKADLRLVYLALAHMIKERGHFLYEGDL
KAENTDVQALFKDFVEEYDKTIEESHLSEITVDALSILTEKVSKSSRLENLIAHYPTEKKN
TLFGNLIALSLDLHPNEKTNFQLSEDAKLQFSKDTYEEDLEGELGEVGDEYADLFASAKNL
YDAILLSGILTVDDNSTKAPLSASMVKRYEEHQKDLKKLKDFIKVNAPDQYNAIFKDKNKK
GYASYIESGVKQDEFYKYLKGILLKINGSGDFLDKIDREDFLRKQRTFDNGSIPHQIHLQE
MHAILRRQGEHYPFLKENQDKIEKILTFRIPYYVGPLARKGSRFAWAEYKADEKITPWNED
DILDKEKSAEKFITRMTLNDLYLPEEKVLPKHSPLYEAFTVYNELTKVKYVNEQGEAKFFD
TNMKQEIFDHVFKENRKVTKDKLLNYLNKEFEEFRIVNLTGLDKENKAFNSSLGTYHDLRK
ILDKSFLDDKANEKTIEDIIQTLTLFEDREMIRQRLQKYSDIFTKAQLKKLERRHYTGWGR
LSYKLINGIRNKENKKTILDYLIDDGYANRNEMQLINDDALSFKEEIARAQIIDDVDDIAN
VVHDLPGSPAIKKGILQSVKIVDELVKVMGHNPANIIIEMARENQTTDKGRRNSQQRLKLL
QDSLKNLDNPVNIKNVENQQLQNDRLFLYYIQNGKDMYTGETLDINNLSQYDIDHIIPQAF
IKDNSLDNRVLTRSDKNRGKSDDVPSIEVVHEMKSFWSKLLSVKLITQRKEDNLTKAERGG
LTEEDKAGFIKRQLVETRQITKHVAQILDERENTEFDGNKRRIRNVKIITLKSNLVSNFRK
EFELYKVREINDYHHAHDAYLNAVVGNALLLKYPQLEPEFVYGEYPKYNSYRSRKSATEKF
LFYSNILRFFKKEDIQTNEDGEIAWNKEKHIKILRKVLSYPQVNIVKKTEEQTGGFSKESI
LPKGESDKLIPRKTKNSYWDPKKYGGFMQPVVAYSILVFADVEKGKSKKLRKVQDMVGITI
MEKKRFEKNPVDFLEQRGYRNVRLEKIIKLPKYSLFELENKRRRLLASAKFLQKGNELVIP
QRFTTLLYHSYRIEKDYEPEHREYVEKHKDEFKELLEYISVFSRKYVLADNNLTKIEMLES
KNKDAEVSSLAKSFISLLTFTAFGAPRAFNFFGENIARKEYRSVTECLNATLIHQSITGLY
ETRIDLSKLGEDGEGADKRTADGSEFESPKKKRKV (SEQ ID NO: 95)
Sequence of ScoCas9 with Nuclear Localization Signal (NLS) and Linker
(ScoCas9-NGC-v2)
MPKKKRKVGMGKPYSIGLDIGTNSVGWAVVTDDYKVPAKKMKVLGNTDKQSIKKNLLGALL
FDSGETAEATRLKRTARRRYTRRKNRLRYLQEIFTGEMNKVDENFFQRLDDSELVDEDKRG
EHHPIFGNIAAEVKYHDDFPTIYHLRRHLADTSKKADLRLVYLALAHMIKERGHELYEGDL
KAENTDVQALFKDFVEEYDKTIEESHLSEITVDALSILTEKVSKSSRLENLIAHYPTEKKN
TLFGNLIALSLDLHPNEKTNFQLSEDAKLQFSKDTYEEDLEGFLGEVGDEYADLFASAKNL
YDAILLSGILTVDDNSTKAPLSASMVKRYEEHQKDLKKLKDFIKVNAPDQYNAIFKDKNKK
GYASYIESGVKQDEFYKYLKGILLKINGSGDFLDKIDREDFLRKQRTEDNGIIPHQIHLQE
MHAILRRQGEHYPFLKENQDKIEKILTFRIPYYVGPLARKGSRFAWAEYKADEKITPWNED
DILDKEKSAEKFITRMTLNDLYLPEEKVLPKHSPLYEAFTVYNELTKVKYVNEQGEAKFFD
TNMKQEIFDHVFKENRKVTKDKLLNYLNKEFEEFRIVNLTGLDKENKAFNSSLGTYHDLRK
ILDKSFLDDKANEKTIEDIIQTLTLFEDREMIRQRLQKYSDIFTKAQLKKLERLHYTGWGR
LSYKLINGIRNKENKKTILDYLIDDGYANRNFMQLINDDALSFKEEIARAQIIDDVDDIAN
VVHDLPGSPAIKKGILQSVKIVDELVKVMGHNPANIIIEMARENQTTDKGRRNSQQRLKLL
QDSLKNLDNPVNIKNVENQQLQNDRLFLYYIQNGKDMYTGETLDINNLSQYDIDHIIPQAF
IKDNSLDNRVLTRSDKNRGKSDDVPSIEVVHEMKSFWSKLLSVKLITQRKEDNLTKAERGG
LTEEDKAGFIKRQLVETRQITKHVAQILDERENTEFDGNKRRIRNVKIITLKSNLVSNERK
EFELYKVREINDYHHAHDAYLNAVVGNALLLKYPQLEPEFVYGEYPKYNSYRSRKSATEKF
LFYSNILRFFKKEDIQTNEDGEIAWNKEKHIKILRKVLSYPQVNIVKKTEEQTGGFSKESI
LPKGESDKLIPRKTKNSYWDPKKYGGFMQPVVAYSILVFADVEKGKSKKLRKVQDMVGITI
MEKKRFEKNPVDFLEQRGYRNVRLEKIIKLPKYSLFELENKRRRLLASAKFLQKGNELVIP
QRFTTLLYHSYRIEKDYEPEHREYVEKHKDEFKELLEYISVFSRKYVLADNNLTKIEMLES
KNKDAEVSSLAKSFISLLTFTAFGAPRAFNFFGENIARKEYRSVTECLNATLIHQSITGLY
ETRIDLSKLGEDGEGADKRTADGSEFESPKKKRKV (SEQ ID NO: 96)

NLS (bold), can be substituted with different NLSs
Linker (underlined), can be removed or extended

In some embodiments, the engineered non-naturally occurring Cas9 comprises a tag. A variety of tags may be fused to the Cas9 variant (e.g., 3×HA tag), depending on purpose, as will be apparent to a skilled person.

Various species exhibit codon bias (i.e. differences in codon usage by organisms) which correlates with the efficiency of translation of messenger RNA (mRNA) by utilizing codons in mRNA that correspond with the abundance of tRNA species for that codon in a particular organism. Various methods in the art can be used for computer optimization, including for example through use of software. In some embodiments, codon optimization refers to modification of nucleic acid sequences for enhanced expression in the host cells of interest by replacing at least one codon (e.g. 1, 2, 3, 4, 5, 10, 15, 20, 25, 50 or more codons) of the native sequence with codons that are more frequently used or most frequently used in the genes of the host cell while maintaining the native amino acid sequence.

In some embodiments, the Cas9 protein described herein is codon optimized. This type of optimization is known in the art and entails the mutation of foreign-derived DNA to mimic the codon preferences of the intended host organism or cell while encoding the same protein. Thus, the codons are changed, but the encoded protein remains unchanged. Codon optimization improves soluble protein levels and increases activity and editing efficiency in a given species. Codon optimization also results in increased translation and protein expression.

In some embodiments, the Cas9 protein is codon optimized for expression in eukaryotic cells. In some embodiments, the Cas9 protein is codon optimized for expression in human cells.

Protospacer Adjacent Motif (PAM)

Each Cas endonuclease binds to its target sequence only in the presence of a specific sequence, known as a protospacer adjacent motif (PAM), on the non-targeted i.e. complementary DNA strand. Cas nucleases isolated from different bacterial species recognize different PAM sequences. For example, the SpCas9 nuclease (from Staphylococcus pyogenes) cuts upstream of the PAM sequence 5′-NGG-3′ (where “N” can be any nucleotide base), SaCas9 (from Staphylococcus aureus) recognizes the PAM sequence 5′-NNGRR (N)-3′ in the target. Thus, the locations in the genome that can be targeted by different Cas proteins are limited by the locations of unique PAM sequences.

Disclosed herein Cas9 proteins engineered from Streptococcus constellatus and Ezakiella peruensis and Peptoniphilus sp. Marseille-P 3761 species recognize the consensus PAM sequence 5′-NGG-3′. Disclosed herein Cas9 proteins engineered from Streptococcus constellatus and Ezakiella peruensis and Peptoniphilus sp. Marseille-P3761 species recognize the consensus PAM sequence 5′-NGG-3′. In some embodiments, Cas9 proteins disclosed herein are engineered to recognize the consensus PAM sequence 5′-NGC-3′. Exemplary embodiments are described below and should be nonlimiting. In some embodiments, Cas9 proteins from Streptococcus constellatus are engineered to recognize the consensus PAM sequence 5′-NGC-3′. In some embodiments, the NGC PAM variant includes one or more amino acid substitutions selected from or corresponding to D1117M, S118Q, E1201F, A1299R, D1309A, R1312E, and T1314R (collectively termed “MQFRAER”) with reference to ScoCas9 (SEQ ID NO: 1). In some embodiments, the NGC PAM variant includes one or more amino acid substitutions selected from or corresponding to D1135M, S1136Q, G1218K, E1219F, A1322R, D1332A, R1335E, and T1337R (collectively termed “MQKFRAER”) with reference to a naturally occurring SpyCas9 (SEQ ID NO: 173). In some embodiments, similar or corresponding amino acid substitutions can be made to SirCas9, VapCas9, EpeCas9, LfeCas9, or PmaCas9.

Streptococcus pyogenes Cas9 (SpyCas9; GenBank: QSG91308.1)
(SEQ ID NO: 173)
MDKKYSIGLDIGTNSVGWAVITDDYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNEDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLEKTNRK
VTVKQLKEDYFKKIECFDSVEISGVEDRENASLGTYHDLLKIIKDKDELDNEENEDILEDIV
LTLTLFEDREMIEERLKTYAHLEDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDERKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYEDTTIDRKRYTSTKEVLDATLIHQSITGLYETRIDLSQ
LGGD.

In some embodiments, the Cas9 protein described herein does not bind or exhibit activity with any other PAM sequences.

### RNA Guides

An RNA guide comprises a polynucleotide sequence with complementarity to a target sequence. The RNA guide hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the RNA guides are about 5, 10, 11, 12, 13, 14, 15, 16, 17, 18, 19, 20, 21, 22, 23, 24, 25, 26, 27, 28, 29, 30, 35, 40, 45, 50, 75 or more nucleotides in length. In some embodiments, the RNA guides are about 18-24 nucleotides in length. In some embodiments, the RNA guide is complementary to about 18-24 nucleotides in the target nucleic acid sequence. For example, the RNA guide is complementary to about 18, 19, 20, 21, 22, 23, or 24 nucleotides in the target nucleic acid sequence. In some embodiments, the RNA guide is complementary to about 18-22 nucleotides. In some embodiments, the RNA guide is complementary to about 18-21 nucleotides. In some embodiments, the RNA guide is complementary to about 18-20 nucleotides. In some embodiments, the RNA guide is complementary to 20 nucleotides in the target nucleic acid sequence.

An RNA guide can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, an RNA guide is targeted to a unique target sequence within the genome of a cell. In some embodiments, an RNA guide is designed to lack a PAM sequence. In some embodiments, an RNA guide sequence is designed to have optimal secondary structure using a folding algorithm including mFold or Geneious. In some embodiments, expression of RNA guides may be under an inducible promoter, e.g. hormone inducible, tetracycline or doxycycline inducible, arabinose inducible, or light inducible.

In some embodiments, the CRISPR system includes one or more RNA guides e.g. crRNA, tracrRNA, and/or sgRNA. Accordingly, in some embodiments the RNA guide comprises a crRNA. In some embodiments, the RNA guide comprises a tracrRNA. In some embodiments, the RNA guide comprises a sgRNA. In some embodiments, the CRISPR system includes multiple RNA guides, comprising 1, 2, 3, 4, 5, 6, 7, 8, 9, 10, 15 or more RNA guides.

In some embodiments, the RNA guide includes a crRNA. In some embodiments, the CRISPR system includes multiple crRNAs comprising 2-15 crRNAs. In some embodiments, the crRNA is a precursor crRNA (pre-crRNA), which includes a direct repeat sequence, a spacer sequence and a direct repeat sequence. In some embodiments, the crRNA is a processed or mature crRNA which includes a truncated direct repeat sequence.

In some embodiments, a CRISPR associated protein cleaves the pre-crRNA to form processed or mature crRNA.

In some embodiments, a CRISPR associated protein forms a complex with the mature crRNA and the spacer sequence targets the complex to a complementary sequence in the target nucleic acid. In some embodiments, an RNA guide comprises a direct repeat sequence and a spacer sequence capable of hybridizing under appropriate conditions to a target nucleic acid.

In some embodiments, the spacer length of crRNAs can range from about 15 to 50 nucleotides. In some embodiments, the spacer length of an RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer.

In some embodiments, the RNA guide comprises a direct repeat (DR) sequence of between about 16 and 26 nucleotides long. For example, in some embodiments, the DR is about 16 nucleotides long. In some embodiments, the DR is about 17 nucleotides long. In some embodiments, the DR is about 18 nucleotides long. In some embodiments, the DR is about 19 nucleotides long. In some embodiments, the DR is about 20 nucleotides long. In some embodiments, the DR is about 21 nucleotides long. In some embodiments, the DR is about 22 nucleotides long. In some embodiments, the DR is about 23 nucleotides long. In some embodiments, the DR is about 24 nucleotides long. In some embodiments, the DR is about 25 nucleotides long. In some embodiments, the DR is about 26 nucleotides long.

In some embodiments, the crRNA comprises a nucleotide guide sequence and a DR sequence. The nucleotide guide sequence can be between about 18 and 24 nucleotides long. Accordingly, in some embodiments, the nucleotide guide sequence is about 18 nucleotides long. In some embodiments, the nucleotide guide sequence is about 19 nucleotides long. In some embodiments, the nucleotide guide sequence is about 20 nucleotides long. In some embodiments, the nucleotide guide sequence is about 21 nucleotides long. In some embodiments, the nucleotide guide sequence is about 22 nucleotides long. In some embodiments, the crRNA comprises a nucleotide guide sequence of about 22 nucleotides long and a direct repeat of about 22 nucleotides long.

In some embodiments, the crRNA sequences can be modified to “dead crRNAs,” “dead guides,” or “dead guide sequences” that can form a complex with a CRISPR-associated protein and bind specific targets without any substantial nuclease activity.

In some embodiments, the crRNA may be chemically modified in the sugar phosphate backbone or base. In some embodiments, the crRNA maybe modified using 2′O-methyl, 2′-F or locked nucleic acids to improve nuclease resistance or base pairing. In some embodiments, the crRNA may contain modified bases such as 2-thiouridiene or N6-methyladenosine.

In some embodiments, the crRNA is conjugated with other oligonucleotides, peptides, proteins, tags, dyes, or polyethylene glycol.

In some embodiments, the crRNA may include aptamer or riboswitch sequences that can bind specific target molecules due to their three-dimensional structure.

In some embodiments, a trans-activating RNA (tracrRNA) is associated with crRNA to facilitate formation of a complex with Cas9 protein. In some embodiments, the tracrRNA sequence is about or more than about 5, 6, 7, 8, 9, 10, 11, 12, 13, 14, 15, 16, 17, 18, 19, 20, 25, 30, 40, 50, 60, 70, 80, 90, 100 or more nucleotides in length. In some embodiments, the tracrRNA is about 70 nucleotides in length.

In some embodiments, the tracrRNA and crRNA are contained in a single transcript called single guide RNA (sgRNA). In some embodiments, the sgRNA includes a loop between the tracrRNA and sgRNA.

In some embodiments, the loop forming sequences are 3, 4, 5 or more nucleotides in length. In some embodiments, the loop has the sequence GAAA, AAAG, CAAA, AAAC, UUUU, UUAUAU, UUA, UUU and/or AAUCA. In some embodiments, the loop has the sequence GAAA. In some embodiments, the loop has the sequence AAAG. In some embodiments, the loop has the sequence CAAA. In some embodiments, the loop has the sequence AAAC. In some embodiments, the loop has the sequence AAUCA. In some embodiments, the loop has the sequence UUUU. In some embodiments, the loop has the sequence UUAUAU. In some embodiments, the loop has the sequence UUA. In some embodiments, the loop has the sequence UUU. In some embodiments, the loop has the sequence AAUCA.

In some embodiments, the tracrRNA and crRNA form a hairpin loop. In some embodiments, sgRNA has at least two or more hairpins. In some embodiments, sgRNA has two, three, four or five hairpins.

In some embodiments, sgRNA includes a transcription termination sequence, which includes a polyT sequences comprising six nucleotides.

In some embodiments, the sgRNA comprises a sequence having at least 80% identity to

(SEQ ID NO: 3)
5′-GUUUUAGAGCUGUGCUGUUUAAACAACACAGCAAGUUAAAAUAAGG
CUUUGUCCGUACUC for ScoCas9,
(SEQ ID NO: 7)
5′-GUUUUAGAGUUGUGUUAUUGAAAAAUAACACAACGAGUUAAAAUAA
AGCUUAUGCUUAAAUGCCAGCUUUGCUGGUGUCAUUUAGAUGACUUUAC
UAAGGUUGCUUCGGCAACCUUUUU-3′ for SirCas9,
(SEQ ID NO: 13)
5′-GUUUGAGAGUAGUGUGAAAACAUUACGAGUUCAAAUACAAAUUAAU
UUACAAUGCCUUCGGGCUGCCCGACGUAGGGCACCUACUCUCAAUUCUU
CGGAAUUGAGUU-3′ for VapCas9,
(SEQ ID NO: 19)
5′-GUUUGAGAGUUAUGUAAUUGAAAAAUUACAUGACGAGUUCAAAUAA
AAAUUUAUUCAAACCGCCUAUUUAUAGGCCGCAGAUGUUCUGCAUUAUG
CUUGCUAUUGCAAGCUU-3′ for EpeCas9,
(SEQ ID NO: 95)
5′-GUCUUGGAUGAGUGUGAAAACACUCAUAGUCAAGAUCAAACGAGUG
GUUUUCCACGAGUUAUUACUUUUGAGGUCUUAUAUGGCCCAUACAUAAA
AAGGAGUCGGAAUUUCCGGCUCCUUUUCUU-3′ for LfeCas9,
and
(SEQ ID NO: 96)
5′-GUUUUAGAGCCAUGUAGAAAUACAUUGCAAGUUAAAAUAAGGCUUU
GUCCGUAAUCAACUUGAAAAAGUGGCGCUGUUUCGGCGCUUU-3′ for
PmaCas9.

The guide RNA is added to the 5′ end of the Cas9. In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 3, 7, 13 19, 95 or 96. In some embodiments, the sgRNA comprises a sequence identical to SEQ ID NO: 3, 7, 13, 19, 95 or 96.

In some embodiments, the tracrRNA is a separate transcript, not contained with crRNA sequence in the same transcript.

### Cas9 Fusion Proteins

In some embodiments, the Cas9 enzyme is fused to one or more heterologous protein domains. In some embodiments, the Cas9 enzyme is fused to more than about 1, 2, 3, 4, 5, 6, 7, 8, 9, 10 or more protein domains. In some embodiments, the heterologous protein domain is fused to the C-terminus of the Cas9 enzyme. In some embodiments, the heterologous protein domain is fused to the N-terminus of the Cas9 enzyme. In some embodiments, the heterologous protein domain is fused internally, between the C-terminus and the N-terminus of the Cas9 enzyme. In some embodiments, the internal fusion is made within the Cas9 RuvCI, RuvC II, RuvCIII, HNH, REC I, or PAM interacting domain.

A Cas9 protein may be directly or indirectly linked to another protein domain. In some embodiments, a suitable CRISPR system contains a linker or spacer that joins a Cas9 protein and a heterologous protein. An amino acid linker or spacer is generally designed to be flexible or to interpose a structure, such as an alpha-helix, between the two protein moieties. A linker or spacer can be relatively short, or can be longer. Typically, a linker or spacer contains for example 1-100 (e.g., 1-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 1, 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues.

In some embodiments, a Cas9 protein is fused to cellular localization signals, epitope tags, reporter genes, and protein domains with enzymatic activity, epigenetic modifying activity, RNA cleavage activity, nucleic acid binding activity, transcription modulation activity. In some embodiments, the Cas9 protein is fused to a nuclear localization sequence (NLS), a FLAG tag, a HIS tag, and/or a HA tag.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein). In some embodiments, the Cas9 protein is fused to a histone demethylase, a transcriptional activator or a deaminase.

Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In particular embodiments, a Cas9 is fused to a cytidine or adenosine deaminase domain, e.g., for use in base editing. In some embodiments, Cas9 is fused to a adenine and cytosine base editor (ACBE or CABE), wherein ACBE or CABE is generated by fusing a heterodimer of TadA and an activation-induced cytidine deaminase (AID) to the N- and C-terminals of Cas9 nickase (nCas9). In some embodiments, the ACBE or CABE simultaneously induces C-to-T and A-to-G base editing at the same target site. Xie, J et al. ACBE, a new base editor for simultaneous C-to-T and A-to-G substitutions in mammalian systems. BMC Biology (18: 131), 2020)

In some embodiments, the terms “cytidine deaminase” and “cytosine deaminase” can be used interchangeably. In certain embodiments, the cytidine deaminase domain may have sequence identity of 70%, 75%, 80%, 85%, 90%, 91%, 92%, 93%, 94%, 95%, 96%, 97%, 98%, 99% or more to any cytidine deaminase described herein. In some embodiments, the cytidine deaminase domain has cytidine deaminase activity, (e.g., converting C to U). In certain embodiments, the adenosine deaminase domain may have sequence identity of 70%, 75%, 80%, 85%, 90%, 91%, 92%, 93%, 94%, 95%, 96%, 97%, 98%, 99% or more to any adenosine deaminase described herein. In some embodiments, the adenosine deaminase domain has adenosine deaminase activity, (e.g., converting A to I). In some embodiments, the terms “adenosine deaminase” and “adenine deaminase” can be used interchangeably.

In some embodiments, a cytidine deaminase can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine or cytosine) deaminase. In some embodiments, a deaminase incorporated into a fusion protein comprises all or a portion of an APOBEC1 deaminase. In some embodiments, a deaminase incorporated into a fusion protein comprises all or a portion of APOBEC2 deaminase. In some embodiments, a deaminase incorporated into a fusion protein comprises all or a portion of is an APOBEC3 deaminase. In some embodiments, a deaminase incorporated into a fusion protein comprises all or a portion of an APOBEC3A deaminase. In some embodiments, a deaminase incorporated into a fusion protein comprises all or a portion of APOBEC3B deaminase. In some embodiments, a deaminase incorporated into a fusion protein comprises all or a portion of APOBEC3C deaminase. In some embodiments, a deaminase incorporated into a fusion protein comprises all or a portion of APOBEC3D deaminase. In some embodiments, a deaminase incorporated into a fusion protein comprises all or a portion of APOBEC3E deaminase. In some embodiments, a deaminase incorporated into a fusion protein comprises all or a portion of APOBEC3F deaminase. In some embodiments, a deaminase incorporated into a fusion protein comprises all or a portion of APOBEC3G deaminase. In some embodiments, a deaminase incorporated into a fusion protein comprises all or a portion of APOBEC3H deaminase. In some embodiments, a deaminase incorporated into a fusion protein comprises all or a portion of APOBEC4 deaminase. In some embodiments, a deaminase incorporated into a fusion protein comprises all or a portion of activation-induced deaminase (AID). In some embodiments a deaminase incorporated into a fusion protein comprises all or a portion of cytidine deaminase 1 (CDA1). It should be appreciated that a fusion protein can comprise a deaminase from any suitable organism (e.g., a human or a rat). In some embodiments, a deaminase domain of a fusion protein is from a human, chimpanzee, gorilla, monkey, cow, dog, rat, or mouse. In some embodiments, the deaminase domain of the fusion protein is derived from rat (e.g., rat APOBEC1). In some embodiments, the deaminase domain is human APOBEC1. In some embodiments, the deaminase domain is pmCDA1. Sequences of exemplary cytidine deaminases are provided below.

pmCDA1 (Petromyzon marinus)
(SEQ ID NO: 22)
MTDAEYVRIHEKLDIYTFKKQFFNNKKSVSHRCYVLFELKRRGERRACFWGYAVNK
PQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQ
LNENRWLEKTLKRAEKRRSELSIMIQVKILHTTKSPAV
Human AID:
(SEQ ID NO: 23)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKAPV
Human AID:
(SEQ ID NO: 24)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSRQLRRILLPLYEVDDLRDAFRTLGL (underline: nuclear localization sequence;
double underline: nuclear export signal)
Mouse AID:
(SEQ ID NO: 25)
MDSLLMKQKKFLYHFKNVRWAKGRHETYLCYVVKRRDSATSCSLDFGHLRNKSGC
HVELLFLRYISDWDLDPGRCYRVTWFTSWSPCYDCARHVAEFLRWNPNLSLRIFTAR
LYFCEDRKAEPEGLRRLHRAGVQIGIMTFKDYFYCWNTFVENRERTFKAWEGLHEN
SVRLTRQLRRILLPLYEVDDLRDAFRMLGE (underline: nuclear localization sequence;
double underline: nuclear export signal)
Canine AID:
(SEQ ID NO: 26)
MDSLLMKQRKFLYHFKNVRWAKGRHETYLCYVVKRRDSATSFSLDFGHLRNKSGC
HVELLFLRYISDWDLDPGRCYRVTWFTSWSPCYDCARHVADFLRGYPNLSLRIFAAR
LYFCEDRKAEPEGLRRLHRAGVQIAIMTFKDYFYCWNTFVENREKTFKAWEGLHEN
SVRLSRQLRRILLPLYEVDDLRDAFRTLGL (underline: nuclear localization sequence;
double underline: nuclear export signal)
Bovine AID:
(SEQ ID NO: 27)
MDSLLKKQRQFLYQFKNVRWAKGRHETYLCYVVKRRDSPTSFSLDFGHLRNKAGC
HVELLFLRYISDWDLDPGRCYRVTWFTSWSPCYDCARHVADFLRGYPNLSLRIFTAR
LYFCDKERKAEPEGLRRLHRAGVQIAIMTFKDYFYCWNTFVENHERTFKAWEGLHE
NSVRLSRQLRRILLPLYEVDDLRDAFRTLGL (underline: nuclear localization sequence;
double underline: nuclear export signal)
Rat AID:
(SEQ ID NO: 28)
MAVGSKPKAALVGPHWERERIWCFLCSTGLGTQQTGQTSRWLRPAATQDPVSPPRS
LLMKQRKFLYHFKNVRWAKGRHETYLCYVVKRRDSATSFSLDFGYLRNKSGCHVE
LLFLRYISDWDLDPGRCYRVTWFTSWSPCYDCARHVADFLRGNPNLSLRIFTARLTG
WGALPAGLMSPARPSDYFYCWNTFVENHERTEKAWEGLHENSVRLSRRLRRILLPL
YEVDDLRDAFRTLGL
(underline: nuclear localization sequence; double underline: nuclear export signal)
clAID (Canis lupus familiaris):
(SEQ ID NO: 29)
MDSLLMKQRKFLYHFKNVRWAKGRHETYLCYVVKRRDSATSFSLDFGHLRNKSGC
HVELLFLRYISDWDLDPGRCYRVTWFTSWSPCYDCARHVADFLRGYPNLSLRIFAAR
LYFCEDRKAEPEGLRRLHRAGVQIAIMTFKDYFYCWNTFVENREKTFKAWEGLHEN
SVRLSRQLRRILLPLYEVDDLRDAFRTLGL
btAID (Bos taurus):
(SEQ ID NO: 30)
MDSLLKKQRQFLYQFKNVRWAKGRHETYLCYVVKRRDSPTSFSLDFGHLRNKAGC
HVELLFLRYISDWDLDPGRCYRVTWFTSWSPCYDCARHVADFLRGYPNLSLRIFTAR
LYFCDKERKAEPEGLRRLHRAGVQIAIMTFKDYFYCWNTFVENHERTFKAWEGLHE
NSVRLSRQLRRILLPLYEVDDLRDAFRTLGL
mAID (Mus musculus):
(SEQ ID NO: 31)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSRQLRRILLPLYEVDDLRDAFRTLGL
rAPOBEC-1 (Rattus norvegicus):
(SEQ ID NO: 32)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
maAPOBEC-1 (Mesocricetus auratus):
(SEQ ID NO: 33)
MSSETGPVVVDPTLRRRIEPHEFDAFFDQGELRKETCLLYEIRWGGRHNIWRHTGQN
TSRHVEINFIEKFTSERYFYPSTRCSIVWFLSWSPCGECSKAITEFLSGHPNVTLFIYAA
RLYHHTDQRNRQGLRDLISRGVTIRIMTEQEYCYCWRNFVNYPPSNEVYWPRYPNL
WMRLYALELYCIHLGLPPCLKIKRRHQYPLTFFRLNLQSCHYQRIPPHILWATGFI
ppAPOBEC-1 (Pongo pygmaeus):
(SEQ ID NO: 34)
MTSEKGPSTGDPTLRRRIESWEFDVFYDPRELRKETCLLYEIKWGMSRKIWRSSGKN
TTNHVEVNFIKKFTSERRFHSSISCSITWFLSWSPCWECSQAIREFLSQHPGVTLVIYV
ARLFWHMDQRNRQGLRDLVNSGVTIQIMRASEYYHCWRNFVNYPPGDEAHWPQYP
PLWMMLYALELHCIILSLPPCLKISRRWQNHLAFFRLHLQNCHYQTIPPHILLATGLIH
PSVTWR
oCAPOBEC1 (Oryctolagus cuniculus):
(SEQ ID NO: 35)
MASEKGPSNKDYTLRRRIEPWEFEVFFDPQELRKEACLLYEIKWGASSKTWRSSGKN
TTNHVEVNFLEKLTSEGRLGPSTCCSITWFLSWSPCWECSMAIREFLSQHPGVTLIIFV
ARLFQHMDRRNRQGLKDLVTSGVTVRVMSVSEYCYCWENFVNYPPGKAAQWPRY
PPRWMLMYALELYCIILGLPPCLKISRRHQKQLTFFSLTPQYCHYKMIPPYILLATGLL
QPSVPWR
mdAPOBEC-1 (Monodelphis domestica):
(SEQ ID NO: 36)
MNSKTGPSVGDATLRRRIKPWEFVAFFNPQELRKETCLLYEIKWGNQNIWRHSNQN
TSQHAEINFMEKFTAERHFNSSVRCSITWFLSWSPCWECSKAIRKFLDHYPNVTLAIFI
SRLYWHMDQQHRQGLKELVHSGVTIQIMSYSEYHYCWRNFVDYPQGEEDYWPKYP
YLWIMLYVLELHCIILGLPPCLKISGSHSNQLALFSLDLQDCHYQKIPYNVLVATGLV
QPFVTWR
ppAPOBEC-2 (Pongo pygmaeus):
(SEQ ID NO: 37)
MAQKEEAAAATEAASQNGEDLENLDDPEKLKELIELPPFEIVTGERLPANFFKFQFRN
VEYSSGRNKTFLCYVVEAQGKGGQVQASRGYLEDEHAAAHAEEAFFNTILPAFDPA
LRYNVTWYVSSSPCAACADRIIKTLSKTKNLRLLILVGRLFMWEELEIQDALKKLKE
AGCKLRIMKPQDFEYVWQNFVEQEEGESKAFQPWEDIQENFLYYEEKLADILK
btAPOBEC-2 (Bos taurus):
(SEQ ID NO: 38)
MAQKEEAAAAAEPASQNGEEVENLEDPEKLKELIELPPFEIVTGERLPAHYFKFQFRN
VEYSSGRNKTFLCYVVEAQSKGGQVQASRGYLEDEHATNHAEEAFFNSIMPTFDPA
LRYMVTWYVSSSPCAACADRIVKTLNKTKNLRLLILVGRLFMWEEPEIQAALRKLKE
AGCRLRIMKPQDFEYIWQNFVEQEEGESKAFEPWEDIQENFLYYEEKLADILK
mAPOBEC-3-(1) (Mus musculus):
(SEQ ID NO: 39)
MQPQRLGPRAGMGPFCLGCSHRKCYSPIRNLISQETFKFHFKNLGYAKGRKDTFLCY
EVTRKDCDSPVSLHHGVFKNKDNIHAEICFLYWFHDKVLKVLSPREEFKITWYMSW
SPCFECAEQIVRFLATHHNLSLDIFSSRLYNVQDPETQQNLCRLVQEGAQVAAMDLY
EFKKCWKKFVDNGGRRFRPWKRLLTNFRYQDSKLQEILRPCYISVPSSSSSTLSNICL
TKGLPETRFWVEGRRMDPLSEEEFYSQFYNQRVKHLCYYHRMKPYLCYQLEQFNG
QAPLKGCLLSEKGKQHAEILFLDKIRSMELSQVTITCYLTWSPCPNCAWQLAAFKRD
RPDLILHIYTSRLYFHWKRPFQKGLCSLWQSGILVDVMDLPQFTDCWTNFVNPKRPF
WPWKGLEIISRRTQRRLRRIKESWGLQDLVNDFGNLQLGPPMS
Mouse APOBEC-3-(2):
(SEQ ID NO: 40)
MGPFCLGCSHRKCYSPIRNLISQETFKFHFKNLGYAKGRKDTFLCYEVTRKDCDSPV
SLHHGVFKNKDNIHAEICFLYWFHDKVLKVLSPREEFKITWYMSWSPCFECAEQIVRFL
ATHHNLSLDIFSSRLYNVQDPETQQNLCRLVQEGAQVAAMDLYEFKKCWKKFVDN
GGRRFRPWKRLLTNFRYQDSKLQEILRPCYIPVPSSSSSTLSNICLTKGLPETRFCVEG
RRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHW
KRPFQKGLCSLWQSGILVDVMDLPQFTDCWTNFVNPKRPFWPWKGLEIISRRTQRRL
RRIKESWGLQDLVNDFGNLQLGPPMS (italic: nucleic acid editing domain)
Rat APOBEC-3:
(SEQ ID NO: 41)
MGPFCLGCSHRKCYSPIRNLISQETFKFHFKNRLRYAIDRKDTFLCYEVTRKDCDSPV
SLHHGVFKNKDNIHAEICFLYWFHDKVLKVLSPREEFKITWYMSWSPCFECAEQVLRFL
ATHHNLSLDIFSSRLYNIRDPENQQNLCRLVQEGAQVAAMDLYEFKKCWKKFVDNG
GRRFRPWKKLLTNFRYQDSKLQEILRPCYIPVPSSSSSTLSNICLTKGLPETRFCVERR
RVHLLSEEEFYSQFYNQRVKHLCYYHGVKPYLCYQLEQFNGQAPLKGCLLSEKGKQ
HAEILFLDKIRSMELSQVIITCYLTWSPCPNCAWQLAAFKRDRPDLILHIYTSRLYFHWK
RPFQKGLCSLWQSGILVDVMDLPQFTDCWTNFVNPKRPFWPWKGLEIISRRTQRRLH
RIKESWGLQDLVNDFGNLQLGPPMS (italic: nucleic acid editing domain)
hAPOBEC-3A (Homo sapiens):
(SEQ ID NO: 42)
MEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAF
LQENTHVRLRIFAARIYDYDPLYKEALQMLRDAGAQVSIMTYDEFKHCWDTFVDHQ
GCPFQPWDGLDEHSQALSGRLRAILQNQGN
hAPOBEC-3F (Homo sapiens):
(SEQ ID NO: 43)
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKLAEFLAEHPNV
TLTISAARLYYYWERDYRRALCRLSQAGARVKIMDDEEFAYCWENFVYSEGQPFMP
WYKFDDNYAFLHRTLKEILRNPMEAMYPHIFYFHFKNLRKAYGRNESWLCFTMEV
VKHHSPVSWKRGVFRNQVDPETHCHAERCFLSWFCDDILSPNTNYEVTWYTSWSPC
PECAGEVAEFLARHSNVNLTIFTARLYYFWDTDYQEGLRSLSQEGASVEIMGYKDFK
YCWENFVYNDDEPFKPWKGLKYNFLFLDSKLQEILE
Rhesus macaque APOBEC-3G:
(SEQ ID NO: 44)
MVEPMDPRTFVSNFNNRPILSGLNTVWLCCEVKTKDPSGPPLDAKIFQGKVYSKAKY
HPEMRFLRWFHKWRQLHHDQEYKVTWYVSWSPCTRCANSVATFLAKDPKVTLTIF
VARLYYFWKPDYQQALRILCQKRGGPHATMKIMNYNEFQDCWNKFVDGRGKPFKP
RNNLPKHYTLLQATLGELLRHLMDPGTFTSNFNNKPWVSGQHETYLCYKVERLHND
TWVPLNQHRGFLRNQAPNIHGFPKGRHAELCFLDLIPFWKLDGQQYRVTCFTSWSPC
FSCAQEMAKFISNNEHVSLCIFAARIYDDQGRYQEGLRALHRDGAKIAMMNYSEFEY
CWDTFVDRQGRPFQPWDGLDEHSQALSGRLRAI (italic: nucleic acid editing domain;
underline: cytoplasmic localization signal
Chimpanzee APOBEC-3G:
(SEQ ID NO: 45)
MKPHFRNPVERMYQDTFSDNFYNRPILSHRNTVWLCYEVKTKGPSRPPLDAKIFRGQ
VYSKLKYHPEMRFFHWFSKWRKLHRDQEYEVTWYISWSPCTKCTRDVATFLAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTSNFNNELWVRGRHETYLCYEVERL
HNDTWVLLNQRRGFLCNQAPHKHGFLEGRHAELCFLDVIPFWKLDLHQDYRVTCFTS
WSPCFSCAQEMAKFISNNKHVSLCIFAARIYDDQGRCQEGLRTLAKAGAKISIMTYSE
FKHCWDTFVDHQGCPFQPWDGLEEHSQALSGRLRAILQNQGN
(italic: nucleic acid editing domain; underline: cytoplasmic localization signal)
Green monkey APOBEC-3G:
(SEQ ID NO: 46)
MNPQIRNMVEQMEPDIFVYYFNNRPILSGRNTVWLCYEVKTKDPSGPPLDANIFQGK
LYPEAKDHPEMKFLHWFRKWRQLHRDQEYEVTWYVSWSPCTRCANSVATFLAEDPKV
TLTIFVARLYYFWKPDYQQALRILCQERGGPHATMKIMNYNEFQHCWNEFVDGQG
KPFKPRKNLPKHYTLLHATLGELLRHVMDPGTFTSNFNNKPWVSGQRETYLCYKVE
RSHNDTWVLLNQHRGFLRNQAPDRHGFPKGRHAELCFLDLIPFWKLDDQQYRVTCFT
SWSPCFSCAQKMAKFISNNKHVSLCIFAARIYDDQGRCQEGLRTLHRDGAKIAVMNY
SEFEYCWDTFVDRQGRPFQPWDGLDEHSQALSGRLRAI
(italic: nucleic acid editing domain; underline: cytoplasmic localization signal)
Human APOBEC-3G:
(SEQ ID NO: 47)
MKPHFRNTVERMYRDTFSYNFYNRPILSRRNTVWLCYEVKTKGPSRPPLDAKIFRGQ
VYSELKYHPEMRFFHWFSKWRKLHRDQEYEVTWYISWSPCTKCTRDMATFLAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTFNFNNEPWVRGRHETYLCYEVERM
HNDTWVLLNQRRGFLCNQAPHKHGFLEGRHAELCFLDVIPFWKLDLDQDYRVTCFTS
WSPCFSCAQEMAKFISKNKHVSLCIFTARIYDDQGRCQEGLRTLAEAGAKISIMTYSE
FKHCWDTFVDHQGCPFQPWDGLDEHSQDLSGRLRAILQNQEN
(italic: nucleic acid editing domain; underline: cytoplasmic localization signal)
Human APOBEC-3F:
(SEQ ID NO: 48)
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKLAEFLAEHPNVTL
TISAARLYYYWERDYRRALCRLSQAGARVKIMDDEEFAYCWENFVYSEGQPFMPW
YKFDDNYAFLHRTLKEILRNPMEAMYPHIFYFHFKNLRKAYGRNESWLCFTMEVVK
HHSPVSWKRGVFRNQVDPETHCHAERCFLSWFCDDILSPNTNYEVTWYTSWSPCPECA
GEVAEFLARHSNVNLTIFTARLYYFWDTDYQEGLRSLSQEGASVEIMGYKDFKYCW
ENFVYNDDEPFKPWKGLKYNFLFLDSKLQEILE
(italic: nucleic acid editing domain)
Human APOBEC-3B:
(SEQ ID NO: 49)
MNPQIRNPMERMYRDTFYDNFENEPILYGRSYTWLCYEVKIKRGRSNLLWDTGVFR
GQVYFKPQYHAEMCFLSWFCGNQLPAYKCFQITWFVSWTPCPDCVAKLAEFLSEHPN
VTLTISAARLYYYWERDYRRALCRLSQAGARVTIMDYEEFAYCWENFVYNEGQQF
MPWYKFDENYAFLHRTLKEILRYLMDPDTFTFNFNNDPLVLRRRQTYLCYEVERLD
NGTWVLMDQHMGFLCNEAKNLLCGFYGRHAELRFLDLVPSLQLDPAQIYRVTWFISWS
PCFSWGCAGEVRAFLQENTHVRLRIFAARIYDYDPLYKEALQMLRDAGAQVSIMTY
DEFEYCWDTFVYRQGCPFQPWDGLEEHSQALSGRLRAILQNQGN
(italic: nucleic acid editing domain)
Rat APOBEC-3B:
(SEQ ID NO: 50)
MQPQGLGPNAGMGPVCLGCSHRRPYSPIRNPLKKLYQQTFYFHFKNVRYAWGRKN
NFLCYEVNGMDCALPVPLRQGVFRKQGHIHAELCFIYWFHDKVLRVLSPMEEFKVT
WYMSWSPCSKCAEQVARFLAAHRNLSLAIFSSRLYYYLRNPNYQQKLCRLIQEGVH
VAAMDLPEFKKCWNKFVDNDGQPFRPWMRLRINFSFYDCKLQEIFSRMNLLREDVF
YLQFNNSHRVKPVQNRYYRRKSYLCYQLERANGQEPLKGYLLYKKGEQHVEILFLE
KMRSMELSQVRITCYLTWSPCPNCARQLAAFKKDHPDLILRIYTSRLYFWRKKFQKG
LCTLWRSGIHVDVMDLPQFADCWTNFVNPQRPFRPWNELEKNSWRIQRRLRRIKES
WGL
Bovine APOBEC-3B:
(SEQ ID NO: 51)
MDGWEVAFRSGTVLKAGVLGVSMTEGWAGSGHPGQGACVWTPGTRNTMNLLREV
LFKQQFGNQPRVPAPYYRRKTYLCYQLKQRNDLTLDRGCFRNKKQRHAERFIDKIN
SLDLNPSQSYKIICYITWSPCPNCANELVNFITRNNHLKLEIFASRLYFHWIKSFKMGL
QDLQNAGISVAVMTHTEFEDCWEQFVDNQSRPFQPWDKLEQYSASIRRRLQRILTAP
I
Chimpanzee APOBEC-3B:
(SEQ ID NO: 52)
MNPQIRNPMEWMYQRTFYYNFENEPILYGRSYTWLCYEVKIRRGHSNLLWDTGVFR
GQMYSQPEHHAEMCFLSWFCGNQLSAYKCFQITWFVSWTPCPDCVAKLAKFLAEH
PNVTLTISAARLYYYWERDYRRALCRLSQAGARVKIMDDEEFAYCWENFVYNEGQP
FMPWYKFDDNYAFLHRTLKEIIRHLMDPDTFTFNFNNDPLVLRRHQTYLCYEVERLD
NGTWVLMDQHMGFLCNEAKNLLCGFYGRHAELRFLDLVPSLQLDPAQIYRVTWFIS
WSPCFSWGCAGQVRAFLQENTHVRLRIFAARIYDYDPLYKEALQMLRDAGAQVSIM
TYDEFEYCWDTFVYRQGCPFQPWDGLEEHSQALSGRLRAILQVRASSLCMVPHRPPP
PPQSPGPCLPLCSEPPLGSLLPTGRPAPSLPFLLTASFSFPPPASLPPLPSLSLSPGHLPVP
SFHSLTSCSIQPPCSSRIRETEGWASVSKEGRDLG
Human APOBEC-3C:
(SEQ ID NO: 53)
MNPQIRNPMKAMYPGTFYFQFKNLWEANDRNETWLCFTVEGIKRRSVVSWKTGVF
RNQVDSETHCHAERCFLSWFCDDILSPNTKYQVTWYTSWSPCPDCAGEVAEFLARHSN
VNLTIFTARLYYFQYPCYQEGLRSLSQEGVAVEIMDYEDFKYCWENFVYNDNEPFKP
WKGLKTNFRLLKRRLRESLQ
(italic: nucleic acid editing domain)
Gorilla APOBEC-3C
(SEQ ID NO: 54)
MNPQIRNPMKAMYPGTFYFQFKNLWEANDRNETWLCFTVEGIKRRSVVSWKTGVF
RNQVDSETHCHAERCFLSWECDDILSPNTNYQVTWYTSWSPCPECAGEVAEFLARHSN
VNLTIFTARLYYFQDTDYQEGLRSLSQEGVAVKIMDYKDFKYCWENFVYNDDEPFK
PWKGLKYNFRFLKRRLQEILE
(italic: nucleic acid editing domain)
Human APOBEC-3A:
(SEQ ID NO: 55)
MEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAFLQ
ENTHVRLRIFAARIYDYDPLYKEALQMLRDAGAQVSIMTYDEFKHCWDTFVDHQGC
PFQPWDGLDEHSQALSGRLRAILQNQGN
(italic: nucleic acid editing domain)
Rhesus macaque APOBEC-3A:
(SEQ ID NO: 56)
MDGSPASRPRHLMDPNTFTFNFNNDLSVRGRHQTYLCYEVERLDNGTWVPMDERR
GFLCNKAKNVPCGDYGCHVELRFLCEVPSWQLDPAQTYRVTWFISWSPCFRRGCAGQ
VRVFLQENKHVRLRIFAARIYDYDPLYQEALRTLRDAGAQVSIMTYEEFKHCWDTF
VDRQGRPFQPWDGLDEHSQALSGRLRAILQNQGN
(italic: nucleic acid editing domain)
Bovine APOBEC-3A:
(SEQ ID NO: 57)
MDEYTFTENFNNQGWPSKTYLCYEMERLDGDATIPLDEYKGFVRNKGLDQPEKPCH
AELYFLGKIHSWNLDRNQHYRLTCFISWSPCYDCAQKLTTFLKENHHISLHILASRIYTH
NRFGCHQSGLCELQAAGARITIMTFEDFKHCWETFVDHKGKPFQPWEGLNVKSQAL
CTELQAILKTQQN
(italic: nucleic acid editing domain)
Human APOBEC-3H:
(SEQ ID NO: 58)
MALLTAETFRLQFNNKRRLRRPYYPRKALLCYQLTPQNGSTPTRGYFENKKKCHAEI
CFINEIKSMGLDETQCYQVTCYLTWSPCSSCAWELVDFIKAHDHLNLGIFASRLYYHWC
KPQQKGLRLLCGSQVPVEVMGFPKFADCWENFVDHEKPLSFNPYKMLEELDKNSRA
IKRRLERIKIPGVRAQGRYMDILCDAEV
(italic: nucleic acid editing domain)
Rhesus macaque APOBEC-3H:
(SEQ ID NO: 59)
MALLTAKTFSLQFNNKRRVNKPYYPRKALLCYQLTPQNGSTPTRGHLKNKKKDHAE
IRFINKIKSMGLDETQCYQVTCYLTWSPCPSCAGELVDFIKAHRHLNLRIFASRLYYH
WRPNYQEGLLLLCGSQVPVEVMGLPEFTDCWENFVDHKEPPSFNPSEKLEELDKNS
QAIKRRLERIKSRSVDVLENGLRSLQLGPVTPSSSIRNSR
Human APOBEC-3D:
(SEQ ID NO: 60)
MNPQIRNPMERMYRDTFYDNFENEPILYGRSYTWLCYEVKIKRGRSNLLWDTGVFR
GPVLPKRQSNHRQEVYFRFENHAEMCFLSWFCGNRLPANRRFQITWFVSWNPCLPCVV
KVTKFLAEHPNVTLTISAARLYYYRDRDWRWVLLRLHKAGARVKIMDYEDFAYCW
ENFVCNEGQPFMPWYKFDDNYASLHRTLKEILRNPMEAMYPHIFYFHFKNLLKACG
RNESWLCFTMEVTKHHSAVFRKRGVFRNQVDPETHCHAERCFLSWFCDDILSPNTNY
EVTWYTSWSPCPECAGEVAEFLARHSNVNLTIFTARLCYFWDTDYQEGLCSLSQEGAS
VKIMGYKDFVSCWKNFVYSDDEPFKPWKGLQTNFRLLKRRLREILQ
(italic: nucleic acid editing domain)
Human APOBEC-1:
(SEQ ID NO: 61)
MTSEKGPSTGDPTLRRRIEPWEFDVFYDPRELRKEACLLYEIKWGMSRKIWRSSGKN
TTNHVEVNFIKKFTSERDFHPSMSCSITWFLSWSPCWECSQAIREFLSRHPGVTLVIYV
ARLFWHMDQQNRQGLRDLVNSGVTIQIMRASEYYHCWRNFVNYPPGDEAHWPQY
PPLWMMLYALELHCIILSLPPCLKISRRWQNHLTFFRLHLQNCHYQTIPPHILLATGLI
HPSVAWR
Mouse APOBEC-1:
(SEQ ID NO: 62)
MSSETGPVAVDPTLRRRIEPHEFEVFFDPRELRKETCLLYEINWGGRHSVWRHTSQN
TSNHVEVNFLEKFTTERYFRPNTRCSITWFLSWSPCGECSRAITEFLSRHPYVTLFIYIA
RLYHHTDQRNRQGLRDLISSGVTIQIMTEQEYCYCWRNFVNYPPSNEAYWPRYPHL
WVKLYVLELYCIILGLPPCLKILRRKQPQLTFFTITLQTCHYQRIPPHLLWATGLK
Rat APOBEC-1:
(SEQ ID NO: 63)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
Human APOBEC-2:
(SEQ ID NO: 64)
MAQKEEAAVATEAASQNGEDLENLDDPEKLKELIELPPFEIVTGERLPANFFKFQFRN
VEYSSGRNKTFLCYVVEAQGKGGQVQASRGYLEDEHAAAHAEEAFFNTILPAFDPA
LRYNVTWYVSSSPCAACADRIIKTLSKTKNLRLLILVGRLFMWEEPEIQAALKKLKE
AGCKLRIMKPQDFEYVWQNFVEQEEGESKAFQPWEDIQENFLYYEEKLADILK
Mouse APOBEC-2:
(SEQ ID NO: 65)
MAQKEEAAEAAAPASQNGDDLENLEDPEKLKELIDLPPFEIVTGVRLPVNFFKFQFR
NVEYSSGRNKTFLCYVVEVQSKGGQAQATQGYLEDEHAGAHAEEAFFNTILPAFDP
ALKYNVTWYVSSSPCAACADRILKTLSKTKNLRLLILVSRLFMWEEPEVQAALKKLK
EAGCKLRIMKPQDFEYIWQNFVEQEEGESKAFEPWEDIQENFLYYEEKLADILK
Rat APOBEC-2:
(SEQ ID NO: 66)
MAQKEEAAEAAAPASQNGDDLENLEDPEKLKELIDLPPFEIVTGVRLPVNFFKFQFR
NVEYSSGRNKTFLCYVVEAQSKGGQVQATQGYLEDEHAGAHAEEAFFNTILPAFDP
ALKYNVTWYVSSSPCAACADRILKTLSKTKNLRLLILVSRLFMWEEPEVQAALKKLK
EAGCKLRIMKPQDFEYLWQNFVEQEEGESKAFEPWEDIQENFLYYEEKLADILK
Bovine APOBEC-2:
(SEQ ID NO: 67)
MAQKEEAAAAAEPASQNGEEVENLEDPEKLKELIELPPFEIVTGERLPAHYFKFQFRN
VEYSSGRNKTFLCYVVEAQSKGGQVQASRGYLEDEHATNHAEEAFFNSIMPTFDPA
LRYMVTWYVSSSPCAACADRIVKTLNKTKNLRLLILVGRLFMWEEPEIQAALRKLKE
AGCRLRIMKPQDFEYIWQNFVEQEEGESKAFEPWEDIQENFLYYEEKLADILK
Petromyzon marinus CDA1 (pmCDA1):
(SEQ ID NO: 68)
MTDAEYVRIHEKLDIYTFKKQFFNNKKSVSHRCYVLFELKRRGERRACFWGYAVNK
PQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQ
LNENRWLEKTLKRAEKRRSELSFMIQVKILHTTKSPAV
Human APOBEC3G D316R D317R:
(SEQ ID NO: 69)
MKPHFRNTVERMYRDTFSYNFYNRPILSRRNTVWLCYEVKTKGPSRPPLDAKIFRGQ
VYSELKYHPEMRFFHWFSKWRKLHRDQEYEVTWYISWSPCTKCTRDMATFLAEDP
KVTLTIFVARLYYFWDPDYQEALRSLCQKRDGPRATMKFNYDEFQHCWSKFVYSQ
RELFEPWNNLPKYYILLHFMLGEILRHSMDPPTFTFNFNNEPWVRGRHETYLCYEVE
RMHNDTWVLLNQRRGFLCNQAPHKHGFLEGRHAELCFLDVIPFWKLDLDQDYRVT
CFTSWSPCFSCAQEMAKFISKKHVSLCIFTARIYRRQGRCQEGLRTLAEAGAKISFTY
SEFKHCWDTFVDHQGCPFQPWDGLDEHSQDLSGRLRAILQNQEN
Human APOBEC3G chain A:
(SEQ ID NO: 70)
MDPPTFTFNFNNEPWWGRHETYLCYEVERMHNDTWVLLNQRRGFLCNQAPHKHGF
LEGRHAELCFLDVIPFWKLDLDQDYRVTCFTSWSPCFSCAQEMAKFISKNKHVSLCIF
TARIYDDQGRCQEGLRTLAEAGAKISFTYSEFKHCWDTFVDHQGCPFQPWDGLD
EHSQDLSGRLRAILQ
Human APOBEC3G chain A D120R D121R:
(SEQ ID NO: 71)
MDPPTFTFNFNNEPWVRGRHETYLCYEVERMHNDTWVLLNQRRGFLCNQAPHKHG
FLEGRHAELCFLDVIPFWKLDLDQDYRVTCFTSWSPCFSCAQEMAKFISKNKHVSLCI
FTARIYRRQGRCQEGLRTLAEAGAKISFMTYSEFKHCWDTFVDHQGCPFQPWDGLD
EHSQDLSGRLRAILQ
hAPOBEC-4 (Homo sapiens):
(SEQ ID NO: 72)
MEPIYEEYLANHGTIVKPYYWLSFSLDCSNCPYHIRTGEEARVSLTEFCQIFGFPYGTT
FPQTKHLTFYELKTSSGSLVQKGHASSCTGNYIHPESMLFEMNGYLDSAIYNNDSIRH
IILYSNNSPCNEANHCCISKMYNFLITYPGITLSIYFSQLYHTEMDFPASAWNREALRS
LASLWPRVVLSPISGGIWHSVLHSFISGVSGSHVFQPILTGRALADRHNAYEINAITGV
KPYFTDVLLQTKRNPNTKAQEALESYPLNNAFPGQFFQMPSGQLQPNLPPDLRAPVV
FVLVPLRDLPPMHMGQNPNKPRNIVRHLNMPQMSFQETKDLGRLPTGRSVEIVEITE
QFASSKEADEKKKKKGKK
mAPOBEC-4 (Mus musculus):
(SEQ ID NO: 73)
MDSLLMKQKKFLYHFKNVRWAKGRHETYLCYVVKRRDSATSCSLDFGHLRNKSGC
HVELLFLRYISDWDLDPGRCYRVTWFTSWSPCYDCARHVAEFLRWNPNLSLRIFTAR
LYFCEDRKAEPEGLRRLHRAGVQIGIMTFKDYFYCWNTFVENRERTFKAWEGLHEN
SVRLTRQLRRILLPLYEVDDLRDAFRMLGF
rAPOBEC-4 (Rattus norvegicus):
(SEQ ID NO: 74)
MEPLYEEYLTHSGTIVKPYYWLSVSLNCTNCPYHIRTGEEARVPYTEFHQTFGFPWST
YPQTKHLTFYELRSSSGNLIQKGLASNCTGSHTHPESMLFERDGYLDSLIFHDSNIRHI
ILYSNNSPCDEANHCCISKMYNFLMNYPEVTLSVFFSQLYHTENQFPTSAWNREALR
GLASLWPQVTLSAISGGIWQSILETFVSGISEGLTAVRPFTAGRTLTDRYNAYEINCIT
EVKPYFTDALHSWQKENQDQKVWAASENQPLHNTTPAQWQPDMSQDCRTPAVFM
LVPYRDLPPIHVNPSPQKPRTVVRHLNTLQLSASKVKALRKSPSGRPVKKEEARKGS
TRSQEANETNKSKWKKQTLFIKSNICHLLEREQKKIGILSSWSV
mfAPOBEC-4 (Macaca fascicularis):
(SEQ ID NO: 75)
MEPTYEEYLANHGTIVKPYYWLSFSLDCSNCPYHIRTGEEARVSLTEFCQIFGFPYGT
TYPQTKHLTFYELKTSSGSLVQKGHASSCTGNYIHPESMLFEMNGYLDSAIYNNDSIR
HIILYCNNSPCNEANHCCISKVYNFLITYPGITLSIYFSQLYHTEMDFPASAWNREALR
SLASLWPRVVLSPISGGIWHSVLHSFVSGVSGSHVFQPILTGRALTDRYNAYEINAITG
VKPFFTDVLLHTKRNPNTKAQMALESYPLNNAFPGQSFQMTSGIPPDLRAPVVFVLL
PLRDLPPMHMGQDPNKPRNIIRHLNMPQMSFQETKDLERLPTRRSVETVEITERFASS
KQAEEKTKKKKGKK
pmCDA-1 (Petromyzon marinus):
(SEQ ID NO: 76)
MAGYECVRVSEKLDFDTFEFQFENLHYATERHRTYVIFDVKPQSAGGRSRRLWGYII
NNPNVCHAELILMSMIDRHLESNPGVYAMTWYMSWSPCANCSSKLNPWLKNLLEE
QGHTLTMHFSRIYDRDREGDHRGLRGLKHVSNSFRMGVVGRAEVKECLAEYVEAS
RRTLTWLDTTESMAAKMRRKLFCILVRCAGMRESGIPLHLFTLQTPLLSGRVVWWR
V
pmCDA-2 (Petromyzon marinus):
(SEQ ID NO: 77)
MELREVVDCALASCVRHEPLSRVAFLRCFAAPSQKPRGTVILFYVEGAGRGVTGGH
AVNYNKQGTSIHAEVLLLSAVRAALLRRRRCEDGEEATRGCTLHCYSTYSPCRDCVE
YIQEFGASTGVRVVIHCCRLYELDVNRRRSEAEGVLRSLSRLGRDFRLMGPRDAIAL
LLGGRLANTADGESGASGNAWVTETNVVEPLVDMTGFGDEDLHAQVQRNKQIREA
YANYASAVSLMLGELHVDPDKFPFLAEFLAQTSVEPSGTPRETRGRPRGASSRGPEIG
RQRPADFERALGAYGLFLHPRIVSREADREEIKRDLIVVMRKHNYQGP
pmCDA-5 (Petromyzon marinus):
(SEQ ID NO: 78)
MAGDENVRVSEKLDFDTFEFQFENLHYATERHRTYVIFDVKPQSAGGRSRRLWGYII
NNPNVCHAELILMSMIDRHLESNPGVYAMTWYMSWSPCANCSSKLNPWLKNLLEE
QGHTLMMHFSRIYDRDREGDHRGLRGLKHVSNSFRMGVVGRAEVKECLAEYVEAS
RRTLTWLDTTESMAAKMRRKLFCILVRCAGMRESGMPLHLFT
yCD (Saccharomyces cerevisiae):
(SEQ ID NO: 79)
MVTGGMASKWDQKGMDIAYEEAALGYKEGGVPIGGCLINNKDGSVLGRGHNMRF
QKGSATLHGEISTLENCGRLEGKVYKDTTLYTTLSPCDMCTGAIIMYGIPRCVVGEN
VNFKSKGEKYLQTRGHEVVVVDDERCKKIMKQFIDERPQDWFEDIGE
rAPOBEC-1 (delta 177-186):
(SEQ ID NO: 80)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRGLPPCLNILRRKQPQLTFFTIALQSCHYQRLPPHILWATGLK
rAPOBEC-1 (delta 202-213):
(SEQ ID NO: 81)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQHYQRLPPHILWATGLK
Mouse APOBEC-3:
(SEQ ID NO: 82)
MGPFCLGCSHRKCYSPIRNLISQETFKFHFKNLGYAKGRKDTFLCYEVTRKDCDSPV
SLHHGVFKNKDNIHAEICFLYWFHDKVLKVLSPREEFKITWYMSWSPCFECAEQIVRFL
ATHHNLSLDIFSSRLYNVQDPETQQNLCRLVQEGAQVAAMDLYEFKKCWKKFVDN
GGRRFRPWKRLLTNFRYQDSKLQEILRPCYIPVPSSSSSTLSNICLTKGLPETRFCVEG
RRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHW
KRPFQKGLCSLWQSGILVDVMDLPQFTDCWTNFVNPKRPFWPWKGLEIISRRTQRRL
RRIKESWGLQDLVNDFGNLQLGPPMS
(italic: nucleic acid editing domain)

In some embodiments, an adenosine deaminase can comprise all or a portion of an adenosine deaminase ADAR (e.g., ADAR1 or ADAR2). In another embodiment, an adenosine deaminase can comprise all or a portion of an adenosine deaminase ADAT. In some embodiments, an adenosine deaminase can comprise all or a portion of an ADAT from Escherichia coli (EcTadA) comprising one or more of the following mutations: D108N, A106V, D147Y, E155V, L84F, H123Y, I157F, or a corresponding mutation in another adenosine deaminase. The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly. In particular embodiments, the TadA is any one of the TadA described in PCT/US2017/045381 (WO 2018/027078), which is incorporated herein by reference in its entirety. Mutations were identified through rounds of evolution and selection (e.g., TadA*7.10=variant 10 from seventh round of evolution) having desirable adenosine deaminase activity on single stranded DNA as shown in Table 3.

TABLE 3
Genotypes of TadA Variants

| TadA | 23 | 26 | 36 | 37 | 48 | 49 | 51 | 72 | 84 | 87 | 105 | 108 | 123 | 125 | 142 | 145 | 147 | 152 | 155 | 156 | 157 | 16 |
|---|---|---|---|---|---|---|---|---|---|---|---|---|---|---|---|---|---|---|---|---|---|---|
| 0.1 | W | R | H | N | P | | R | N | L | S | A | D | H | G | A | S | D | R | E | I | K | K |
| 0.2 | W | R | H | N | P | | R | N | L | S | A | D | H | G | A | S | D | R | E | I | K | K |
| 1.1 | W | R | H | N | P | | R | N | L | S | A | N | H | G | A | S | Y | R | E | I | K | K |
| 1.2 | W | R | H | N | P | | R | N | L | S | V | N | H | G | A | S | Y | R | E | I | K | K |
| 2.1 | W | R | H | N | P | | R | N | L | S | V | N | H | G | A | S | Y | R | V | I | K | K |
| 2.2 | W | R | H | N | P | | R | N | L | S | V | N | H | G | A | S | Y | R | V | I | K | K |
| 2.3 | W | R | H | N | P | | R | N | L | S | V | N | H | G | A | S | Y | R | V | I | K | K |
| 2.4 | W | R | H | N | P | | R | N | L | S | V | N | H | G | A | S | Y | R | V | I | K | K |
| 2.5 | W | R | H | N | P | | R | N | L | S | V | N | H | G | A | S | Y | R | V | I | K | K |
| 2.6 | W | R | H | N | P | | R | N | L | S | V | N | H | G | A | S | Y | R | V | I | K | K |
| 2.7 | W | R | H | N | P | | R | N | L | S | V | N | H | G | A | S | Y | R | V | I | K | K |
| 2.8 | W | R | H | N | P | | R | N | L | S | V | N | H | G | A | S | Y | R | V | I | K | K |
| 2.9 | W | R | H | N | P | | R | N | L | S | V | N | H | G | A | S | Y | R | V | I | K | K |
| 2.10 | W | R | H | N | P | | R | N | L | S | V | N | H | G | A | S | Y | R | V | I | K | K |
| 2.11 | W | R | H | N | P | | R | N | L | S | V | N | H | G | A | S | Y | R | V | I | K | K |
| 2.12 | W | R | H | N | P | | R | N | L | S | V | N | H | G | A | S | Y | R | V | I | K | K |
| 3.1 | W | R | H | N | P | | R | N | F | S | V | N | Y | G | A | S | Y | R | V | F | K | K |
| 3.2 | W | R | H | N | P | | R | N | F | S | V | N | Y | G | A | S | Y | R | V | F | K | K |
| 3.3 | W | R | H | N | P | | R | N | F | S | V | N | Y | G | A | S | Y | R | V | F | K | K |
| 3.4 | W | R | H | N | P | | R | N | F | S | V | N | Y | G | A | S | Y | R | V | F | K | K |
| 3.5 | W | R | H | N | P | | R | N | F | S | V | N | Y | G | A | S | Y | R | V | F | K | K |
| 3.6 | W | R | H | N | P | | R | N | F | S | V | N | Y | G | A | S | Y | R | V | F | K | K |
| 3.7 | W | R | H | N | P | | R | N | F | S | V | N | Y | G | A | S | Y | R | V | F | K | K |
| 3.8 | W | R | H | N | P | | R | N | F | S | V | N | Y | G | A | S | Y | R | V | F | K | K |
| 4.1 | W | R | H | N | P | | R | N | L | S | V | N | H | G | N | S | Y | R | V | I | K | K |
| 4.2 | W | G | H | N | P | | R | N | L | S | V | N | H | G | N | S | Y | R | V | I | K | K |
| 4.3 | W | R | H | N | P | | R | N | F | S | V | N | Y | G | N | S | Y | R | V | F | K | K |
| 5.1 | W | R | L | N | P | | L | N | F | S | V | N | Y | G | A | C | Y | R | V | F | N | K |
| 5.2 | W | R | H | S | P | | R | N | F | S | V | N | Y | G | A | S | Y | R | V | F | K | T |
| 5.3 | W | R | L | N | P | | L | N | I | S | V | N | Y | G | A | C | Y | R | V | I | N | K |
| 5.4 | W | R | H | S | P | | R | N | F | S | V | N | Y | G | A | S | Y | R | V | F | K | T |
| 5.5 | W | R | L | N | P | | L | N | F | S | V | N | Y | G | A | C | Y | R | V | F | N | K |
| 5.6 | W | R | L | N | P | | L | N | F | S | V | N | Y | G | A | C | Y | R | V | F | N | K |
| 5.7 | W | R | L | N | P | | L | N | F | S | V | N | Y | G | A | C | Y | R | V | F | N | K |
| 5.8 | W | R | L | N | P | | L | N | F | S | V | N | Y | G | A | C | Y | R | V | F | N | K |
| 5.9 | W | R | L | N | P | | L | N | F | S | V | N | Y | G | A | C | Y | R | V | F | N | K |
| 5.10 | W | R | L | N | P | | L | N | F | S | V | N | Y | G | A | C | Y | R | V | F | N | K |
| 5.11 | W | R | L | N | P | | L | N | F | S | V | N | Y | G | A | C | Y | R | V | F | N | K |
| 5.12 | W | R | L | N | P | | L | N | F | S | V | N | Y | G | A | C | Y | R | V | F | N | K |
| 5.13 | W | R | H | N | P | | L | D | F | S | V | N | Y | A | A | S | Y | R | V | F | K | K |
| 5.14 | W | R | H | N | S | | L | N | F | C | V | N | Y | G | A | S | Y | R | V | F | K | K |
| 6.1 | W | R | H | N | S | | L | N | F | S | V | N | Y | G | N | S | Y | R | V | F | K | K |
| 6.2 | W | R | H | N | T | V | L | N | F | S | V | N | Y | G | N | S | Y | R | V | F | N | K |
| 6.3 | W | R | L | N | S | | L | N | F | S | V | N | Y | G | A | C | Y | R | V | F | N | K |
| 6.4 | W | R | L | N | S | | L | N | F | S | V | N | Y | G | N | C | Y | R | V | F | N | K |
| 6.5 | W | R | L | N | I | V | L | N | F | S | V | N | Y | G | A | C | Y | R | V | F | N | K |
| 6.6 | W | R | L | N | T | V | L | N | F | S | V | N | Y | G | N | C | Y | R | V | F | N | K |
| 7.1 | W | R | L | N | A | | L | N | F | S | V | N | Y | G | A | C | Y | R | V | F | N | K |
| 7.2 | W | R | L | N | A | | L | N | F | S | V | N | Y | G | N | C | Y | R | V | F | N | K |
| 7.3 | I | R | L | N | A | | L | N | F | S | V | N | Y | G | A | C | Y | R | V | F | N | K |
| 7.4 | R | R | L | N | A | | L | N | F | S | V | N | Y | G | A | C | Y | R | V | F | N | K |
| 7.5 | W | R | L | N | A | | L | N | F | S | V | N | Y | G | A | C | Y | H | V | F | N | K |
| 7.6 | W | R | L | N | A | | L | N | I | S | V | N | Y | G | A | C | Y | P | V | I | N | K |
| 7.7 | L | R | L | N | A | | L | N | F | S | V | N | Y | G | A | C | Y | P | V | F | N | K |
| 7.8 | I | R | L | | A | | L | N | F | S | V | N | Y | G | N | C | Y | R | V | F | N | K |
| 7.9 | L | R | L | N | A | | L | N | F | S | V | N | Y | G | N | C | Y | P | V | F | N | K |
| 7.10 | R | R | L | N | A | | L | N | F | S | V | N | Y | G | A | C | Y | P | V | F | N | K |

In some embodiments, the TadA is provided as a monomer or dimer (e.g., a heterodimer of wild-type E. coli TadA and an engineered TadA variant). In some embodiments, the adenosine deaminase is an eighth generation TadA*8 variant as shown in Table 4 below.

TABLE 4
TadA8* Adenosine Deaminase Variants

| Adenosine | Adenosine Deaminase |
|---|---|
| Deaminase | Description |
| TadA*8.1 | Monomer_TadA*7.10 + Y147T |
| TadA*8.2 | Monomer_TadA*7.10 + Y147R |
| TadA*8.3 | Monomer_TadA*7.10 + Q154S |
| TadA*8.4 | Monomer_TadA*7.10 + Y123H |
| TadA*8.5 | Monomer_TadA*7.10 + V82S |
| TadA*8.6 | Monomer_TadA*7.10 + T166R |
| TadA*8.7 | Monomer_TadA*7.10 + Q154R |
| TadA*8.8 | Monomer_TadA*7.10 + Y147R_Q154R_Y123H |
| TadA*8.9 | Monomer_TadA*7.10 + Y147R_Q154R_I76Y |
| TadA*8.10 | Monomer_TadA*7.10 + Y147R_Q154R_T166R |
| TadA*8.11 | Monomer_TadA*7.10 + Y147T_Q154R |
| TadA*8.12 | Monomer_TadA*7.10 + Y147T_Q154S |
| TadA*8.13 | Monomer_TadA*7.10 + H123H_Y147R_Q154R_I76Y |
| TadA*8.14 | Heterodimer_(WT) + (TadA*7.10 + Y147T) |
| TadA*8.15 | Heterodimer_(WT) + (TadA*7.10 + Y147R) |
| TadA*8.16 | Heterodimer_(WT) + (TadA*7.10 + Q154S) |
| TadA*8.17 | Heterodimer_(WT) + (TadA*7.10 + Y123H) |
| TadA*8.18 | Heterodimer_(WT) + (TadA*7.10 + V82S) |
| TadA*8.19 | Heterodimer_(WT) + (TadA*7.10 + T166R) |
| TadA*8.20 | Heterodimer_(WT) + (TadA*7.10 + Q154R) |
| TadA*8.21 | Heterodimer_(WT) + (TadA*7.10 + |
| | Y147R_Q154R_Y123H) |
| TadA*8.22 | Heterodimer_(WT) + (TadA*7.10 + |
| | Y147R_Q154R_176Y) |
| TadA*8.23 | Heterodimer_(WT) + (TadA*7.10 + |
| | Y147R_Q154R_T166R) |
| TadA*8.24 | Heterodimer_(WT) + (TadA*7.10 + Y147T_Q154R) |
| TadA*8.25 | Heterodimer_(WT) + (TadA*7.10 + Y147T_Q154S) |
| TadA*8.26 | Heterodimer_(WT) + (TadA*7.10 + |
| | H123H_Y147T_Q154R_I76Y) |

In some embodiments, the adenosine deaminase is a ninth generation TadA*9 variant containing an alteration at an amino acid position selected from the following: 21, 23, 25, 38, 51, 54, 70, 71, 72, 72, 94, 124, 133, 138, 139, 146, and 158 of a TadA variant as shown in the reference sequence below:

```
(SEQ ID NO: 83)
                10 20 30 40
MSEVEFSHEY WMRHALTLAK RARDEREVPV GAVLVLNNRV
                50 60 70 80
IGEGWNRAIG LHDPTAHAEI MALRQGGLVM QNYRLIDATL
                90 100 110 120
YVTFEPCVMC AGAMIHSRIG RVVFGVRNAK TGAAGSLMDV
              130 140 150 160
LHYPGMNHRV EITEGILADE CAALLCYFFR MPRQVENAQK
KAQSSTD
```

In one embodiment, the adenosine deaminase variant contains alterations at two or more amino acid positions selected from the following: 21, 23, 25, 38, 51, 54, 70, 71, 72, 94, 124, 133, 138, 139, 146, and 158 of the TadA reference sequence above. In another embodiment, the adenosine deaminase variant contains one or more (e.g., 2, 3, 4) alterations selected from the following: R21N, R23H, E25F, N38G, L51W, P54C, M70V, Q71M, N72K, Y73S, M94V, P124W, T133K, D139L, D139M, C146R, and A158K of SEQ ID NO. 1. In other embodiments, the adenosine deaminase variant further contains one or more of the following alterations: Y147T, Y147R, Q154S, Y123H, and Q154R. In still other embodiments, the adenosine deaminase variant contains a combination of alterations relative to the above TadA reference sequence selected from the following: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; M70V+V82S+M94V+Y123H+Y147R+Q154R; Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D138M+Y147R+Q154R; Y72S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D138M+Y147R+Q154R; Y72S+I76Y+V82S+Y123H+Y147R+Q154R; and V82S+Q154R; N72K V82S+Y123H+Y147R+Q154R; Q71M V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K V82S+Y123H+Y147R+Q154R; Q71M V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, Cas9 is fused to nuclear localization sequences, including an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mx1, human poly(ADP-ribose) polymerase, steroid hormone receptor (human) glucocorticoid.

In some embodiments, a Cas9 protein is fused to epitope tags including, but not limited to hemagglutinin (HA) tags, histidine (His) tags, FLAG tags, Myc tags, V5 tags, VSV-G tags, SNAP tags, thioredoxin (Trx) tags.

In some embodiments, Cas9 is fused to reporter genes including, but not limited to glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered Cas9 protein is increased by fusion with heterologous proteins such as a human serum albumin protein, transferrin protein, human IgG and/or sialylated peptide, such as the carboxy-terminal peptide (CTP, of chorionic gonadotropin β chain).

In some embodiments, serum half-life of an engineered Cas9 protein is decreased by fusion with destabilizing domains, including but not limited to geminin, ubiquitin, FKBP12-L106P, and/or dihydrofolate reductase.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cas9 polypeptide with controllable stability such that the variant Cas9 polypeptide can be turned “on” (i.e., stable) or “off (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off, degraded) above the threshold temperature. As another example, if the degron is a drug inducible degron, the presence or absence of drug can switch the protein from an “off (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing dead Cas9 to a degron sequence produces a “tunable” and “inducible” dead Cas9 polypeptide.

Any of the fusion partners described herein can be used in any desirable combination. As one non-limiting example to illustrate this point, a Cas9 fusion protein can comprise a YFP sequence for detection, a degron sequence for stability, and transcription activator sequence to increase transcription of the target DNA. Furthermore, the number of fusion partners that can be used in a dCas9 fusion protein is unlimited. In some cases, a Cas9 fusion protein comprises one or more (e.g. two or more, three or more, four or more, or five or more) heterologous sequences.

### Target Nucleic Acids

A target nucleic acid is a DNA molecule, RNA molecule, which is single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases either deoxyribonucleotides, ribonucleotides, or analogs thereof. Target nucleic acids may have three-dimensional structure, may include coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. In some embodiments, a target nucleic acid may be interspersed with non-nucleic acid components.

A target nucleic acid is recognized by CRISPR-Cas9 system and binds Cas9. In some embodiments, it is modified or cleaved or has altered expression due to the binding of Cas9. A target nucleic acid contains a specific recognizable PAM motif, for example, 5′-NGG-3′, 5′-NGC-3′, 5′-NAGHC-3′, 5′-NRHRRH-3′ or 5′-NNAAA-3′ (H=A, C or T; R=A or G).

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered Cas9 enzyme described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

In some embodiments, a nucleotide sequence encoding a DNA-targeting RNA and/or Cas9 protein is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a DNA-targeting RNA and/or a novel Cas9 protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and/or a human HI promoter (HI).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter, an aromatic amino acid decarboxylase (AADC) promoter, a neurofilament promoter, a synapsin promoter, a thy-1 promoter, a serotonin receptor promoter, a tyrosine hydroxylase promoter (TH), a GnRH promoter, an L7 promoter, a DNMT promoter, an enkephalin promoter, a myelin basic protein (MBP) promoter, a Ca²⁺-calmodulin-dependent protein kinase II-alpha (CamKIIa) promoter and/or a CMV enhancer/platelet-derived growth factor-β promoter.

Adipocyte-specific spatially restricted promoters include, but are not limited to aP2 gene promoter/enhancer, e.g., a region from −5.4 kb to +21 bp of a human aP2 gene, a glucose transporter-4 (GLUT4) promoter, a fatty acid translocase (FAT/CD36) promoter, a stearoyl-CoA desaturase-1 (SCD1) promoter, a leptin promoter, and an adiponectin promoter, an adipsin promoter and/or a resistin promoter.

Cardiomyocyte-specific spatially restricted promoters include, but are not limited to control sequences derived from the following genes: myosin light chain-2, a-myosin heavy chain, AE3, cardiac troponin C, and/or cardiac actin.

Smooth muscle-specific spatially restricted promoters include, but are not limited to an SM22a promoter, a smoothelin promoter, and/or an a-smooth muscle actin promoter.

Photoreceptor-specific spatially restricted promoters include, but are not limited to, a rhodopsin promoter, a rhodopsin kinase promoter, a beta phosphodiesterase gene promoter, a retinitis pigmentosa gene promoter, an interphotoreceptor retinoid-binding protein (IRBP) gene enhancer, and/or an IRBP gene promoter.

### Gene Editing Uses of CRISPR-Cas9

The CRISPR-Cas9 system described herein can be used for gene editing, which can result in a gene silencing event, or an alteration of the expression (e.g., an increase or a decrease) in the expression of a desired target gene. Accordingly, in some embodiments, the CRISPR-Cas9 system described herein is used in a method of altering the expression of a target nucleic acid. In some embodiments the CRISPR-Cas9 system described herein is used in a method of modifying a target nucleic acid in a desired target cell. In some embodiments, the invention provides methods for site-specific modification of a target nucleic acid in eukaryotic cells to effectuate a desired modification in gene expression.

In some embodiments, the invention provides an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1, 4, 8, 14, 84 or 86, and wherein the Cas protein is capable of binding to the RNA guide and of causing a break in the target nucleic acid sequence complementary to the RNA guide.

In some embodiments, the invention provides engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1, 4, 8, 14, 84 or 86; wherein the Cas protein is fused to a deaminase, and wherein the Cas protein fusion is capable of binding to the RNA guide and of editing the target nucleic acid sequence complementary to the RNA guide.

In some embodiments, provided herein is an engineered, non-naturally occurring CRISPR-Cas system comprising a codon-optimized CRISPR-associated (Cas) protein, further comprising a nuclear localization sequence (NLS) and/or a FLAG, HIS or HA tag.

In some embodiments, provided herein is an engineered, non-naturally occurring Cas9 fusion protein further comprising a nuclear localization sequence (NLS) and/or a FLAG, HIS or HA tag.

In some embodiments, provided herein is an engineered, non-naturally occurring Cas9 fusion protein having at least 80% identity to SEQ ID NOs: 2, 5, 9, 15, 85, 87, 95 or 96.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the invention provides a method of modifying a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

Accordingly, in some embodiments, the Cas protein has about 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1, 4, 8, 14, 84 or 86. In some embodiments, the Cas protein is identical to SEQ ID NO: 1, 4, 8, 14, 84 or 86.

Suitable guide RNA, Cas9 mutations and fusion proteins for use in the CRISPR-Cas9 system and method are as described throughout this disclosure.

In one aspect, the method comprises binding of the CRISPR-Cas9 to a target nucleic acid and effecting cleavage of a target nucleic acids. In some embodiments, the CRISPR-Cas9 system cleaves target DNA or RNA duplexes by introducing double-stranded breaks. In some embodiments, the CRISPR-Cas9 system cleaves target DNA or RNA by introducing single-stranded breaks or nicks.

In some embodiments, the CRISPR-Cas9 method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the CRISPR-Cas9 method or system comprises a fusion protein with enzymes that can edit DNA sequences by chemically modifying nucleotide bases, including deaminase enzymes that can modify adenosine or cytosine bases and function as site-specific base editors. For example, APOBEC1 cytidine deaminase, which usually uses RNA as a substrate, can be targeted to single-stranded and double-stranded DNA when it is fused to Cas9, converting cytidine to uridine directly, and ADAR enzymes deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19(12): 770-788, the contents of which are incorporated herein. Accordingly, in some embodiments, the Cas9 enzymes (ScoCas9, SirCas9, VapCas9, EpeCas9, LfeCas9, PmaCas9) described herein is a component of a nucleobase editor. In some embodiments, the base editor is the adenine deaminase TadA8 or TadA9.

In some embodiments, base editing results in the introduction of stop codons to silence genes. In some embodiments, base editing results in altered protein function by altering amino acid sequences.

In some embodiments, the CRISPR-Cas9 method or system comprises epigenetic modification of target DNA by fusion with a histone. In some embodiments, the CRISPR-Cas9 system comprises epigenetic modification of target DNA by fusion with an epigenetic modifying enzyme such as a reader, writer or eraser protein. In some embodiments, the CRISPR-Cas9 system comprises fusion with a histone modifying enzyme to alter the histone modification pattern in a selected region of target DNA. Histone modifications can occur in many different ways including methylation, acetylation, ubiquitination, phosphorylation, and in many different combinations, leading to structural changes in DNA. In some embodiments, histone modification leads to transcriptional repression or activation.

In some embodiments, the CRISPR-Cas9 method or system modulates transcription of target DNA by increasing or decreasing transcription through fusion with transcriptional activator proteins or transcriptional repressor proteins, small molecule/drug-responsive transcriptional regulators, inducible transcription regulators. In some embodiments, the CRISPR-Cas9 system is used to control the expression of a target coding mRNA (i.e. a protein encoding gene) where binding results in increased or decreased gene expression.

In some embodiments, the CRISPR-Cas9 method or system is used to control gene regulation by editing genetic regulatory elements such as promoters or enhancers.

In some embodiments, the CRISPR-Cas9 method or system is used to control the expression of a target non-coding RNA, including tRNA, rRNA, snoRNA, siRNA, miRNA, and long ncRNA.

In some embodiments, the CRISPR-Cas9 method or system is used for targeted engineering of chromatin loop structures. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, CRISPR-Cas9 is used for live cell imaging. Fluorescently labelled Cas9 is targeted to repetitive genomic regions such as centromeres and telomeres to track native chromatin loci throughout the cell cycle and determine differential positioning of transcriptionally active and inactive regions in the 3D nuclear space.

In some embodiments, the CRISPR-Cas9 method or system is used for correction of pathogenic mutations by insertion of beneficial clinical variants or suppressor mutations.

### Nucleobase Editors

Disclosed herein, are novel base editors or nucleobase editors for editing, modifying or altering a target nucleotide sequence of a polynucleotide comprising a Cas9. Described herein is a nucleobase editor or a base editor comprising a polynucleotide programmable nucleotide binding domain (e.g., Cas9) and a nucleobase editing domain (e.g., adenosine deaminase). A polynucleotide programmable nucleotide binding domain (e.g., Cas9), when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid. As most of the known genetic variations associated with human disease are point mutations, methods that can more efficiently and cleanly make precise point mutations are needed. Base editing systems as provided herein provide a new way to provide genome editing without generating double-strand DNA breaks, without requiring a donor DNA template, and without inducing an excess of stochastic insertions and deletions.

The base editors provided herein are capable of modifying a specific nucleotide base without generating a significant proportion of indels. The term “indel(s)”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the target nucleotide sequence. In certain embodiments, any of the base editors provided herein are capable of generating a greater proportion of intended modifications (e.g., point mutations or deaminations) versus indels.

In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, any of the base editors provided herein are capable of generating at least 0.01% of intended mutations (i.e. at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

### Therapeutic Applications

The CRISPR-Cas9 methods or systems described herein can have various therapeutic applications. Accordingly, in some embodiments, a method of treating a disorder or a disease in a subject in need thereof is provided, the method comprising administering to the subject a CRISPR-Cas9 system comprising a Cas9 as described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the CRISPR-Cas9 methods or systems can be used to treat various diseases and disorders, e.g., genetic disorders (e.g., monogenetic diseases), diseases that can be treated by nuclease activity, and various cancers, etc.

In some embodiments, the CRISPR methods or systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). In some embodiments, the CRISPR methods or systems described herein comprise a nucleobase editor. For example, in some embodiments, the Cas9 proteins described herein are fused to a polypeptide having nucleobase editing activity.

In one aspect, the CRISPR methods or systems described herein can be used for treating a disease caused by overexpression of RNAs, toxic RNAs, and/or mutated RNAs (e.g., splicing defects or truncations).

In some embodiments, the CRISPR methods or systems described herein can also target trans-acting mutations affecting RNA-dependent functions that cause various diseases.

In some embodiments, the CRISPR methods or systems described herein can also be used to target mutations disrupting the cis-acting splicing codes that can cause splicing defects and diseases.

The CRISPR methods or systems described herein can further be used for antiviral activity, in particular against RNA viruses. The CRISPR-associated proteins can target the viral RNAs using suitable RNA guides selected to target viral RNA sequences.

The CRISPR methods or systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

Further, the CRISPR methods or systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The CRISPR systems described herein, or components thereof, nucleic acid molecules thereof, and/or nucleic acid molecules encoding or providing components thereof, CRISPR-associated proteins, or RNA guides, can be delivered by various delivery systems such as vectors, e.g., plasmids and delivery vectors. Exemplary embodiments are described below. The CRISPR systems (e.g., including the Cas9 comprising nucleobase editor described herein) can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the CRISPR system of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing CRISPR systems including the Cas9 described herein can be useful for generating CRISPR systems capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a CRISPR system (e.g., including the Cas9 disclosed herein) of the present disclosure is of sufficient size so as to enable efficient packaging and delivery into a target cell via a retroviral vector. In some cases, a Cas9 is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A CRISPR system (e.g., including the Cas9 disclosed herein) described herein can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for CRISPR are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 5 (below).

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioley1-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecy1-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1 -Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 6 lists exemplary polymers for use in gene transfer and/or nanoparticle formulations.

TABLE 6
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 7 summarizes delivery methods for a polynucleotide encoding a Cas9 described herein.

TABLE 7

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, optionally fused to a polypeptide having biological activity (e.g., a nucleobase editor), and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the Cas9 described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a Cas9 of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAVS or any combination thereof. One can select the type of AAV with regard to the cells to be targeted; e.g., one can select AAV serotypes 1, 2, 5 or a hybrid capsid AAV1, AAV2, AAVS or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi. 2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising CRISPR system (e.g., including Cas9 disclosed herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) a sequence that is hybridized to the tracr sequence; and/or (b) a second regulatory element operably linked to an enzyme-coding sequence encoding said CRISPR enzyme comprising a nuclear localization sequence. Elements may be provide individually or in combinations, and may be provided in any suitable container, such as a vial, a bottle, or a tube. In some embodiments, the kit includes instructions in one or more languages, for example in more than one language.

In some embodiments, the kit comprises a nucleobase editor. For example, in some embodiments, the kit includes a nucleobase editor comprising the Cas9 enzymes (ScoCas9, SirCas9, VapCas9, EpeCas9, LfeCas9, PmaCas9) described herein.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Screening for Novel Cas9 Enzymes, Discovery and Optimization of Novel Cas9 Enzymes

This example describes a screen for the discovery of novel Cas9 enzymes. As described herein, using this screen novel Cas9 enzymes from Streptococcus constellatus, Sharpea spp. isolate RUG017, Veillonella parvula, Ezakiella peruensis, Lactobacillus fermentum strain AF15-40LB and Peptoniphilus sp. Marseille-P3761 bacteria were isolated and optimized.

In a search to discover new Cas9 enzymes which recognize novel PAM sequences, a bioinformatics screen was used to search for additional enzymes to expand CRISPR's targeting range. The screen utilized seed sequences of Cas9 from S. pyogenes, S. aureus, S. thermophilus, and F. novicida. Bioinformatics was carried out using the tblastn variant of BLAST with an e-value threshold of 1e-6 for considering BLAST hits. Briefly, loci selected for testing were loci that remained intact in the presence of Cas9 proteins from other species. Loci were selected that had greater than three spacers within the CRISPR array and greater than 1 kb endogenous sequence 5′ of Cas9 and greater than 300 nt 3′ of the CRISPR array. Using this approach, novel Cas9 enzymes were identified from different bacterial species and codon optimized for expression in human cells. The novel engineered Cas9 enzymes were then recombinantly produced and tested.

### Example 2. Identifying 3′ PAM Consensus Motif for Novel Cas9 Enzymes from Streptococcus constellatus, Sharpea Spp. Isolate RUG017, Veillonella parvula, Ezakiella peruensis, Lactobacillus fermentum Strain AF15-40LB and Peptoniphilus sp. Marseille P3761 Bacteria

This example illustrates the identification of the protospacer adjacent motif (PAM) sequence for human codon-optimized Cas9 originally isolated from Streptococcus constellatus, Sharpea spp. isolate RUG017, Veillonella parvula, Ezakiella peruensis, Lactobacillus fermentum strain AF15-40LB and Peptoniphilus sp. Marseille-P3761 species.

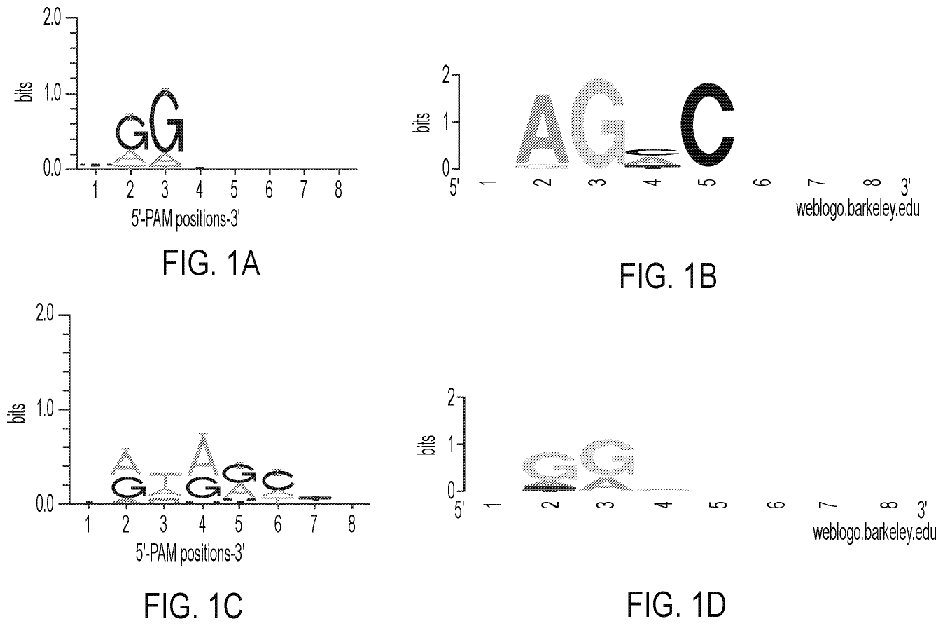

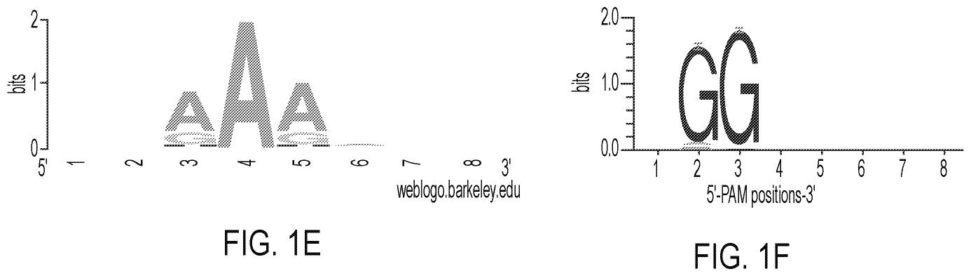

The human, codon-optimized Cas9 was tested for its recognition of a PAM sequence using an in vitro PAM identification assay. A library of plasmids bearing randomized PAM sequences were incubated with Cas9 isolated from different bacteria. Uncleaved plasmid was purified and sequenced to identify specific PAM motifs that were cleaved. The consensus PAM sequence recognized by Streptococcus constellatus Cas9 was identified as 5′-NGG-3′ (FIG. 1A). The consensus PAM sequence recognized by Sharpea spp. isolate RUG017 Cas9 was identified as 5′-NAGHC-3′ (FIG. 1B). The consensus PAM sequence recognized by Veillonella parvula Cas9 was identified as 5′-NRHRRH-3′ (H=A, C or T; R=A or G) (FIG. 1C). The consensus PAM sequence recognized by Ezakiella peruensis Cas9 was identified as 5′-NGG-3′ (FIG. 1D). The consensus PAM sequence recognized by Lactobacillus fermentum strain AF15-40LB Cas9 was identified as 5′-NNAAA-3′ (FIG. 1E). The consensus PAM sequence recognized by Peptoniphilus sp. Marseille-P3761 Cas9 was identified as 5′-NGG-3′ (FIG. 1F).

### Example 3. Predicting RNA Folding Structure of sgRNA for Novel Cas9 Enzymes from Streptococcus constellatus, Sharpea Spp. Isolate RUG017, Veillonella parvula, Ezakiella peruensis, Lactobacillus Fermentum Strain AF15-40LB and Peptoniphilus sp. Marseille-P3761 Bacteria

This example demonstrates the predicted RNA folding structure of exemplary sgRNA comprising crRNA and tracrRNA for use with novel Cas9 enzymes.

Small RNA sequencing was carried out on RNA derived from an E. coli strain heterologously expressing Cas9 Crispr loci. Briefly, RNA was isolated from stationary phase bacteria by first resuspending the E. coli in Trizol, then homogenizing the bacteria with zirconia/silica beads in a homogenizer for three 1 min cycles. Total RNA was purified from homogenized samples, DNAse treated and 3′ dephosphorylated with T4 polynucleotide kinase and rRNA was removed. RNA libraries were prepared from rRNA-depleted RNA, and size selected for small RNA.

For RNA sequencing, transcripts were poly-A tailed with E. coli Poly (A) polymerase, ligated with 5′ RNA adapters using T4 RNA ligase 1 and reverse transcribed, followed by PCR amplification of cDNA with barcoded primers, and sequencing on a MiSeq. Reads from each sample were identified on the basis of their associated barcode and aligned to a reference sequence using BWA. Paired-end alignments were used to extract transcript sequences using Picard tools and the sequences were analyzed using Geneious software.

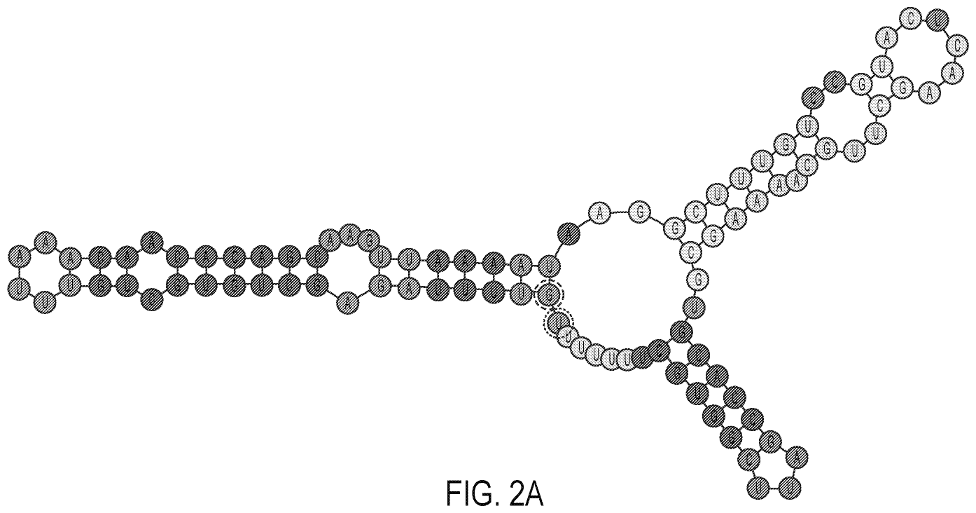

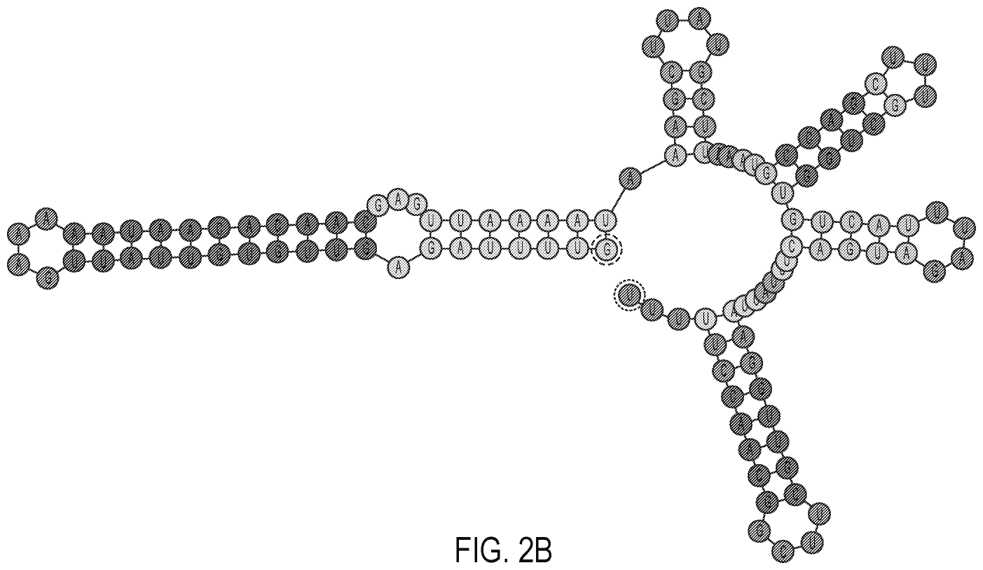

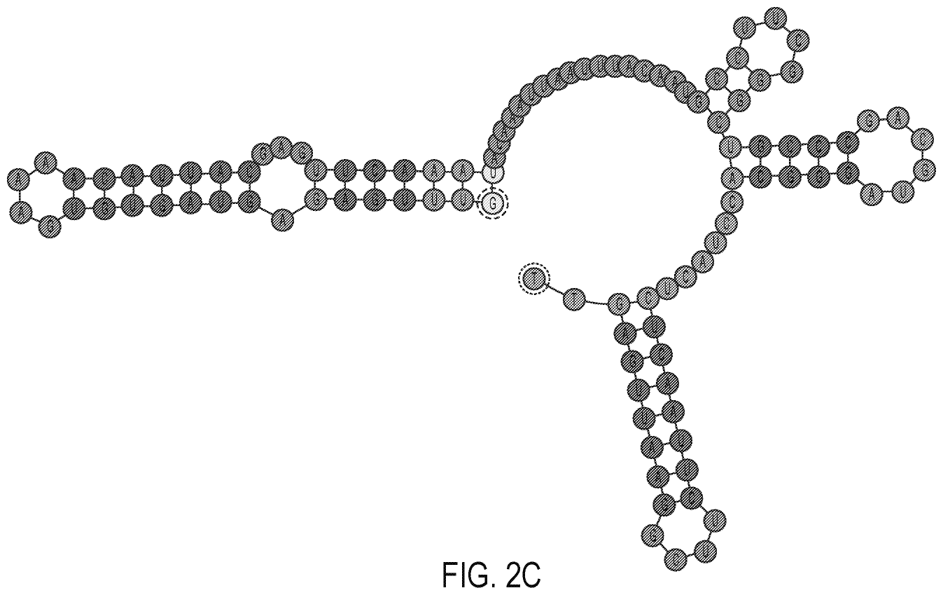

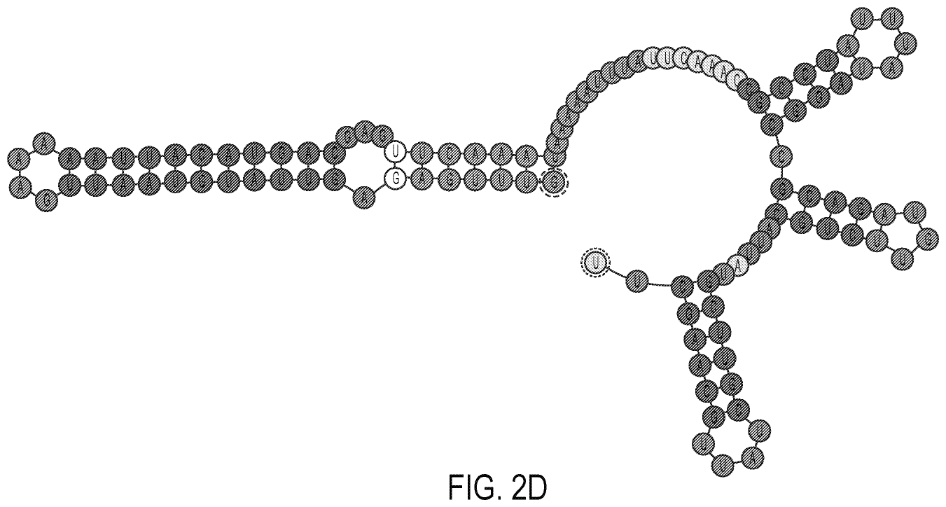

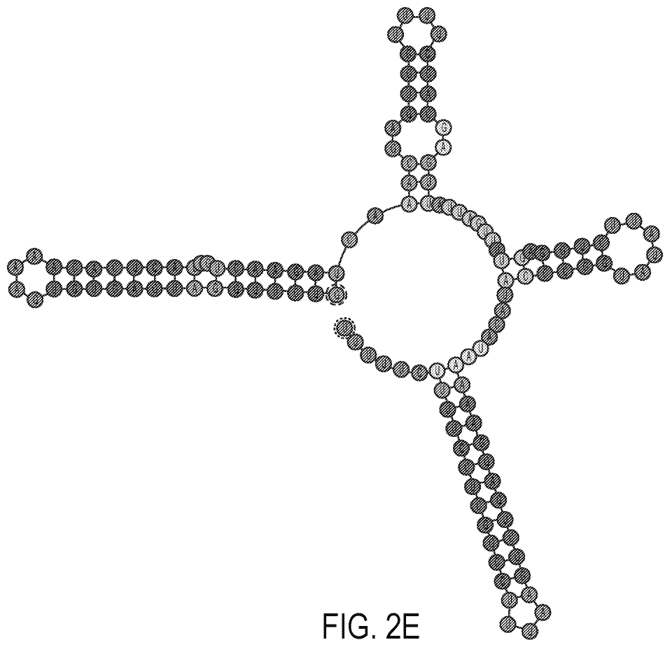

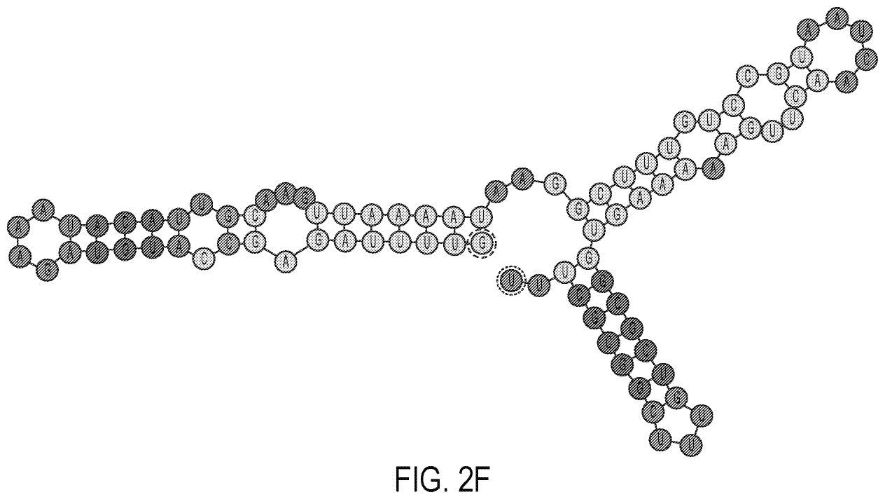

RNA folding was based on prediction from Geneious 11.1.2 software. The single sgRNA transcript fuses the crRNA to tracrRNA mimicking the dual RNA structure required to guide site-specific Cas9 activity. The predicted RNA folding structure for the chimeric sgRNA for use with ScoCas9 from Streptococcus constellatus is shown in FIG. 2A, sgRNA for use with SirCas9 from Sharpea spp. isolate RUG017 is shown in FIG. 2B, sgRNA for use with VapCas9 from Veillonella parvula is shown in FIG. 2C, sgRNA for use with EpeCas9 from Ezakiella peruensis is shown in FIG. 2D, sgRNA for use with LfeCas9 from Lactobacillus fermentum strain AF15-40LB is shown in FIG. 2E and sgRNA for use with PmaCas9 from Peptoniphilus sp. Marseille-P3761 is shown in FIG. 2F.

### Example 4. Ex Vivo Cleavage Activity by WT ScoCas9 in HEK293T Cells

This example illustrates ex vivo nucleic acid cleavage activity by WT ScoCas9 from Streptococcus constellatus in HEK293T cells.

HEK293T cells were plated in a 96-well plate. Cells were transfected with expression vectors containing Cas9 and guide RNAs (Table 10), 24 hours after plating. Cells were harvested 72 hours post-transfection and total DNA was extracted.

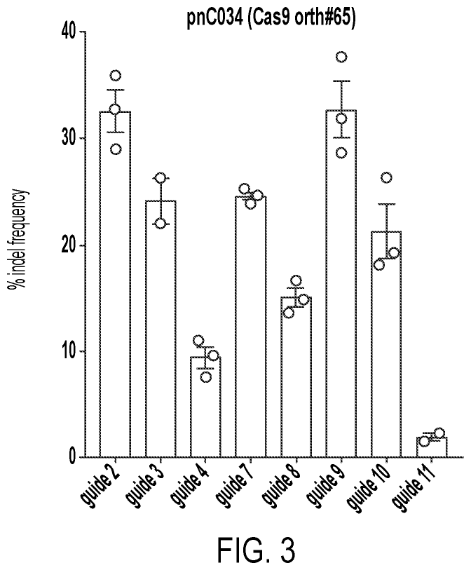

Deep sequencing was carried out to characterize indel patterns in the HEK293T cells. Briefly, exemplary targets (Table 8) were amplified using a two-round PCR to add Illumina adapters as well as unique barcodes to the target amplicons. PCR products were run on a 2% gel and gel extracted. Samples were pooled, quantified and cDNA libraries were prepared and sequenced on MiSeq. Indel frequency was determined by deep sequencing (FIG. 3).

TABLE 8
Exemplary Guide RNA Sequences and PAM Sequences

| ID (Sco/Pma) | 5′→3′ guide sequence | 3′ PAM |
|---|---|---|
| guide 2 | GAAACAATGATAACAAGACC (SEQ ID NO: 97) | TGG |
| guide 3 | GTGGCCCCTGTGCCCAGCCC (SEQ ID NO: 98) | TGG |
| guide 4 | GTCCCAAATATGTAGCTGTT (SEQ ID NO: 99) | TGG |
| guide 6 | GCTCCCATCACATCAACCGG (SEQ ID NO: 100) | TGG |
| guide 7 | GATGTCACCTCCAATGACTA (SEQ ID NO: 101) | GGG |
| guide 9 | GTTGAAGATGAAGCCCAGAG (SEQ ID NO: 102) | CGG |
| guide 10 | GCCAACACCAACCAGAACTT (SEQ ID NO: 103) | GGG |
| guide 11 | TGCTGCACACAGCAGGCCTT (SEQ ID NO: 104) | TGG |

The data showed that that WT ScoCas9 achieved between 2-32% indel frequency. Guide RNAs 2 and 9 resulted in greater than 30% indel mutations, while guide RNA 11 resulted in about 2% indel mutations.

### Example 5. Base Editing by Cas9 Enzyme with an N-Terminal Fusion of an Adenine Base Editor (ABE) or a Cytidine Base Editor (CBE)

This example illustrates base conversion efficiency of a Cas9 enzyme fused to an adenine base editor (ABE), or to a cytidine base editor (CBE).

Briefly, 25,000 HEK293T cells were plated per 96-well. 100 ng of Cas9 expression plasmid and 100 ng of guide expression plasmid were transfected 24 h after plating. Cells were harvested 5 days after transfection and DNA was extracted.

Deep sequencing was carried out to characterize A-to-G conversion or C-to-T conversion in the HEK293T cells. Exemplary targets were amplified using a two-round PCR region to add Illumina adapters as well as unique barcodes to the target amplicons. PCR products were run on a 2% gel and gel extracted. Samples were pooled, quantified and cDNA libraries were prepared and sequenced on MiSeq. The percent A-to-G conversion was determined by deep sequencing for the N-terminal as well as the C-terminal TadA8 fusion constructs. The percent C-to-T conversion was determined by deep sequencing for the N-terminal as well as the C-terminal ppAPOBEC1 fusion constructs.

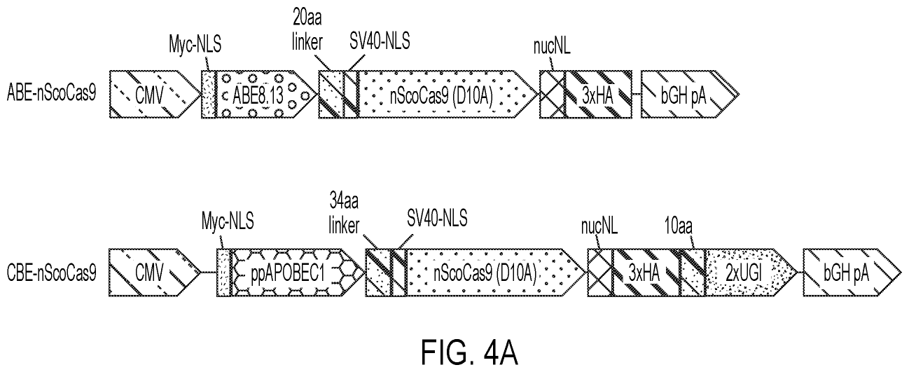

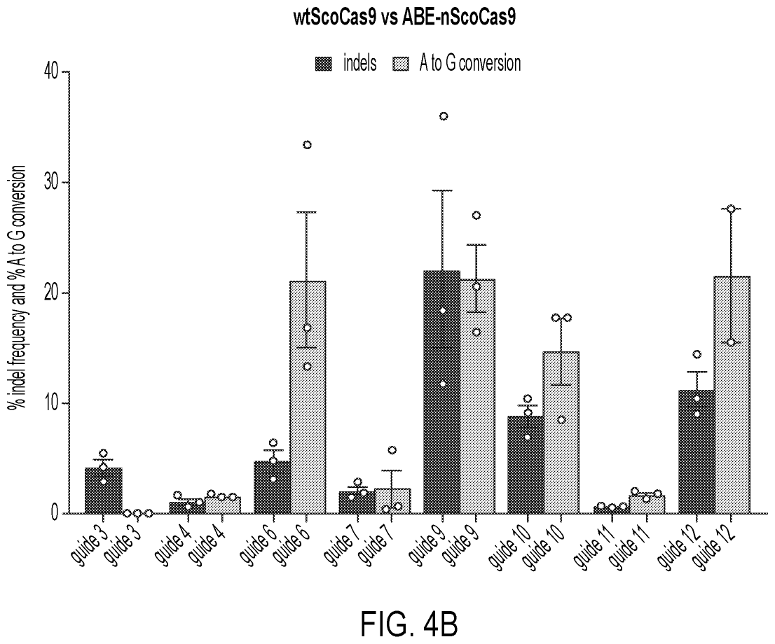

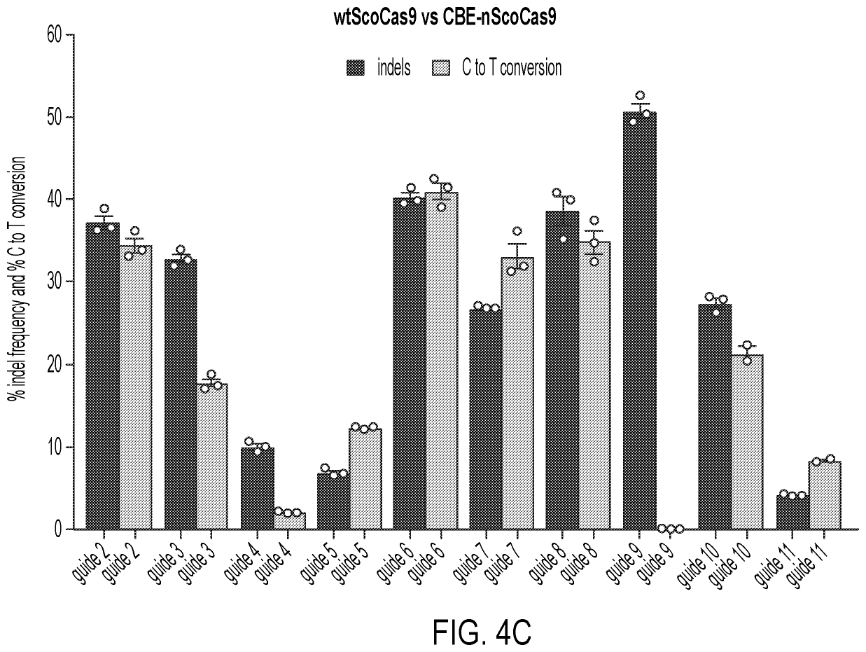

FIG. 4A shows a schematic diagram of constructs of ScoCas9 fused to ABE or CBE at the N-terminal. Table 9 shows the guide RNA sequences used with ScoCas9. FIG. 4B shows a graph of indel mutations and targeted adenine to guanine conversion percentage achieved with an N-terminal fusion of ScoCas9 to an adenine base editor (ABE) (FIG. 4B), which are directed to genomic sites in a human cell line (HEK293T). FIG. 4C shows a graph of indel mutations and targeted cytosine to thymine conversion percentage achieved with an N-terminal fusion of ScoCas9 to a cytidine base editor (FIG. 4C), which are directed to genomic sites in a human cell line (HEK293T).

TABLE 9
Guide RNA Sequences and PAM Sequences used with ScoCas9

| ID (Sco) | 5′→3′ guide sequence | 3′ PAM |
|---|---|---|
| guide 1 | GAACACAAAGCATAGACTGC (SEQ ID NO: 105) | GGG |
| guide 2 | GAAACAATGATAACAAGACC (SEQ ID NO: 106) | TGG |
| guide 3 | GTGGCCCCTGTGCCCAGCCC (SEQ ID NO: 107) | TGG |
| guide 4 | GTCCCAAATATGTAGCTGTT (SEQ ID NO: 108) | TGG |
| guide 5 | AGAGGGACACACAGATCTAT (SEQ ID NO: 109) | TGG |
| guide 6 | GCTCCCATCACATCAACCGG (SEQ ID NO: 110) | TGG |
| guide 7 | GATGTCACCTCCAATGACTA (SEQ ID NO: 111) | GGG |
| guide 8 | GGGCAACCACAAACCCACGA (SEQ ID NO: 112) | GGG |
| guide 9 | GTTGAAGATGAAGCCCAGAG (SEQ ID NO: 113) | CGG |
| guide 10 | GCCAACACCAACCAGAACTT (SEQ ID NO: 114) | GGG |
| guide 11 | TGCTGCACACAGCAGGCCTT (SEQ ID NO: 115) | TGG |
| guide 12 | GTGCCAGAAACAGGGGTGAC (SEQ ID NO: 116) | GGG |

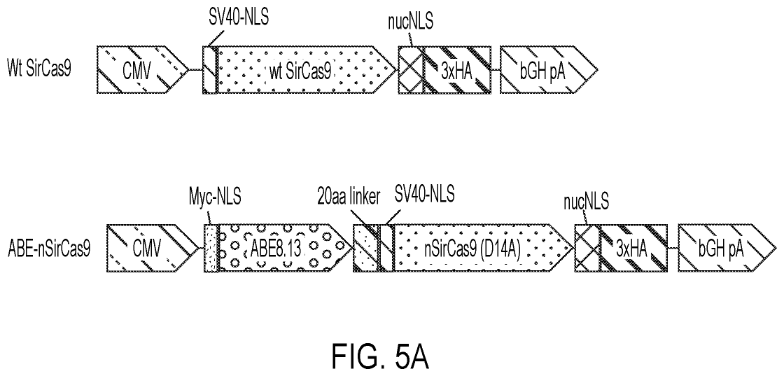

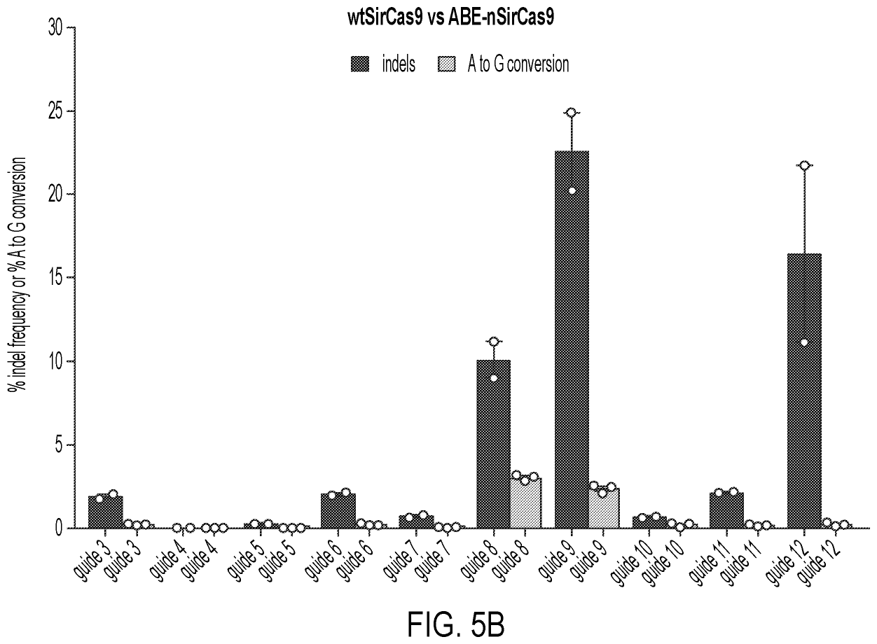

FIG. 5A shows a schematic diagram of constructs of WT SirCas9 as well as SirCas9 (“D14A” mutant) fused to an ABE at the N-terminal. Table 10 shows the exemplary NAGMC guide RNA sequences used with SirCas9. FIG. 5B shows a graph of indel mutations and targeted adenine to guanine conversion percentage achieved with an N-terminal fusion of SirCas9 to an adenine base editor (ABE) (FIG. 5B), which are directed to genomic sites in a human cell line (HEK293T).

TABLE 10
Guide RNA Sequences and PAM Sequences used with SirCas9

| ID (Sir) | 5′→3′ sequence | 3′ PAM |
|---|---|---|
| guide 1 | CCTGCCTCAGCTGCTCACTT (SEQ ID NO: 117) | GAGCC |
| guide 2 | AAACGGTCCCCAGAGGGTTC (SEQ ID NO: 118) | TAGAC |
| guide 3 | GCCACCGGTTGATGTGATGG (SEQ ID NO: 119) | GAGCC |
| guide 4 | AAGTGGTCCCAGGCCTCAGC (SEQ ID NO: 120) | CAGCC |
| guide 5 | AGAGAAAATGAAACTTTCAA (SEQ ID NO: 121) | AAGCC |
| guide 6 | CCAAACCCAACTCCATCTAC (SEQ ID NO: 122) | CAGCC |
| guide 7 | GGTCCTTGAATTGCAGTATC (SEQ ID NO: 123) | TAGCC |
| guide 8 | GCATAGACTGCGGGGGGGGC (SEQ ID NO: 124) | CAGCC |
| guide 9 | GGAAACTGGAACACAAAGCA (SEQ ID NO: 125) | TAGAC |
| guide 10 | GACAGCATGTGGTAATTTTC (SEQ ID NO: 126) | CAGCC |
| guide 11 | GCCCCCGGAAACTCTGTCCA (SEQ ID NO: 127) | GAGAC |
| guide 12 | TCGACCCCCACCAAGGTTCA (SEQ ID NO: 128) | CAGCC |

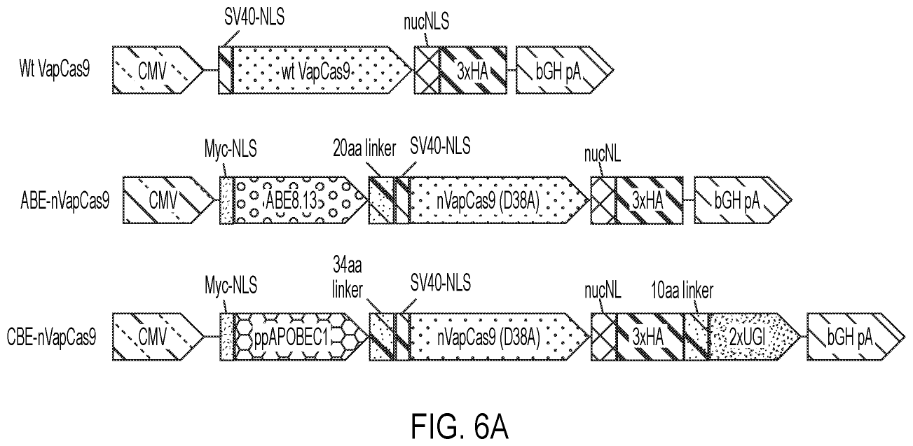

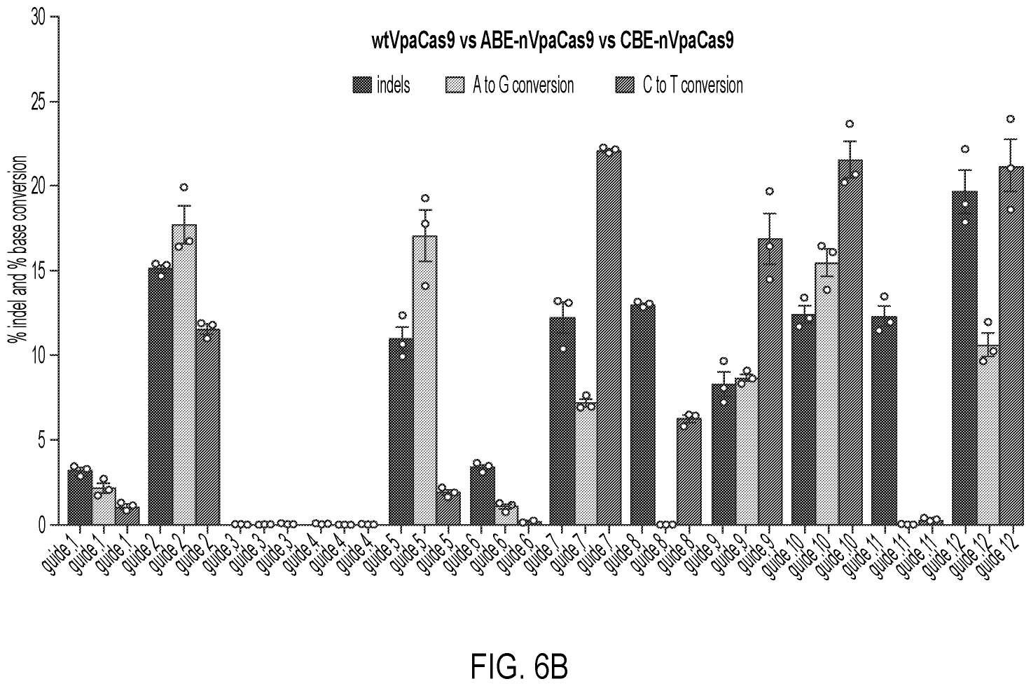

FIG. 6A shows a schematic diagram of constructs showing WT VapCas9, as well as VapCas9 (“D38A” mutant) fused to an ABE or CBE at the N-terminal. Table 11 shows the exemplary NRHRRH [wherein H is adenine, cytosine or thymine, and R is adenine or guanine] guide RNA sequences used with VapCas9. FIG. 6B shows a graph of indel mutations and targeted adenine to guanine conversion percentage achieved with an N-terminal fusion of VapCas9 to an adenine base editor (ABE) as well as targeted cytosine to thymine conversion percentage achieved with an N-terminal fusion of VapCas9 to a cytidine base editor (CBE) (FIG. 6B), which are directed to genomic sites in a human cell line (HEK293T).

TABLE 11
Guide RNA Sequences and PAM Sequences for use with VapCas9

| ID (Vap) | 5′→3′ sequence | 3′ PAM |
|---|---|---|
| guide 1 | TGTTAACAGCTGACCCAATA (SEQ ID NO: 129) | AGTGGC |
| guide 2 | GTTACTCGCCTGTCAAGTGG (SEQ ID NO: 130) | CGTGAC |
| guide 3 | GGGCTCCCATCACATCAACC (SEQ ID NO: 131) | GGTGGC |
| guide 4 | GCTTTGGGGAGGCCTGGAGT (SEQ ID NO: 132) | CATGGC |
| guide 5 | TAGCTGCCAATGACTATAGC (SEQ ID NO: 133) | AATAGC |
| guide 6 | TTAAAATAGGATCTACATCA (SEQ ID NO: 134) | CGTAAC |
| guide 7 | GAATCCTGCCATACACTTTG (SEQ ID NO: 135) | AATAGC |
| guide 8 | CTGCGGGGGGGGCCAGCCTG (SEQ ID NO: 136) | AATAGC |
| guide 9 | ACATTGTCAGAGGGACACAC (SEQ ID NO: 137) | TGTGGC |
| guide 10 | AGCAACTCCAGTCCCAAATA (SEQ ID NO: 138) | TGTAGC |
| guide 11 | GTGGTGGCCGAGCGCCCCCT (SEQ ID NO: 139) | AGTGAC |
| guide 12 | CATTCACCCAGCTTCCCTGT (SEQ ID NO: 140) | GGTGGC |

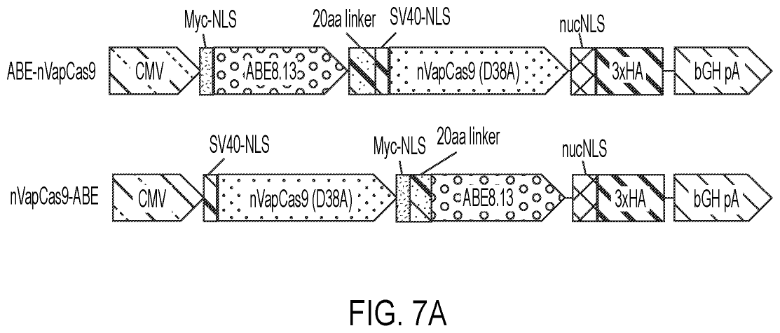

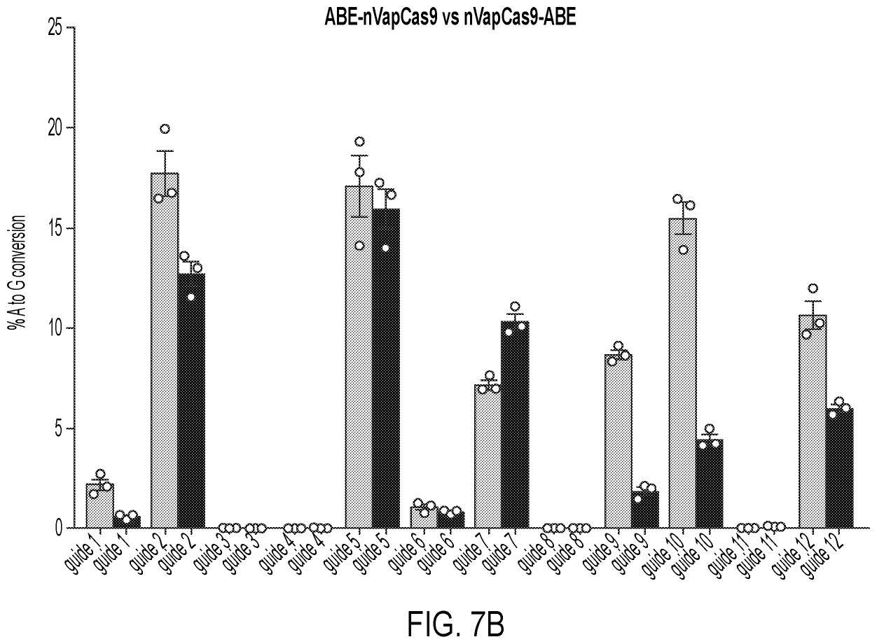

FIG. 7A shows a schematic diagram of constructs showing an N-terminal fusion of ABE and a C-terminal fusion of ABE to VapCas9. FIG. 7B shows a graph of targeted adenine to guanine conversion percentage achieved with an N-terminal fusion and C-terminal fusion to an adenine base editor (ABE).

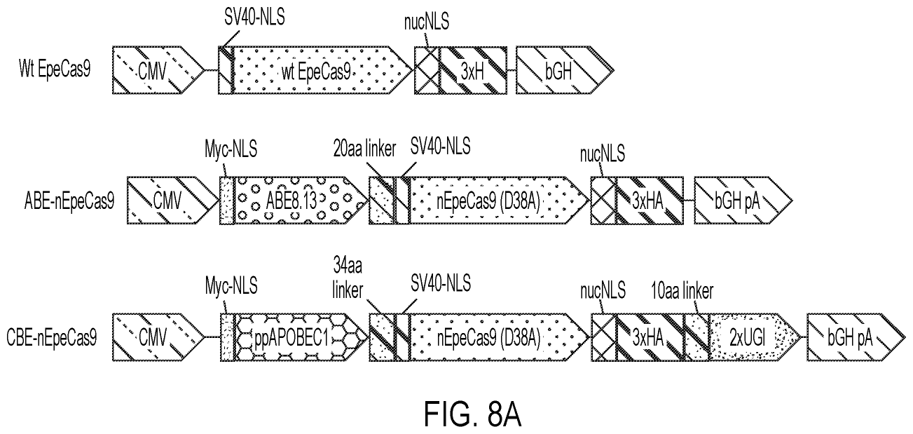

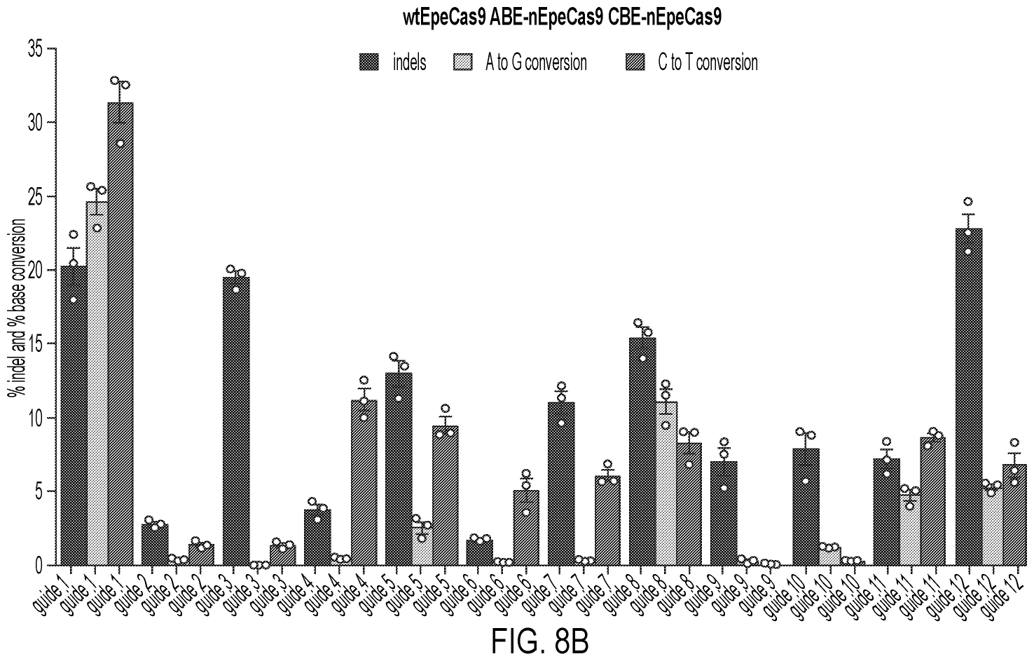

FIG. 8A shows a schematic diagram of constructs showing an N-terminal fusion of ABE and CBE to EpeCas9. Table 12 shows the exemplary guide RNA sequences used with EpeCas9. FIG. 8B shows a graph of indel mutations, a graph of targeted adenine to guanine conversion percentage achieved with an N-terminal fusion to an ABE and targeted cytosine to thymine conversion percentage achieved with an N-terminal fusion to a CBE.

TABLE 12
Guide RNA Sequences and PAM Sequences for use with EpeCas9

| ID (Epe) | Sequence | PAM |
|---|---|---|
| guide 1 | GAACACAAAGCATAGACTGC (SEQ ID NO: 141) | GGG |
| guide 2 | GAAACAATGATAACAAGACC (SEQ ID NO: 142) | TGG |
| guide 3 | GTGGCCCCTGTGCCCAGCCC (SEQ ID NO: 143) | TGG |
| guide 4 | GTCCCAAATATGTAGCTGTT (SEQ ID NO: 144) | TGG |
| guide 5 | AGAGGGACACACAGATCTAT (SEQ ID NO: 145) | TGG |
| guide 6 | GCTCCCATCACATCAACCGG (SEQ ID NO: 146) | TGG |
| guide 7 | GATGTCACCTCCAATGACTA (SEQ ID NO: 147) | GGG |
| guide 8 | GGGCAACCACAAACCCACGA (SEQ ID NO: 148) | GGG |
| guide 9 | GTTGAAGATGAAGCCCAGAG (SEQ ID NO: 149) | CGG |
| guide 10 | GCCAACACCAACCAGAACTT (SEQ ID NO: 150) | GGG |
| guide 11 | TGCTGCACACAGCAGGCCTT (SEQ ID NO: 151) | TGG |
| guide 12 | GTGCCAGAAACAGGGGTGAC (SEQ ID NO: 152) | GGG |

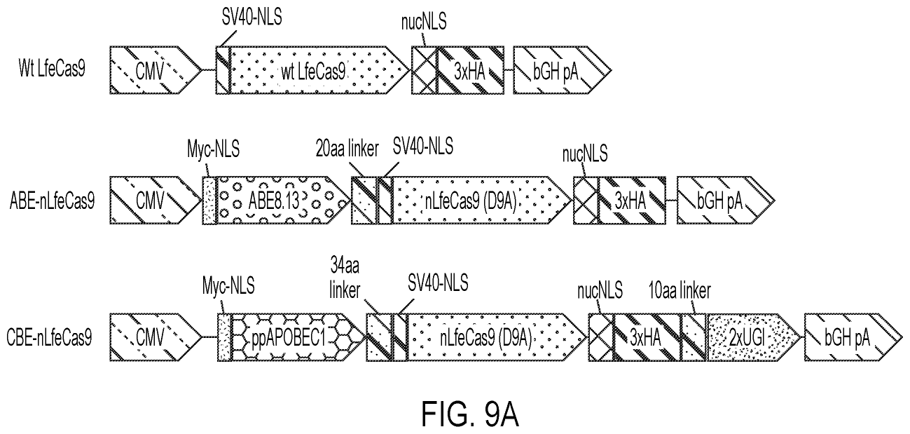

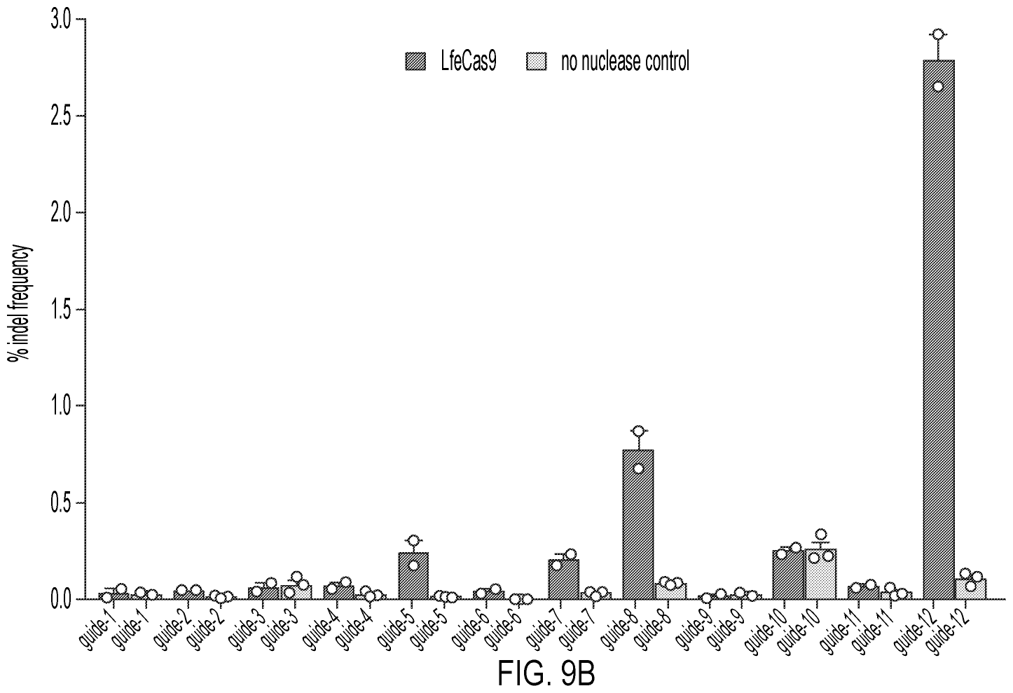

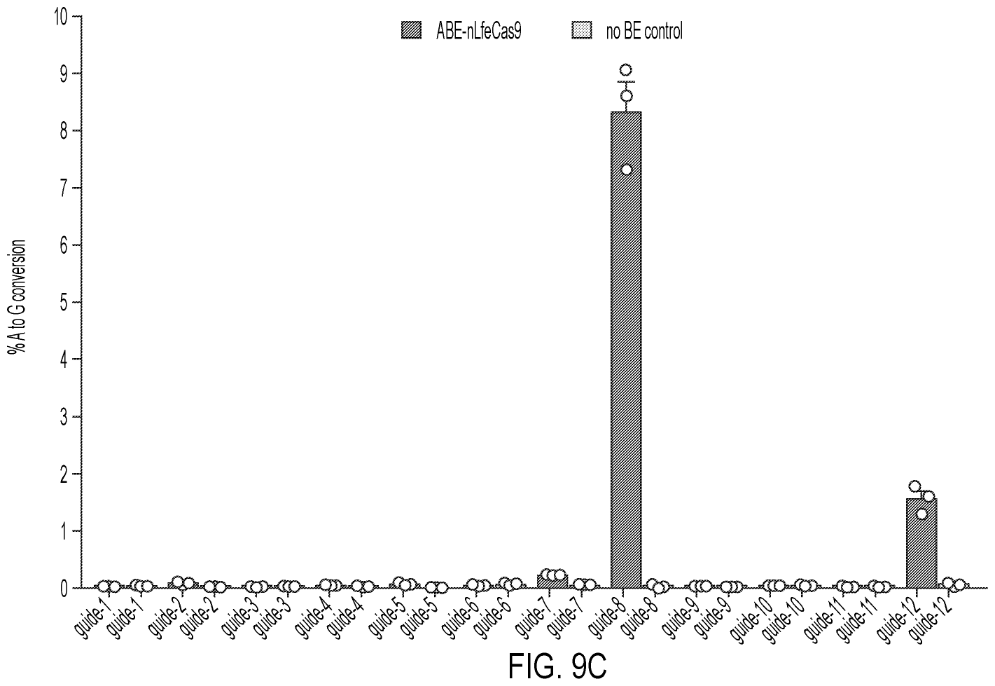

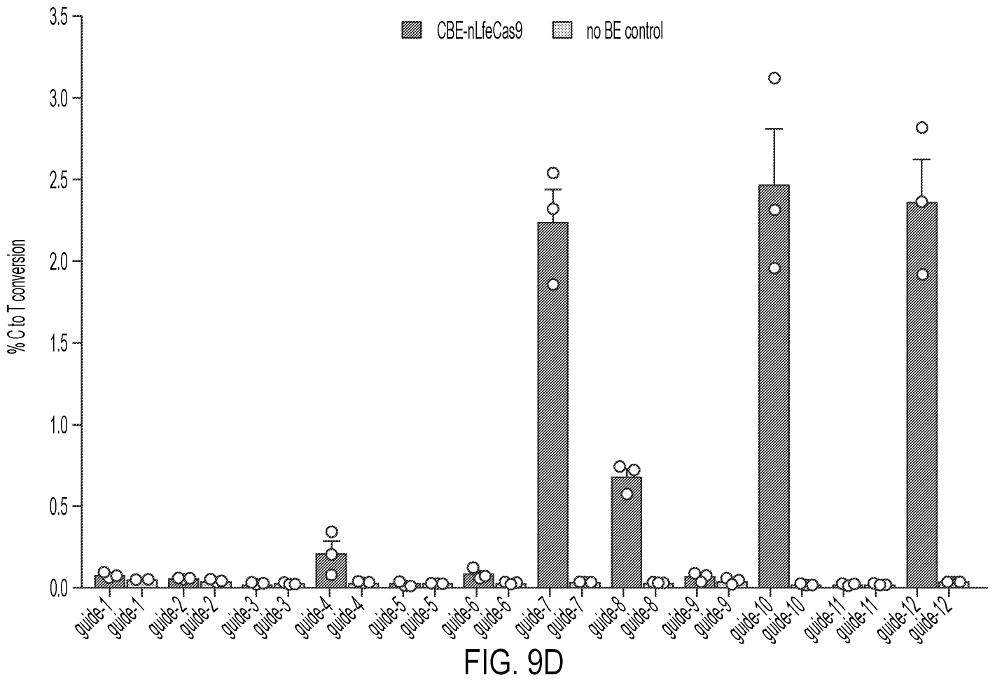

FIG. 9A shows a schematic diagram of constructs showing WT LfeCas9 and LfeCas9 D9A mutant fused at the N-terminus to an ABE and a CBE. Table 13 shows the exemplary guide RNA sequences used with LfeCas9. FIG. 9B shows a graph that shows results of the indel mutation frequency achieved with LfeCas9. FIG. 9C shows a graph of targeted adenine to guanine conversion achieved with an N-terminal fusion of LfeCas9 to an adenine base editor. FIG. 9D shows a graph of targeted cytosine to thymine conversion achieved with a base editor comprising a CBE fused to the N-terminus of an LfeCas9 D9A mutant.

TABLE 13
Guide RNA Sequences and PAM Sequences for use with LfeCas9

| ID (Lfe) | Sequence | PAM |
|---|---|---|
| guide 1 | TCACGGAGACTGAACACTCC (SEQ ID NO: 153) | TCAAA |
| guide 2 | GTAACAGACATGGACCATCA (SEQ ID NO: 154) | GGAAA |
| guide 3 | GGGAGGGAGGGGCACAGATG (SEQ ID NO: 155) | AGAAA |
| guide 4 | TGTGGTTCCAGAACCGGAGG (SEQ ID NO: 156) | ACAAA |
| guide 5 | AATGAGAGAAAATGAAACTT (SEQ ID NO: 157) | TCAAA |
| guide 6 | GGCCATCAAGGATGCCCACG (SEQ ID NO: 158) | AGAAA |
| guide 7 | AAATTGTCCAGCCCCATCTG (SEQ ID NO: 159) | TCAAA |
| guide 8 | CCTGTAAAGGAAACTGGAAC (SEQ ID NO: 160) | ACAAA |
| guide 9 | TACATGAAGCAACTCCAGTC (SEQ ID NO: 161) | CCAAA |
| guide 10 | AAACTCCCCCCACCCCCTTT (SEQ ID NO: 162) | CCAAA |
| guide 11 | GAGTTGGGTTTGGTGCTCAA (SEQ ID NO: 163) | TGAAA |
| guide 12 | GCGGGCCAGCCTGAATAGCT (SEQ ID NO: 164) | GCAAA |

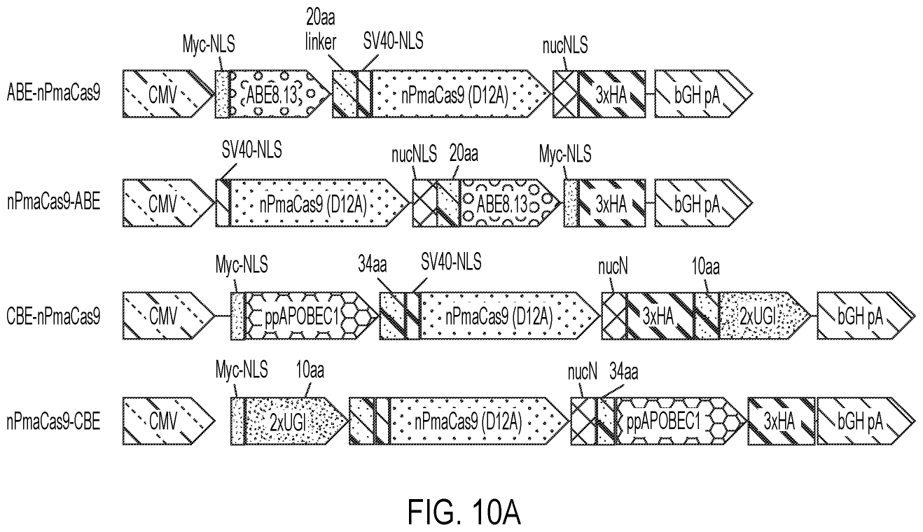

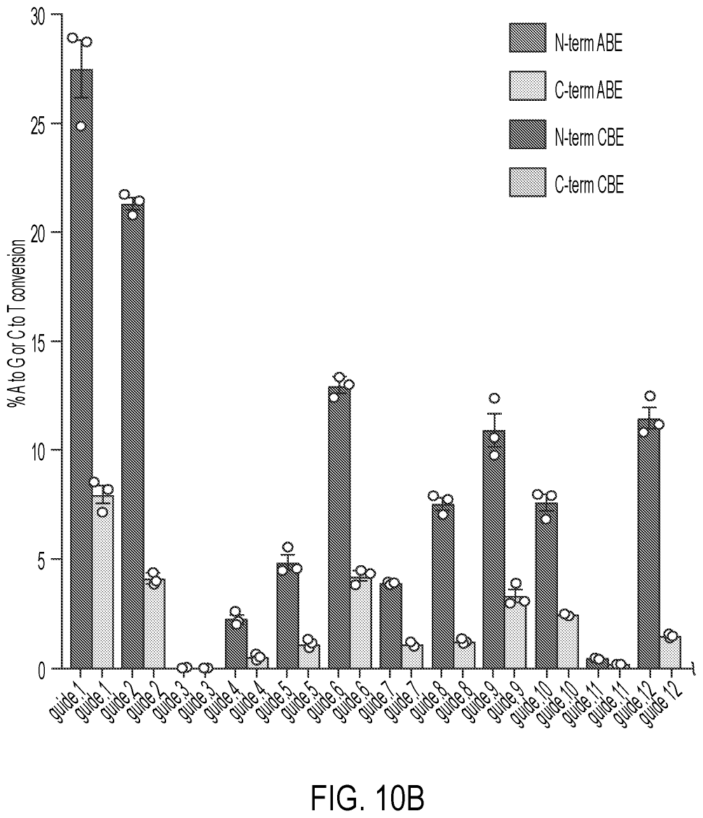

FIG. 10A shows a schematic of constructs showing WT PmaCas9 and PmaCas9 D12A mutant fused at the N-terminus and C-terminus to an ABE and a CBE. FIG. 10B shows a graph that shows results of A-to-G or C-to-T conversion achieved with a base editor comprising an ABE or a CBE fused to the N-terminus or C-terminus of an PmaCas9 D12A mutant.

TABLE 14
Guide RNA Sequences and PAM Sequences for use with PmaCas9

| ID (Pma) | Sequence | PAM |
|---|---|---|
| guide 2 | GAAACAATGATAACAAGACC (SEQ ID NO: 165) | TGG |
| guide 3 | GTGGCCCCTGTGCCCAGCCC (SEQ ID NO: 166) | TGG |
| guide 4 | GTCCCAAATATGTAGCTGTT (SEQ ID NO: 167) | TGG |
| guide 6 | GCTCCCATCACATCAACCGG (SEQ ID NO: 168) | TGG |
| guide 7 | GATGTCACCTCCAATGACTA (SEQ ID NO: 169) | GGG |
| guide 9 | GTTGAAGATGAAGCCCAGAG (SEQ ID NO: 170) | CGG |
| guide 10 | GCCAACACCAACCAGAACTT (SEQ ID NO: 171) | GGG |
| guide 11 | TGCTGCACACAGCAGGCCTT (SEQ ID NO: 172) | TGG |

Table 15 discloses sequences for exemplary Cas9 adenosine or adenine and cytosine or cytidine base editors for base editing functions.

TABLE 15
Sequences of exemplary Cas9 adenosine or adenine and cytosine or cytidine
base editors

| | |
|---|---|
| Sequence ID No. | |
| (description) | Components of DNA cleavage assay |

Amino Acid Sequence of Adenine Deaminase, TadA8.13m-nickase fused to the N-terminal
of nickase ScoCas9 (ABE-nScoCas9, D10A mutant)
MPAAKRVKLDGSEVEFSHEYWMRHALTLAKRARDEREVPVGAVLVLNNRVIGEGWNRAIGLHDP
TAHAEIMALRQGGLVMQNYRLYDATLYVTFEPCVMCAGAMIHSRIGRVVEGVRNAKTGAAGSLM
DVLHHPGMNHRVEITEGILADECAALLCRFFRMPRRVENAQKKAQSSTDGSSGSETPGTSESAT
PESSGPKKKRKVGGKPYSIGLAIGTNSVGWAVVTDDYKVPAKKMKVLGNTDKQSIKKNLLGALL
FDSGETAEATRLKRTARRRYTRRKNRLRYLQEIFTGEMNKVDENFFQRLDDSELVDEDKRGEHH
PIFGNIAAEVKYHDDFPTIYHLRRHLADTSKKADLRLVYLALAHMIKFRGHFLYEGDLKAENTD
VQALFKDEVEEYDKTIEESHLSEITVDALSILTEKVSKSSRLENLIAHYPTEKKNTLFGNLIAL
SLDLHPNEKTNFQLSEDAKLQFSKDTYEEDLEGELGEVGDEYADLFASAKNLYDAILLSGILTV
DDNSTKAPLSASMVKRYEEHQKDLKKLKDFIKVNAPDQYNAIFKDKNKKGYASYIESGVKQDEF
YKYLKGILLKINGSGDFLDKIDREDFLRKQRTFDNGSIPHQIHLQEMHAILRRQGEHYPELKEN
QDKIEKILTFRIPYYVGPLARKGSRFAWAEYKADEKITPWNEDDILDKEKSAEKFITRMTLNDL
YLPEEKVLPKHSPLYEAFTVYNELTKVKYVNEQGEAKFFDTNMKQEIFDHVFKENRKVTKDKLL
NYLNKEFEEFRIVNLTGLDKENKAFNSSLGTYHDLRKILDKSFLDDKANEKTIEDIIQTLTLFE
DREMIRQRLQKYSDIFTKAQLKKLERRHYTGWGRLSYKLINGIRNKENKKTILDYLIDDGYANR
NFMQLINDDALSFKEEIARAQIIDDVDDIANVVHDLPGSPAIKKGILQSVKIVDELVKVMGHNP
ANIIIEMARENQTTDKGRRNSQQRLKLLQDSLKNLDNPVNIKNVENQQLQNDRLFLYYIQNGKD
MYTGETLDINNLSQYDIDHIIPQAFIKDNSLDNRVLTRSDKNRGKSDDVPSIEVVHEMKSFWSK
LLSVKLITQRKFDNLTKAERGGLTEEDKAGFIKRQLVETRQITKHVAQILDERENTEFDGNKRR
IRNVKIITLKSNLVSNFRKEFELYKVREINDYHHAHDAYLNAVVGNALLLKYPQLEPEFVYGEY
PKYNSYRSRKSATEKFLFYSNILRFFKKEDIQTNEDGEIAWNKEKHIKILRKVLSYPQVNIVKK
TEEQTGGESKESILPKGESDKLIPRKTKNSYWDPKKYGGFDSPVVAYSILVFADVEKGKSKKLR
KVQDMVGITIMEKKRFEKNPVDFLEQRGYRNVRLEKIIKLPKYSLFELENKRRRLLASAKELQK
GNELVIPQRFTTLLYHSYRIEKDYEPEHREYVEKHKDEFKELLEYISVFSRKYVLADNNLTKIE
MLFSKNKDAEVSSLAKSFISLLTFTAFGAPAAFNFFGENIDRKRYTSVTECLNATLIHQSITGL
YETRIDLSKLGEDGKRPAATKKAGQAKKKKGSYPYDVPDYAYPYDVPDYAYPYDVPDYA (SEQ
ID NO: 20)
Amino Acid Sequence of Cytidine Deaminase, ppAPOBEC1 fused to the N-terminus of
nickase nScoCas9 (CBE-nScoCas9, D10A mutant)
MPAAKRVKLDTSEKGPSTGDPTLRRRIESWEFDVFYDPRELRKETCLLYEIKWGMSRKIWRSSG
KNTTNHVEVNFIKKFTSERRFHSSISCSITWELSWSPCWECSQAIREFLSQHPGVTLVIYVARL
FWHMDQRNRQGLRDLVNSGVTIQIMRASEYYHCWRNFVNYPPGDEAHWPQYPPLWMMLYALELH
CIILSLPPCLKISRRWQNHLAFFRLHLQNCHYQTIPPHILLATGLIHPSVTWRLKSGGSSGGSS
GSETPGTSESATPESSGGSSGGSPKKKRKVGGKPYSIGLAIGTNSVGWAVVTDDYKVPAKKMKV
LGNTDKQSIKKNLLGALLFDSGETAEATRLKRTARRRYTRRKNRLRYLQEIFTGEMNKVDENFF
QRLDDSFLVDEDKRGEHHPIFGNIAAEVKYHDDFPTIYHLRRHLADTSKKADLRLVYLALAHMI
KFRGHFLYEGDLKAENTDVQALFKDFVEEYDKTIEESHLSEITVDALSILTEKVSKSSRLENLI
AHYPTEKKNTLFGNLIALSLDLHPNEKTNFQLSEDAKLQFSKDTYEEDLEGFLGEVGDEYADLE
ASAKNLYDAILLSGILTVDDNSTKAPLSASMVKRYEEHQKDLKKLKDFIKVNAPDQYNAIFKDK
NKKGYASYIESGVKQDEFYKYLKGILLKINGSGDELDKIDREDFLRKQRTEDNGSIPHQIHLQE
MHAILRRQGEHYPFLKENQDKIEKILTFRIPYYVGPLARKGSRFAWAEYKADEKITPWNEDDIL
DKEKSAEKFITRMTLNDLYLPEEKVLPKHSPLYEAFTVYNELTKVKYVNEQGEAKFEDTNMKQE
IFDHVFKENRKVTKDKLLNYLNKEFEEFRIVNLTGLDKENKAFNSSLGTYHDLRKILDKSFLDD
KANEKTIEDIIQTLTLFEDREMIRQRLQKYSDIFTKAQLKKLERRHYTGWGRLSYKLINGIRNK
ENKKTILDYLIDDGYANRNFMQLINDDALSFKEEIARAQIIDDVDDIANVVHDLPGSPAIKKGI
LQSVKIVDELVKVMGHNPANIIIEMARENQTTDKGRRNSQQRLKLLQDSLKNLDNPVNIKNVEN
QQLQNDRLFLYYIQNGKDMYTGETLDINNLSQYDIDHIIPQAFIKDNSLDNRVLTRSDKNRGKS
DDVPSIEVVHEMKSFWSKLLSVKLITQRKFDNLTKAERGGLTEEDKAGFIKRQLVETRQITKHV
AQILDERENTEFDGNKRRIRNVKIITLKSNLVSNFRKEFELYKVREINDYHHAHDAYLNAVVGN
ALLLKYPQLEPEFVYGEYPKYNSYRSRKSATEKFLFYSNILRFFKKEDIQTNEDGEIAWNKEKH
IKILRKVLSYPQVNIVKKTEEQTGGFSKESILPKGESDKLIPRKTKNSYWDPKKYGGFDSPVVA
YSILVFADVEKGKSKKLRKVQDMVGITIMEKKRFEKNPVDFLEQRGYRNVRLEKIIKLPKYSLF
ELENKRRRLLASAKELQKGNELVIPQRFTTLLYHSYRIEKDYEPEHREYVEKHKDEFKELLEYI
SVFSRKYVLADNNLTKIEMLESKNKDAEVSSLAKSFISLLTFTAFGAPAAFNFFGENIDRKRYT
SVTECLNATLIHQSITGLYETRIDLSKLGEDGKRPAATKKAGQAKKKKGSSGGSGGSGGSTNLS
DIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLYPYDVPDYAYPYDVPDYAYP
YDVPDYA (SEQ ID NO: 21)
Amino Acid Sequence of Adenine Deaminase, TadA8.13m fused to the N-terminal of
nickase SirCas9 (ABE-nSirCas9, D14A mutant).
MPAAKRVKLDGSEVEFSHEYWMRHALTLAKRARDEREVPVGAVLVLNNRVIGEGWNRAIGLHDP
TAHAEIMALRQGGLVMQNYRLYDATLYVTFEPCVMCAGAMIHSRIGRVVFGVRNAKTGAAGSLM
DVLHHPGMNHRVEITEGILADECAALLCRFFRMPRRVENAQKKAQSSTDGSSGSETPGTSESAT
PESSGPKKKRKVGAKNKDIRYSIGLAIGTNSVGWAVMDEHYELLKKGNHHMWGSRLFDAAEPAA
TRRASRSIRRRYNKRRERIRLLRDLLGDMVMEVDPTFFIRLLNVSFLDEEDKQKNLGNDYKDNY
NLFIEKDFNDKTYYDKYPTIYHLRKELCENKEKADPRLIYLALHHIVKYRGNFLKEGQSFAKVY
EDIEEKLDNTLKKEMSLNDLDNLFVDNDINSMITVLSKIYQRSKKADDLLKIMNPTKEERAAYK
EFTKALVGLKFNVSKMILAQEVKKDDKDIELDFSNVDYDSTVDGLQAELGEYIEFIEMLHSINS
WVELQDILGNNSTISAAMVERYEEHKNDLRVLKKVIREELPDKYNEVFREDNPKLHNYLGYIKY
PKNTPVEEFYEYIKRLLAKVDTGEAREILERIDLEKEMLKQNSRINGSIPYQMQKDEMIQIIDN
QSVYYPQLKENREKLISILEFRIPYYFGPLNTHSEFAWIKKFEDKQKERILPWNYDQIVDIDAT
AEGFIERMQNTGTYFPDKPVMAKNSLTVSKFEVLNELNKIRINGKLIPVETKKELLSDLFMKNK
TITDKKLKDWLVTHQYYDTNEELKIEGYQKDLQFSTSLAPWIDFTKIFGEINASNYQLIEKIIY
DISIFEDKKILKRRLKKVYQLDDLLVDKILKLNYTGWSRLSEKLLTGIKSKNSKETILSILENS
NMNLMEIINDESLGFKQIIEESNKKDIEGPFRYDEVKKLAGSPAIKRGIWQALLVVQEITKFMK
HEPSHIYIEFAREEQEKVRTESRIAKLQKIYKDLNLQTKEDQLVYESLKKEDAKKKIDTDALYL
YYLQMGKSMYSGKPLDIDKLSTYHIDHILPRSLIKDDSLDNRVLVLPKENEWKLDSETVPFEIR
NKMMGFWQKLHENGLMSNKKFFSLIRTDENEKDKKRFINRQLVETRQIIKNVAVIINDHYTNTN
VVTVRAELSHQFRERYKIYKNRDLNDLHHAHDAYIACILGQFIHQNFGNMDVNMIYGQYKKNYK
KDVQEHNNYGFILNSMNHIHENDDNSVIWDPSYIGKIKSCFCYKDVYVTKKLEQNDAKLEDLTI
LPSDKNSENGVTKAKIPVNKYRKDVNKYGGFSGDAPIMLAIEADKGKKHVRQVIAFPLRLKNYN
DEERIKFIEKEKNLKNVKILTEVKKNQLILINHQYFFITGTNELVNATQLKLSAKNTKNLENLV
DANKHNKLESIDDANFNEVIQELICKLQEPIYSRYNSIGKEFEDSYEKINAVTKQDKLYIIEYL
IAIMSAKATQGYIKPELAREIGTNGKNKGRIKSFTIDLNKTTFISTSVTGLFSKKYKLGKRPAA
TKKAGQAKKKKGSYPYDVPDYAYPYDVPDYAYPYDVPDYA (SEQ ID NO: 6)
Amino Acid Sequence of Adenine Deaminase, TadA8.13m fused to the N-terminal of
nickase VapCas9 (ABE-nVapCas9, D38A mutant)
MPAAKRVKLDGSEVEFSHEYWMRHALTLAKRARDEREVPVGAVLVLNNRVIGEGWNRAIGLHDP
TAHAEIMALRQGGLVMQNYRLYDATLYVTFEPCVMCAGAMIHSRIGRVVFGVRNAKTGAAGSLM
DVLHHPGMNHRVEITEGILADECAALLCRFFRMPRRVENAQKKAQSSTDGSSGSETPGTSESAT
PESSGPKKKRKVGSIINFQRRGLMETQASNQLISSHLKGYPIKDYFVGLAIGTSSVGWAVINKA
YELLKFRSHKMWGSRLFDEGESAVARRGFRSMRRRLERRKLRLKLLEELFADAMAQVDPTFFMR
LRESKYHYEDKTTGHSSKHILFIDKNYNDQDYFKEYPTVYHLRSELMKSGTDDIRKLFLAVHHI
LKYRGNFLYEGATFDSNASTLDDVIKQALENITENCFDCNSAISSIGQILMEAGKTKSDKAKAI
EHLVDTYIATDTVDTSSKTQKDQVKEDKKRLKAFANLVLGLNASLIDLFGSVEELEEDLKKLQI
TGDTYDDKRDELAKAWSDEIYIIDDCKSVYDAIILLSIKEPGLTISESKVKAFNKHKDDLAILK
SLLKSDRSIYNTMFKVDEKGLHNYVHYIKQGRTEETSCNREDFYKYTKKIVEGLSDSKDKEYIL
SQIELQILLPLQRIKDNGVIPYQLHLEELKAILAKCGPKFPFLNEVADGESVAEKLIKMLEFRI
PYYVGPLNTHHNVDNGGFAWAVRKASGRVTPWNEDDKIDREKSAAAFIKNLTNKCTYLLGEDVL
PKSSLLYSEFMLLNELNNVRIDGKPLEKVVKEHLIEAVFKQDHKKMTKNRIEQFLKDNGYISET
HKHEITGLDGEIKNDLASYRDMVRILGDGFDRSMAEEIITDITIFGESKKMLRETLRKKFASCL
DDEAIKKLTKLRYRDWGRLSQKLLNGIEGCDKAGDGTPETIIILMRNFSYNLMELLGDKESFME
RIQEINAKLTEGQIVNPHDIIDDLALSPAVKRAVWQALRIVDEVAHIKKALPARIFVEVTRSNK
NEKKKKDSRQKRLSDLYAAIKKDDVLLNGLNNEIFGELKSSLAKYDDAALRSKKLYLYYTQMGR
CAYTGEIIELSLLNTDNYDIDHIYPRSLTKDDSFDNLVLCKRTANAQKSDAYPISEEIQKTQKP
FWTFLKQQGLISERKYERLTRITPLTADDLSGFIARQLVETNQSVKAATTLLRRLYPGVDVVFV
KAENVTDERHDNNFIKVRSLNHHHHAKDAYLNIVVGNVYHERFTRNFRAFFKKNGANRTYNLAK
MFNYDVNCTNAKDGKAWDVKTSMDTVKKMMDSNDVRVTKRLLEQTGALADATIYKATVAGKAKD
GAYIGMKTKSSVFADVSKYGGMTKIKNAYSIIVQYTGKKGEVIKEIVPLPIYLTNRNTTDQDLI
NYVASIIPQAKDISIIYGKLCINQLVKVNGFYYYLGGKTNSKFCIDNAIQVIVSNEWIPYLKVL
EKFNNMRKDNKDLKANVVSTRALDNKHTIEVRIVEEKNIEFFDYLVSKLKMPIYQKMKGNKAAE
LSEKGYGLFKKMSLEEQSIHLIELLNLLTNQKTTFEVKPLGITASRSTVGSKISNQDEFKVINE
SITGLYSNEVTIVGKRPAATKKAGQAKKKKGSYPYDVPDYAYPYDVPDYAYPYDVPDYA (SEQ
ID NO: 10)
Amino Acid Sequence of Adenine Deaminase, TadA8.13m fused to the C-terminal of
nickase VapCas9 (nVapCas9-ABE8, D38A mutant)
MPKKKRKVSIINFQRRGLMETQASNQLISSHLKGYPIKDYFVGLAIGTSSVGWAVINKAYELLK
FRSHKMWGSRLFDEGESAVARRGERSMRRRLERRKLRLKLLEELFADAMAQVDPTFFMRLRESK
YHYEDKTTGHSSKHILFIDKNYNDQDYFKEYPTVYHLRSELMKSGTDDIRKLFLAVHHILKYRG
NFLYEGATFDSNASTLDDVIKQALENITENCEDCNSAISSIGQILMEAGKTKSDKAKAIEHLVD
TYIATDTVDTSSKTQKDQVKEDKKRLKAFANLVLGLNASLIDLFGSVEELEEDLKKLQITGDTY
DDKRDELAKAWSDEIYIIDDCKSVYDAIILLSIKEPGLTISESKVKAFNKHKDDLAILKSLLKS
DRSIYNTMFKVDEKGLHNYVHYIKQGRTEETSCNREDFYKYTKKIVEGLSDSKDKEYILSQIEL
QILLPLQRIKDNGVIPYQLHLEELKAILAKCGPKFPFLNEVADGFSVAEKLIKMLEFRIPYYVG
PLNTHHNVDNGGFAWAVRKASGRVTPWNEDDKIDREKSAAAFIKNLTNKCTYLLGEDVLPKSSL
LYSEFMLLNELNNVRIDGKPLEKVVKEHLIEAVFKQDHKKMTKNRIEQFLKDNGYISETHKHEI
TGLDGEIKNDLASYRDMVRILGDGFDRSMAEEIITDITIFGESKKMLRETLRKKFASCLDDEAI
KKLTKLRYRDWGRLSQKLLNGIEGCDKAGDGTPETIIILMRNFSYNLMELLGDKESEMERIQEI
NAKLTEGQIVNPHDIIDDLALSPAVKRAVWQALRIVDEVAHIKKALPARIFVEVTRSNKNEKKK
KDSRQKRLSDLYAAIKKDDVLLNGLNNEIFGELKSSLAKYDDAALRSKKLYLYYTQMGRCAYTG
EIIELSLLNTDNYDIDHIYPRSLTKDDSFDNLVLCKRTANAQKSDAYPISEEIQKTQKPFWTFL
KQQGLISERKYERLTRITPLTADDLSGFIARQLVETNQSVKAATTLLRRLYPGVDVVFVKAENV
TDFRHDNNFIKVRSLNHHHHAKDAYLNIVVGNVYHERFTRNFRAFFKKNGANRTYNLAKMENYD
VNCTNAKDGKAWDVKTSMDTVKKMMDSNDVRVTKRLLEQTGALADATIYKATVAGKAKDGAYIG
MKTKSSVFADVSKYGGMTKIKNAYSIIVQYTGKKGEVIKEIVPLPIYLTNRNTTDQDLINYVAS
IIPQAKDISIIYGKLCINQLVKVNGFYYYLGGKTNSKFCIDNAIQVIVSNEWIPYLKVLEKENN
MRKDNKDLKANVVSTRALDNKHTIEVRIVEEKNIEFFDYLVSKLKMPIYQKMKGNKAAELSEKG
YGLFKKMSLEEQSIHLIELLNLLTNQKTTFEVKPLGITASRSTVGSKISNQDEFKVINESITGL
YSNEVTIVKRPAATKKAGQAKKKKSGSETPGTSESATPESSGSEVEFSHEYWMRHALTLAKRAR
DEREVPVGAVLVLNNRVIGEGWNRAIGLHDPTAHAEIMALRQGGLVMQNYRLYDATLYVTFEPC
VMCAGAMIHSRIGRVVEGVRNAKTGAAGSLMDVLHHPGMNHRVEITEGILADECAALLCRFFRM
PRRVFNAQKKAQSSTDPAAKRVKLDGSYPYDVPDYAYPYDVPDYAYPYDVPDYA (SEQ ID NO:
11)
Amino Acid Sequence of Cytidine Deaminase, ppAPOBEC1 fused to the N-terminal of
nickase VapCas9 (CBE-nVapCas9, D38A mutant)
MPAAKRVKLDTSEKGPSTGDPTLRRRIESWEFDVFYDPRELRKETCLLYEIKWGMSRKIWRSSG
KNTTNHVEVNFIKKFTSERRFHSSISCSITWFLSWSPCWECSQAIREFLSQHPGVTLVIYVARL
FWHMDQRNRQGLRDLVNSGVTIQIMRASEYYHCWRNFVNYPPGDEAHWPQYPPLWMMLYALELH
CIILSLPPCLKISRRWQNHLAFFRLHLQNCHYQTIPPHILLATGLIHPSVTWRLKSGGSSGGSS
GSETPGTSESATPESSGGSSGGSPKKKRKVGSIINFQRRGLMETQASNQLISSHLKGYPIKDYF
VGLAIGTSSVGWAVINKAYELLKFRSHKMWGSRLFDEGESAVARRGFRSMRRRLERRKLRLKLL
EELFADAMAQVDPTFFMRLRESKYHYEDKTTGHSSKHILFIDKNYNDQDYFKEYPTVYHLRSEL
MKSGTDDIRKLFLAVHHILKYRGNFLYEGATFDSNASTLDDVIKQALENITENCFDCNSAISSI
GQILMEAGKTKSDKAKAIEHLVDTYIATDTVDTSSKTQKDQVKEDKKRLKAFANLVLGLNASLI
DLFGSVEELEEDLKKLQITGDTYDDKRDELAKAWSDEIYIIDDCKSVYDAIILLSIKEPGLTIS
ESKVKAFNKHKDDLAILKSLLKSDRSIYNTMFKVDEKGLHNYVHYIKQGRTEETSCNREDFYKY
TKKIVEGLSDSKDKEYILSQIELQILLPLQRIKDNGVIPYQLHLEELKAILAKCGPKFPELNEV
ADGFSVAEKLIKMLEFRIPYYVGPLNTHHNVDNGGFAWAVRKASGRVTPWNEDDKIDREKSAAA
FIKNLTNKCTYLLGEDVLPKSSLLYSEFMLLNELNNVRIDGKPLEKVVKEHLIEAVFKQDHKKM
TKNRIEQFLKDNGYISETHKHEITGLDGEIKNDLASYRDMVRILGDGEDRSMAEEIITDITIFG
ESKKMLRETLRKKFASCLDDEAIKKLTKLRYRDWGRLSQKLLNGIEGCDKAGDGTPETIIILMR
NFSYNLMELLGDKFSFMERIQEINAKLTEGQIVNPHDIIDDLALSPAVKRAVWQALRIVDEVAH
IKKALPARIFVEVTRSNKNEKKKKDSRQKRLSDLYAAIKKDDVLLNGLNNEIFGELKSSLAKYD
DAALRSKKLYLYYTQMGRCAYTGEIIELSLLNTDNYDIDHIYPRSLTKDDSFDNLVLCKRTANA
QKSDAYPISEEIQKTQKPFWTFLKQQGLISERKYERLTRITPLTADDLSGFIARQLVETNQSVK
AATTLLRRLYPGVDVVFVKAENVTDERHDNNFIKVRSLNHHHHAKDAYLNIVVGNVYHERFTRN
FRAFFKKNGANRTYNLAKMFNYDVNCTNAKDGKAWDVKTSMDTVKKMMDSNDVRVTKRLLEQTG
ALADATIYKATVAGKAKDGAYIGMKTKSSVFADVSKYGGMTKIKNAYSIIVQYTGKKGEVIKEI
VPLPIYLTNRNTTDQDLINYVASIIPQAKDISIIYGKLCINQLVKVNGFYYYLGGKTNSKFCID
NAIQVIVSNEWIPYLKVLEKENNMRKDNKDLKANVVSTRALDNKHTIEVRIVEEKNIEFFDYLV
SKLKMPIYQKMKGNKAAELSEKGYGLFKKMSLEEQSIHLIELLNLLTNQKTTFEVKPLGITASR
STVGSKISNQDEFKVINESITGLYSNEVTIVGKRPAATKKAGQAKKKKGSSGGSGGSGGSTNLS
DIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLYPYDVPDYAYPYDVPDYAY
(SEQ ID NO: 12)
Amino Acid Sequence of Adenine Deaminase, TadA8.13m fused to the N-terminal of
nickase EpeCas9 (ABE-nEpeCas9, D12A mutant)
MPAAKRVKLDGSEVEFSHEYWMRHALTLAKRARDEREVPVGAVLVLNNRVIGEGWNRAIGLHDP
TAHAEIMALRQGGLVMQNYRLYDATLYVTFEPCVMCAGAMIHSRIGRVVFGVRNAKTGAAGSLM
DVLHHPGMNHRVEITEGILADECAALLCRFFRMPRRVENAQKKAQSSTDGSSGSETPGTSESAT
PESSGPKKKRKVGTKVKDYYIGLAIGTSSVGWAVTDEAYNVLKENSKKMWGVRLFDDAKTAEER
RGQRGARRRLDRKKERLSLLQDFFAEEVAKVDPNFFLRLDNSDLYMEDKDQKLKSKYTLENDKD
FKDKNFHKKYPTIHHLLMDLIEDDSKKDIRLVYLACHYLLKNRGHFIFEGQKFDTKSSFENSLN
ELKVHLNDEYGLDLEFDNENLINILTDPKLNKTAKKKELKSVIGDTKFLKAVSAIMIGSSQKLV
DLFENPEDFDDSAIKSVDFSTTSFDDKYSDYELALGDKIALVNILKEIYDSSILENLLKEADKS
KDGNKYISNAFVKKYNKHGQDLKEFKRLVRQYHKSAYFDIFRSEKVNDNYVSYTKSSISNNKRV
KANKFTDQEAFYKFAKKHLETIKYKINKVNGSKADLELIDGMLRDMEFKNFMPKIKSSDNGVIP
YQLKLMELNKILENQSKHHEFLNVSDEYGSVCDKIASIMEFRIPYYVGPLNPNSKYAWIKKQKD
SEITPWNFKDVVDLDSSREEFIDSLIGRCTYLKDEKVLPKASLLYNEYMVLNELNNLKLNDLPI
TEEMKKKIFDQLFKTRKKVTLKAVANLLKKEFNINGEILLSGTDGDFKQGLNSYNDFKAIVGDK
VDSDDYRDKIEEIIKLIVLYGDDKSYLQKKIKAGYGKYFTDSEIKKMAGLNYKDWGRLSKKLLT
GLEGANKITGERGSIIHFMREYNLNLMELMSASFTFTEEIQKLNPVDDRKLSYEMVDELYLSPS
VKRMLWQSLRIVDEIKNIMGTDSKKIFIEMARGKEEVKARKESRKNQLLKFYKDGKKAFISEIG
EERYSYLLSEIEGEEENKFRWDNLYLYYTQLGRCMYSLEPIDISELSSKNIYDQDHIYPKSKIY
DDSIENRVLVKKDLNSKKGNSYPIPDEILNKNCYAYWKILYDKGLIGQKKYTRLTRRTGFTDDE
LVQFISRQIVETRQATKETANLLKTICKNSEIVYSKAENASRFRQEFDIVKCRAVNDLHHMHDA
YINIIVGNVYNTKFTKDPMNFVKKQEKARSYNLENMEKYDVKRGGYTAWIADDEKGTVKNASIK
RIRKELEGTNYRFTRMNYIESGALFNATLQRKNKGSRPLKDKGPKSSIEKYGGYTNINKACFAV
LDIKSKNKIERKLMPVEREIYAKQKNDKKLSDEIFSKYLKDRFGIEDYRVVYPVVKMRTLLKID
GSYYFITGGSDKTLELRSALQLILPKKNEWAIKQIDKSSENDYLTIERIQDLTEELVYNTEDII
VNKFKTSVFKKSFLNLFQDDKIENIDFKFKSMDFKEKCKTLLMLVKAIRASGVRQDLKSIDLKS
DYGRLSSKTNNIGNYQEFKIINQSITGLFENEVDLLKLGKRPAATKKAGQAKKKKGSYPYDVPD
YAYPYDVPDYAYPYDVPDYA (SEQ ID NO: 16)
Amino Acid Sequence of Adenine Deaminase, TadA8.13m fused to the C-terminal of
nickase EpeCas9 (nEpeCas9-ABE8, D12A mutant)
MPKKKRKVTKVKDYYIGLAIGTSSVGWAVTDEAYNVLKFNSKKMWGVRLEDDAKTAEERRGQRG
ARRRLDRKKERLSLLQDFFAEEVAKVDPNFFLRLDNSDLYMEDKDQKLKSKYTLENDKDFKDKN
FHKKYPTIHHLLMDLIEDDSKKDIRLVYLACHYLLKNRGHFIFEGQKFDTKSSFENSLNELKVH
LNDEYGLDLEFDNENLINILTDPKLNKTAKKKELKSVIGDTKFLKAVSAIMIGSSQKLVDLFEN
PEDFDDSAIKSVDESTTSFDDKYSDYELALGDKIALVNILKEIYDSSILENLLKEADKSKDGNK
YISNAFVKKYNKHGQDLKEFKRLVRQYHKSAYFDIFRSEKVNDNYVSYTKSSISNNKRVKANKE
TDQEAFYKFAKKHLETIKYKINKVNGSKADLELIDGMLRDMEFKNFMPKIKSSDNGVIPYQLKL
MELNKILENQSKHHEFLNVSDEYGSVCDKIASIMEFRIPYYVGPLNPNSKYAWIKKQKDSEITP
WNFKDVVDLDSSREEFIDSLIGRCTYLKDEKVLPKASLLYNEYMVLNELNNLKLNDLPITEEMK
KKIFDQLFKTRKKVTLKAVANLLKKEFNINGEILLSGTDGDFKQGLNSYNDFKAIVGDKVDSDD
YRDKIEEIIKLIVLYGDDKSYLQKKIKAGYGKYFTDSEIKKMAGLNYKDWGRLSKKLLTGLEGA
NKITGERGSIIHFMREYNLNLMELMSASFTFTEEIQKLNPVDDRKLSYEMVDELYLSPSVKRML
WQSLRIVDEIKNIMGTDSKKIFIEMARGKEEVKARKESRKNQLLKFYKDGKKAFISEIGEERYS
YLLSEIEGEEENKFRWDNLYLYYTQLGRCMYSLEPIDISELSSKNIYDQDHIYPKSKIYDDSIE
NRVLVKKDLNSKKGNSYPIPDEILNKNCYAYWKILYDKGLIGQKKYTRLTRRTGFTDDELVQFI
SRQIVETRQATKETANLLKTICKNSEIVYSKAENASRFRQEFDIVKCRAVNDLHHMHDAYINII
VGNVYNTKFTKDPMNFVKKQEKARSYNLENMFKYDVKRGGYTAWIADDEKGTVKNASIKRIRKE
LEGTNYRFTRMNYIESGALFNATLQRKNKGSRPLKDKGPKSSIEKYGGYTNINKACFAVLDIKS
KNKIERKLMPVEREIYAKQKNDKKLSDEIFSKYLKDRFGIEDYRVVYPVVKMRTLLKIDGSYYF
ITGGSDKTLELRSALQLILPKKNEWAIKQIDKSSENDYLTIERIQDLTEELVYNTFDIIVNKFK
TSVFKKSFLNLFQDDKIENIDFKFKSMDFKEKCKTLLMLVKAIRASGVRQDLKSIDLKSDYGRL
SSKTNNIGNYQEFKIINQSITGLFENEVDLLKLKRPAATKKAGQAKKKKSGSETPGTSESATPE
SSGSEVEFSHEYWMRHALTLAKRARDEREVPVGAVLVLNNRVIGEGWNRAIGLHDPTAHAEIMA
LRQGGLVMQNYRLYDATLYVTFEPCVMCAGAMIHSRIGRVVFGVRNAKTGAAGSLMDVLHHPGM
NHRVEITEGILADECAALLCRFFRMPRRVENAQKKAQSSTDPAAKRVKLDGSYPYDVPDYAYPY
DVPDYAYPYDVPDYA (SEQ ID NO: 17)
Amino Acid Sequence of Adenine Deaminase, TadA8.13m fused to the C-terminal of
nickase EpeCas9 (nEpeCas9-ABE8, D12A mutant)
MPAAKRVKLDTSEKGPSTGDPTLRRRIESWEFDVFYDPRELRKETCLLYEIKWGMSRKIWRSSG
KNTTNHVEVNFIKKFTSERRFHSSISCSITWELSWSPCWECSQAIREFLSQHPGVTLVIYVARL
FWHMDQRNRQGLRDLVNSGVTIQIMRASEYYHCWRNFVNYPPGDEAHWPQYPPLWMMLYALELH
CIILSLPPCLKISRRWQNHLAFFRLHLQNCHYQTIPPHILLATGLIHPSVTWRLKSGGSSGGSS
GSETPGTSESATPESSGGSSGGSPKKKRKVGTKVKDYYIGLAIGTSSVGWAVTDEAYNVLKENS
KKMWGVRLFDDAKTAEERRGQRGARRRLDRKKERLSLLQDFFAEEVAKVDPNFFLRLDNSDLYM
EDKDQKLKSKYTLFNDKDFKDKNFHKKYPTIHHLLMDLIEDDSKKDIRLVYLACHYLLKNRGHF
IFEGQKFDTKSSFENSLNELKVHLNDEYGLDLEFDNENLINILTDPKLNKTAKKKELKSVIGDT
KFLKAVSAIMIGSSQKLVDLFENPEDFDDSAIKSVDFSTTSFDDKYSDYELALGDKIALVNILK
EIYDSSILENLLKEADKSKDGNKYISNAFVKKYNKHGQDLKEFKRLVRQYHKSAYFDIFRSEKV
NDNYVSYTKSSISNNKRVKANKFTDQEAFYKFAKKHLETIKYKINKVNGSKADLELIDGMLRDM
EFKNFMPKIKSSDNGVIPYQLKLMELNKILENQSKHHEFLNVSDEYGSVCDKIASIMEFRIPYY
VGPLNPNSKYAWIKKQKDSEITPWNFKDVVDLDSSREEFIDSLIGRCTYLKDEKVLPKASLLYN
EYMVLNELNNLKLNDLPITEEMKKKIFDQLFKTRKKVTLKAVANLLKKEFNINGEILLSGTDGD
FKQGLNSYNDFKAIVGDKVDSDDYRDKIEEIIKLIVLYGDDKSYLQKKIKAGYGKYFTDSEIKK
MAGLNYKDWGRLSKKLLTGLEGANKITGERGSIIHEMREYNLNLMELMSASFTFTEEIQKLNPV
DDRKLSYEMVDELYLSPSVKRMLWQSLRIVDEIKNIMGTDSKKIFIEMARGKEEVKARKESRKN
QLLKFYKDGKKAFISEIGEERYSYLLSEIEGEEENKFRWDNLYLYYTQLGRCMYSLEPIDISEL
SSKNIYDQDHIYPKSKIYDDSIENRVLVKKDLNSKKGNSYPIPDEILNKNCYAYWKILYDKGLI
GQKKYTRLTRRTGFTDDELVQFISRQIVETRQATKETANLLKTICKNSEIVYSKAENASRERQE
FDIVKCRAVNDLHHMHDAYINIIVGNVYNTKFTKDPMNFVKKQEKARSYNLENMFKYDVKRGGY
TAWIADDEKGTVKNASIKRIRKELEGTNYRFTRMNYIESGALFNATLQRKNKGSRPLKDKGPKS
SIEKYGGYTNINKACFAVLDIKSKNKIERKLMPVEREIYAKQKNDKKLSDEIFSKYLKDREGIE
DYRVVYPVVKMRTLLKIDGSYYFITGGSDKTLELRSALQLILPKKNEWAIKQIDKSSENDYLTI
ERIQDLTEELVYNTFDIIVNKFKTSVFKKSFLNLFQDDKIENIDFKFKSMDFKEKCKTLLMLVK
AIRASGVRQDLKSIDLKSDYGRLSSKINNIGNYQEFKIINQSITGLFENEVDLLKLGKRPAATK
KAGQAKKKKGSSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDIIEKETGKQ
LVIQESILMLPEEVEEVIGNKPESDILVHTAYDESTDENVMLLTSDAPEYKPWALVIQDSNGEN
KIKMLYPYDVPDYAYPYDVPDYAY (SEQ ID NO: 18)
Amino Acid Sequence of Adenine Deaminase, TadA8.13m-nickase fused to the N-terminal
of nickase LfeCas9 (ABE-nLfeCas9, D9A mutant)
MPAAKRVKLDGSEVEFSHEYWMRHALTLAKRARDEREVPVGAVLVLNNRVIGEGWNRAIGLHDPT
AHAEIMALRQGGLVMQNYRLYDATLYVTFEPCVMCAGAMIHSRIGRVVFGVRNAKTGAAGSLMDV
LHHPGMNHRVEITEGILADECAALLCRFFRMPRRVENAQKKAQSSTDGSSGSETPGTSESATPES
SGPKKKRKVGKEYHIGLAIGTSSIGWAVTDSQFKLMRIKGKTAIGVRLFEEGKTAAERRTFRTTR
RRLKRRKWRLHYLDEIFAPHLQEVDENFLRRLKQSNIHPEDPAKNQAFIGKLLFPDLLKKNERGY
PTLIKMRDELPVEQRAHYPVTNIYKLREAMINEDRQFDLREVYLAVHHIVKYRGHELNNASVDKF
KVGRIDFDKSFNVLNEAYEELQNGEGSFTIEPSKVEKIGQLLLDTKMRKLDRQKAVAKLLEVKVA
DKEETKRNKQIATAMSKLVLGYKADFATVAMANGNEWKIDLSSETSEDEIEKFREELSDAQNDIL
TEITSLESQIMLNEIVPNGMSISESMMDRYWTHERQLAEVKEYLATQPASARKEFDQVYNKYIGQ
APKEKGFDLEKGLKKILSKKENWKEIDELLKAGDFLPKQRTSANGVIPHQMHQQELDRIIEKQAK
YYPWLATENPATGERDRHQAKYELDQLVSFRIPYYVGPLVTPEVQKATSGAKFAWAKRKEDGEIT
PWNLWDKIDRAESAEAFIKRMTVKDTYLLNEDVLPANSLLYQKYNVLNELNNVRVNGRRLSVGIK
QDIYTELFKKKKTVKAGDVASLVMAKTRGVNKPSVEGLSDPKKENSNLATYLDLKSIVGDKVDDN
RYQMDLENIIEWRSVFEDGEIFADKLTEVEWLTDEQRSALVKKRYKGWGRLSKKLLTGIVDENGQ
RIIDLMWNTDQNEMQIVNQPVFKEQIDQLNQKAITNDGMTLRERVESVLDDAYTSPQNKKAIWQV
VRVVEDIVKAVGNAPKSISIEFARNEGNKGEITRSRRTQLQKLFEDQAHELVKDTSLTEELEKAP
DLSDRYYFYFTQGGKDMYTGDPINFDEISTKYDIDHILPQSFVKDDSLDNRVLVSRAENNKKSDR
VPAKLYAAKMKPYWNQLLKQGLITQRKFENLTMDVDQTIKYRSLGFVKRQLVETRQVIKLTANIL
GSMYQEAGTDIIETRAGLTKQLREEFDLPKVREVNDYHHAVDAYLTTFAGQYLNRRYPKLRSFFV
YGEYMKFKHGSDLKLRNFNFFHELMEGDKSQGKVVDQQTGELITTRDEVADYFDWVINLKVMLIS
NETYEETGKYFDASHESSSLYLKNQNKKSKLVVPLKNKLQPEYYGAYTGITQGYMVILKLLDKKG
GFGVYRIPRYAADILNKCHDEVAYRNKIAEIISSDPRAPKSFEVVVPRVLKGTFLVDGEEKFILS
SYRYKVNATQLILPVSDIKLIQDNFKALKKLNVEMQTKKLIEIYDNILRQVDKYYKLYDINKFRA
KLHDGRSKFVELDDFGQDASKEKVIIKILRGLHFGSDLQNLKEIGFGTTPLGQFQVSEAGIRLSN
TAFIIFKSPTGLFNRKLYLKNLGKRPAATKKAGQAKKKKGSYPYDVPDYAYPYDVPDYAYPYDVP
DYA (SEQ ID NO: 88)
Amino Acid Sequence of Adenine Deaminase, TadA8.13m-nickase fused to the C-terminal
of nickase LfeCas9 (nLfeCas9-ABE, D9A mutant)
MPKKKRKVGKEYHIGLAIGTSSIGWAVTDSQFKLMRIKGKTAIGVRLFEEGKTAAERRTFRTTR
RRLKRRKWRLHYLDEIFAPHLQEVDENFLRRLKQSNIHPEDPAKNQAFIGKLLFPDLLKKNERG
YPTLIKMRDELPVEQRAHYPVTNIYKLREAMINEDRQFDLREVYLAVHHIVKYRGHELNNASVD
KFKVGRIDFDKSFNVLNEAYEELQNGEGSFTIEPSKVEKIGQLLLDTKMRKLDRQKAVAKLLEV
KVADKEETKRNKQIATAMSKLVLGYKADFATVAMANGNEWKIDLSSETSEDEIEKFREELSDAQ
NDILTEITSLESQIMLNEIVPNGMSISESMMDRYWTHERQLAEVKEYLATQPASARKEFDQVYN
KYIGQAPKEKGFDLEKGLKKILSKKENWKEIDELLKAGDFLPKQRTSANGVIPHQMHQQELDRI
IEKQAKYYPWLATENPATGERDRHQAKYELDQLVSFRIPYYVGPLVTPEVQKATSGAKFAWAKR
KEDGEITPWNLWDKIDRAESAEAFIKRMTVKDTYLLNEDVLPANSLLYQKYNVLNELNNVRVNG
RRLSVGIKQDIYTELFKKKKTVKAGDVASLVMAKTRGVNKPSVEGLSDPKKFNSNLATYLDLKS
IVGDKVDDNRYQMDLENIIEWRSVFEDGEIFADKLTEVEWLTDEQRSALVKKRYKGWGRLSKKL
LTGIVDENGQRIIDLMWNTDQNFMQIVNQPVFKEQIDQLNQKAITNDGMTLRERVESVLDDAYT
SPQNKKAIWQVVRVVEDIVKAVGNAPKSISIEFARNEGNKGEITRSRRTQLQKLFEDQAHELVK
DTSLTEELEKAPDLSDRYYFYFTQGGKDMYTGDPINFDEISTKYDIDHILPQSFVKDDSLDNRV
LVSRAENNKKSDRVPAKLYAAKMKPYWNQLLKQGLITQRKFENLTMDVDQTIKYRSLGFVKRQL
VETRQVIKLTANILGSMYQEAGTDIIETRAGLTKQLREEFDLPKVREVNDYHHAVDAYLTTFAG
QYLNRRYPKLRSFFVYGEYMKEKHGSDLKLRNENFFHELMEGDKSQGKVVDQQTGELITTRDEV
ADYFDWVINLKVMLISNETYEETGKYFDASHESSSLYLKNQNKKSKLVVPLKNKLQPEYYGAYT
GITQGYMVILKLLDKKGGFGVYRIPRYAADILNKCHDEVAYRNKIAEIISSDPRAPKSFEVVVP
RVLKGTFLVDGEEKFILSSYRYKVNATQLILPVSDIKLIQDNFKALKKLNVEMQTKKLIEIYDN
ILRQVDKYYKLYDINKFRAKLHDGRSKFVELDDFGQDASKEKVIIKILRGLHFGSDLQNLKEIG
FGTTPLGQFQVSEAGIRLSNTAFIIFKSPTGLFNRKLYLKNLKRPAATKKAGQAKKKKSGSETP
GTSESATPESSGSEVEFSHEYWMRHALTLAKRARDEREVPVGAVLVLNNRVIGEGWNRAIGLHD
PTAHAEIMALRQGGLVMQNYRLYDATLYVTFEPCVMCAGAMIHSRIGRVVEGVRNAKTGAAGSL
MDVLHHPGMNHRVEITEGILADECAALLCRFFRMPRRVENAQKKAQSSTDPAAKRVKLDGSYPY
DVPDYAYPYDVPDYAYPYDVPDYA (SEQ ID NO: 89)
Amino Acid Sequence of Cytidine Deaminase, ppAPOBEC1 fused to the N-terminal of
nickase LfeCas9 (CBE-nLfeCas9, D9A mutant)
MPAAKRVKLDTSEKGPSTGDPTLRRRIESWEFDVFYDPRELRKETCLLYEIKWGMSRKIWRSSG
KNTTNHVEVNFIKKFTSERRFHSSISCSITWELSWSPCWECSQAIREFLSQHPGVTLVIYVARL
FWHMDQRNRQGLRDLVNSGVTIQIMRASEYYHCWRNFVNYPPGDEAHWPQYPPLWMMLYALELH
CIILSLPPCLKISRRWQNHLAFFRLHLQNCHYQTIPPHILLATGLIHPSVTWRLKSGGSSGGSS
GSETPGTSESATPESSGGSSGGSPKKKRKVGKEYHIGLAIGTSSIGWAVTDSQFKLMRIKGKTA
IGVRLFEEGKTAAERRTFRTTRRRLKRRKWRLHYLDEIFAPHLQEVDENFLRRLKQSNIHPEDP
AKNQAFIGKLLFPDLLKKNERGYPTLIKMRDELPVEQRAHYPVTNIYKLREAMINEDRQFDLRE
VYLAVHHIVKYRGHFLNNASVDKFKVGRIDFDKSFNVLNEAYEELQNGEGSFTIEPSKVEKIGQ
LLLDTKMRKLDRQKAVAKLLEVKVADKEETKRNKQIATAMSKLVLGYKADFATVAMANGNEWKI
DLSSETSEDEIEKFREELSDAQNDILTEITSLESQIMLNEIVPNGMSISESMMDRYWTHERQLA
EVKEYLATQPASARKEFDQVYNKYIGQAPKEKGFDLEKGLKKILSKKENWKEIDELLKAGDELP
KQRTSANGVIPHQMHQQELDRIIEKQAKYYPWLATENPATGERDRHQAKYELDQLVSFRIPYYV
GPLVTPEVQKATSGAKFAWAKRKEDGEITPWNLWDKIDRAESAEAFIKRMTVKDTYLLNEDVLP
ANSLLYQKYNVLNELNNVRVNGRRLSVGIKQDIYTELFKKKKTVKAGDVASLVMAKTRGVNKPS
VEGLSDPKKFNSNLATYLDLKSIVGDKVDDNRYQMDLENIIEWRSVFEDGEIFADKLTEVEWLT
DEQRSALVKKRYKGWGRLSKKLLTGIVDENGQRIIDLMWNTDQNFMQIVNQPVFKEQIDQLNQK
AITNDGMTLRERVESVLDDAYTSPQNKKAIWQVVRVVEDIVKAVGNAPKSISIEFARNEGNKGE
ITRSRRTQLQKLFEDQAHELVKDTSLTEELEKAPDLSDRYYFYFTQGGKDMYTGDPINFDEIST
KYDIDHILPQSFVKDDSLDNRVLVSRAENNKKSDRVPAKLYAAKMKPYWNQLLKQGLITQRKFE
NLTMDVDQTIKYRSLGFVKRQLVETRQVIKLTANILGSMYQEAGTDIIETRAGLTKQLREEFDL
PKVREVNDYHHAVDAYLTTFAGQYLNRRYPKLRSFFVYGEYMKEKHGSDLKLRNFNFFHELMEG
DKSQGKVVDQQTGELITTRDEVADYFDWVINLKVMLISNETYEETGKYFDASHESSSLYLKNQN
KKSKLVVPLKNKLQPEYYGAYTGITQGYMVILKLLDKKGGFGVYRIPRYAADILNKCHDEVAYR
NKIAEIISSDPRAPKSFEVVVPRVLKGTFLVDGEEKFILSSYRYKVNATQLILPVSDIKLIQDN
FKALKKLNVEMQTKKLIEIYDNILRQVDKYYKLYDINKFRAKLHDGRSKEVELDDFGQDASKEK
VIIKILRGLHFGSDLQNLKEIGFGTTPLGQFQVSEAGIRLSNTAFIIFKSPTGLFNRKLYLKNL
GKRPAATKKAGQAKKKKGSSGGSGGSGGSTNLSDIIEKETGKQLVIQESILMLPEEVEEVIGNK
PESDILVHTAYDESTDENVMLLTSDAPEYKPWALVIQDSNGENKIKMLSGGSGGSGGSTNLSDI
IEKETGKQLVIQESILMLPEEVEEVIGNKPESDILVHTAYDESTDENVMLLTSDAPEYKPWALV
IQDSNGENKIKMLYPYDVPDYAYPYDVPDYAY (SEQ ID NO: 90)
Amino Acid Sequence of Adenine Deaminase, TadA8.13m-nickase fused to the N-terminal
of nickase PmaCas9 (ABE-PmaCas9, D12A mutant)
MPAAKRVKLDGSEVEFSHEYWMRHALTLAKRARDEREVPVGAVLVLNNRVIGEGWNRAIGLHDP
TAHAEIMALRQGGLVMQNYRLYDATLYVTFEPCVMCAGAMIHSRIGRVVEGVRNAKTGAAGSLM
DVLHHPGMNHRVEITEGILADECAALLCRFFRMPRRVENAQKKAQSSTDGSSGSETPGTSESAT
PESSGPKKKRKVGEKKTNYTIGLAIGTDSVGWAVVKDDLELVKKRMKVLGNTETNYIKKNLWGS
LLFESGQTAKDRRLKRVARRRYERRRNRLTELQKIFAPAIDEVDENFFERLNESFLVPEDKAFS
KNPIFGTLGEDKTYYKTYPTIYHLRQHLADSEEKADVRLIYLALAHMIKYRGHFLIEGKLDTEH
IAINENLEQFFESYNALFSEEPIELRKEELIAIENILREKNSRTVKEKRITSFLKDIGRANKQS
PMMAFITLIVGKKAKFKAAFNLEEEISLNLTDDSYDENLEILLNTIGSDFADLEDHAQRVYNAV
ELAGILSGDVKNTHAKLSAQMVAMYERHKEQLKEYKSFIKANLPDQYDMTFVAPKDAQKKDLKG
YAGYIDGNMSQDSFYKFVKDQLKEVPGSEKFLDSIEKEDFLRKQRSFYNGVIPNQVHLAEMEAI
LDRQENYYPWLKENREKIISLLTFRIPYYVGPLADGQSEFAWLERKSDEKIKPWNFSDVVDLDR
SAEKFIEQLIGRDTYLPDEYVLPKKSLIYQKYMVFNELTKIAYLDERQKRMNLSSVEKKEIFET
LFKKRSKVTEKQLVKFFENYLQIDNPTIFGIEDAFNADYSTYVELAKVPGMKSMMDDPDNEDLM
EEIVKILTVFEDRKMRRKQLEKYKERLSPEQIKELAKKHYTGWGRLSKKLLVGIRDKETQKTIL
DYLVEDDNHSGGRQHLNRNLMQLINDDRLSFKKTIAELQMIDPSADLYAQVQEIAGSPAIKKGI
LLGLKIVDEIIRVMGEKPENIVIEMARENQTTARGKALSKRREAKIKEGLAALGSSLLKENLPG
NADLSQRKIYLYYTQNGKDIYLDEPLDEDRLSQYDEDHIIPQSFTVDNSLDNLVLTNSSQNRGN
KKDDVPSLEVVNRQLAYWRSLKDAGLMTQRKEDNLTKAMRGGLTDKDRERFIQRQLVETRQITK
NVAKLLDMRLNDKKDEAGNKIRETNIVLLKSAMASEFRKMFRLYKVRELNDYHHAHDAYLNAAI
AINLLALYPYMADDFVYGEFRYKKKPQAEKATYEKLRQWNLIKRFGEKQLFTPDHEDCWNKERD
IKTIKKVMGYRQVNVVKKAEERTGMLFKETINGKTNKGSRIPIKKDLDPSKYGGYIEEKMAYYA
VISYEDKKKKPGKTIVGISIMDKKEFEYDSISYLGKLGFSNPVVQIILKNYSLIAYPDGRRRYI
TGATKTTKGKVELQKANQIAMEQDLVNFIYHLKNYDEISHPESYAFVQSHTDYFDRLEDSIEHY
TRRFLDAETNINRLRRIYEEEKKKDPVDIEALVASFIELLKLTSAGAPADFIFMGEAISRRRYN
SMTGLFDGQVIYQSLTGLYETRMRFEDGKRPAATKKAGQAKKKKGSYPYDVPDYAYPYDVPDYA
YPYDVPDYA (SEQ ID NO: 91)
Amino Acid Sequence of Adenine Deaminase, TadA8.13m-nickase fused to the C-terminal
of nickase PmaCas9 (nPmaCas9-ABE, D12A mutant)
MPKKKRKVEKKTNYTIGLAIGTDSVGWAVVKDDLELVKKRMKVLGNTETNYIKKNLWGSLLFES
GQTAKDRRLKRVARRRYERRRNRLTELQKIFAPAIDEVDENFFERLNESELVPEDKAFSKNPIF
GTLGEDKTYYKTYPTIYHLRQHLADSEEKADVRLIYLALAHMIKYRGHFLIEGKLDTEHIAINE
NLEQFFESYNALFSEEPIELRKEELIAIENILREKNSRTVKEKRITSFLKDIGRANKQSPMMAF
ITLIVGKKAKFKAAFNLEEEISLNLTDDSYDENLEILLNTIGSDFADLFDHAQRVYNAVELAGI
LSGDVKNTHAKLSAQMVAMYERHKEQLKEYKSFIKANLPDQYDMTFVAPKDAQKKDLKGYAGYI
DGNMSQDSFYKFVKDQLKEVPGSEKFLDSIEKEDFLRKQRSFYNGVIPNQVHLAEMEAILDRQE
NYYPWLKENREKIISLLTFRIPYYVGPLADGQSEFAWLERKSDEKIKPWNFSDVVDLDRSAEKF
IEQLIGRDTYLPDEYVLPKKSLIYQKYMVFNELTKIAYLDERQKRMNLSSVEKKEIFETLFKKR
SKVTEKQLVKFFENYLQIDNPTIFGIEDAFNADYSTYVELAKVPGMKSMMDDPDNEDLMEEIVK
ILTVFEDRKMRRKQLEKYKERLSPEQIKELAKKHYTGWGRLSKKLLVGIRDKETQKTILDYLVE
DDNHSGGRQHLNRNLMQLINDDRLSFKKTIAELQMIDPSADLYAQVQEIAGSPAIKKGILLGLK
IVDEIIRVMGEKPENIVIEMARENQTTARGKALSKRREAKIKEGLAALGSSLLKENLPGNADLS
QRKIYLYYTQNGKDIYLDEPLDEDRLSQYDEDHIIPQSFTVDNSLDNLVLTNSSQNRGNKKDDV
PSLEVVNRQLAYWRSLKDAGLMTQRKFDNLTKAMRGGLTDKDRERFIQRQLVETRQITKNVAKL
LDMRLNDKKDEAGNKIRETNIVLLKSAMASEFRKMFRLYKVRELNDYHHAHDAYLNAAIAINLL
ALYPYMADDEVYGEFRYKKKPQAEKATYEKLRQWNLIKRFGEKQLFTPDHEDCWNKERDIKTIK
KVMGYRQVNVVKKAEERTGMLFKETINGKTNKGSRIPIKKDLDPSKYGGYIEEKMAYYAVISYE
DKKKKPGKTIVGISIMDKKEFEYDSISYLGKLGFSNPVVQIILKNYSLIAYPDGRRRYITGATK
TTKGKVELQKANQIAMEQDLVNFIYHLKNYDEISHPESYAFVQSHTDYFDRLEDSIEHYTRREL
DAETNINRLRRIYEEEKKKDPVDIEALVASFIELLKLTSAGAPADFIFMGEAISRRRYNSMTGL
FDGQVIYQSLTGLYETRMRFEDKRPAATKKAGQAKKKKSGSETPGTSESATPESSGSEVEFSHE
YWMRHALTLAKRARDEREVPVGAVLVLNNRVIGEGWNRAIGLHDPTAHAEIMALRQGGLVMQNY
RLYDATLYVTFEPCVMCAGAMIHSRIGRVVFGVRNAKTGAAGSLMDVLHHPGMNHRVEITEGIL
ADECAALLCRFFRMPRRVENAQKKAQSSTDPAAKRVKLDGSYPYDVPDYAYPYDVPDYAYPYDV
PDYA (SEQ ID NO: 92)
Amino Acid Sequence of Cytidine Deaminase, ppAPOBEC1 fused to the N-terminal of
nickase PmaCas9 (CBE-nPmaCas9, D12A mutant)
MPAAKRVKLDTSEKGPSTGDPTLRRRIESWEFDVFYDPRELRKETCLLYEIKWGMSRKIWRSSG
KNTTNHVEVNFIKKFTSERRFHSSISCSITWELSWSPCWECSQAIREFLSQHPGVTLVIYVARL
FWHMDQRNRQGLRDLVNSGVTIQIMRASEYYHCWRNFVNYPPGDEAHWPQYPPLWMMLYALELH
CIILSLPPCLKISRRWQNHLAFFRLHLQNCHYQTIPPHILLATGLIHPSVTWRLKSGGSSGGSS
GSETPGTSESATPESSGGSSGGSPKKKRKVGEKKTNYTIGLAIGTDSVGWAVVKDDLELVKKRM
KVLGNTETNYIKKNLWGSLLFESGQTAKDRRLKRVARRRYERRRNRLTELQKIFAPAIDEVDEN
FFFRLNESFLVPEDKAFSKNPIFGTLGEDKTYYKTYPTIYHLRQHLADSEEKADVRLIYLALAH
MIKYRGHFLIEGKLDTEHIAINENLEQFFESYNALFSEEPIELRKEELIAIENILREKNSRTVK
EKRITSFLKDIGRANKQSPMMAFITLIVGKKAKFKAAFNLEEEISLNLTDDSYDENLEILLNTI
GSDFADLFDHAQRVYNAVELAGILSGDVKNTHAKLSAQMVAMYERHKEQLKEYKSFIKANLPDQ
YDMTFVAPKDAQKKDLKGYAGYIDGNMSQDSFYKFVKDQLKEVPGSEKELDSIEKEDELRKQRS
FYNGVIPNQVHLAEMEAILDRQENYYPWLKENREKIISLLTFRIPYYVGPLADGQSEFAWLERK
SDEKIKPWNESDVVDLDRSAEKFIEQLIGRDTYLPDEYVLPKKSLIYQKYMVFNELTKIAYLDE
RQKRMNLSSVEKKEIFETLFKKRSKVTEKQLVKFFENYLQIDNPTIFGIEDAFNADYSTYVELA
KVPGMKSMMDDPDNEDLMEEIVKILTVFEDRKMRRKQLEKYKERLSPEQIKELAKKHYTGWGRL
SKKLLVGIRDKETQKTILDYLVEDDNHSGGRQHLNRNLMQLINDDRLSFKKTIAELQMIDPSAD
LYAQVQEIAGSPAIKKGILLGLKIVDEIIRVMGEKPENIVIEMARENQTTARGKALSKRREAKI
KEGLAALGSSLLKENLPGNADLSQRKIYLYYTQNGKDIYLDEPLDEDRLSQYDEDHIIPQSFTV
DNSLDNLVLTNSSQNRGNKKDDVPSLEVVNRQLAYWRSLKDAGLMTQRKFDNLTKAMRGGLTDK
DRERFIQRQLVETRQITKNVAKLLDMRLNDKKDEAGNKIRETNIVLLKSAMASEFRKMFRLYKV
RELNDYHHAHDAYLNAAIAINLLALYPYMADDFVYGEFRYKKKPQAEKATYEKLRQWNLIKREG
EKQLFTPDHEDCWNKERDIKTIKKVMGYRQVNVVKKAEERTGMLFKETINGKTNKGSRIPIKKD
LDPSKYGGYIEEKMAYYAVISYEDKKKKPGKTIVGISIMDKKEFEYDSISYLGKLGFSNPVVQI
ILKNYSLIAYPDGRRRYITGATKTTKGKVELQKANQIAMEQDLVNFIYHLKNYDEISHPESYAF
VQSHTDYFDRLEDSIEHYTRRELDAETNINRLRRIYEEEKKKDPVDIEALVASFIELLKLTSAG
APADFIFMGEAISRRRYNSMTGLEDGQVIYQSLTGLYETRMRFEDGKRPAATKKAGQAKKKKGS
SGGSGGSGGSTNLSDIIEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQLVIQESILMLP
EEVEEVIGNKPESDILVHTAYDESTDENVMLLTSDAPEYKPWALVIQDSNGENKIKMLYPYDVP
DYAYPYDVPDYAY (SEQ ID NO: 93)
Amino Acid Sequence of Cytidine Deaminase, ppAPOBEC1 fused to the C-terminal of
nickase PmaCas9 (nPmaCas9-CBE, D12A mutant)
MPAAKRVKLDTNLSDIIEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQLVIQESILMLP
EEVEEVIGNKPESDILVHTAYDESTDENVMLLTSDAPEYKPWALVIQDSNGENKIKMLSGGSGG
SGGSPKKKRKVEKKTNYTIGLAIGTDSVGWAVVKDDLELVKKRMKVLGNTETNYIKKNLWGSLL
FESGQTAKDRRLKRVARRRYERRRNRLTELQKIFAPAIDEVDENFFFRLNESFLVPEDKAFSKN
PIFGTLGEDKTYYKTYPTIYHLRQHLADSEEKADVRLIYLALAHMIKYRGHFLIEGKLDTEHIA
INENLEQFFESYNALFSEEPIELRKEELIAIENILREKNSRTVKEKRITSFLKDIGRANKQSPM
MAFITLIVGKKAKFKAAFNLEEEISLNLTDDSYDENLEILLNTIGSDFADLFDHAQRVYNAVEL
AGILSGDVKNTHAKLSAQMVAMYERHKEQLKEYKSFIKANLPDQYDMTFVAPKDAQKKDLKGYA
GYIDGNMSQDSFYKFVKDQLKEVPGSEKFLDSIEKEDELRKQRSFYNGVIPNQVHLAEMEAILD
RQENYYPWLKENREKIISLLTFRIPYYVGPLADGQSEFAWLERKSDEKIKPWNFSDVVDLDRSA
EKFIEQLIGRDTYLPDEYVLPKKSLIYQKYMVFNELTKIAYLDERQKRMNLSSVEKKEIFETLF
KKRSKVTEKQLVKFFENYLQIDNPTIFGIEDAFNADYSTYVELAKVPGMKSMMDDPDNEDLMEE
IVKILTVFEDRKMRRKQLEKYKERLSPEQIKELAKKHYTGWGRLSKKLLVGIRDKETQKTILDY
LVEDDNHSGGRQHLNRNLMQLINDDRLSFKKTIAELQMIDPSADLYAQVQEIAGSPAIKKGILL
GLKIVDEIIRVMGEKPENIVIEMARENQTTARGKALSKRREAKIKEGLAALGSSLLKENLPGNA
DLSQRKIYLYYTQNGKDIYLDEPLDEDRLSQYDEDHIIPQSFTVDNSLDNLVLTNSSQNRGNKK
DDVPSLEVVNRQLAYWRSLKDAGLMTQRKFDNLTKAMRGGLTDKDRERFIQRQLVETRQITKNV
AKLLDMRLNDKKDEAGNKIRETNIVLLKSAMASEFRKMFRLYKVRELNDYHHAHDAYLNAAIAI
NLLALYPYMADDFVYGEFRYKKKPQAEKATYEKLRQWNLIKRFGEKQLFTPDHEDCWNKERDIK
TIKKVMGYRQVNVVKKAEERTGMLFKETINGKTNKGSRIPIKKDLDPSKYGGYIEEKMAYYAVI
SYEDKKKKPGKTIVGISIMDKKEFEYDSISYLGKLGFSNPVVQIILKNYSLIAYPDGRRRYITG
ATKTTKGKVELQKANQIAMEQDLVNFIYHLKNYDEISHPESYAFVQSHTDYFDRLEDSIEHYTR
RFLDAETNINRLRRIYEEEKKKDPVDIEALVASFIELLKLTSAGAPADFIFMGEAISRRRYNSM
TGLFDGQVIYQSLTGLYETRMRFEDKRPAATKKAGQAKKKKGSSGGSSGGSSGSETPGTSESAT
PESSGGSSGGSTSEKGPSTGDPTLRRRIESWEFDVFYDPRELRKETCLLYEIKWGMSRKIWRSS
GKNTTNHVEVNFIKKFTSERRFHSSISCSITWELSWSPCWECSQAIREFLSQHPGVTLVIYVAR
LEWHMDQRNRQGLRDLVNSGVTIQIMRASEYYHCWRNFVNYPPGDEAHWPQYPPLWMMLYALEL
HCIILSLPPCLKISRRWQNHLAFFRLHLQNCHYQTIPPHILLATGLIHPSVTWRYPYDVPDYAY
PYDVPDYAYPYDVPDYA (SEQ ID NO: 94)
Linker (underlined, no italics or bolding)
TadA8 (ABE) or ppABOBEC1 (CBE) (italics and underlined)
Nickase mutation: D10A mutation in ScoCas9, D14A mutation in SirCas9, D38A in
VapCas9, D12A in EpeCas9, D9A in LfeCas9, D12A in PmaCas9 (bold and italics)
2xUGI (bold, italics and underlined)
3xHA tag (italics), can be substituted with different tags

### Example 6. Engineered Streptococcus constellatus (ScoCas9) NGC PAM Variants

This example illustrates the engineering of ScoCas9 variants that recognize NGC PAM variants.

Briefly, two variants were engineered, ScoCas9-NGC-v1, which contains amino acid substitutions for NGC PAM recognition and ScoCas9-NGC-v2, which contains amino acid substitutions for NGC PAM recognition and additional amino acid substitutions that enhance SpyCas9 activity. The amino acid residues were identified by structural comparison between S. pyogenes SpyCas9 and S. constellatus ScoCas9. The amino acid sequence of ScoCas9-NGC-v1 (SEQ ID NO: 95) comprised the following mutations from wild type ScoCas9 sequence: D1117M, S118Q, E1201F, A1299R, D1309A, R1312E, T1314R. The amino acid sequence of ScoCas9-NGC-v2 (SEQ ID NO: 96) comprised the following mutations from wild type ScoCas9 sequence: S409I, R655L, D1117M, S118Q, E1201F, A1299R, D1309A, R1312E, T1314R.

Amino acid sequence of Streptococcus constellatus (ScoCas9) variant (ScoCas9-NGC-v1)
MPKKKRKVGMGKPYSIGLDIGTNSVGWAVVTDDYKVPAKKMKVLGNTDKQSIKKNLLGALLEDS
GETAEATRLKRTARRRYTRRKNRLRYLQEIFTGEMNKVDENFFQRLDDSELVDEDKRGEHHPIF
GNIAAEVKYHDDFPTIYHLRRHLADTSKKADLRLVYLALAHMIKERGHFLYEGDLKAENTDVQA
LFKDFVEEYDKTIEESHLSEITVDALSILTEKVSKSSRLENLIAHYPTEKKNTLFGNLIALSLD
LHPNFKTNFQLSEDAKLQFSKDTYEEDLEGFLGEVGDEYADLFASAKNLYDAILLSGILTVDDN
STKAPLSASMVKRYEEHQKDLKKLKDFIKVNAPDQYNAIFKDKNKKGYASYIESGVKQDEFYKY
LKGILLKINGSGDFLDKIDREDFLRKQRTFDNGSIPHQIHLQEMHAILRRQGEHYPFLKENQDK
IEKILTFRIPYYVGPLARKGSRFAWAEYKADEKITPWNEDDILDKEKSAEKFITRMTLNDLYLP
EEKVLPKHSPLYEAFTVYNELTKVKYVNEQGEAKFFDTNMKQEIFDHVFKENRKVTKDKLLNYL
NKEFEEFRIVNLTGLDKENKAFNSSLGTYHDLRKILDKSFLDDKANEKTIEDIIQTLTLFEDRE
MIRQRLQKYSDIFTKAQLKKLERRHYTGWGRLSYKLINGIRNKENKKTILDYLIDDGYANRNFM
QLINDDALSFKEEIARAQIIDDVDDIANVVHDLPGSPAIKKGILQSVKIVDELVKVMGHNPANI
IIEMARENQTTDKGRRNSQQRLKLLQDSLKNLDNPVNIKNVENQQLQNDRLFLYYIQNGKDMYT
GETLDINNLSQYDIDHIIPQAFIKDNSLDNRVLTRSDKNRGKSDDVPSIEVVHEMKSFWSKLLS
VKLITQRKFDNLTKAERGGLTEEDKAGFIKRQLVETRQITKHVAQILDERENTEFDGNKRRIRN
VKIITLKSNLVSNFRKEFELYKVREINDYHHAHDAYLNAVVGNALLLKYPQLEPEFVYGEYPKY
NSYRSRKSATEKFLFYSNILRFFKKEDIQTNEDGEIAWNKEKHIKILRKVLSYPQVNIVKKTEE
QTGGFSKESILPKGESDKLIPRKTKNSYWDPKKYGGFMQPVVAYSILVFADVEKGKSKKLRKVQ
DMVGITIMEKKRFEKNPVDFLEQRGYRNVRLEKIIKLPKYSLFELENKRRRLLASAKFLQKGNE
LVIPQRFTTLLYHSYRIEKDYEPEHREYVEKHKDEFKELLEYISVFSRKYVLADNNLTKIEMLF
SKNKDAEVSSLAKSFISLLTFTAFGAPRAFNFFGENIARKEYRSVTECLNATLIHQSITGLYET
RIDLSKLGEDGEGADKRTADGSEFESPKKKRKV (SEQ ID NO: 95)
Amino acid sequence of Streptococcus constellatus (ScoCas9) variant (ScoCas9-NGC-v2)
MPKKKRKVGMGKPYSIGLDIGTNSVGWAVVTDDYKVPAKKMKVLGNTDKQSIKKNLLGALLFDS
GETAEATRLKRTARRRYTRRKNRLRYLQEIFTGEMNKVDENFFQRLDDSFLVDEDKRGEHHPIF
GNIAAEVKYHDDFPTIYHLRRHLADTSKKADLRLVYLALAHMIKFRGHELYEGDLKAENTDVQA
LFKDFVEEYDKTIEESHLSEITVDALSILTEKVSKSSRLENLIAHYPTEKKNTLFGNLIALSLD
LHPNFKTNFQLSEDAKLQFSKDTYEEDLEGELGEVGDEYADLFASAKNLYDAILLSGILTVDDN
STKAPLSASMVKRYEEHQKDLKKLKDFIKVNAPDQYNAIFKDKNKKGYASYIESGVKQDEFYKY
LKGILLKINGSGDFLDKIDREDFLRKQRTFDNGIIPHQIHLQEMHAILRRQGEHYPFLKENQDK
IEKILTFRIPYYVGPLARKGSRFAWAEYKADEKITPWNEDDILDKEKSAEKFITRMTLNDLYLP
EEKVLPKHSPLYEAFTVYNELTKVKYVNEQGEAKFFDTNMKQEIFDHVFKENRKVTKDKLLNYL
NKEFEEFRIVNLTGLDKENKAFNSSLGTYHDLRKILDKSFLDDKANEKTIEDIIQTLTLFEDRE
MIRQRLQKYSDIFTKAQLKKLERLHYTGWGRLSYKLINGIRNKENKKTILDYLIDDGYANRNEM
QLINDDALSFKEEIARAQIIDDVDDIANVVHDLPGSPAIKKGILQSVKIVDELVKVMGHNPANI
IIEMARENQTTDKGRRNSQQRLKLLQDSLKNLDNPVNIKNVENQQLQNDRLFLYYIQNGKDMYT
GETLDINNLSQYDIDHIIPQAFIKDNSLDNRVLTRSDKNRGKSDDVPSIEVVHEMKSFWSKLLS
VKLITQRKFDNLTKAERGGLTEEDKAGFIKRQLVETRQITKHVAQILDERENTEFDGNKRRIRN
VKIITLKSNLVSNERKEFELYKVREINDYHHAHDAYLNAVVGNALLLKYPQLEPEFVYGEYPKY
NSYRSRKSATEKFLFYSNILRFFKKEDIQTNEDGEIAWNKEKHIKILRKVLSYPQVNIVKKTEE
QTGGFSKESILPKGESDKLIPRKTKNSYWDPKKYGGFMQPVVAYSILVFADVEKGKSKKLRKVQ
DMVGITIMEKKRFEKNPVDFLEQRGYRNVRLEKIIKLPKYSLFELENKRRRLLASAKFLQKGNE
LVIPQRFTTLLYHSYRIEKDYEPEHREYVEKHKDEFKELLEYISVFSRKYVLADNNLTKIEMLF
SKNKDAEVSSLAKSFISLLTFTAFGAPRAFNFFGENIARKEYRSVTECLNATLIHQSITGLYET
RIDLSKLGEDGEGADKRTADGSEFESPKKKRKV (SEQ ID NO: 96)
NLS (bold italics)
Linker (bold underlined)

ScoCas8-NGC variants were used to target a genomic locus that was randomly integrated into the genome of HEK293T cells by lentivirus mediated insertion and tested for nuclease and base editing activities.

Briefly, HEK293T cells were plated in a 96-well plate. Cells were transfected with expression vectors containing ScoCas9-NGC variants, and guide RNA sequence ATCGACAAGAAAGGGACTGA (SEQ ID NO: 97), 24 hours after plating. The ScoCas9 variants recognized an exemplary NGC 3′ PAM sequence, AGC. Cells were harvested 72 hours post-transfection and total DNA was extracted.

Deep sequencing was carried out to characterize indel patterns in the HEK293T cells. Exemplary targets were amplified using a two-round PCR to add Illumina adapters as well as unique barcodes to the target amplicons. PCR products were run on a 2% gel and gel extracted. Samples were pooled, quantified and cDNA libraries were prepared and sequenced on MiSeq. Indel frequency was determined by deep sequencing 4 days after transfection.

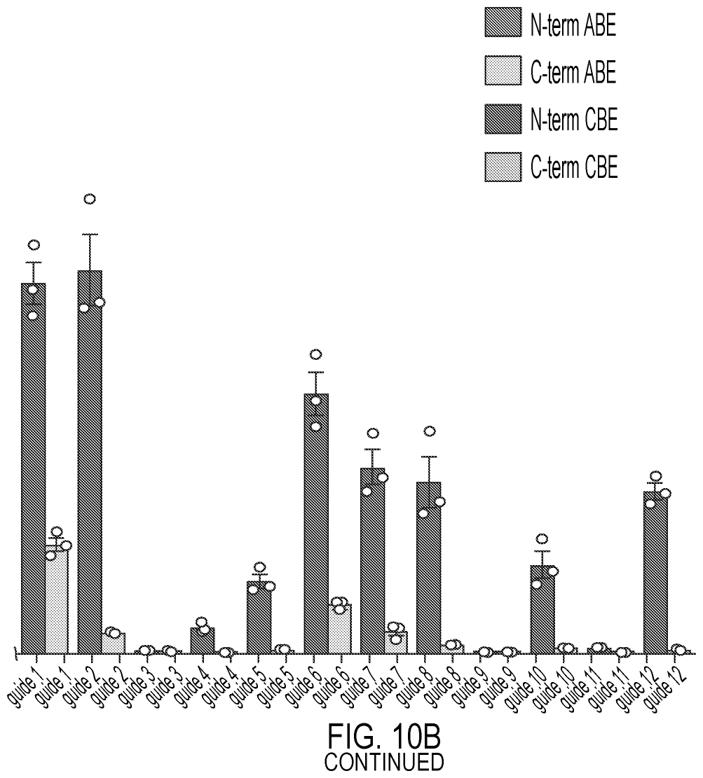

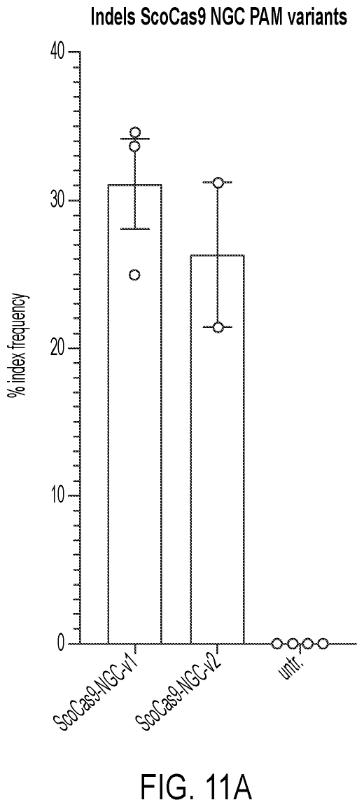

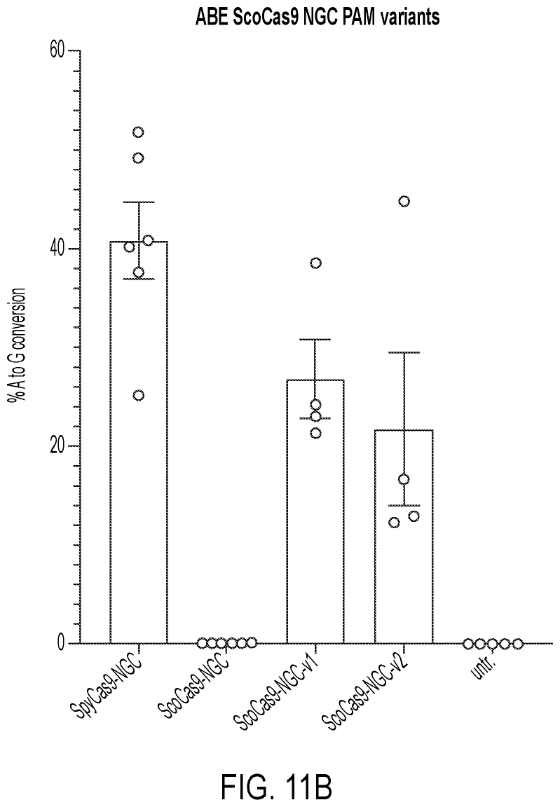

The results showed nuclease activity of both ScoCas9-NGC variants. An indel frequency of between about 20-35% was achieved with ScoCas9-NGC-v1 and ScoCas9-NGC-v2 (FIG. 11A).

Fusions were constructed of ScoCas9-NGC variants with ABE base editors.

Amino acid sequence of a ScoCas9 variant fused to an adenine base editor
(ABE-nScoCas9-NGC-v1)
MSEVEFSHEYWMRHALTLAKRARDEREVPVGAVLVLNNRVIGEGWNRAIGLHDPTAHAEIMALR
QGGLVMQNYRLYDATLYSTFEPCVMCAGAMIHSRIGRVVEGVRNAKTGAAGSLMDVLHHPGMNH
RVEITEGILADECAALLCRFFRMPRRVENAQKKAQSSTDSGGSSGGSSGSETPGTSESATPESS
GGSSGGSGKPYSIGLAIGTNSVGWAVVTDDYKVPAKKMKVLGNTDKQSIKKNLLGALLFDSGET
AEATRLKRTARRRYTRRKNRLRYLQEIFTGEMNKVDENFFQRLDDSFLVDEDKRGEHHPIFGNI
AAEVKYHDDFPTIYHLRRHLADTSKKADLRLVYLALAHMIKFRGHELYEGDLKAENTDVQALFK
DFVEEYDKTIEESHLSEITVDALSILTEKVSKSSRLENLIAHYPTEKKNTLFGNLIALSLDLHP
NFKTNFQLSEDAKLQFSKDTYEEDLEGFLGEVGDEYADLFASAKNLYDAILLSGILTVDDNSTK
APLSASMVKRYEEHQKDLKKLKDFIKVNAPDQYNAIFKDKNKKGYASYIESGVKQDEFYKYLKG
ILLKINGSGDFLDKIDREDFLRKQRTFDNGSIPHQIHLQEMHAILRRQGEHYPFLKENQDKIEK
ILTFRIPYYVGPLARKGSRFAWAEYKADEKITPWNEDDILDKEKSAEKFITRMTLNDLYLPEEK
VLPKHSPLYEAFTVYNELTKVKYVNEQGEAKFFDTNMKQEIFDHVFKENRKVTKDKLLNYLNKE
FEEFRIVNLTGLDKENKAFNSSLGTYHDLRKILDKSFLDDKANEKTIEDIIQTLTLFEDREMIR
QRLQKYSDIFTKAQLKKLERRHYTGWGRLSYKLINGIRNKENKKTILDYLIDDGYANRNFMQLI
NDDALSFKEEIARAQIIDDVDDIANVVHDLPGSPAIKKGILQSVKIVDELVKVMGHNPANIIIE
MARENQTTDKGRRNSQQRLKLLQDSLKNLDNPVNIKNVENQQLQNDRLFLYYIQNGKDMYTGET
LDINNLSQYDIDHIIPQAFIKDNSLDNRVLTRSDKNRGKSDDVPSIEVVHEMKSFWSKLLSVKL
ITQRKFDNLTKAERGGLTEEDKAGFIKRQLVETRQITKHVAQILDERENTEFDGNKRRIRNVKI
ITLKSNLVSNFRKEFELYKVREINDYHHAHDAYLNAVVGNALLLKYPQLEPEFVYGEYPKYNSY
RSRKSATEKFLFYSNILRFFKKEDIQTNEDGEIAWNKEKHIKILRKVLSYPQVNIVKKTEEQTG
GFSKESILPKGESDKLIPRKTKNSYWDPKKYGGFMQPVVAYSILVFADVEKGKSKKLRKVQDMV
GITIMEKKRFEKNPVDFLEQRGYRNVRLEKIIKLPKYSLFELENKRRRLLASAKFLQKGNELVI
PQRFTTLLYHSYRIEKDYEPEHREYVEKHKDEFKELLEYISVFSRKYVLADNNLTKIEMLESKN
KDAEVSSLAKSFISLLTFTAFGAPRAFNFFGENIARKEYRSVTECLNATLIHQSITGLYETRID
LSKLGEDGEGADKRTADGSEFESPKKKRKV (SEQ ID NO: 98)
Amino acid sequence of a ScoCas9 variant fused to an adenine base editor
(ABE-nScoCas9-NGC-v2)
MSEVEFSHEYWMRHALTLAKRARDEREVPVGAVLVLNNRVIGEGWNRAIGLHDPTAHAEIMALR
QGGLVMQNYRLYDATLYSTFEPCVMCAGAMIHSRIGRVVFGVRNAKTGAAGSLMDVLHHPGMNH
RVEITEGILADECAALLCRFFRMPRRVENAQKKAQSSTDSGGSSGGSSGSETPGTSESATPESS
GGSSGGSGKPYSIGLAIGTNSVGWAVVTDDYKVPAKKMKVLGNTDKQSIKKNLLGALLFDSGET
AEATRLKRTARRRYTRRKNRLRYLQEIFTGEMNKVDENFFQRLDDSELVDEDKRGEHHPIFGNI
AAEVKYHDDFPTIYHLRRHLADTSKKADLRLVYLALAHMIKFRGHFLYEGDLKAENTDVQALFK
DFVEEYDKTIEESHLSEITVDALSILTEKVSKSSRLENLIAHYPTEKKNTLFGNLIALSLDLHP
NFKTNFQLSEDAKLQFSKDTYEEDLEGFLGEVGDEYADLFASAKNLYDAILLSGILTVDDNSTK
APLSASMVKRYEEHQKDLKKLKDFIKVNAPDQYNAIFKDKNKKGYASYIESGVKQDEFYKYLKG
ILLKINGSGDFLDKIDREDFLRKQRTFDNGIIPHQIHLQEMHAILRRQGEHYPFLKENQDKIEK
ILTFRIPYYVGPLARKGSRFAWAEYKADEKITPWNEDDILDKEKSAEKFITRMTLNDLYLPEEK
VLPKHSPLYEAFTVYNELTKVKYVNEQGEAKFFDTNMKQEIFDHVFKENRKVTKDKLLNYLNKE
FEEFRIVNLTGLDKENKAFNSSLGTYHDLRKILDKSFLDDKANEKTIEDIIQTLTLFEDREMIR
QRLQKYSDIFTKAQLKKLERLHYTGWGRLSYKLINGIRNKENKKTILDYLIDDGYANRNFMQLI
NDDALSFKEEIARAQIIDDVDDIANVVHDLPGSPAIKKGILQSVKIVDELVKVMGHNPANIIIE
MARENQTTDKGRRNSQQRLKLLQDSLKNLDNPVNIKNVENQQLQNDRLFLYYIQNGKDMYTGET
LDINNLSQYDIDHIIPQAFIKDNSLDNRVLTRSDKNRGKSDDVPSIEVVHEMKSFWSKLLSVKL
ITQRKFDNLTKAERGGLTEEDKAGFIKRQLVETRQITKHVAQILDERENTEFDGNKRRIRNVKI
ITLKSNLVSNFRKEFELYKVREINDYHHAHDAYLNAVVGNALLLKYPQLEPEFVYGEYPKYNSY
RSRKSATEKFLFYSNILRFFKKEDIQTNEDGEIAWNKEKHIKILRKVLSYPQVNIVKKTEEQTG
GFSKESILPKGESDKLIPRKTKNSYWDPKKYGGFMQPVVAYSILVFADVEKGKSKKLRKVQDMV
GITIMEKKRFEKNPVDFLEQRGYRNVRLEKIIKLPKYSLFELENKRRRLLASAKFLQKGNELVI
PQRFTTLLYHSYRIEKDYEPEHREYVEKHKDEFKELLEYISVESRKYVLADNNLTKIEMLESKN
KDAEVSSLAKSFISLLTFTAFGAPRAFNFFGENIARKEYRSVTECLNATLIHQSITGLYETRID
LSKLGEDGEGADKRTADGSEFESPKKKRKV (SEQ ID NO: 99)
Linker (bold underlined)
TadA8 (ABE) (italics)
NLS (bold italics)

Deep sequencing was also carried out to characterize A-to-G conversion in the HEK293T cells (FIG. 11B). Adenine-to-Guanine (A-to-G) conversions were measured by NGS 4 days post transfection. The results showed base editing activity by both ABE-nScoCas9-NGC variants. Both variants showed between about 20-30% A-to-G conversion. ScoCas9 that recognized NGG was used as a negative control and showed no base editing. SpyCas9 was used as a positive control and showed about 40% A-to-G conversion.

Overall, the results showed that ScoCas9 variants engineered to recognize NGC PAM sequences could carry out nuclease as well as base editing activities.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. An engineered, non-naturally occurring Cas9 protein modified from Streptococcus constellatus Cas9, Sharpea Cas9, Veillonella parvula Cas9, Ezakiella peruensis Cas9, Lactobacillus fermentum strain AF15-40LB Cas9, or Peptoniphilus sp. Marseille-P3761 Cas9.

2. The Cas9 protein of claim 1, wherein the Streptococcus constellatus Cas9 has at least 80% sequence identity to
(SEQ ID NO: 1)
MGKPYSIGLDIGTNSVGWAVVTDDYKVPAKKMKVLGNTDKQSIKKNLLGALLEDSGETAEAT
RLKRTARRRYTRRKNRLRYLQEIFTGEMNKVDENFFQRLDDSELVDEDKRGEHHPIFGNIAA
EVKYHDDFPTIYHLRRHLADTSKKADLRLVYLALAHMIKERGHELYEGDLKAENTDVQALFK
DFVEEYDKTIEESHLSEITVDALSILTEKVSKSSRLENLIAHYPTEKKNTLFGNLIALSLDL
HPNFKTNFQLSEDAKLQFSKDTYEEDLEGFLGEVGDEYADLFASAKNLYDAILLSGILTVDD
NSTKAPLSASMVKRYEEHQKDLKKLKDFIKVNAPDQYNAIFKDKNKKGYASYIESGVKQDEF
YKYLKGILLKINGSGDFLDKIDREDFLRKQRTFDNGSIPHQIHLQEMHAILRRQGEHYPFLK
ENQDKIEKILTFRIPYYVGPLARKGSRFAWAEYKADEKITPWNEDDILDKEKSAEKFITRMT
LNDLYLPEEKVLPKHSPLYEAFTVYNELTKVKYVNEQGEAKFFDTNMKQEIFDHVFKENRKV
TKDKLLNYLNKEFEEFRIVNLTGLDKENKAFNSSLGTYHDLRKILDKSFLDDKANEKTIEDI
IQTLTLFEDREMIRQRLQKYSDIFTKAQLKKLERRHYTGWGRLSYKLINGIRNKENKKTILD
YLIDDGYANRNFMQLINDDALSFKEEIARAQIIDDVDDIANVVHDLPGSPAIKKGILQSVKI
VDELVKVMGHNPANIIIEMARENQTTDKGRRNSQQRLKLLQDSLKNLDNPVNIKNVENQQLQ
NDRLFLYYIQNGKDMYTGETLDINNLSQYDIDHIIPQAFIKDNSLDNRVLTRSDKNRGKSDD
VPSIEVVHEMKSFWSKLLSVKLITQRKEDNLTKAERGGLTEEDKAGFIKRQLVETRQITKHV
AQILDERENTEFDGNKRRIRNVKIITLKSNLVSNFRKEFELYKVREINDYHHAHDAYLNAVV
GNALLLKYPQLEPEFVYGEYPKYNSYRSRKSATEKFLFYSNILRFFKKEDIQTNEDGEIAWN
KEKHIKILRKVLSYPQVNIVKKTEEQTGGFSKESILPKGESDKLIPRKTKNSYWDPKKYGGF
DSPVVAYSILVFADVEKGKSKKLRKVQDMVGITIMEKKRFEKNPVDFLEQRGYRNVRLEKII
KLPKYSLFELENKRRRLLASAKELQKGNELVIPQRFTTLLYHSYRIEKDYEPEHREYVEKHK
DEFKELLEYISVFSRKYVLADNNLTKIEMLFSKNKDAEVSSLAKSFISLLTFTAFGAPAAEN
FFGENIDRKRYTSVTECLNATLIHQSITGLYETRIDLSKLGED.

3. The Cas9 protein of claim 1, wherein the Sharpea Cas9 has at least 80% sequence identity to
(SEQ ID NO: 4)
MAKNKDIRYSIGLDIGTNSVGWAVMDEHYELLKKGNHHMWGSRLFDAAEPAATRRASRSIRR
RYNKRRERIRLLRDLLGDMVMEVDPTFFIRLLNVSFLDEEDKQKNLGNDYKDNYNLFIEKDE
NDKTYYDKYPTIYHLRKELCENKEKADPRLIYLALHHIVKYRGNFLKEGQSFAKVYEDIEEK
LDNTLKKFMSLNDLDNLFVDNDINSMITVLSKIYQRSKKADDLLKIMNPTKEERAAYKEFTK
ALVGLKFNVSKMILAQEVKKDDKDIELDESNVDYDSTVDGLQAELGEYIEFIEMLHSINSWV
ELQDILGNNSTISAAMVERYEEHKNDLRVLKKVIREELPDKYNEVFREDNPKLHNYLGYIKY
PKNTPVEEFYEYIKRLLAKVDTGEAREILERIDLEKFMLKQNSRINGSIPYQMQKDEMIQII
DNQSVYYPQLKENREKLISILEFRIPYYFGPLNTHSEFAWIKKFEDKQKERILPWNYDQIVD
IDATAEGFIERMQNTGTYFPDKPVMAKNSLTVSKFEVLNELNKIRINGKLIPVETKKELLSD
LFMKNKTITDKKLKDWLVTHQYYDTNEELKIEGYQKDLQFSTSLAPWIDFTKIFGEINASNY
QLIEKIIYDISIFEDKKILKRRLKKVYQLDDLLVDKILKLNYTGWSRLSEKLLTGIKSKNSK
ETILSILENSNMNLMEIINDESLGFKQIIEESNKKDIEGPFRYDEVKKLAGSPAIKRGIWQA
LLVVQEITKFMKHEPSHIYIEFAREEQEKVRTESRIAKLQKIYKDLNLQTKEDQLVYESLKK
EDAKKKIDTDALYLYYLQMGKSMYSGKPLDIDKLSTYHIDHILPRSLIKDDSLDNRVLVLPK
ENEWKLDSETVPFEIRNKMMGFWQKLHENGLMSNKKFFSLIRTDENEKDKKRFINRQLVETR
QIIKNVAVIINDHYTNTNVVTVRAELSHQFRERYKIYKNRDLNDLHHAHDAYIACILGQFIH
QNFGNMDVNMIYGQYKKNYKKDVQEHNNYGFILNSMNHIHENDDNSVIWDPSYIGKIKSCFC
YKDVYVTKKLEQNDAKLEDLTILPSDKNSENGVTKAKIPVNKYRKDVNKYGGFSGDAPIMLA
IEADKGKKHVRQVIAFPLRLKNYNDEERIKFIEKEKNLKNVKILTEVKKNQLILINHQYFFI
TGTNELVNATQLKLSAKNTKNLFNLVDANKHNKLESIDDANFNEVIQELICKLQEPIYSRYN
SIGKEFEDSYEKINAVTKQDKLYIIEYLIAIMSAKATQGYIKPELAREIGTNGKNKGRIKSF
TIDLNKTTFISTSVTGLESKKYKL.

4. The Cas9 protein of claim 1, wherein the Veillonella parvula Cas9 has at least 80% sequence identity to
(SEQ ID NO: 8)
MSIINFQRRGLMETQASNQLISSHLKGYPIKDYFVGLDIGTSSVGWAVTNKAYELLKERSHK
MWGSRLFDEGESAVARRGERSMRRRLERRKLRLKLLEELFADAMAQVDPTFFMRLRESKYHY
EDKTTGHSSKHILFIDKNYNDQDYFKEYPTVYHLRSELMKSGTDDIRKLFLAVHHILKYRGN
FLYEGATFDSNASTLDDVIKQALENITENCEDCNSAISSIGQILMEAGKTKSDKAKAIEHLV
DTYIATDTVDTSSKTQKDQVKEDKKRLKAFANLVLGLNASLIDLFGSVEELEEDLKKLQITG
DTYDDKRDELAKAWSDEIYIIDDCKSVYDAIILLSIKEPGLTISESKVKAFNKHKDDLAILK
SLLKSDRSIYNTMFKVDEKGLHNYVHYIKQGRTEETSCNREDFYKYTKKIVEGLSDSKDKEY
ILSQIELQILLPLQRIKDNGVIPYQLHLEELKAILAKCGPKFPFLNEVADGESVAEKLIKML
EFRIPYYVGPLNTHHNVDNGGFAWAVRKASGRVTPWNEDDKIDREKSAAAFIKNLINKCTYL
LGEDVLPKSSLLYSEFMLLNELNNVRIDGKPLEKVVKEHLIEAVFKQDHKKMTKNRIEQFLK
DNGYISETHKHEITGLDGEIKNDLASYRDMVRILGDGEDRSMAEEIITDITIFGESKKMLRE
TLRKKFASCLDDEAIKKLTKLRYRDWGRLSQKLLNGIEGCDKAGDGTPETIIILMRNESYNL
MELLGDKFSFMERIQEINAKLTEGQIVNPHDIIDDLALSPAVKRAVWQALRIVDEVAHIKKA
LPARIFVEVTRSNKNEKKKKDSRQKRLSDLYAAIKKDDVLLNGLNNEIFGELKSSLAKYDDA
ALRSKKLYLYYTQMGRCAYTGEIIELSLLNTDNYDIDHIYPRSLTKDDSFDNLVLCKRTANA
QKSDAYPISEEIQKTQKPFWTFLKQQGLISERKYERLTRITPLTADDLSGFIARQLVETNQS
VKAATTLLRRLYPGVDVVFVKAENVTDERHDNNFIKVRSLNHHHHAKDAYLNIVVGNVYHER
FTRNFRAFFKKNGANRTYNLAKMENYDVNCTNAKDGKAWDVKTSMDTVKKMMDSNDVRVTKR
LLEQTGALADATIYKATVAGKAKDGAYIGMKTKSSVFADVSKYGGMTKIKNAYSIIVQYTGK
KGEVIKEIVPLPIYLTNRNTTDQDLINYVASIIPQAKDISIIYGKLCINQLVKVNGFYYYLG
GKTNSKFCIDNAIQVIVSNEWIPYLKVLEKENNMRKDNKDLKANVVSTRALDNKHTIEVRIV
EEKNIEFFDYLVSKLKMPIYQKMKGNKAAELSEKGYGLFKKMSLEEQSIHLIELLNLLTNQK
TTFEVKPLGITASRSTVGSKISNQDEFKVINESITGLYSNEVTIV.

5. The Cas9 protein of claim 1, wherein the Ezakiella peruensis Cas9 has at least 80% sequence identity to
(SEQ ID NO: 14)
MTKVKDYYIGLDIGTSSVGWAVTDEAYNVLKFNSKKMWGVRLEDDAKTAEERRGQRGARRRL
DRKKERLSLLQDFFAEEVAKVDPNFFLRLDNSDLYMEDKDQKLKSKYTLFNDKDFKDKNFHK
KYPTIHHLLMDLIEDDSKKDIRLVYLACHYLLKNRGHFIFEGQKFDTKSSFENSLNELKVHL
NDEYGLDLEFDNENLINILTDPKLNKTAKKKELKSVIGDTKFLKAVSAIMIGSSQKLVDLFE
NPEDFDDSAIKSVDFSTTSFDDKYSDYELALGDKIALVNILKEIYDSSILENLLKEADKSKD
GNKYISNAFVKKYNKHGQDLKEFKRLVRQYHKSAYFDIFRSEKVNDNYVSYTKSSISNNKRV
KANKFTDQEAFYKFAKKHLETIKYKINKVNGSKADLELIDGMLRDMEFKNEMPKIKSSDNGV
IPYQLKLMELNKILENQSKHHEFLNVSDEYGSVCDKIASIMEFRIPYYVGPLNPNSKYAWIK
KQKDSEITPWNFKDVVDLDSSREEFIDSLIGRCTYLKDEKVLPKASLLYNEYMVLNELNNLK
LNDLPITEEMKKKIFDQLFKTRKKVTLKAVANLLKKEFNINGEILLSGTDGDFKQGLNSYND
FKAIVGDKVDSDDYRDKIEEIIKLIVLYGDDKSYLQKKIKAGYGKYFTDSEIKKMAGLNYKD
WGRLSKKLLTGLEGANKITGERGSIIHEMREYNLNLMELMSASFTFTEEIQKLNPVDDRKLS
YEMVDELYLSPSVKRMLWQSLRIVDEIKNIMGTDSKKIFIEMARGKEEVKARKESRKNQLLK
FYKDGKKAFISEIGEERYSYLLSEIEGEEENKERWDNLYLYYTQLGRCMYSLEPIDISELSS
KNIYDQDHIYPKSKIYDDSIENRVLVKKDLNSKKGNSYPIPDEILNKNCYAYWKILYDKGLI
GQKKYTRLTRRTGFTDDELVQFISRQIVETRQATKETANLLKTICKNSEIVYSKAENASRER
QEFDIVKCRAVNDLHHMHDAYINIIVGNVYNTKFTKDPMNFVKKQEKARSYNLENMEKYDVK
RGGYTAWIADDEKGTVKNASIKRIRKELEGTNYRFTRMNYIESGALFNATLQRKNKGSRPLK
DKGPKSSIEKYGGYTNINKACFAVLDIKSKNKIERKLMPVEREIYAKQKNDKKLSDEIFSKY
LKDRFGIEDYRVVYPVVKMRTLLKIDGSYYFITGGSDKTLELRSALQLILPKKNEWAIKQID
KSSENDYLTIERIQDLTEELVYNTFDIIVNKFKTSVEKKSFLNLFQDDKIENIDFKEKSMDF
KEKCKTLLMLVKAIRASGVRQDLKSIDLKSDYGRLSSKINNIGNYQEFKIINQSITGLFENE
VDLLKL.

6. The Cas9 protein of claim 1, wherein the Lactobacillus fermentum strain AF15-40LB Cas9 has at least 80% sequence identity to
(SEQ ID NO: 84)
MKEYHIGLDIGTSSIGWAVTDSQFKLMRIKGKTAIGVRLFEEGKTAAERRTFRTTRRRLKRR
KWRLHYLDEIFAPHLQEVDENFLRRLKQSNIHPEDPAKNQAFIGKLLFPDLLKKNERGYPTL
IKMRDELPVEQRAHYPVTNIYKLREAMINEDRQFDLREVYLAVHHIVKYRGHELNNASVDKF
KVGRIDFDKSFNVLNEAYEELQNGEGSFTIEPSKVEKIGQLLLDTKMRKLDRQKAVAKLLEV
KVADKEETKRNKQIATAMSKLVLGYKADFATVAMANGNEWKIDLSSETSEDEIEKFREELSD
AQNDILTEITSLESQIMLNEIVPNGMSISESMMDRYWTHERQLAEVKEYLATQPASARKEFD
QVYNKYIGQAPKEKGFDLEKGLKKILSKKENWKEIDELLKAGDELPKQRTSANGVIPHQMHQ
QELDRIIEKQAKYYPWLATENPATGERDRHQAKYELDQLVSFRIPYYVGPLVTPEVQKATSG
AKFAWAKRKEDGEITPWNLWDKIDRAESAEAFIKRMTVKDTYLLNEDVLPANSLLYQKYNVL
NELNNVRVNGRRLSVGIKQDIYTELFKKKKTVKAGDVASLVMAKTRGVNKPSVEGLSDPKKF
NSNLATYLDLKSIVGDKVDDNRYQMDLENIIEWRSVFEDGEIFADKLTEVEWLTDEQRSALV
KKRYKGWGRLSKKLLTGIVDENGQRIIDLMWNTDQNFMQIVNQPVFKEQIDQLNQKAITNDG
MTLRERVESVLDDAYTSPQNKKAIWQVVRVVEDIVKAVGNAPKSISIEFARNEGNKGEITRS
RRTQLQKLFEDQAHELVKDTSLTEELEKAPDLSDRYYFYFTQGGKDMYTGDPINFDEISTKY
DIDHILPQSFVKDDSLDNRVLVSRAENNKKSDRVPAKLYAAKMKPYWNQLLKQGLITQRKFE
NLTMDVDQTIKYRSLGFVKRQLVETRQVIKLTANILGSMYQEAGTDIIETRAGLTKQLREEF
DLPKVREVNDYHHAVDAYLTTFAGQYLNRRYPKLRSFFVYGEYMKFKHGSDLKLRNENFFHE
LMEGDKSQGKVVDQQTGELITTRDEVADYFDWVINLKVMLISNETYEETGKYFDASHESSSL
YLKNQNKKSKLVVPLKNKLQPEYYGAYTGITQGYMVILKLLDKKGGFGVYRIPRYAADILNK
CHDEVAYRNKIAEIISSDPRAPKSFEVVVPRVLKGTFLVDGEEKFILSSYRYKVNATQLILP
VSDIKLIQDNFKALKKLNVEMQTKKLIEIYDNILRQVDKYYKLYDINKFRAKLHDGRSKFVE
LDDFGQDASKEKVIIKILRGLHFGSDLQNLKEIGFGTTPLGQFQVSEAGIRLSNTAFIIFKS
PTGLENRKLYLKNL.

7. The Cas9 protein of claim 1, wherein the Peptoniphilus sp. Marseille P3761 Cas9 has at least 80% sequence identity to
(SEQ ID NO: 86)
MEKKTNYTIGLDIGTDSVGWAVVKDDLELVKKRMKVLGNTETNYIKKNLWGSLLFESGQTAK
DRRLKRVARRRYERRRNRLTELQKIFAPAIDEVDENFFFRLNESFLVPEDKAFSKNPIFGTL
GEDKTYYKTYPTIYHLRQHLADSEEKADVRLIYLALAHMIKYRGHFLIEGKLDTEHIAINEN
LEQFFESYNALFSEEPIELRKEELIAIENILREKNSRTVKEKRITSFLKDIGRANKQSPMMA
FITLIVGKKAKFKAAFNLEEEISLNLTDDSYDENLEILLNTIGSDFADLFDHAQRVYNAVEL
AGILSGDVKNTHAKLSAQMVAMYERHKEQLKEYKSFIKANLPDQYDMTFVAPKDAQKKDLKG
YAGYIDGNMSQDSFYKFVKDQLKEVPGSEKFLDSIEKEDELRKQRSFYNGVIPNQVHLAEME
AILDRQENYYPWLKENREKIISLLTFRIPYYVGPLADGQSEFAWLERKSDEKIKPWNFSDVV
DLDRSAEKFIEQLIGRDTYLPDEYVLPKKSLIYQKYMVFNELTKIAYLDERQKRMNLSSVEK
KEIFETLFKKRSKVTEKQLVKFFENYLQIDNPTIFGIEDAFNADYSTYVELAKVPGMKSMMD
DPDNEDLMEEIVKILTVFEDRKMRRKQLEKYKERLSPEQIKELAKKHYTGWGRLSKKLLVGI
RDKETQKTILDYLVEDDNHSGGRQHLNRNLMQLINDDRLSFKKTIAELQMIDPSADLYAQVQ
EIAGSPAIKKGILLGLKIVDEIIRVMGEKPENIVIEMARENQTTARGKALSKRREAKIKEGL
AALGSSLLKENLPGNADLSQRKIYLYYTQNGKDIYLDEPLDEDRLSQYDEDHIIPQSFTVDN
SLDNLVLTNSSQNRGNKKDDVPSLEVVNRQLAYWRSLKDAGLMTQRKFDNLTKAMRGGLTDK
DRERFIQRQLVETRQITKNVAKLLDMRLNDKKDEAGNKIRETNIVLLKSAMASEFRKMERLY
KVRELNDYHHAHDAYLNAAIAINLLALYPYMADDEVYGEFRYKKKPQAEKATYEKLRQWNLI
KRFGEKQLFTPDHEDCWNKERDIKTIKKVMGYRQVNVVKKAEERTGMLFKETINGKTNKGSR
IPIKKDLDPSKYGGYIEEKMAYYAVISYEDKKKKPGKTIVGISIMDKKEFEYDSISYLGKLG
FSNPVVQIILKNYSLIAYPDGRRRYITGATKTTKGKVELQKANQIAMEQDLVNFIYHLKNYD
EISHPESYAFVQSHTDYFDRLEDSIEHYTRRFLDAETNINRLRRIYEEEKKKDPVDIEALVA
SFIELLKLTSAGAPADFIFMGEAISRRRYNSMTGLEDGQVIYQSLTGLYETRMRFED.

8. The Cas9 protein of any one of claims 2-7 comprising an amino acid sequence that is at least 85%, at least 90%, at least 92%, at least 95%, at least 96%, at least 97%, at least 98%, or at least 99% identical to SEQ ID NOs: 1, 4, 8, 14, 84 or 86.

9. The Cas9 protein of any one of the preceding claims, further comprising a nuclear localization sequence (NLS) and/or a FLAG, HIS or HA tag.

10. The Cas9 protein of claim 9, wherein the Streptococcus constellatus Cas9 has an amino acid sequence at least 80% identical to
(SEQ ID NO: 2)
MPKKKRKVGGKPYSIGLDIGTNSVGWAVVTDDYKVPAKKMKVLGNTDKQSIKKNLLGALLFD
SGETAEATRLKRTARRRYTRRKNRLRYLQEIFTGEMNKVDENFFQRLDDSELVDEDKRGEHH
PIFGNIAAEVKYHDDFPTIYHLRRHLADTSKKADLRLVYLALAHMIKERGHFLYEGDLKAEN
TDVQALFKDFVEEYDKTIEESHLSEITVDALSILTEKVSKSSRLENLIAHYPTEKKNTLFGN
LIALSLDLHPNFKTNFQLSEDAKLQFSKDTYEEDLEGFLGEVGDEYADLFASAKNLYDAILL
SGILTVDDNSTKAPLSASMVKRYEEHQKDLKKLKDFIKVNAPDQYNAIFKDKNKKGYASYIE
SGVKQDEFYKYLKGILLKINGSGDFLDKIDREDFLRKQRTEDNGSIPHQIHLQEMHAILRRQ
GEHYPFLKENQDKIEKILTFRIPYYVGPLARKGSRFAWAEYKADEKITPWNEDDILDKEKSA
EKFITRMTLNDLYLPEEKVLPKHSPLYEAFTVYNELTKVKYVNEQGEAKFEDTNMKQEIFDH
VFKENRKVTKDKLLNYLNKEFEEFRIVNLTGLDKENKAFNSSLGTYHDLRKILDKSFLDDKA
NEKTIEDIIQTLTLFEDREMIRQRLQKYSDIFTKAQLKKLERRHYTGWGRLSYKLINGIRNK
ENKKTILDYLIDDGYANRNFMQLINDDALSFKEEIARAQIIDDVDDIANVVHDLPGSPAIKK
GILQSVKIVDELVKVMGHNPANIIIEMARENQTTDKGRRNSQQRLKLLQDSLKNLDNPVNIK
NVENQQLQNDRLFLYYIQNGKDMYTGETLDINNLSQYDIDHIIPQAFIKDNSLDNRVLTRSD
KNRGKSDDVPSIEVVHEMKSFWSKLLSVKLITQRKFDNLTKAERGGLTEEDKAGFIKRQLVE
TRQITKHVAQILDERENTEFDGNKRRIRNVKIITLKSNLVSNFRKEFELYKVREINDYHHAH
DAYLNAVVGNALLLKYPQLEPEFVYGEYPKYNSYRSRKSATEKFLFYSNILRFFKKEDIQTN
EDGEIAWNKEKHIKILRKVLSYPQVNIVKKTEEQTGGFSKESILPKGESDKLIPRKTKNSYW
DPKKYGGFDSPVVAYSILVFADVEKGKSKKLRKVQDMVGITIMEKKRFEKNPVDFLEQRGYR
NVRLEKIIKLPKYSLFELENKRRRLLASAKELQKGNELVIPQRFTTLLYHSYRIEKDYEPEH
REYVEKHKDEFKELLEYISVESRKYVLADNNLTKIEMLESKNKDAEVSSLAKSFISLLTFTA
FGAPAAFNFFGENIDRKRYTSVTECLNATLIHQSITGLYETRIDLSKLGEDGKRPAATKKAG
QAKKKKGSYPYDVPDYAYPYDVPDYAYPYDVPDYA.

10b. The Cas9 protein of claim 9, wherein the Streptococcus constellatus Cas9 has an amino acid sequence at least 80% identical to
(SEQ ID NO: 95)
MPKKKRKVGMGKPYSIGLDIGTNSVGWAVVTDDYKVPAKKMKVLGNTDKQSIKKNLLGALLF
DSGETAEATRLKRTARRRYTRRKNRLRYLQEIFTGEMNKVDENFFQRLDDSFLVDEDKRGEH
HPIFGNIAAEVKYHDDFPTIYHLRRHLADTSKKADLRLVYLALAHMIKERGHFLYEGDLKAE
NTDVQALFKDFVEEYDKTIEESHLSEITVDALSILTEKVSKSSRLENLIAHYPTEKKNTLFG
NLIALSLDLHPNEKTNFQLSEDAKLQFSKDTYEEDLEGELGEVGDEYADLFASAKNLYDAIL
LSGILTVDDNSTKAPLSASMVKRYEEHQKDLKKLKDFIKVNAPDQYNAIFKDKNKKGYASYI
ESGVKQDEFYKYLKGILLKINGSGDFLDKIDREDFLRKQRTFDNGSIPHQIHLQEMHAILRR
QGEHYPFLKENQDKIEKILTFRIPYYVGPLARKGSRFAWAEYKADEKITPWNEDDILDKEKS
AEKFITRMTLNDLYLPEEKVLPKHSPLYEAFTVYNELTKVKYVNEQGEAKFFDTNMKQEIFD
HVFKENRKVTKDKLLNYLNKEFEEFRIVNLTGLDKENKAFNSSLGTYHDLRKILDKSFLDDK
ANEKTIEDIIQTLTLFEDREMIRQRLQKYSDIFTKAQLKKLERRHYTGWGRLSYKLINGIRN
KENKKTILDYLIDDGYANRNFMQLINDDALSFKEEIARAQIIDDVDDIANVVHDLPGSPAIK
KGILQSVKIVDELVKVMGHNPANIIIEMARENQTTDKGRRNSQQRLKLLQDSLKNLDNPVNI
KNVENQQLQNDRLFLYYIQNGKDMYTGETLDINNLSQYDIDHIIPQAFIKDNSLDNRVLTRS
DKNRGKSDDVPSIEVVHEMKSFWSKLLSVKLITQRKEDNLTKAERGGLTEEDKAGFIKRQLV
ETRQITKHVAQILDERENTEFDGNKRRIRNVKIITLKSNLVSNFRKEFELYKVREINDYHHA
HDAYLNAVVGNALLLKYPQLEPEFVYGEYPKYNSYRSRKSATEKFLFYSNILRFFKKEDIQT
NEDGEIAWNKEKHIKILRKVLSYPQVNIVKKTEEQTGGFSKESILPKGESDKLIPRKTKNSY
WDPKKYGGFMQPVVAYSILVFADVEKGKSKKLRKVQDMVGITIMEKKRFEKNPVDFLEQRGY
RNVRLEKIIKLPKYSLFELENKRRRLLASAKFLQKGNELVIPQRFTTLLYHSYRIEKDYEPE
HREYVEKHKDEFKELLEYISVESRKYVLADNNLTKIEMLESKNKDAEVSSLAKSFISLLTFT
AFGAPRAFNFFGENIARKEYRSVTECLNATLIHQSITGLYETRIDLSKLGEDGEGADKRTAD
GSEFESPKKKRKV.

10c. The Cas9 protein of claim 9, wherein the Streptococcus constellatus Cas9 has an amino acid sequence at least 80% identical to
(SEQ ID NO: 96)
MPKKKRKVGMGKPYSIGLDIGTNSVGWAVVTDDYKVPAKKMKVLGNTDKQSIKKNLLGALLE
DSGETAEATRLKRTARRRYTRRKNRLRYLQEIFTGEMNKVDENFFQRLDDSELVDEDKRGEH
HPIFGNIAAEVKYHDDFPTIYHLRRHLADTSKKADLRLVYLALAHMIKFRGHELYEGDLKAE
NTDVQALFKDEVEEYDKTIEESHLSEITVDALSILTEKVSKSSRLENLIAHYPTEKKNTLFG
NLIALSLDLHPNFKTNFQLSEDAKLQFSKDTYEEDLEGFLGEVGDEYADLFASAKNLYDAIL
LSGILTVDDNSTKAPLSASMVKRYEEHQKDLKKLKDFIKVNAPDQYNAIFKDKNKKGYASYI
ESGVKQDEFYKYLKGILLKINGSGDELDKIDREDELRKQRTEDNGIIPHQIHLQEMHAILRR
QGEHYPFLKENQDKIEKILTFRIPYYVGPLARKGSRFAWAEYKADEKITPWNEDDILDKEKS
AEKFITRMTLNDLYLPEEKVLPKHSPLYEAFTVYNELTKVKYVNEQGEAKFFDTNMKQEIFD
HVFKENRKVTKDKLLNYLNKEFEEFRIVNLTGLDKENKAFNSSLGTYHDLRKILDKSFLDDK
ANEKTIEDIIQTLTLFEDREMIRQRLQKYSDIFTKAQLKKLERLHYTGWGRLSYKLINGIRN
KENKKTILDYLIDDGYANRNEMQLINDDALSFKEEIARAQIIDDVDDIANVVHDLPGSPAIK
KGILQSVKIVDELVKVMGHNPANIIIEMARENQTTDKGRRNSQQRLKLLQDSLKNLDNPVNI
KNVENQQLQNDRLFLYYIQNGKDMYTGETLDINNLSQYDIDHIIPQAFIKDNSLDNRVLTRS
DKNRGKSDDVPSIEVVHEMKSFWSKLLSVKLITQRKEDNLTKAERGGLTEEDKAGFIKRQLV
ETRQITKHVAQILDERENTEFDGNKRRIRNVKIITLKSNLVSNFRKEFELYKVREINDYHHA
HDAYLNAVVGNALLLKYPQLEPEFVYGEYPKYNSYRSRKSATEKFLFYSNILRFFKKEDIQT
NEDGEIAWNKEKHIKILRKVLSYPQVNIVKKTEEQTGGFSKESILPKGESDKLIPRKTKNSY
WDPKKYGGFMQPVVAYSILVFADVEKGKSKKLRKVQDMVGITIMEKKRFEKNPVDFLEQRGY
RNVRLEKIIKLPKYSLFELENKRRRLLASAKFLQKGNELVIPQRFTTLLYHSYRIEKDYEPE
HREYVEKHKDEFKELLEYISVFSRKYVLADNNLTKIEMLFSKNKDAEVSSLAKSFISLLTFT
AFGAPRAFNFFGENIARKEYRSVTECLNATLIHQSITGLYETRIDLSKLGEDGEGADKRTAD
GSEFESPKKKRKV.

11. The Cas9 protein of claim 9, wherein the Sharpea Cas9 has an amino acid sequence at least 80% identical to
(SEQ ID NO: 5)
MPKKKRKVGAKNKDIRYSIGLDIGTNSVGWAVMDEHYELLKKGNHHMWGSRLFDAAEPAATR
RASRSIRRRYNKRRERIRLLRDLLGDMVMEVDPTFFIRLLNVSFLDEEDKQKNLGNDYKDNY
NLFIEKDENDKTYYDKYPTIYHLRKELCENKEKADPRLIYLALHHIVKYRGNELKEGQSFAK
VYEDIEEKLDNTLKKEMSLNDLDNLFVDNDINSMITVLSKIYQRSKKADDLLKIMNPTKEER
AAYKEFTKALVGLKFNVSKMILAQEVKKDDKDIELDFSNVDYDSTVDGLQAELGEYIEFIEM
LHSINSWVELQDILGNNSTISAAMVERYEEHKNDLRVLKKVIREELPDKYNEVFREDNPKLH
NYLGYIKYPKNTPVEEFYEYIKRLLAKVDTGEAREILERIDLEKEMLKQNSRINGSIPYQMQ
KDEMIQIIDNQSVYYPQLKENREKLISILEFRIPYYFGPLNTHSEFAWIKKFEDKQKERILP
WNYDQIVDIDATAEGFIERMQNTGTYFPDKPVMAKNSLTVSKFEVLNELNKIRINGKLIPVE
TKKELLSDLFMKNKTITDKKLKDWLVTHQYYDTNEELKIEGYQKDLQFSTSLAPWIDFTKIF
GEINASNYQLIEKIIYDISIFEDKKILKRRLKKVYQLDDLLVDKILKLNYTGWSRLSEKLLT
GIKSKNSKETILSILENSNMNLMEIINDESLGFKQIIEESNKKDIEGPFRYDEVKKLAGSPA
IKRGIWQALLVVQEITKFMKHEPSHIYIEFAREEQEKVRTESRIAKLQKIYKDLNLQTKEDQ
LVYESLKKEDAKKKIDTDALYLYYLQMGKSMYSGKPLDIDKLSTYHIDHILPRSLIKDDSLD
NRVLVLPKENEWKLDSETVPFEIRNKMMGFWQKLHENGLMSNKKFFSLIRTDENEKDKKRFI
NRQLVETRQIIKNVAVIINDHYTNTNVVTVRAELSHQFRERYKIYKNRDLNDLHHAHDAYIA
CILGQFIHQNFGNMDVNMIYGQYKKNYKKDVQEHNNYGFILNSMNHIHENDDNSVIWDPSYI
GKIKSCFCYKDVYVTKKLEQNDAKLEDLTILPSDKNSENGVTKAKIPVNKYRKDVNKYGGFS
GDAPIMLAIEADKGKKHVRQVIAFPLRLKNYNDEERIKFIEKEKNLKNVKILTEVKKNQLIL
INHQYFFITGTNELVNATQLKLSAKNTKNLFNLVDANKHNKLESIDDANFNEVIQELICKLQ
EPIYSRYNSIGKEFEDSYEKINAVTKQDKLYIIEYLIAIMSAKATQGYIKPELAREIGTNGK
NKGRIKSFTIDLNKTTFISTSVTGLESKKYKLGKRPAATKKAGQAKKKKGSYPYDVPDYAYP
YDVPDYAYPYDVPDYA.

12. The Cas9 protein of claim 9, wherein the Veillonella parvula Cas9 has an amino acid sequence at least 80% identical to
(SEQ ID NO: 9)
MPKKKRKVGSIINFQRRGLMETQASNQLISSHLKGYPIKDYFVGLDIGTSSVGWAVINKAYE
LLKFRSHKMWGSRLFDEGESAVARRGFRSMRRRLERRKLRLKLLEELFADAMAQVDPTFFMR
LRESKYHYEDKTTGHSSKHILFIDKNYNDQDYFKEYPTVYHLRSELMKSGTDDIRKLFLAVH
HILKYRGNFLYEGATFDSNASTLDDVIKQALENITENCFDCNSAISSIGQILMEAGKTKSDK
AKAIEHLVDTYIATDTVDTSSKTQKDQVKEDKKRLKAFANLVLGLNASLIDLFGSVEELEED
LKKLQITGDTYDDKRDELAKAWSDEIYIIDDCKSVYDAIILLSIKEPGLTISESKVKAFNKH
KDDLAILKSLLKSDRSIYNTMFKVDEKGLHNYVHYIKQGRTEETSCNREDFYKYTKKIVEGL
SDSKDKEYILSQIELQILLPLQRIKDNGVIPYQLHLEELKAILAKCGPKFPFLNEVADGESV
AEKLIKMLEFRIPYYVGPLNTHHNVDNGGFAWAVRKASGRVTPWNEDDKIDREKSAAAFIKN
LTNKCTYLLGEDVLPKSSLLYSEFMLLNELNNVRIDGKPLEKVVKEHLIEAVFKQDHKKMTK
NRIEQFLKDNGYISETHKHEITGLDGEIKNDLASYRDMVRILGDGEDRSMAEEIITDITIFG
ESKKMLRETLRKKFASCLDDEAIKKLTKLRYRDWGRLSQKLLNGIEGCDKAGDGTPETIIIL
MRNFSYNLMELLGDKFSFMERIQEINAKLTEGQIVNPHDIIDDLALSPAVKRAVWQALRIVD
EVAHIKKALPARIFVEVTRSNKNEKKKKDSRQKRLSDLYAAIKKDDVLLNGLNNEIFGELKS
SLAKYDDAALRSKKLYLYYTQMGRCAYTGEIIELSLLNTDNYDIDHIYPRSLTKDDSFDNLV
LCKRTANAQKSDAYPISEEIQKTQKPFWTELKQQGLISERKYERLTRITPLTADDLSGFIAR
QLVETNQSVKAATTLLRRLYPGVDVVFVKAENVTDERHDNNFIKVRSLNHHHHAKDAYLNIV
VGNVYHERFTRNFRAFFKKNGANRTYNLAKMFNYDVNCTNAKDGKAWDVKTSMDTVKKMMDS
NDVRVTKRLLEQTGALADATIYKATVAGKAKDGAYIGMKTKSSVFADVSKYGGMTKIKNAYS
IIVQYTGKKGEVIKEIVPLPIYLTNRNTTDQDLINYVASIIPQAKDISIIYGKLCINQLVKV
NGFYYYLGGKTNSKFCIDNAIQVIVSNEWIPYLKVLEKENNMRKDNKDLKANVVSTRALDNK
HTIEVRIVEEKNIEFFDYLVSKLKMPIYQKMKGNKAAELSEKGYGLFKKMSLEEQSIHLIEL
LNLLTNQKTTFEVKPLGITASRSTVGSKISNQDEFKVINESITGLYSNEVTIVGKRPAATKK
AGQAKKKKGSYPYDVPDYAYPYDVPDYAYPYDVPDYA.

13. The Cas9 protein of claim 9, wherein the Ezakiella peruensis Cas9 has an amino acid sequence at least 80% identical to
(SEQ ID NO: 15)
MPKKKRKVGTKVKDYYIGLDIGTSSVGWAVTDEAYNVLKENSKKMWGVRLEDDAKTAEERRG
QRGARRRLDRKKERLSLLQDFFAEEVAKVDPNFFLRLDNSDLYMEDKDQKLKSKYTLENDKD
FKDKNFHKKYPTIHHLLMDLIEDDSKKDIRLVYLACHYLLKNRGHFIFEGQKEDTKSSFENS
LNELKVHLNDEYGLDLEFDNENLINILTDPKLNKTAKKKELKSVIGDTKFLKAVSAIMIGSS
QKLVDLFENPEDEDDSAIKSVDESTTSFDDKYSDYELALGDKIALVNILKEIYDSSILENLL
KEADKSKDGNKYISNAFVKKYNKHGQDLKEFKRLVRQYHKSAYFDIFRSEKVNDNYVSYTKS
SISNNKRVKANKFTDQEAFYKFAKKHLETIKYKINKVNGSKADLELIDGMLRDMEFKNFMPK
IKSSDNGVIPYQLKLMELNKILENQSKHHEFLNVSDEYGSVCDKIASIMEFRIPYYVGPLNP
NSKYAWIKKQKDSEITPWNFKDVVDLDSSREEFIDSLIGRCTYLKDEKVLPKASLLYNEYMV
LNELNNLKLNDLPITEEMKKKIFDQLFKTRKKVTLKAVANLLKKEFNINGEILLSGTDGDEK
QGLNSYNDFKAIVGDKVDSDDYRDKIEEIIKLIVLYGDDKSYLQKKIKAGYGKYFTDSEIKK
MAGLNYKDWGRLSKKLLTGLEGANKITGERGSIIHFMREYNLNLMELMSASFTFTEEIQKLN
PVDDRKLSYEMVDELYLSPSVKRMLWQSLRIVDEIKNIMGTDSKKIFIEMARGKEEVKARKE
SRKNQLLKFYKDGKKAFISEIGEERYSYLLSEIEGEEENKFRWDNLYLYYTQLGRCMYSLEP
IDISELSSKNIYDQDHIYPKSKIYDDSIENRVLVKKDLNSKKGNSYPIPDEILNKNCYAYWK
ILYDKGLIGQKKYTRLTRRTGFTDDELVQFISRQIVETRQATKETANLLKTICKNSEIVYSK
AENASRFRQEFDIVKCRAVNDLHHMHDAYINIIVGNVYNTKFTKDPMNFVKKQEKARSYNLE
NMFKYDVKRGGYTAWIADDEKGTVKNASIKRIRKELEGTNYRFTRMNYIESGALFNATLQRK
NKGSRPLKDKGPKSSIEKYGGYTNINKACFAVLDIKSKNKIERKLMPVEREIYAKQKNDKKL
SDEIFSKYLKDRFGIEDYRVVYPVVKMRTLLKIDGSYYFITGGSDKTLELRSALQLILPKKN
EWAIKQIDKSSENDYLTIERIQDLTEELVYNTFDIIVNKFKTSVEKKSFLNLFQDDKIENID
FKFKSMDFKEKCKTLLMLVKAIRASGVRQDLKSIDLKSDYGRLSSKINNIGNYQEFKIINQS
ITGLFENEVDLLKLGKRPAATKKAGQAKKKKGSYPYDVPDYAYPYDVPDYAYPYDVPDYA
(D12A mutant in bold).

14. The Cas9 protein of claim 9, wherein the Lactobacillus fermentum strain AF15-40LB Cas9 has an amino acid sequence at least 80% identical to
(SEQ ID NO: 85)
MPKKKRKVGKEYHIGLDIGTSSIGWAVTDSQFKLMRIKGKTAIGVRLFEEGKTAAERRTFRT
TRRRLKRRKWRLHYLDEIFAPHLQEVDENFLRRLKQSNIHPEDPAKNQAFIGKLLFPDLLKK
NERGYPTLIKMRDELPVEQRAHYPVTNIYKLREAMINEDRQFDLREVYLAVHHIVKYRGHEL
NNASVDKFKVGRIDFDKSFNVLNEAYEELQNGEGSFTIEPSKVEKIGQLLLDTKMRKLDRQK
AVAKLLEVKVADKEETKRNKQIATAMSKLVLGYKADFATVAMANGNEWKIDLSSETSEDEIE
KFREELSDAQNDILTEITSLESQIMLNEIVPNGMSISESMMDRYWTHERQLAEVKEYLATQP
ASARKEFDQVYNKYIGQAPKEKGFDLEKGLKKILSKKENWKEIDELLKAGDELPKQRTSANG
VIPHQMHQQELDRIIEKQAKYYPWLATENPATGERDRHQAKYELDQLVSFRIPYYVGPLVTP
EVQKATSGAKFAWAKRKEDGEITPWNLWDKIDRAESAEAFIKRMTVKDTYLLNEDVLPANSL
LYQKYNVLNELNNVRVNGRRLSVGIKQDIYTELFKKKKTVKAGDVASLVMAKTRGVNKPSVE
GLSDPKKENSNLATYLDLKSIVGDKVDDNRYQMDLENIIEWRSVFEDGEIFADKLTEVEWLT
DEQRSALVKKRYKGWGRLSKKLLTGIVDENGQRIIDLMWNTDQNFMQIVNQPVFKEQIDQLN
QKAITNDGMTLRERVESVLDDAYTSPQNKKAIWQVVRVVEDIVKAVGNAPKSISIEFARNEG
NKGEITRSRRTQLQKLFEDQAHELVKDTSLTEELEKAPDLSDRYYFYFTQGGKDMYTGDPIN
FDEISTKYDIDHILPQSFVKDDSLDNRVLVSRAENNKKSDRVPAKLYAAKMKPYWNQLLKQG
LITQRKFENLTMDVDQTIKYRSLGFVKRQLVETRQVIKLTANILGSMYQEAGTDIIETRAGL
TKQLREEFDLPKVREVNDYHHAVDAYLTTFAGQYLNRRYPKLRSFFVYGEYMKFKHGSDLKL
RNFNFFHELMEGDKSQGKVVDQQTGELITTRDEVADYFDWVINLKVMLISNETYEETGKYFD
ASHESSSLYLKNQNKKSKLVVPLKNKLQPEYYGAYTGITQGYMVILKLLDKKGGFGVYRIPR
YAADILNKCHDEVAYRNKIAEIISSDPRAPKSFEVVVPRVLKGTFLVDGEEKFILSSYRYKV
NATQLILPVSDIKLIQDNFKALKKLNVEMQTKKLIEIYDNILRQVDKYYKLYDINKFRAKLH
DGRSKFVELDDFGQDASKEKVIIKILRGLHFGSDLQNLKEIGFGTTPLGQFQVSEAGIRLSN
TAFIIFKSPTGLFNRKLYLKNLGKRPAATKKAGQAKKKKGSYPYDVPDYAYPYDVPDYAYPY
DVPDYA.

15. The Cas9 protein of claim 9, wherein the Peptoniphilus sp. Marseille-P3761 Cas9 has an amino acid sequence at least 80% identical to
(SEQ ID NO: 87)
MPKKKRKVGEKKTNYTIGLDIGTDSVGWAVVKDDLELVKKRMKVLGNTETNYIKKNLWGSLL
FESGQTAKDRRLKRVARRRYERRRNRLTELQKIFAPAIDEVDENFFERLNESFLVPEDKAFS
KNPIFGTLGEDKTYYKTYPTIYHLRQHLADSEEKADVRLIYLALAHMIKYRGHFLIEGKLDT
EHIAINENLEQFFESYNALFSEEPIELRKEELIAIENILREKNSRTVKEKRITSFLKDIGRA
NKQSPMMAFITLIVGKKAKFKAAFNLEEEISLNLTDDSYDENLEILLNTIGSDFADLEDHAQ
RVYNAVELAGILSGDVKNTHAKLSAQMVAMYERHKEQLKEYKSFIKANLPDQYDMTFVAPKD
AQKKDLKGYAGYIDGNMSQDSFYKFVKDQLKEVPGSEKELDSIEKEDELRKQRSFYNGVIPN
QVHLAEMEAILDRQENYYPWLKENREKIISLLTFRIPYYVGPLADGQSEFAWLERKSDEKIK
PWNFSDVVDLDRSAEKFIEQLIGRDTYLPDEYVLPKKSLIYQKYMVFNELTKIAYLDERQKR
MNLSSVEKKEIFETLFKKRSKVTEKQLVKFFENYLQIDNPTIFGIEDAFNADYSTYVELAKV
PGMKSMMDDPDNEDLMEEIVKILTVFEDRKMRRKQLEKYKERLSPEQIKELAKKHYTGWGRL
SKKLLVGIRDKETQKTILDYLVEDDNHSGGRQHLNRNLMQLINDDRLSFKKTIAELQMIDPS
ADLYAQVQEIAGSPAIKKGILLGLKIVDEIIRVMGEKPENIVIEMARENQTTARGKALSKRR
EAKIKEGLAALGSSLLKENLPGNADLSQRKIYLYYTQNGKDIYLDEPLDEDRLSQYDEDHII
PQSFTVDNSLDNLVLTNSSQNRGNKKDDVPSLEVVNRQLAYWRSLKDAGLMTQRKEDNLTKA
MRGGLTDKDRERFIQRQLVETRQITKNVAKLLDMRLNDKKDEAGNKIRETNIVLLKSAMASE
FRKMFRLYKVRELNDYHHAHDAYLNAAIAINLLALYPYMADDEVYGEFRYKKKPQAEKATYE
KLRQWNLIKRFGEKQLFTPDHEDCWNKERDIKTIKKVMGYRQVNVVKKAEERTGMLFKETIN
GKTNKGSRIPIKKDLDPSKYGGYIEEKMAYYAVISYEDKKKKPGKTIVGISIMDKKEFEYDS
ISYLGKLGFSNPVVQIILKNYSLIAYPDGRRRYITGATKTTKGKVELQKANQIAMEQDLVNE
IYHLKNYDEISHPESYAFVQSHTDYFDRLEDSIEHYTRRELDAETNINRLRRIYEEEKKKDP
VDIEALVASFIELLKLTSAGAPADFIFMGEAISRRRYNSMTGLEDGQVIYQSLTGLYETRMR
FEDGKRPAATKKAGQAKKKKGSYPYDVPDYAYPYDVPDYAYPYDVPDYA.

16. The Cas9 protein of any one of the preceding claims, wherein the amino acid sequence of the Cas9 protein comprises at least one, at least two, at least three, at least four, at least five, at least six, at least seven, at least eight, at least nine, or at least 10 mutations in SEQ ID NO: 1, 4, 8, 14, 84 or 86.

17. The Cas9 protein of claim 16, wherein the mutation is an amino acid substitution.

18. The Cas9 protein of any one of the preceding claims, wherein the Cas9 protein has nickase activity.

18b. The Cas9 protein of claim 18, wherein the nickase mutation at an amino acid positions corresponds to one or more amino acids 10, 12, 17, 762, 840, 854, 863, 982, 983, 984, 986, 987 of wild type SpCas9.

19. The Cas9 protein of claim 16, wherein the at least one mutation results in an inactive Cas9 (dCas9).

20. The Cas9 protein of any one of the preceding claims, wherein the Cas9 protein comprises at least one amino acid mutation in PAM Interacting, HNH and/or RuvC domain.

20b. The Cas9 protein of claim 20, wherein the mutation at an amino acid position corresponds to amino acid 14 in the RuvC domain of SirCas9.

20c. The Cas9 protein of claim 20, wherein the mutation at an amino acid position corresponds to amino acid 12 in the RuvC domain of EpeCas9.

20d. The Cas9 protein of claim 20, wherein the mutation at an amino acid position corresponds to amino acid 9 in the RuvC domain of LfeCas9.

20e. The Cas9 protein of claim 20, wherein the mutation at an amino acid position corresponds to amino acid 12 in the RuvC domain of PmaCas9.

20f. The Cas9 protein of claim 20, wherein the Cas9 protein is a hyper-accurate Cas9.

20g. The Cas9 protein of claim 20, wherein the Cas9 protein comprises mutations corresponding to N692A, M694A, Q695A and/or H698A with reference to SpyCas9 (SEQ ID NO: 173).

20h. The Cas9 protein of claim 20, wherein the Cas9 protein is a high-fidelity Cas9.

20i. The Cas9 protein of claim 20, wherein the Cas9 protein comprises mutations corresponding to N467A, R661A, Q695A and/or Q926A with reference to SpyCas9 (SEQ ID NO: 173).

20j. The Cas9 protein of claim 20, wherein the Cas9 protein is a SuperFi-Cas9.

20k. The Cas9 protein of claim 20, wherein Y1016, R1019, Y1010, Y1013, K1031, Q1027 and/or V1018 residues corresponding to SpyCas9 are mutated to aspartic acid.

21. An engineered, non-naturally occurring Cas9 fusion protein comprising a Cas9 protein having at least 80% identity to SEQ ID NOs: 1, 4, 8, 14, 84 or 86 and wherein the Cas9 protein is fused to a histone demethylase, a transcriptional activator, or to a deaminase.

21b. The engineered, non-naturally occurring Cas9 fusion protein of claim 21 further comprising a nuclear localization sequence (NLS) and/or a FLAG, HIS or HA tag.

21c. The engineered, non-naturally occurring Cas9 fusion protein of claim 22 having at least 80% identity to SEQ ID NOs: 2, 5, 9, 15, 85, 87, 95 or 96.

22. The Cas9 protein of claim 21, wherein the Cas9 protein is fused to a cytosine deaminase or to an adenosine deaminase.

23. The Cas9 protein of claim 22, wherein the Cas9 protein is fused to a adenosine deaminase and has an amino acid sequence at least 80% identical to
(a)
(SEQ ID NO: 20)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGGKPYSIGLAIGTNSVGWAVVTDDYKVPAKKMKVLGNTDKQSIK
KNLLGALLFDSGETAEATRLKRTARRRYTRRKNRLRYLQEIFTGEMNKVDENFFQRLDDSEL
VDEDKRGEHHPIFGNIAAEVKYHDDFPTIYHLRRHLADTSKKADLRLVYLALAHMIKFRGHF
LYEGDLKAENTDVQALFKDFVEEYDKTIEESHLSEITVDALSILTEKVSKSSRLENLIAHYP
TEKKNTLFGNLIALSLDLHPNEKTNFQLSEDAKLQFSKDTYEEDLEGELGEVGDEYADLFAS
AKNLYDAILLSGILTVDDNSTKAPLSASMVKRYEEHQKDLKKLKDFIKVNAPDQYNAIFKDK
NKKGYASYIESGVKQDEFYKYLKGILLKINGSGDFLDKIDREDFLRKQRTFDNGSIPHQIHL
QEMHAILRRQGEHYPFLKENQDKIEKILTFRIPYYVGPLARKGSRFAWAEYKADEKITPWNF
DDILDKEKSAEKFITRMTLNDLYLPEEKVLPKHSPLYEAFTVYNELTKVKYVNEQGEAKFFD
TNMKQEIFDHVFKENRKVTKDKLLNYLNKEFEEFRIVNLTGLDKENKAFNSSLGTYHDLRKI
LDKSFLDDKANEKTIEDIIQTLTLFEDREMIRQRLQKYSDIFTKAQLKKLERRHYTGWGRLS
YKLINGIRNKENKKTILDYLIDDGYANRNFMQLINDDALSFKEEIARAQIIDDVDDIANVVH
DLPGSPAIKKGILQSVKIVDELVKVMGHNPANIIIEMARENQTTDKGRRNSQQRLKLLQDSL
KNLDNPVNIKNVENQQLQNDRLFLYYIQNGKDMYTGETLDINNLSQYDIDHIIPQAFIKDNS
LDNRVLTRSDKNRGKSDDVPSIEVVHEMKSFWSKLLSVKLITQRKEDNLTKAERGGLTEEDK
AGFIKRQLVETRQITKHVAQILDERENTEFDGNKRRIRNVKIITLKSNLVSNERKEFELYKV
REINDYHHAHDAYLNAVVGNALLLKYPQLEPEFVYGEYPKYNSYRSRKSATEKFLFYSNILR
FFKKEDIQTNEDGEIAWNKEKHIKILRKVLSYPQVNIVKKTEEQTGGFSKESILPKGESDKL
IPRKTKNSYWDPKKYGGFDSPVVAYSILVFADVEKGKSKKLRKVQDMVGITIMEKKRFEKNP
VDFLEQRGYRNVRLEKIIKLPKYSLFELENKRRRLLASAKELQKGNELVIPQRFTTLLYHSY
RIEKDYEPEHREYVEKHKDEFKELLEYISVFSRKYVLADNNLTKIEMLFSKNKDAEVSSLAK
SFISLLTFTAFGAPAAFNFFGENIDRKRYTSVTECLNATLIHQSITGLYETRIDLSKLGEDG
KRPAATKKAGQAKKKKGSYPYDVPDYAYPYDVPDYAYPYDVPDYA;
(b)
(SEQ ID NO: 6)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGAKNKDIRYSIGLAIGTNSVGWAVMDEHYELLKKGNHHMWGSRL
FDAAEPAATRRASRSIRRRYNKRRERIRLLRDLLGDMVMEVDPTFFIRLLNVSFLDEEDKQK
NLGNDYKDNYNLFIEKDENDKTYYDKYPTIYHLRKELCENKEKADPRLIYLALHHIVKYRGN
FLKEGQSFAKVYEDIEEKLDNTLKKFMSLNDLDNLFVDNDINSMITVLSKIYQRSKKADDLL
KIMNPTKEERAAYKEFTKALVGLKFNVSKMILAQEVKKDDKDIELDFSNVDYDSTVDGLQAE
LGEYIEFIEMLHSINSWVELQDILGNNSTISAAMVERYEEHKNDLRVLKKVIREELPDKYNE
VFREDNPKLHNYLGYIKYPKNTPVEEFYEYIKRLLAKVDTGEAREILERIDLEKFMLKQNSR
TNGSIPYQMQKDEMIQIIDNQSVYYPQLKENREKLISILEFRIPYYFGPLNTHSEFAWIKKE
EDKQKERILPWNYDQIVDIDATAEGFIERMQNTGTYFPDKPVMAKNSLTVSKFEVLNELNKI
RINGKLIPVETKKELLSDLFMKNKTITDKKLKDWLVTHQYYDTNEELKIEGYQKDLQFSTSL
APWIDFTKIFGEINASNYQLIEKIIYDISIFEDKKILKRRLKKVYQLDDLLVDKILKLNYTG
WSRLSEKLLTGIKSKNSKETILSILENSNMNLMEIINDESLGFKQIIEESNKKDIEGPFRYD
EVKKLAGSPAIKRGIWQALLVVQEITKFMKHEPSHIYIEFAREEQEKVRTESRIAKLQKIYK
DLNLQTKEDQLVYESLKKEDAKKKIDTDALYLYYLQMGKSMYSGKPLDIDKLSTYHIDHILP
RSLIKDDSLDNRVLVLPKENEWKLDSETVPFEIRNKMMGFWQKLHENGLMSNKKFFSLIRTD
FNEKDKKRFINRQLVETRQIIKNVAVIINDHYTNTNVVTVRAELSHQFRERYKIYKNRDLND
LHHAHDAYIACILGQFIHQNFGNMDVNMIYGQYKKNYKKDVQEHNNYGFILNSMNHIHENDD
NSVIWDPSYIGKIKSCFCYKDVYVTKKLEQNDAKLEDLTILPSDKNSENGVTKAKIPVNKYR
KDVNKYGGFSGDAPIMLAIEADKGKKHVRQVIAFPLRLKNYNDEERIKFIEKEKNLKNVKIL
TEVKKNQLILINHQYFFITGTNELVNATQLKLSAKNTKNLFNLVDANKHNKLESIDDANENE
VIQELICKLQEPIYSRYNSIGKEFEDSYEKINAVTKQDKLYIIEYLIAIMSAKATQGYIKPE
LAREIGTNGKNKGRIKSFTIDLNKTTFISTSVTGLFSKKYKLGKRPAATKKAGQAKKKKGSY
PYDVPDYAYPYDVPDYAYPYDVPDYA;
(c)
(SEQ ID NO: 10)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVFNAQKKAQSSTDGSSGSETPG
TSESATPESSGPKKKRKVGSIINFQRRGLMETQASNQLISSHLKGYPIKDYFVGLAIGTSSV
GWAVTNKAYELLKFRSHKMWGSRLFDEGESAVARRGFRSMRRRLERRKLRLKLLEELFADAM
AQVDPTFFMRLRESKYHYEDKTTGHSSKHILFIDKNYNDQDYFKEYPTVYHLRSELMKSGTD
DIRKLFLAVHHILKYRGNFLYEGATFDSNASTLDDVIKQALENITENCFDCNSAISSIGQIL
MEAGKTKSDKAKAIEHLVDTYIATDTVDTSSKTQKDQVKEDKKRLKAFANLVLGLNASLIDL
FGSVEELEEDLKKLQITGDTYDDKRDELAKAWSDEIYIIDDCKSVYDAIILLSIKEPGLTIS
ESKVKAFNKHKDDLAILKSLLKSDRSIYNTMFKVDEKGLHNYVHYIKQGRTEETSCNREDFY
KYTKKIVEGLSDSKDKEYILSQIELQILLPLQRIKDNGVIPYQLHLEELKAILAKCGPKFPF
LNEVADGFSVAEKLIKMLEFRIPYYVGPLNTHHNVDNGGFAWAVRKASGRVTPWNEDDKIDR
EKSAAAFIKNLTNKCTYLLGEDVLPKSSLLYSEFMLLNELNNVRIDGKPLEKVVKEHLIEAV
FKQDHKKMTKNRIEQFLKDNGYISETHKHEITGLDGEIKNDLASYRDMVRILGDGEDRSMAE
EIITDITIFGESKKMLRETLRKKFASCLDDEAIKKLTKLRYRDWGRLSQKLLNGIEGCDKAG
DGTPETIIILMRNFSYNLMELLGDKFSFMERIQEINAKLTEGQIVNPHDIIDDLALSPAVKR
AVWQALRIVDEVAHIKKALPARIFVEVTRSNKNEKKKKDSRQKRLSDLYAAIKKDDVLLNGL
NNEIFGELKSSLAKYDDAALRSKKLYLYYTQMGRCAYTGEIIELSLLNTDNYDIDHIYPRSL
TKDDSFDNLVLCKRTANAQKSDAYPISEEIQKTQKPFWTFLKQQGLISERKYERLTRITPLT
ADDLSGFIARQLVETNQSVKAATTLLRRLYPGVDVVFVKAENVTDERHDNNFIKVRSLNHHH
HAKDAYLNIVVGNVYHERFTRNFRAFFKKNGANRTYNLAKMENYDVNCTNAKDGKAWDVKTS
MDTVKKMMDSNDVRVTKRLLEQTGALADATIYKATVAGKAKDGAYIGMKTKSSVFADVSKYG
GMTKIKNAYSIIVQYTGKKGEVIKEIVPLPIYLTNRNTTDQDLINYVASIIPQAKDISIIYG
KLCINQLVKVNGFYYYLGGKTNSKFCIDNAIQVIVSNEWIPYLKVLEKENNMRKDNKDLKAN
VVSTRALDNKHTIEVRIVEEKNIEFFDYLVSKLKMPIYQKMKGNKAAELSEKGYGLFKKMSL
EEQSIHLIELLNLLTNQKTTFEVKPLGITASRSTVGSKISNQDEFKVINESITGLYSNEVTI
VGKRPAATKKAGQAKKKKGSYPYDVPDYAYPYDVPDYAYPYDVPDYA;
(d)
(SEQ ID NO: 11)
MPKKKRKVSIINFQRRGLMETQASNQLISSHLKGYPIKDYFVGLAIGTSSVGWAVINKAYEL
LKFRSHKMWGSRLFDEGESAVARRGFRSMRRRLERRKLRLKLLEELFADAMAQVDPTFFMRL
RESKYHYEDKTTGHSSKHILFIDKNYNDQDYFKEYPTVYHLRSELMKSGTDDIRKLFLAVHH
ILKYRGNFLYEGATFDSNASTLDDVIKQALENITENCEDCNSAISSIGQILMEAGKTKSDKA
KAIEHLVDTYIATDTVDTSSKTQKDQVKEDKKRLKAFANLVLGLNASLIDLFGSVEELEEDL
KKLQITGDTYDDKRDELAKAWSDEIYIIDDCKSVYDAIILLSIKEPGLTISESKVKAFNKHK
DDLAILKSLLKSDRSIYNTMFKVDEKGLHNYVHYIKQGRTEETSCNREDFYKYTKKIVEGLS
DSKDKEYILSQIELQILLPLQRIKDNGVIPYQLHLEELKAILAKCGPKFPFLNEVADGESVA
EKLIKMLEFRIPYYVGPLNTHHNVDNGGFAWAVRKASGRVTPWNEDDKIDREKSAAAFIKNL
TNKCTYLLGEDVLPKSSLLYSEFMLLNELNNVRIDGKPLEKVVKEHLIEAVFKQDHKKMTKN
RIEQFLKDNGYISETHKHEITGLDGEIKNDLASYRDMVRILGDGFDRSMAEEIITDITIFGE
SKKMLRETLRKKFASCLDDEAIKKLTKLRYRDWGRLSQKLLNGIEGCDKAGDGTPETIIILM
RNFSYNLMELLGDKFSFMERIQEINAKLTEGQIVNPHDIIDDLALSPAVKRAVWQALRIVDE
VAHIKKALPARIFVEVTRSNKNEKKKKDSRQKRLSDLYAAIKKDDVLLNGLNNEIFGELKSS
LAKYDDAALRSKKLYLYYTQMGRCAYTGEIIELSLLNTDNYDIDHIYPRSLTKDDSFDNLVL
CKRTANAQKSDAYPISEEIQKTQKPFWTFLKQQGLISERKYERLTRITPLTADDLSGFIARQ
LVETNQSVKAATTLLRRLYPGVDVVFVKAENVTDFRHDNNFIKVRSLNHHHHAKDAYLNIVV
GNVYHERFTRNFRAFFKKNGANRTYNLAKMENYDVNCTNAKDGKAWDVKTSMDTVKKMMDSN
DVRVTKRLLEQTGALADATIYKATVAGKAKDGAYIGMKTKSSVFADVSKYGGMTKIKNAYSI
IVQYTGKKGEVIKEIVPLPIYLTNRNTTDQDLINYVASIIPQAKDISIIYGKLCINQLVKVN
GFYYYLGGKTNSKFCIDNAIQVIVSNEWIPYLKVLEKENNMRKDNKDLKANVVSTRALDNKH
TIEVRIVEEKNIEFFDYLVSKLKMPIYQKMKGNKAAELSEKGYGLFKKMSLEEQSIHLIELL
NLLTNQKTTFEVKPLGITASRSTVGSKISNQDEFKVINESITGLYSNEVTIVKRPAATKKAG
QAKKKKSGSETPGTSESATPESSGSEVEFSHEYWMRHALTLAKRARDEREVPVGAVLVLNNR
VIGEGWNRAIGLHDPTAHAEIMALRQGGLVMQNYRLYDATLYVTFEPCVMCAGAMIHSRIGR
VVFGVRNAKTGAAGSLMDVLHHPGMNHRVEITEGILADECAALLCRFFRMPRRVENAQKKAQ
SSTDPAAKRVKLDGSYPYDVPDYAYPYDVPDYAYPYDVPDYA;
(e)
(SEQ ID NO: 16)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVENAQKKAQSSTDGSSGSETPG
TSESATPESSGPKKKRKVGTKVKDYYIGLAIGTSSVGWAVTDEAYNVLKFNSKKMWGVRLFD
DAKTAEERRGQRGARRRLDRKKERLSLLQDFFAEEVAKVDPNFFLRLDNSDLYMEDKDQKLK
SKYTLFNDKDFKDKNFHKKYPTIHHLLMDLIEDDSKKDIRLVYLACHYLLKNRGHFIFEGQK
FDTKSSFENSLNELKVHLNDEYGLDLEFDNENLINILTDPKLNKTAKKKELKSVIGDTKFLK
AVSAIMIGSSQKLVDLFENPEDFDDSAIKSVDFSTTSFDDKYSDYELALGDKIALVNILKEI
YDSSILENLLKEADKSKDGNKYISNAFVKKYNKHGQDLKEFKRLVRQYHKSAYEDIFRSEKV
NDNYVSYTKSSISNNKRVKANKFTDQEAFYKFAKKHLETIKYKINKVNGSKADLELIDGMLR
DMEFKNFMPKIKSSDNGVIPYQLKLMELNKILENQSKHHEFLNVSDEYGSVCDKIASIMEFR
IPYYVGPLNPNSKYAWIKKQKDSEITPWNFKDVVDLDSSREEFIDSLIGRCTYLKDEKVLPK
ASLLYNEYMVLNELNNLKLNDLPITEEMKKKIFDQLFKTRKKVTLKAVANLLKKEFNINGEI
LLSGTDGDFKQGLNSYNDFKAIVGDKVDSDDYRDKIEEIIKLIVLYGDDKSYLQKKIKAGYG
KYFTDSEIKKMAGLNYKDWGRLSKKLLTGLEGANKITGERGSIIHEMREYNLNLMELMSASF
TFTEEIQKLNPVDDRKLSYEMVDELYLSPSVKRMLWQSLRIVDEIKNIMGTDSKKIFIEMAR
GKEEVKARKESRKNQLLKFYKDGKKAFISEIGEERYSYLLSEIEGEEENKERWDNLYLYYTQ
LGRCMYSLEPIDISELSSKNIYDQDHIYPKSKIYDDSIENRVLVKKDLNSKKGNSYPIPDEI
LNKNCYAYWKILYDKGLIGQKKYTRLTRRTGFTDDELVQFISRQIVETRQATKETANLLKTI
CKNSEIVYSKAENASRFRQEFDIVKCRAVNDLHHMHDAYINIIVGNVYNTKFTKDPMNFVKK
QEKARSYNLENMFKYDVKRGGYTAWIADDEKGTVKNASIKRIRKELEGTNYRFTRMNYIESG
ALFNATLQRKNKGSRPLKDKGPKSSIEKYGGYTNINKACFAVLDIKSKNKIERKLMPVEREI
YAKQKNDKKLSDEIFSKYLKDRFGIEDYRVVYPVVKMRTLLKIDGSYYFITGGSDKTLELRS
ALQLILPKKNEWAIKQIDKSSENDYLTIERIQDLTEELVYNTFDIIVNKFKTSVEKKSFLNL
FQDDKIENIDFKFKSMDFKEKCKTLLMLVKAIRASGVRQDLKSIDLKSDYGRLSSKINNIGN
YQEFKIINQSITGLFENEVDLLKLGKRPAATKKAGQAKKKKGSYPYDVPDYAYPYDVPDYAY
PYDVPDYA;
(f)
(SEQ ID NO: 17)
MPKKKRKVTKVKDYYIGLAIGTSSVGWAVTDEAYNVLKENSKKMWGVRLEDDAKTAEERRGQ
RGARRRLDRKKERLSLLQDFFAEEVAKVDPNFFLRLDNSDLYMEDKDQKLKSKYTLFNDKDF
KDKNFHKKYPTIHHLLMDLIEDDSKKDIRLVYLACHYLLKNRGHFIFEGQKEDTKSSFENSL
NELKVHLNDEYGLDLEFDNENLINILTDPKLNKTAKKKELKSVIGDTKFLKAVSAIMIGSSQ
KLVDLFENPEDFDDSAIKSVDFSTTSFDDKYSDYELALGDKIALVNILKEIYDSSILENLLK
EADKSKDGNKYISNAFVKKYNKHGQDLKEFKRLVRQYHKSAYFDIFRSEKVNDNYVSYTKSS
ISNNKRVKANKFTDQEAFYKFAKKHLETIKYKINKVNGSKADLELIDGMLRDMEFKNEMPKI
KSSDNGVIPYQLKLMELNKILENQSKHHEFLNVSDEYGSVCDKIASIMEFRIPYYVGPLNPN
SKYAWIKKQKDSEITPWNFKDVVDLDSSREEFIDSLIGRCTYLKDEKVLPKASLLYNEYMVL
NELNNLKLNDLPITEEMKKKIFDQLFKTRKKVTLKAVANLLKKEFNINGEILLSGTDGDFKQ
GLNSYNDFKAIVGDKVDSDDYRDKIEEIIKLIVLYGDDKSYLQKKIKAGYGKYFTDSEIKKM
AGLNYKDWGRLSKKLLTGLEGANKITGERGSIIHFMREYNLNLMELMSASFTFTEEIQKLNP
VDDRKLSYEMVDELYLSPSVKRMLWQSLRIVDEIKNIMGTDSKKIFIEMARGKEEVKARKES
RKNQLLKFYKDGKKAFISEIGEERYSYLLSEIEGEEENKFRWDNLYLYYTQLGRCMYSLEPI
DISELSSKNIYDQDHIYPKSKIYDDSIENRVLVKKDLNSKKGNSYPIPDEILNKNCYAYWKI
LYDKGLIGQKKYTRLTRRTGFTDDELVQFISRQIVETRQATKETANLLKTICKNSEIVYSKA
ENASRFRQEFDIVKCRAVNDLHHMHDAYINIIVGNVYNTKFTKDPMNFVKKQEKARSYNLEN
MFKYDVKRGGYTAWIADDEKGTVKNASIKRIRKELEGTNYRFTRMNYIESGALENATLQRKN
KGSRPLKDKGPKSSIEKYGGYTNINKACFAVLDIKSKNKIERKLMPVEREIYAKQKNDKKLS
DEIFSKYLKDRFGIEDYRVVYPVVKMRTLLKIDGSYYFITGGSDKTLELRSALQLILPKKNE
WAIKQIDKSSENDYLTIERIQDLTEELVYNTFDIIVNKFKTSVFKKSFLNLFQDDKIENIDE
KFKSMDFKEKCKTLLMLVKAIRASGVRQDLKSIDLKSDYGRLSSKINNIGNYQEFKIINQSI
TGLFENEVDLLKLKRPAATKKAGQAKKKKSGSETPGTSESATPESSGSEVEFSHEYWMRHAL
TLAKRARDEREVPVGAVLVLNNRVIGEGWNRAIGLHDPTAHAEIMALRQGGLVMQNYRLYDA
TLYVTFEPCVMCAGAMIHSRIGRVVFGVRNAKTGAAGSLMDVLHHPGMNHRVEITEGILADE
CAALLCRFFRMPRRVFNAQKKAQSSTDPAAKRVKLDGSYPYDVPDYAYPYDVPDYAYPYDVP
DYA;
(g)
(SEQ ID NO: 88)
MPAAKRVKLDGSEVEFSHEYWMRHALTLAKRARDEREVPVGAVLVLNNRVIGEGWNRAIGLH
DPTAHAEIMALRQGGLVMQNYRLYDATLYVTFEPCVMCAGAMIHSRIGRVVFGVRNAKTGAA
GSLMDVLHHPGMNHRVEITEGILADECAALLCRFFRMPRRVFNAQKKAQSSTDGSSGSETPG
TSESATPESSGPKKKRKVGKEYHIGLAIGTSSIGWAVTDSQFKLMRIKGKTAIGVRLFEEGK
TAAERRTFRTTRRRLKRRKWRLHYLDEIFAPHLQEVDENFLRRLKQSNIHPEDPAKNQAFIG
KLLFPDLLKKNERGYPTLIKMRDELPVEQRAHYPVTNIYKLREAMINEDRQFDLREVYLAVH
HIVKYRGHFLNNASVDKFKVGRIDFDKSFNVLNEAYEELQNGEGSFTIEPSKVEKIGQLLLD
TKMRKLDRQKAVAKLLEVKVADKEETKRNKQIATAMSKLVLGYKADFATVAMANGNEWKIDL
SSETSEDEIEKFREELSDAQNDILTEITSLESQIMLNEIVPNGMSISESMMDRYWTHERQLA
EVKEYLATQPASARKEFDQVYNKYIGQAPKEKGFDLEKGLKKILSKKENWKEIDELLKAGDF
LPKQRTSANGVIPHQMHQQELDRIIEKQAKYYPWLATENPATGERDRHQAKYELDQLVSFRI
PYYVGPLVTPEVQKATSGAKFAWAKRKEDGEITPWNLWDKIDRAESAEAFIKRMTVKDTYLL
NEDVLPANSLLYQKYNVLNELNNVRVNGRRLSVGIKQDIYTELFKKKKTVKAGDVASLVMAK
TRGVNKPSVEGLSDPKKFNSNLATYLDLKSIVGDKVDDNRYQMDLENIIEWRSVFEDGEIFA
DKLTEVEWLTDEQRSALVKKRYKGWGRLSKKLLTGIVDENGQRIIDLMWNTDQNFMQIVNQP
VFKEQIDQLNQKAITNDGMTLRERVESVLDDAYTSPQNKKAIWQVVRVVEDIVKAVGNAPKS
ISIEFARNEGNKGEITRSRRTQLQKLFEDQAHELVKDTSLTEELEKAPDLSDRYYFYFTQGG
KDMYTGDPINFDEISTKYDIDHILPQSFVKDDSLDNRVLVSRAENNKKSDRVPAKLYAAKMK
PYWNQLLKQGLITQRKFENLTMDVDQTIKYRSLGFVKRQLVETRQVIKLTANILGSMYQEAG
TDIIETRAGLTKQLREEFDLPKVREVNDYHHAVDAYLTTFAGQYLNRRYPKLRSFFVYGEYM
KFKHGSDLKLRNFNFFHELMEGDKSQGKVVDQQTGELITTRDEVADYFDWVINLKVMLISNE
TYEETGKYFDASHESSSLYLKNQNKKSKLVVPLKNKLQPEYYGAYTGITQGYMVILKLLDKK
GGFGVYRIPRYAADILNKCHDEVAYRNKIAEIISSDPRAPKSFEVVVPRVLKGTFLVDGEEK
FILSSYRYKVNATQLILPVSDIKLIQDNFKALKKLNVEMQTKKLIEIYDNILRQVDKYYKLY
DINKFRAKLHDGRSKFVELDDFGQDASKEKVIIKILRGLHFGSDLQNLKEIGFGTTPLGQFQ
VSEAGIRLSNTAFIIFKSPTGLFNRKLYLKNLGKRPAATKKAGQAKKKKGSYPYDVPDYAYP
YDVPDYAYPYDVPDYA;
(h)
(SEQ ID NO: 89)
MPKKKRKVGKEYHIGLAIGTSSIGWAVTDSQFKLMRIKGKTAIGVRLFEEGKTAAERRTFRT
TRRRLKRRKWRLHYLDEIFAPHLQEVDENFLRRLKQSNIHPEDPAKNQAFIGKLLFPDLLKK
NERGYPTLIKMRDELPVEQRAHYPVTNIYKLREAMINEDRQFDLREVYLAVHHIVKYRGHEL
NNASVDKFKVGRIDFDKSFNVLNEAYEELQNGEGSFTIEPSKVEKIGQLLLDTKMRKLDRQK
AVAKLLEVKVADKEETKRNKQIATAMSKLVLGYKADFATVAMANGNEWKIDLSSETSEDEIE
KFREELSDAQNDILTEITSLESQIMLNEIVPNGMSISESMMDRYWTHERQLAEVKEYLATQP
ASARKEFDQVYNKYIGQAPKEKGFDLEKGLKKILSKKENWKEIDELLKAGDFLPKQRTSANG
VIPHQMHQQELDRIIEKQAKYYPWLATENPATGERDRHQAKYELDQLVSFRIPYYVGPLVTP
EVQKATSGAKFAWAKRKEDGEITPWNLWDKIDRAESAEAFIKRMTVKDTYLLNEDVLPANSL
LYQKYNVLNELNNVRVNGRRLSVGIKQDIYTELFKKKKTVKAGDVASLVMAKTRGVNKPSVE
GLSDPKKENSNLATYLDLKSIVGDKVDDNRYQMDLENIIEWRSVFEDGEIFADKLTEVEWLT
DEQRSALVKKRYKGWGRLSKKLLTGIVDENGQRIIDLMWNTDQNFMQIVNQPVFKEQIDQLN
QKAITNDGMTLRERVESVLDDAYTSPQNKKAIWQVVRVVEDIVKAVGNAPKSISIEFARNEG
NKGEITRSRRTQLQKLFEDQAHELVKDTSLTEELEKAPDLSDRYYFYFTQGGKDMYTGDPIN
FDEISTKYDIDHILPQSFVKDDSLDNRVLVSRAENNKKSDRVPAKLYAAKMKPYWNQLLKQG
LITQRKFENLTMDVDQTIKYRSLGFVKRQLVETRQVIKLTANILGSMYQEAGTDIIETRAGL
TKQLREEFDLPKVREVNDYHHAVDAYLTTFAGQYLNRRYPKLRSFFVYGEYMKFKHGSDLKL
RNFNFFHELMEGDKSQGKVVDQQTGELITTRDEVADYFDWVINLKVMLISNETYEETGKYED
ASHESSSLYLKNQNKKSKLVVPLKNKLQPEYYGAYTGITQGYMVILKLLDKKGGFGVYRIPR
YAADILNKCHDEVAYRNKIAEIISSDPRAPKSFEVVVPRVLKGTFLVDGEEKFILSSYRYKV
NATQLILPVSDIKLIQDNFKALKKLNVEMQTKKLIEIYDNILRQVDKYYKLYDINKFRAKLH
DGRSKFVELDDFGQDASKEKVIIKILRGLHFGSDLQNLKEIGFGTTPLGQFQVSEAGIRLSN
TAFIIFKSPTGLENRKLYLKNLKRPAATKKAGQAKKKKSGSETPGTSESATPESSGSEVEFS
HEYWMRHALTLAKRARDEREVPVGAVLVLNNRVIGEGWNRAIGLHDPTAHAEIMALRQGGLV
MQNYRLYDATLYVTFEPCVMCAGAMIHSRIGRVVFGVRNAKTGAAGSLMDVLHHPGMNHRVE
ITEGILADECAALLCRFFRMPRRVENAQKKAQSSTDPAAKRVKLDGSYPYDVPDYAYPYDVP
DYAYPYDVPDYA;
or
(i)
(SEQ ID NO: 98)
MSEVEFSHEYWMRHALTLAKRARDEREVPVGAVLVLNNRVIGEGWNRAIGLHDPTAHAEIMA
LRQGGLVMQNYRLYDATLYSTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSGKPYSIGLAIGTNSVGWAVVTDDYKVPAKKMKVLGNTDKQSIKKNLLGA
LLFDSGETAEATRLKRTARRRYTRRKNRLRYLQEIFTGEMNKVDENFFQRLDDSFLVDEDKR
GEHHPIFGNIAAEVKYHDDFPTIYHLRRHLADTSKKADLRLVYLALAHMIKERGHFLYEGDL
KAENTDVQALFKDEVEEYDKTIEESHLSEITVDALSILTEKVSKSSRLENLIAHYPTEKKNT
LFGNLIALSLDLHPNEKTNFQLSEDAKLQFSKDTYEEDLEGFLGEVGDEYADLFASAKNLYD
AILLSGILTVDDNSTKAPLSASMVKRYEEHQKDLKKLKDFIKVNAPDQYNAIFKDKNKKGYA
SYIESGVKQDEFYKYLKGILLKINGSGDFLDKIDREDFLRKQRTFDNGSIPHQIHLQEMHAI
LRRQGEHYPFLKENQDKIEKILTFRIPYYVGPLARKGSRFAWAEYKADEKITPWNEDDILDK
EKSAEKFITRMTLNDLYLPEEKVLPKHSPLYEAFTVYNELTKVKYVNEQGEAKFFDTNMKQE
IFDHVFKENRKVTKDKLLNYLNKEFEEFRIVNLTGLDKENKAFNSSLGTYHDLRKILDKSFL
DDKANEKTIEDIIQTLTLFEDREMIRQRLQKYSDIFTKAQLKKLERRHYTGWGRLSYKLING
IRNKENKKTILDYLIDDGYANRNFMQLINDDALSFKEEIARAQIIDDVDDIANVVHDLPGSP
AIKKGILQSVKIVDELVKVMGHNPANIIIEMARENQTTDKGRRNSQQRLKLLQDSLKNLDNP
VNIKNVENQQLQNDRLFLYYIQNGKDMYTGETLDINNLSQYDIDHIIPQAFIKDNSLDNRVL
TRSDKNRGKSDDVPSIEVVHEMKSFWSKLLSVKLITQRKEDNLTKAERGGLTEEDKAGFIKR
QLVETRQITKHVAQILDERENTEFDGNKRRIRNVKIITLKSNLVSNFRKEFELYKVREINDY
HHAHDAYLNAVVGNALLLKYPQLEPEFVYGEYPKYNSYRSRKSATEKFLFYSNILRFFKKED
IQTNEDGEIAWNKEKHIKILRKVLSYPQVNIVKKTEEQTGGFSKESILPKGESDKLIPRKTK
NSYWDPKKYGGFMQPVVAYSILVFADVEKGKSKKLRKVQDMVGITIMEKKRFEKNPVDFLEQ
RGYRNVRLEKIIKLPKYSLFELENKRRRLLASAKFLQKGNELVIPQRFTTLLYHSYRIEKDY
EPEHREYVEKHKDEFKELLEYISVESRKYVLADNNLTKIEMLESKNKDAEVSSLAKSFISLL
TFTAFGAPRAFNFFGENIARKEYRSVTECLNATLIHQSITGLYETRIDLSKLGEDGEGADKR
TADGSEFESPKKKRKV;
(j)
(SEQ ID NO: 99)
MSEVEFSHEYWMRHALTLAKRARDEREVPVGAVLVLNNRVIGEGWNRAIGLHDPTAHAEIMA
LRQGGLVMQNYRLYDATLYSTFEPCVMCAGAMIHSRIGRVVFGVRNAKTGAAGSLMDVLHHP
GMNHRVEITEGILADECAALLCRFFRMPRRVENAQKKAQSSTDSGGSSGGSSGSETPGTSES
ATPESSGGSSGGSGKPYSIGLAIGTNSVGWAVVTDDYKVPAKKMKVLGNTDKQSIKKNLLGA
LLFDSGETAEATRLKRTARRRYTRRKNRLRYLQEIFTGEMNKVDENFFQRLDDSFLVDEDKR
GEHHPIFGNIAAEVKYHDDFPTIYHLRRHLADTSKKADLRLVYLALAHMIKFRGHELYEGDL
KAENTDVQALFKDFVEEYDKTIEESHLSEITVDALSILTEKVSKSSRLENLIAHYPTEKKNT
LFGNLIALSLDLHPNFKTNFQLSEDAKLQFSKDTYEEDLEGFLGEVGDEYADLFASAKNLYD
AILLSGILTVDDNSTKAPLSASMVKRYEEHQKDLKKLKDFIKVNAPDQYNAIFKDKNKKGYA
SYIESGVKQDEFYKYLKGILLKINGSGDFLDKIDREDELRKQRTEDNGIIPHQIHLQEMHAI
LRRQGEHYPFLKENQDKIEKILTFRIPYYVGPLARKGSRFAWAEYKADEKITPWNEDDILDK
EKSAEKFITRMTLNDLYLPEEKVLPKHSPLYEAFTVYNELTKVKYVNEQGEAKFEDTNMKQE
IFDHVFKENRKVTKDKLLNYLNKEFEEFRIVNLTGLDKENKAFNSSLGTYHDLRKILDKSFL
DDKANEKTIEDIIQTLTLFEDREMIRQRLQKYSDIFTKAQLKKLERLHYTGWGRLSYKLING
IRNKENKKTILDYLIDDGYANRNFMQLINDDALSFKEEIARAQIIDDVDDIANVVHDLPGSP
AIKKGILQSVKIVDELVKVMGHNPANIIIEMARENQTTDKGRRNSQQRLKLLQDSLKNLDNP
VNIKNVENQQLQNDRLFLYYIQNGKDMYTGETLDINNLSQYDIDHIIPQAFIKDNSLDNRVL
TRSDKNRGKSDDVPSIEVVHEMKSFWSKLLSVKLITQRKEDNLTKAERGGLTEEDKAGFIKR
QLVETRQITKHVAQILDERENTEFDGNKRRIRNVKIITLKSNLVSNFRKEFELYKVREINDY
HHAHDAYLNAVVGNALLLKYPQLEPEFVYGEYPKYNSYRSRKSATEKFLFYSNILRFFKKED
IQTNEDGEIAWNKEKHIKILRKVLSYPQVNIVKKTEEQTGGFSKESILPKGESDKLIPRKTK
NSYWDPKKYGGFMQPVVAYSILVFADVEKGKSKKLRKVQDMVGITIMEKKRFEKNPVDFLEQ
RGYRNVRLEKIIKLPKYSLFELENKRRRLLASAKFLQKGNELVIPQRFTTLLYHSYRIEKDY
EPEHREYVEKHKDEFKELLEYISVFSRKYVLADNNLTKIEMLESKNKDAEVSSLAKSFISLL
TFTAFGAPRAFNFFGENIARKEYRSVTECLNATLIHQSITGLYETRIDLSKLGEDGEGADKR
TADGSEFESPKKKRKV.

24. The Cas9 protein of claim 22, wherein the Cas9 protein is fused to a cytosine deaminase and has an amino acid sequence at least 80% identical to
(a)
(SEQ ID NO: 21)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWELSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGGKPYSIGLAIGTNSVGWAVVTDD
YKVPAKKMKVLGNTDKQSIKKNLLGALLFDSGETAEATRLKRTARRRYTRRKNRLRYLQEIF
TGEMNKVDENFFQRLDDSFLVDEDKRGEHHPIFGNIAAEVKYHDDFPTIYHLRRHLADTSKK
ADLRLVYLALAHMIKFRGHFLYEGDLKAENTDVQALFKDEVEEYDKTIEESHLSEITVDALS
ILTEKVSKSSRLENLIAHYPTEKKNTLFGNLIALSLDLHPNFKTNFQLSEDAKLQFSKDTYE
EDLEGFLGEVGDEYADLFASAKNLYDAILLSGILTVDDNSTKAPLSASMVKRYEEHQKDLKK
LKDFIKVNAPDQYNAIFKDKNKKGYASYIESGVKQDEFYKYLKGILLKINGSGDFLDKIDRE
DFLRKQRTFDNGSIPHOIHLQEMHAILRRQGEHYPFLKENQDKIEKILTFRIPYYVGPLARK
GSRFAWAEYKADEKITPWNEDDILDKEKSAEKFITRMTLNDLYLPEEKVLPKHSPLYEAFTV
YNELTKVKYVNEQGEAKFFDTNMKQEIFDHVFKENRKVTKDKLLNYLNKEFEEFRIVNLTGL
DKENKAFNSSLGTYHDLRKILDKSFLDDKANEKTIEDIIQTLTLFEDREMIRQRLOKYSDIF
TKAQLKKLERRHYTGWGRLSYKLINGIRNKENKKTILDYLIDDGYANRNFMQLINDDALSEK
EEIARAQIIDDVDDIANVVHDLPGSPAIKKGILQSVKIVDELVKVMGHNPANIIIEMARENQ
TTDKGRRNSQQRLKLLQDSLKNLDNPVNIKNVENQQLQNDRLFLYYIQNGKDMYTGETLDIN
NLSQYDIDHIIPQAFIKDNSLDNRVLTRSDKNRGKSDDVPSIEVVHEMKSFWSKLLSVKLIT
QRKFDNLTKAERGGLTEEDKAGFIKRQLVETRQITKHVAQILDERENTEFDGNKRRIRNVKI
ITLKSNLVSNFRKEFELYKVREINDYHHAHDAYLNAVVGNALLLKYPQLEPEFVYGEYPKYN
SYRSRKSATEKFLFYSNILRFFKKEDIQTNEDGEIAWNKEKHIKILRKVLSYPQVNIVKKTE
EQTGGFSKESILPKGESDKLIPRKTKNSYWDPKKYGGFDSPVVAYSILVFADVEKGKSKKLR
KVQDMVGITIMEKKRFEKNPVDFLEQRGYRNVRLEKIIKLPKYSLFELENKRRRLLASAKEL
QKGNELVIPQRFTTLLYHSYRIEKDYEPEHREYVEKHKDEFKELLEYISVESRKYVLADNNL
TKIEMLFSKNKDAEVSSLAKSFISLLTFTAFGAPAAFNFFGENIDRKRYTSVTECLNATLIH
QSITGLYETRIDLSKLGEDGKRPAATKKAGQAKKKKGSSGGSGGSGGSTNLSDIIEKETGKQ
LVIQESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVHTA
YDESTDENVMLLTSDAPEYKPWALVIQDSNGENKIKMLYPYDVPDYAYPYDVPDYAYPYDVP
DYA;
(b)
(SEQ ID NO: 12)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGSIINFQRRGLMETQASNQLISSH
LKGYPIKDYFVGLAIGTSSVGWAVTNKAYELLKFRSHKMWGSRLFDEGESAVARRGERSMRR
RLERRKLRLKLLEELFADAMAQVDPTFFMRLRESKYHYEDKTTGHSSKHILFIDKNYNDQDY
FKEYPTVYHLRSELMKSGTDDIRKLFLAVHHILKYRGNFLYEGATFDSNASTLDDVIKQALE
NITFNCFDCNSAISSIGQILMEAGKTKSDKAKAIEHLVDTYIATDTVDTSSKTQKDQVKEDK
KRLKAFANLVLGLNASLIDLFGSVEELEEDLKKLQITGDTYDDKRDELAKAWSDEIYIIDDC
KSVYDAIILLSIKEPGLTISESKVKAFNKHKDDLAILKSLLKSDRSIYNTMFKVDEKGLHNY
VHYIKQGRTEETSCNREDFYKYTKKIVEGLSDSKDKEYILSQIELQILLPLQRIKDNGVIPY
QLHLEELKAILAKCGPKFPFLNEVADGESVAEKLIKMLEFRIPYYVGPLNTHHNVDNGGFAW
AVRKASGRVTPWNFDDKIDREKSAAAFIKNLTNKCTYLLGEDVLPKSSLLYSEFMLLNELNN
VRIDGKPLEKVVKEHLIEAVFKQDHKKMTKNRIEQFLKDNGYISETHKHEITGLDGEIKNDL
ASYRDMVRILGDGFDRSMAEEIITDITIFGESKKMLRETLRKKFASCLDDEAIKKLTKLRYR
DWGRLSQKLLNGIEGCDKAGDGTPETIIILMRNFSYNLMELLGDKFSFMERIQEINAKLTEG
QIVNPHDIIDDLALSPAVKRAVWQALRIVDEVAHIKKALPARIFVEVTRSNKNEKKKKDSRQ
KRLSDLYAAIKKDDVLLNGLNNEIFGELKSSLAKYDDAALRSKKLYLYYTQMGRCAYTGEII
ELSLLNTDNYDIDHIYPRSLTKDDSFDNLVLCKRTANAQKSDAYPISEEIQKTQKPFWTFLK
QQGLISERKYERLTRITPLTADDLSGFIARQLVETNQSVKAATTLLRRLYPGVDVVFVKAEN
VTDFRHDNNFIKVRSLNHHHHAKDAYLNIVVGNVYHERFTRNFRAFFKKNGANRTYNLAKME
NYDVNCTNAKDGKAWDVKTSMDTVKKMMDSNDVRVTKRLLEQTGALADATIYKATVAGKAKD
GAYIGMKTKSSVFADVSKYGGMTKIKNAYSIIVQYTGKKGEVIKEIVPLPIYLTNRNTTDQD
LINYVASIIPQAKDISIIYGKLCINQLVKVNGFYYYLGGKTNSKFCIDNAIQVIVSNEWIPY
LKVLEKFNNMRKDNKDLKANVVSTRALDNKHTIEVRIVEEKNIEFFDYLVSKLKMPIYQKMK
GNKAAELSEKGYGLFKKMSLEEQSIHLIELLNLLTNQKTTFEVKPLGITASRSTVGSKISNQ
DEFKVINESITGLYSNEVTIVGKRPAATKKAGQAKKKKGSSGGSGGSGGSTNLSDIIEKETG
KQLVIQESILMLPEEVEEVIGNKPESDILVHTAYDESTDENVMLLTSDAPEYKPWALVIQDS
NGENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLYPYDVPDYAYPYDVPDYAY;
(c)
(SEQ ID NO: 18)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGTKVKDYYIGLAIGTSSVGWAVTD
EAYNVLKFNSKKMWGVRLFDDAKTAEERRGQRGARRRLDRKKERLSLLQDFFAEEVAKVDPN
FFLRLDNSDLYMEDKDQKLKSKYTLENDKDEKDKNFHKKYPTIHHLLMDLIEDDSKKDIRLV
YLACHYLLKNRGHFIFEGQKFDTKSSFENSLNELKVHLNDEYGLDLEFDNENLINILTDPKL
NKTAKKKELKSVIGDTKFLKAVSAIMIGSSQKLVDLFENPEDEDDSAIKSVDESTTSFDDKY
SDYELALGDKIALVNILKEIYDSSILENLLKEADKSKDGNKYISNAFVKKYNKHGQDLKEFK
RLVRQYHKSAYFDIFRSEKVNDNYVSYTKSSISNNKRVKANKFTDQEAFYKFAKKHLETIKY
KINKVNGSKADLELIDGMLRDMEFKNFMPKIKSSDNGVIPYQLKLMELNKILENQSKHHEFL
NVSDEYGSVCDKIASIMEFRIPYYVGPLNPNSKYAWIKKQKDSEITPWNFKDVVDLDSSREE
FIDSLIGRCTYLKDEKVLPKASLLYNEYMVLNELNNLKLNDLPITEEMKKKIFDQLFKTRKK
VTLKAVANLLKKEFNINGEILLSGTDGDFKQGLNSYNDFKAIVGDKVDSDDYRDKIEEIIKL
IVLYGDDKSYLQKKIKAGYGKYFTDSEIKKMAGLNYKDWGRLSKKLLTGLEGANKITGERGS
IIHFMREYNLNLMELMSASFTFTEEIQKLNPVDDRKLSYEMVDELYLSPSVKRMLWQSLRIV
DEIKNIMGTDSKKIFIEMARGKEEVKARKESRKNQLLKFYKDGKKAFISEIGEERYSYLLSE
IEGEEENKFRWDNLYLYYTQLGRCMYSLEPIDISELSSKNIYDQDHIYPKSKIYDDSIENRV
LVKKDLNSKKGNSYPIPDEILNKNCYAYWKILYDKGLIGQKKYTRLTRRTGFTDDELVQFIS
RQIVETRQATKETANLLKTICKNSEIVYSKAENASRFRQEFDIVKCRAVNDLHHMHDAYINI
IVGNVYNTKFTKDPMNFVKKQEKARSYNLENMEKYDVKRGGYTAWIADDEKGTVKNASIKRI
RKELEGTNYRFTRMNYIESGALFNATLQRKNKGSRPLKDKGPKSSIEKYGGYTNINKACFAV
LDIKSKNKIERKLMPVEREIYAKQKNDKKLSDEIFSKYLKDREGIEDYRVVYPVVKMRTLLK
IDGSYYFITGGSDKTLELRSALQLILPKKNEWAIKQIDKSSENDYLTIERIQDLTEELVYNT
FDIIVNKFKTSVFKKSFLNLFQDDKIENIDFKFKSMDFKEKCKTLLMLVKAIRASGVRQDLK
SIDLKSDYGRLSSKINNIGNYQEFKIINQSITGLFENEVDLLKLGKRPAATKKAGQAKKKKG
SSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQES
ILMLPEEVEEVIGNKPESDILVHTAYDESTDENVMLLTSDAPEYKPWALVIQDSNGENKIKM
LYPYDVPDYAYPYDVPDYAY;
(d)
(SEQ ID NO: 90)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWELSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGKEYHIGLAIGTSSIGWAVTDSQF
KLMRIKGKTAIGVRLFEEGKTAAERRTFRTTRRRLKRRKWRLHYLDEIFAPHLQEVDENFLR
RLKQSNIHPEDPAKNQAFIGKLLFPDLLKKNERGYPTLIKMRDELPVEQRAHYPVINIYKLR
EAMINEDRQFDLREVYLAVHHIVKYRGHFLNNASVDKFKVGRIDEDKSENVLNEAYEELQNG
EGSFTIEPSKVEKIGQLLLDTKMRKLDRQKAVAKLLEVKVADKEETKRNKQIATAMSKLVLG
YKADFATVAMANGNEWKIDLSSETSEDEIEKFREELSDAQNDILTEITSLESQIMLNEIVPN
GMSISESMMDRYWTHERQLAEVKEYLATQPASARKEFDQVYNKYIGQAPKEKGEDLEKGLKK
ILSKKENWKEIDELLKAGDELPKQRTSANGVIPHQMHQQELDRIIEKQAKYYPWLATENPAT
GERDRHQAKYELDQLVSFRIPYYVGPLVTPEVQKATSGAKFAWAKRKEDGEITPWNLWDKID
RAESAEAFIKRMTVKDTYLLNEDVLPANSLLYQKYNVLNELNNVRVNGRRLSVGIKQDIYTE
LFKKKKTVKAGDVASLVMAKTRGVNKPSVEGLSDPKKENSNLATYLDLKSIVGDKVDDNRYQ
MDLENIIEWRSVFEDGEIFADKLTEVEWLTDEQRSALVKKRYKGWGRLSKKLLTGIVDENGQ
RIIDLMWNTDQNFMQIVNQPVFKEQIDQLNQKAITNDGMTLRERVESVLDDAYTSPQNKKAI
WQVVRVVEDIVKAVGNAPKSISIEFARNEGNKGEITRSRRTQLQKLFEDQAHELVKDTSLTE
ELEKAPDLSDRYYFYFTQGGKDMYTGDPINFDEISTKYDIDHILPQSFVKDDSLDNRVLVSR
AENNKKSDRVPAKLYAAKMKPYWNQLLKQGLITQRKFENLTMDVDQTIKYRSLGFVKRQLVE
TRQVIKLTANILGSMYQEAGTDIIETRAGLTKQLREEFDLPKVREVNDYHHAVDAYLTTFAG
QYLNRRYPKLRSFFVYGEYMKFKHGSDLKLRNENFFHELMEGDKSQGKVVDQQTGELITTRD
EVADYFDWVINLKVMLISNETYEETGKYFDASHESSSLYLKNQNKKSKLVVPLKNKLQPEYY
GAYTGITQGYMVILKLLDKKGGFGVYRIPRYAADILNKCHDEVAYRNKIAEIISSDPRAPKS
FEVVVPRVLKGTFLVDGEEKFILSSYRYKVNATQLILPVSDIKLIQDNFKALKKLNVEMQTK
KLIEIYDNILRQVDKYYKLYDINKFRAKLHDGRSKFVELDDFGQDASKEKVIIKILRGLHFG
SDLQNLKEIGFGTTPLGQFQVSEAGIRLSNTAFIIFKSPTGLENRKLYLKNLGKRPAATKKA
GQAKKKKGSSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDIIEKETG
KQLVIQESILMLPEEVEEVIGNKPESDILVHTAYDESTDENVMLLTSDAPEYKPWALVIQDS
NGENKIKMLYPYDVPDYAYPYDVPDYAY;
(e)
(SEQ ID NO: 93)
MPAAKRVKLDTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQHPGVTLVIY
VARLFWHMDQRNRQGLRDLVNSGVTIQIMRASEYYHCWRNFVNYPPGDEAHWPQYPPLWMML
YALELHCIILSLPPCLKISRRWQNHLAFFRLHLQNCHYQTIPPHILLATGLIHPSVTWRLKS
GGSSGGSSGSETPGTSESATPESSGGSSGGSPKKKRKVGEKKTNYTIGLAIGTDSVGWAVVK
DDLELVKKRMKVLGNTETNYIKKNLWGSLLFESGQTAKDRRLKRVARRRYERRRNRLTELQK
IFAPAIDEVDENFFFRLNESFLVPEDKAFSKNPIFGTLGEDKTYYKTYPTIYHLRQHLADSE
EKADVRLIYLALAHMIKYRGHFLIEGKLDTEHIAINENLEQFFESYNALFSEEPIELRKEEL
IAIENILREKNSRTVKEKRITSFLKDIGRANKQSPMMAFITLIVGKKAKFKAAFNLEEEISL
NLTDDSYDENLEILLNTIGSDFADLEDHAQRVYNAVELAGILSGDVKNTHAKLSAQMVAMYE
RHKEQLKEYKSFIKANLPDQYDMTFVAPKDAQKKDLKGYAGYIDGNMSQDSFYKFVKDQLKE
VPGSEKFLDSIEKEDFLRKQRSFYNGVIPNQVHLAEMEAILDRQENYYPWLKENREKIISLL
TFRIPYYVGPLADGQSEFAWLERKSDEKIKPWNFSDVVDLDRSAEKFIEQLIGRDTYLPDEY
VLPKKSLIYQKYMVFNELTKIAYLDERQKRMNLSSVEKKEIFETLFKKRSKVTEKQLVKFFE
NYLQIDNPTIFGIEDAFNADYSTYVELAKVPGMKSMMDDPDNEDLMEEIVKILTVFEDRKMR
RKQLEKYKERLSPEQIKELAKKHYTGWGRLSKKLLVGIRDKETQKTILDYLVEDDNHSGGRQ
HLNRNLMQLINDDRLSFKKTIAELQMIDPSADLYAQVQEIAGSPAIKKGILLGLKIVDEIIR
VMGEKPENIVIEMARENQTTARGKALSKRREAKIKEGLAALGSSLLKENLPGNADLSQRKIY
LYYTQNGKDIYLDEPLDFDRLSQYDEDHIIPQSFTVDNSLDNLVLTNSSQNRGNKKDDVPSL
EVVNRQLAYWRSLKDAGLMTQRKFDNLTKAMRGGLTDKDRERFIQRQLVETRQITKNVAKLL
DMRLNDKKDEAGNKIRETNIVLLKSAMASEFRKMFRLYKVRELNDYHHAHDAYLNAAIAINL
LALYPYMADDEVYGEFRYKKKPQAEKATYEKLRQWNLIKRFGEKQLFTPDHEDCWNKERDIK
TIKKVMGYRQVNVVKKAEERTGMLFKETINGKTNKGSRIPIKKDLDPSKYGGYIEEKMAYYA
VISYEDKKKKPGKTIVGISIMDKKEFEYDSISYLGKLGFSNPVVQIILKNYSLIAYPDGRRR
YITGATKTTKGKVELQKANQIAMEQDLVNFIYHLKNYDEISHPESYAFVQSHTDYFDRLEDS
IEHYTRRFLDAETNINRLRRIYEEEKKKDPVDIEALVASFIELLKLTSAGAPADFIFMGEAI
SRRRYNSMTGLEDGQVIYQSLTGLYETRMRFEDGKRPAATKKAGQAKKKKGSSGGSGGSGGS
TNLSDIIEKETGKQLVIQESILMLPEEVEEVIGNKPESDILVHTAYDESTDENVMLLTSDAP
EYKPWALVIQDSNGENKIKMLSGGSGGSGGSTNLSDIIEKETGKQLVIQESILMLPEEVEEV
IGNKPESDILVHTAYDESTDENVMLLTSDAPEYKPWALVIQDSNGENKIKMLYPYDVPDYAY
PYDVPDYAY;
(f)
(SEQ ID NO: 94)
MPAAKRVKLDTNLSDIIEKETGKQLVIQESILMLPEEVEEVIGNKPESDILVHTAYDESTDE
NVMLLTSDAPEYKPWALVIQDSNGENKIKMLSGGSGGSGGSTNLSDIIEKETGKQLVIQESI
LMLPEEVEEVIGNKPESDILVHTAYDESTDENVMLLTSDAPEYKPWALVIQDSNGENKIKML
SGGSGGSGGSPKKKRKVEKKTNYTIGLAIGTDSVGWAVVKDDLELVKKRMKVLGNTETNYIK
KNLWGSLLFESGQTAKDRRLKRVARRRYERRRNRLTELQKIFAPAIDEVDENFFERLNESEL
VPEDKAFSKNPIFGTLGEDKTYYKTYPTIYHLRQHLADSEEKADVRLIYLALAHMIKYRGHF
LIEGKLDTEHIAINENLEQFFESYNALFSEEPIELRKEELIAIENILREKNSRTVKEKRITS
FLKDIGRANKQSPMMAFITLIVGKKAKFKAAFNLEEEISLNLTDDSYDENLEILLNTIGSDE
ADLFDHAQRVYNAVELAGILSGDVKNTHAKLSAQMVAMYERHKEQLKEYKSFIKANLPDQYD
MTFVAPKDAQKKDLKGYAGYIDGNMSQDSFYKFVKDQLKEVPGSEKFLDSIEKEDELRKQRS
FYNGVIPNQVHLAEMEAILDRQENYYPWLKENREKIISLLTFRIPYYVGPLADGQSEFAWLE
RKSDEKIKPWNFSDVVDLDRSAEKFIEQLIGRDTYLPDEYVLPKKSLIYQKYMVENELTKIA
YLDERQKRMNLSSVEKKEIFETLFKKRSKVTEKQLVKFFENYLQIDNPTIFGIEDAFNADYS
TYVELAKVPGMKSMMDDPDNEDLMEEIVKILTVFEDRKMRRKQLEKYKERLSPEQIKELAKK
HYTGWGRLSKKLLVGIRDKETQKTILDYLVEDDNHSGGRQHLNRNLMQLINDDRLSFKKTIA
ELQMIDPSADLYAQVQEIAGSPAIKKGILLGLKIVDEIIRVMGEKPENIVIEMARENQTTAR
GKALSKRREAKIKEGLAALGSSLLKENLPGNADLSQRKIYLYYTQNGKDIYLDEPLDEDRLS
QYDEDHIIPQSFTVDNSLDNLVLTNSSQNRGNKKDDVPSLEVVNRQLAYWRSLKDAGLMTQR
KFDNLTKAMRGGLTDKDRERFIQRQLVETRQITKNVAKLLDMRLNDKKDEAGNKIRETNIVL
LKSAMASEFRKMERLYKVRELNDYHHAHDAYLNAAIAINLLALYPYMADDFVYGEFRYKKKP
QAEKATYEKLRQWNLIKRFGEKQLFTPDHEDCWNKERDIKTIKKVMGYRQVNVVKKAEERTG
MLFKETINGKTNKGSRIPIKKDLDPSKYGGYIEEKMAYYAVISYEDKKKKPGKTIVGISIMD
KKEFEYDSISYLGKLGFSNPVVQIILKNYSLIAYPDGRRRYITGATKTTKGKVELQKANQIA
MEQDLVNFIYHLKNYDEISHPESYAFVQSHTDYFDRLEDSIEHYTRRFLDAETNINRLRRIY
EEEKKKDPVDIEALVASFIELLKLTSAGAPADFIFMGEAISRRRYNSMTGLEDGQVIYQSLT
GLYETRMRFEDKRPAATKKAGQAKKKKGSSGGSSGGSSGSETPGTSESATPESSGGSSGGST
SEKGPSTGDPTLRRRIESWEFDVFYDPRELRKETCLLYEIKWGMSRKIWRSSGKNTTNHVEV
NFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILS
LPPCLKISRRWQNHLAFFRLHLQNCHYQTIPPHILLATGLIHPSVTWRYPYDVPDYAYPYDV
PDYAYPYDVPDYA.

25. The Cas9 protein of claim 2, wherein the Cas9 protein recognizes a PAM sequence comprising 5′-NGG-3′.

26. The Cas9 protein of claim 3, wherein the Cas9 protein recognizes a PAM sequence comprising 5′-NAGHC-3′, wherein H is adenine, cytosine, or thymine.

27. The Cas9 protein of claim 4, wherein the Cas9 protein recognizes a PAM sequence comprising 5′-NRHRRH-3′, wherein H is adenine, cytosine or thymine, and R is adenine or guanine.

28. The Cas9 protein of claim 5 or claim 7, wherein the Cas9 protein recognizes a PAM sequence comprising 5′-NGG-3′.

29. The Cas9 protein of claim 6, wherein the Cas9 protein recognizes a PAM sequence comprising 5′-NNAAA-3′.

29b. The Cas9 protein of claim 10b or 10c, wherein the Cas9 protein recognizes a PAM sequence comprising 5′-NGG-3′.

30. A nucleic acid encoding the Cas9 protein of any one of the preceding claims.

31. The nucleic acid of claim 30, wherein the nucleic acid is codon-optimized for expression in mammalian cells.

32. The nucleic acid of claim 31, wherein the nucleic acid is codon-optimized for expression in human cells.

33. A eukaryotic cell comprising the Cas9 protein of any one of claim 29.

34. The eukaryotic cell of claim 33, wherein the cell is a human cell.

35. A method of cleaving a target nucleic acid in a eukaryotic cell comprising:
contacting the cell with a Cas9 of any one of claims 1-29, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and of causing a break in the target nucleic acid sequence complementary to the RNA guide.

36. A method of altering expression of a target nucleic acid in a eukaryotic cell comprising:
contacting the cell with a Cas9 of any one of claims 1-29, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and of causing a break in the target nucleic acid sequence complementary to the RNA guide.

37. A method of altering expression of a target nucleic acid in a eukaryotic cell comprising:
contacting the cell with a Cas9 of any one of claims 1-29, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and editing the target nucleic acid sequence complementary to the RNA guide.

38. A method of modifying a target nucleic acid in a eukaryotic cell comprising:
contacting the cell with a Cas9 of any one of claims 1-29, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and editing the target nucleic acid sequence complementary to the RNA guide.

39. The method of claim 37 or 38, wherein the Cas9 protein is an inactive Cas9 (dCas9).

40. The method of claim 39, wherein the dCas9 is fused to a deaminase.

41. The method of any one of claims 35-40, wherein the RNA guide comprises a crRNA and a tracrRNA.

42. The method of any one of claims 35-39, wherein the RNA guide comprises a sgRNA.

43. The method of claim 42, wherein the sgRNA for use with Streptococcus constellatus Cas9 comprises a scaffold comprising a sequence having at least about 80% identity to
(SEQ ID NO: 3)
5′-GUUUUAGAGCUGUGCUGUUUAAACAACACAGCAAGUUAAAAUAAGGC
UUUGUCCGUACUCAAGCUUGCAAAAGCGUGCACCGAUUCGGUGCU-3′

44. The method of claim 42, wherein the sgRNA for use with Sharpea Cas9 comprises a scaffold comprising a sequence having at least about 80% identity to
(SEQ ID NO: 7)
5′-GUUUUAGAGUUGUGUUAUUGAAAAAUAACACAACGAGUUAAAAUAAA
GCUUAUGCUUAAAUGCCAGCUUUGCUGGUGUCAUUUAGAUGACUUUACUA
AGGUUGCUUCGGCAACCUUUUU-3′

45. The method of claim 42, wherein the sgRNA for use with Veillonella parvula Cas9 comprises a scaffold comprising a sequence having at least about 80% identity to
(SEQ ID NO: 13)
5′-GUUUGAGAGUAGUGUGAAAACAUUACGAGUUCAAAUACAAAUUAAUU
UACAAUGCCUUCGGGCUGCCCGACGUAGGGCACCUACUCUCAAUUCUUCG
GAAUUGAGUU-3′

46. The method of claim 42, wherein the sgRNA for use with Ezakiella peruensis Cas9 comprises a scaffold comprising a sequence having at least about 80% identity to
(SEQ ID NO: 19)
5′-GUUUGAGAGUUAUGUAAUUGAAAAAUUACAUGACGAGUUCAAAUAAA
AAUUUAUUCAAACCGCCUAUUUAUAGGCCGCAGAUGUUCUGCAUUAUGCU
UGCUAUUGCAAGCUU-3′

47. The method of claim 42, wherein the sgRNA for use with Lactobacillus fermentum strain AF15-40LB Cas9 comprises a scaffold comprising a sequence having at least about 80% identity to
(SEQ ID NO: 95)
5′-GUCUUGGAUGAGUGUGAAAACACUCAUAGUCAAGAUCAAACGAGUGG
UUUUCCACGAGUUAUUACUUUUGAGGUCUUAUAUGGCCCAUACAUAAAAA
GGAGUCGGAAUUUCCGGCUCCUUUUCUU-3′

48. The method of claim 42, wherein the sgRNA for use with Peptoniphilus sp. Marseille-P 3761 Cas9 comprises a scaffold comprising a sequence having at least about 80% identity to
(SEQ ID NO: 96)
5′-GUUUUAGAGCCAUGUAGAAAUACAUUGCAAGUUAAAAUAAGGCUUUG
UCCGUAAUCAACUUGAAAAAGUGGCGCUGUUUCGGCGCUUU-3′

49. The method of claim 41, wherein the crRNA comprises a guide sequence of between about 16 and 26 nucleotides long.

50. The method of claim 49, wherein the crRNA comprises a guide sequence between 18 and 24 nucleotides long.

51. The method of claim 35 or 36, wherein the break in the target nucleic acid is a single-stranded or double-stranded break.

52. The method of claim 51, wherein the break in the target nucleic acid is a single-stranded break.

53. The method of claim 34 or 35, wherein the Cas9 protein is a nuclease that cleaves both strands of the target nucleic acid sequence, or is a nickase that cleaves one strand of the target nucleic acid sequence.

54. The method of any one of claims 34-53, wherein the target nucleic acid is 5′ to a protospacer adjacent motif (PAM) sequence.

55. The method of any one of claims 34-54, wherein the Cas9 is operably linked to a promoter sequence for expression in a eukaryotic cell, and wherein the guide RNA is operably linked to a promoter sequence for expression in a eukaryotic cell.

56. The method of claim 55, wherein the eukaryotic cell is a human cell.

57. The method of claim 55, wherein the promoter sequence is a eukaryotic or viral promoter.

58. An engineered, non-naturally occurring CRISPR-Cas system comprising:
an RNA guide or a nucleic acid encoding the RNA guide, wherein the RNA guide comprises a direct repeat sequence and a spacer sequence capable of hybridizing to a target nucleic acid; and
a codon-optimized CRISPR-associated (Cas) protein having at least 80% sequence identity to SEQ ID NOs: 1, 4, 8, 14, 84 or 86, and wherein the Cas protein is capable of binding to the RNA guide and of causing a break in the target nucleic acid sequence complementary to the RNA guide.

58b. The engineered, non-naturally occurring CRISPR-Cas system of claim 58 where the codon-optimized CRISPR-associated (Cas) protein further comprising a nuclear localization sequence (NLS) and/or a FLAG, HIS or HA tag.

58c. The engineered, non-naturally occurring CRISPR-Cas system of claim 59 where the codon-optimized CRISPR-associated (Cas) protein having at least 80% sequence identity to SEQ ID NOs: 2, 5, 9, 15, 85, 87, 95 or 96, and wherein the Cas protein is capable of binding to the RNA guide and of causing a break in the target nucleic acid sequence complementary to the RNA guide.

59. An engineered, non-naturally occurring CRISPR-Cas system comprising:
an RNA guide or a nucleic acid encoding the RNA guide, wherein the RNA guide comprises a direct repeat sequence and a spacer sequence capable of hybridizing to a target nucleic acid; and
a codon-optimized CRISPR-associated (Cas) protein having at least 80% sequence identity to SEQ ID NOs: 1, 4, 8, 14, 84 or 86;
wherein the Cas protein is fused to a deaminase, and wherein the Cas protein fusion is capable of binding to the RNA guide and of editing the target nucleic acid sequence complementary to the RNA guide.

59b. The engineered, non-naturally occurring CRISPR-Cas system of claim 59 where the codon-optimized CRISPR-associated (Cas) protein further comprising a nuclear localization sequence (NLS) and/or a FLAG, HIS or HA tag.

59c. The engineered, non-naturally occurring CRISPR-Cas system of claim 59b where the codon-optimized CRISPR-associated (Cas) protein having at least 80% sequence identity to SEQ ID NOs: 2, 5, 9, 15, 85, 87, 95 or 96, wherein the Cas protein is fused to a deaminase, and wherein the Cas protein fusion is capable of binding to the RNA guide and of editing the target nucleic acid sequence complementary to the RNA guide.

60. The system of claim 59, wherein the Cas9 protein is an inactive Cas9 (dCas9).

61. The system of claim any one of claims 58-60, wherein the RNA guide comprises a crRNA and a tracrRNA.

62. The system of any one of claims 58-60, wherein the RNA guide comprises an sgRNA.

63. The system of claim 62, wherein the sgRNA for use with Streptococcus constellatus Cas9 comprises a scaffold comprising a sequence having at least about 80% identity to
(SEQ ID NO: 3)
5′-GUUUUAGAGCUGUGCUGUUUAAACAACACAGCAAGUUAAAAUAAGGC
UUUGUCCGUACUCAAGCUUGCAAAAGCGUGCACCGAUUCGGUGCU-3′

64. The system of claim 62, the sgRNA for use with Sharpea Cas9 comprises a scaffold comprising a sequence having at least about 80% identity to
(SEQ ID NO: 7)
5′-GUUUUAGAGUUGUGUUAUUGAAAAAUAACACAACGAGUUAAAAUAAA
GCUUAUGCUUAAAUGCCAGCUUUGCUGGUGUCAUUUAGAUGACUUUACUA
AGGUUGCUUCGGCAACCUUUUU-3′

65. The system of claim 62, wherein the sgRNA for use with Veillonella parvula Cas9 comprises a scaffold comprising a sequence having at least about 80% identity to
(SEQ ID NO: 13)
5′-GUUUGAGAGUAGUGUGAAAACAUUACGAGUUCAAAUACAAAUUAAUU
UACAAUGCCUUCGGGCUGCCCGACGUAGGGCACCUACUCUCAAUUCUUCG
GAAUUGAGUU-3′

66. The system of claim 62, wherein the sgRNA for use with Ezakiella peruensis Cas9 comprises a scaffold comprising a sequence having at least about 80% identity to
(SEQ ID NO: 19)
5′-GUUUGAGAGUUAUGUAAUUGAAAAAUUACAUGACGAGUUCAAAUAAA
AAUUUAUUCAAACCGCCUAUUUAUAGGCCGCAGAUGUUCUGCAUUAUGCU
UGCUAUUGCAAGCUU-3′

67. The system of claim 62, wherein the sgRNA for use with Lactobacillus fermentum strain AF15-40LB Cas9 comprises a scaffold comprising a sequence having at least about 80% identity to
(SEQ ID NO: 95)
5′-GUCUUGGAUGAGUGUGAAAACACUCAUAGUCAAGAUCAAACGAGUGG
UUUUCCACGAGUUAUUACUUUUGAGGUCUUAUAUGGCCCAUACAUAAAAA
GGAGUCGGAAUUUCCGGCUCCUUUUCUU-3′

68. The system of claim 62, wherein the sgRNA for use with Peptoniphilus sp. Marseille-P3761 Cas9 comprises a scaffold comprising a sequence having at least about 80% identity to
(SEQ ID NO: 96)
5′-GUUUUAGAGCCAUGUAGAAAUACAUUGCAAGUUAAAAUAAGGCUUUG
UCCGUAAUCAACUUGAAAAAGUGGCGCUGUUUCGGCGCUUU-3′

69. The system of any one of claims 58-68, wherein the Cas protein is operably linked to a promoter sequence for expression in a eukaryotic cell, and wherein the guide RNA is operably linked to a promoter sequence for expression in a eukaryotic cell.

70. The system of claim 69, wherein the eukaryotic cell is a human cell.

71. The system of claim 70, wherein the promoter sequence is a eukaryotic promoter sequence.

72. A nucleic acid encoding the system of any one of claims 58-71.

73. A vector comprising the system of any one of claims 58-72.

74. The vector of claim 73, wherein the vector is a plasmid vector or a viral vector.

75. The vector of claim 74, wherein the viral vector is an adeno associated virus (AAV) vector or a lentiviral vector.

76. The vector of claim 75, wherein the viral vector is an AAV vector.

77. The vector of claim 76, wherein more than one AAV vector is used for packaging the system of claims 59-71.

78. A method of treating a disorder or a disease in a subject in need thereof, the method comprising administering to the subject a system of any one of claims 58-71,
wherein the guide RNA is complementary to at least 10 nucleotides of a target nucleic acid associated with the condition or disease;
wherein the Cas protein associates with the guide RNA;
wherein the guide RNA binds to the target nucleic acid;
wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

79. The method of claim 78, wherein the guide RNA is complementary to about 18-24 nucleotides.

80. The method of claim 79, wherein the guide RNA is complementary to 20 nucleotides.

81. A base editor comprising the fusion protein of any one of claims 16-19.

82. The base editor of claim 81 comprising an adenosine deaminase domain or a cytidine deaminase domain.

82b. The base editor of claim 81 comprising an adenosine deaminase domain and a cytidine deaminase domain.

83. A method of editing a nucleobase of a polynucleotide, the method comprising contacting the polynucleotide with the base editor of claim 81 in complex with one or more guide RNAs, wherein the base editor comprises an adenosine deaminase domain and wherein the one or more guide RNAs target the base editor to effect an A•T to G•C alteration in the polynucleotide.

84. A method of editing a nucleobase of a polynucleotide, the method comprising contacting the polynucleotide with the base editor of claim 81 in complex with one or more guide RNAs, wherein the base editor comprises a cytidine deaminase domain, and wherein the one or more guide RNAs target the base editor to effect an C•G to T•A alteration in the polynucleotide.

85. The method of claim 83 or 84, wherein the editing results in less than 50% indel formation in the target polynucleotide sequence.

86. The method of any one of claims 83-85, wherein the editing generates a point mutation.


SEQUENCE LISTING (182 sequences)

SEQ ID NO: 1 - protein, 1345 aa, Streptococcus constellatus
MGKPYSIGLDIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFDSPVVAYSILVFADVEKGKSKKLRKVQDMVGITIMEKKRFEKNPVDFLEQRGYRNVRLEKIIKLPKYSLFELENKRRRLLASAKELQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPAAFNFFGENIDRKRYTSVTECLNATLIHQSITGLYETRIDLSKLGED

SEQ ID NO: 2 - protein, 1399 aa, Artificial Sequence
  Description of Artificial Sequence Synthetic polypeptide
MPKKKRKVGGKPYSIGLDIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFDSPVVAYSILVFADVEKGKSKKLRKVQDMVGITIMEKKRFEKNPVDFLEQRGYRNVRLEKIIKLPKYSLFELENKRRRLLASAKELQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPAAFNFFGENIDRKRYTSVTECLNATLIHQSITGLYETRIDLSKLGEDGKRPAATKKAGQAKKKKGSYPYDVPDYAYPYDVPDYAYPYDVPDYA

SEQ ID NO: 3 - RNA, 92 nt, Artificial Sequence
  Description of Artificial Sequence Synthetic oligonucleotide
guuuuagagcugugcuguuuaaacaacacagcaaguuaaaauaaggcuuuguccguacucaagcuugcaaaagcgugcaccgauucggugcu

SEQ ID NO: 4 - protein, 1326 aa, Sharpea sp.
MAKNKDIRYSIGLDIGTNSVGWAVMDEHYELLKKGNHHMWGSRLFDAAEPAATRRASRSIRRRYNKRRERIRLLRDLLGDMVMEVDPTFFIRLLNVSFLDEEDKQKNLGNDYKDNYNLFIEKDFNDKTYYDKYPTIYHLRKELCENKEKADPRLIYLALHHIVKYRGNFLKEGQSFAKVYEDIEEKLDNTLKKFMSLNDLDNLFVDNDINSMITVLSKIYQRSKKADDLLKIMNPTKEERAAYKEFTKALVGLKFNVSKMILAQEVKKDDKDIELDFSNVDYDSTVDGLQAELGEYIEFIEMLHSINSWVELQDILGNNSTISAAMVERYEEHKNDLRVLKKVIREELPDKYNEVFREDNPKLHNYLGYIKYPKNTPVEEFYEYIKRLLAKVDTGEAREILERIDLEKFMLKQNSRTNGSIPYQMQKDEMIQIIDNQSVYYPQLKENREKLISILEFRIPYYFGPLNTHSEFAWIKKFEDKQKERILPWNYDQIVDIDATAEGFIERMQNTGTYFPDKPVMAKNSLTVSKFEVLNELNKIRINGKLIPVETKKELLSDLFMKNKTITDKKLKDWLVTHQYYDTNEELKIEGYQKDLQFSTSLAPWIDFTKIFGEINASNYQLIEKIIYDISIFEDKKILKRRLKKVYQLDDLLVDKILKLNYTGWSRLSEKLLTGIKSKNSKETILSILENSNMNLMEIINDESLGFKQIIEESNKKDIEGPFRYDEVKKLAGSPAIKRGIWQALLVVQEITKFMKHEPSHIYIEFAREEQEKVRTESRIAKLQKIYKDLNLQTKEDQLVYESLKKEDAKKKIDTDALYLYYLQMGKSMYSGKPLDIDKLSTYHIDHILPRSLIKDDSLDNRVLVLPKENEWKLDSETVPFEIRNKMMGFWQKLHENGLMSNKKFFSLIRTDFNEKDKKRFINRQLVETRQIIKNVAVIINDHYTNTNVVTVRAELSHQFRERYKIYKNRDLNDLHHAHDAYIACILGQFIHQNFGNMDVNMIYGQYKKNYKKDVQEHNNYGFILNSMNHIHFNDDNSVIWDPSYIGKIKSCFCYKDVYVTKKLEQNDAKLFDLTILPSDKNSENGVTKAKIPVNKYRKDVNKYGGFSGDAPIMLAIEADKGKKHVRQVIAFPLRLKNYNDEERIKFIEKEKNLKNVKILTEVKKNQLILINHQYFFITGTNELVNATQLKLSAKNTKNLFNLVDANKHNKLESIDDANFNEVIQELICKLQEPIYSRYNSIGKEFEDSYEKINAVTKQDKLYIIEYLIAIMSAKATQGYIKPELAREIGTNGKNKGRIKSFTIDLNKTTFISTSVTGLFSKKYKL

SEQ ID NO: 5 - protein, 1380 aa, Artificial Sequence
  Description of Artificial Sequence Synthetic polypeptide
MPKKKRKVGAKNKDIRYSIGLDIGTNSVGWAVMDEHYELLKKGNHHMWGSRLFDAAEPAATRRASRSIRRRYNKRRERIRLLRDLLGDMVMEVDPTFFIRLLNVSFLDEEDKQKNLGNDYKDNYNLFIEKDFNDKTYYDKYPTIYHLRKELCENKEKADPRLIYLALHHIVKYRGNFLKEGQSFAKVYEDIEEKLDNTLKKFMSLNDLDNLFVDNDINSMITVLSKIYQRSKKADDLLKIMNPTKEERAAYKEFTKALVGLKFNVSKMILAQEVKKDDKDIELDFSNVDYDSTVDGLQAELGEYIEFIEMLHSINSWVELQDILGNNSTISAAMVERYEEHKNDLRVLKKVIREELPDKYNEVFREDNPKLHNYLGYIKYPKNTPVEEFYEYIKRLLAKVDTGEAREILERIDLEKFMLKQNSRTNGSIPYQMQKDEMIQIIDNQSVYYPQLKENREKLISILEFRIPYYFGPLNTHSEFAWIKKFEDKQKERILPWNYDQIVDIDATAEGFIERMQNTGTYFPDKPVMAKNSLTVSKFEVLNELNKIRINGKLIPVETKKELLSDLFMKNKTITDKKLKDWLVTHQYYDTNEELKIEGYQKDLQFSTSLAPWIDFTKIFGEINASNYQLIEKIIYDISIFEDKKILKRRLKKVYQLDDLLVDKILKLNYTGWSRLSEKLLTGIKSKNSKETILSILENSNMNLMEIINDESLGFKQIIEESNKKDIEGPFRYDEVKKLAGSPAIKRGIWQALLVVQEITKFMKHEPSHIYIEFAREEQEKVRTESRIAKLQKIYKDLNLQTKEDQLVYESLKKEDAKKKIDTDALYLYYLQMGKSMYSGKPLDIDKLSTYHIDHILPRSLIKDDSLDNRVLVLPKENEWKLDSETVPFEIRNKMMGFWQKLHENGLMSNKKFFSLIRTDFNEKDKKRFINRQLVETRQIIKNVAVIINDHYTNTNVVTVRAELSHQFRERYKIYKNRDLNDLHHAHDAYIACILGQFIHQNFGNMDVNMIYGQYKKNYKKDVQEHNNYGFILNSMNHIHFNDDNSVIWDPSYIGKIKSCFCYKDVYVTKKLEQNDAKLFDLTILPSDKNSENGVTKAKIPVNKYRKDVNKYGGFSGDAPIMLAIEADKGKKHVRQVIAFPLRLKNYNDEERIKFIEKEKNLKNVKILTEVKKNQLILINHQYFFITGTNELVNATQLKLSAKNTKNLFNLVDANKHNKLESIDDANFNEVIQELICKLQEPIYSRYNSIGKEFEDSYEKINAVTKQDKLYIIEYLIAIMSAKATQGYIKPELAREIGTNGKNKGRIKSFTIDLNKTTFISTSVTGLFSKKYKLGKRPAATKKAGQAKKKKGSYPYDVPDYAYPYDVPDYAYPYDVPDYA

SEQ ID NO: 6 - protein, 1576 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGAKNKDIRYSIGLAIGTNSVGWAVMDEHYELLKKGNHHMWGSRLFDAAEPAATRRASRSIRRRYNKRRERIRLLRDLLGDMVMEVDPTFFIRLLNVSFLDEEDKQKNLGNDYKDNYNLFIEKDFNDKTYYDKYPTIYHLRKELCENKEKADPRLIYLALHHIVKYRGNFLKEGQSFAKVYEDIEEKLDNTLKKFMSLNDLDNLFVDNDINSMITVLSKIYQRSKKADDLLKIMNPTKEERAAYKEFTKALVGLKFNVSKMILAQEVKKDDKDIELDFSNVDYDSTVDGLQAELGEYIEFIEMLHSINSWVELQDILGNNSTISAAMVERYEEHKNDLRVLKKVIREELPDKYNEVFREDNPKLHNYLGYIKYPKNTPVEEFYEYIKRLLAKVDTGEAREILERIDLEKFMLKQNSRTNGSIPYQMQKDEMIQIIDNQSVYYPQLKENREKLISILEFRIPYYFGPLNTHSEFAWIKKFEDKQKERILPWNYDQIVDIDATAEGFIERMQNTGTYFPDKPVMAKNSLTVSKFEVLNELNKIRINGKLIPVETKKELLSDLFMKNKTITDKKLKDWLVTHQYYDTNEELKIEGYQKDLQFSTSLAPWIDFTKIFGEINASNYQLIEKIIYDISIFEDKKILKRRLKKVYQLDDLLVDKILKLNYTGWSRLSEKLLTGIKSKNSKETILSILENSNMNLMEIINDESLGFKQIIEESNKKDIEGPFRYDEVKKLAGSPAIKRGIWQALLVVQEITKFMKHEPSHIYIEFAREEQEKVRTESRIAKLQKIYKDLNLQTKEDQLVYESLKKEDAKKKIDTDALYLYYLQMGKSMYSGKPLDIDKLSTYHIDHILPRSLIKDDSLDNRVLVLPKENEWKLDSETVPFEIRNKMMGFWQKLHENGLMSNKKFFSLIRTDFNEKDKKRFINRQLVETRQIIKNVAVIINDHYTNTNVVTVRAELSHQFRERYKIYKNRDLNDLHHAHDAYIACILGQFIHQNFGNMDVNMIYGQYKKNYKKDVQEHNNYGFILNSMNHIHFNDDNSVIWDPSYIGKIKSCFCYKDVYVTKKLEQNDAKLFDLTILPSDKNSENGVTKAKIPVNKYRKDVNKYGGFSGDAPIMLAIEADKGKKHVRQVIAFPLRLKNYNDEERIKFIEKEKNLKNVKILTEVKKNQLILINHQYFFITGTNELVNATQLKLSAKNTKNLFNLVDANKHNKLESIDDANFNEVIQELICKLQEPIYSRYNSIGKEFEDSYEKINAVTKQDKLYIIEYLIAIMSAKATQGYIKPELAREIGTNGKNKGRIKSFTIDLNKTTFISTSVTGLFSKKYKLGKRPAATKKAGQAKKKKGSYPYDVPDYAYPYDVPDYAYPYDVPDYA

SEQ ID NO: 7 - RNA, 119 nt, Artificial Sequence
  Description of Artificial Sequence Synthetic polynucleotide
guuuuagaguuguguuauugaaaaauaacacaacgaguuaaaauaaagcuuaugcuuaaaugccagcuuugcuggugucauuuagaugacuuuacuaagguugcuucggcaaccuuuuu

SEQ ID NO: 8 - protein, 1409 aa, Veillonella parvula
MSIINFQRRGLMETQASNQLISSHLKGYPIKDYFVGLDIGTSSVGWAVTNKAYELLKFRSHKMWGSRLFDEGESAVARRGFRSMRRRLERRKLRLKLLEELFADAMAQVDPTFFMRLRESKYHYEDKTTGHSSKHILFIDKNYNDQDYFKEYPTVYHLRSELMKSGTDDIRKLFLAVHHILKYRGNFLYEGATFDSNASTLDDVIKQALENITFNCFDCNSAISSIGQILMEAGKTKSDKAKAIEHLVDTYIATDTVDTSSKTQKDQVKEDKKRLKAFANLVLGLNASLIDLFGSVEELEEDLKKLQITGDTYDDKRDELAKAWSDEIYIIDDCKSVYDAIILLSIKEPGLTISESKVKAFNKHKDDLAILKSLLKSDRSIYNTMFKVDEKGLHNYVHYIKQGRTEETSCNREDFYKYTKKIVEGLSDSKDKEYILSQIELQILLPLQRIKDNGVIPYQLHLEELKAILAKCGPKFPFLNEVADGFSVAEKLIKMLEFRIPYYVGPLNTHHNVDNGGFAWAVRKASGRVTPWNFDDKIDREKSAAAFIKNLTNKCTYLLGEDVLPKSSLLYSEFMLLNELNNVRIDGKPLEKVVKEHLIEAVFKQDHKKMTKNRIEQFLKDNGYISETHKHEITGLDGEIKNDLASYRDMVRILGDGFDRSMAEEIITDITIFGESKKMLRETLRKKFASCLDDEAIKKLTKLRYRDWGRLSQKLLNGIEGCDKAGDGTPETIIILMRNFSYNLMELLGDKFSFMERIQEINAKLTEGQIVNPHDIIDDLALSPAVKRAVWQALRIVDEVAHIKKALPARIFVEVTRSNKNEKKKKDSRQKRLSDLYAAIKKDDVLLNGLNNEIFGELKSSLAKYDDAALRSKKLYLYYTQMGRCAYTGEIIELSLLNTDNYDIDHIYPRSLTKDDSFDNLVLCKRTANAQKSDAYPISEEIQKTQKPFWTFLKQQGLISERKYERLTRITPLTADDLSGFIARQLVETNQSVKAATTLLRRLYPGVDVVFVKAENVTDFRHDNNFIKVRSLNHHHHAKDAYLNIVVGNVYHERFTRNFRAFFKKNGANRTYNLAKMFNYDVNCTNAKDGKAWDVKTSMDTVKKMMDSNDVRVTKRLLEQTGALADATIYKATVAGKAKDGAYIGMKTKSSVFADVSKYGGMTKIKNAYSIIVQYTGKKGEVIKEIVPLPIYLTNRNTTDQDLINYVASIIPQAKDISIIYGKLCINQLVKVNGFYYYLGGKTNSKFCIDNAIQVIVSNEWIPYLKVLEKFNNMRKDNKDLKANVVSTRALDNKHTIEVRIVEEKNIEFFDYLVSKLKMPIYQKMKGNKAAELSEKGYGLFKKMSLEEQSIHLIELLNLLTNQKTTFEVKPLGITASRSTVGSKISNQDEFKVINESITGLYSNEVTIV

SEQ ID NO: 9 - protein, 1463 aa, Artificial Sequence
  Description of Artificial Sequence Synthetic polypeptide
MPKKKRKVGSIINFQRRGLMETQASNQLISSHLKGYPIKDYFVGLDIGTSSVGWAVTNKAYELLKFRSHKMWGSRLFDEGESAVARRGFRSMRRRLERRKLRLKLLEELFADAMAQVDPTFFMRLRESKYHYEDKTTGHSSKHILFIDKNYNDQDYFKEYPTVYHLRSELMKSGTDDIRKLFLAVHHILKYRGNFLYEGATFDSNASTLDDVIKQALENITFNCFDCNSAISSIGQILMEAGKTKSDKAKAIEHLVDTYIATDTVDTSSKTQKDQVKEDKKRLKAFANLVLGLNASLIDLFGSVEELEEDLKKLQITGDTYDDKRDELAKAWSDEIYIIDDCKSVYDAIILLSIKEPGLTISESKVKAFNKHKDDLAILKSLLKSDRSIYNTMFKVDEKGLHNYVHYIKQGRTEETSCNREDFYKYTKKIVEGLSDSKDKEYILSQIELQILLPLQRIKDNGVIPYQLHLEELKAILAKCGPKFPFLNEVADGFSVAEKLIKMLEFRIPYYVGPLNTHHNVDNGGFAWAVRKASGRVTPWNFDDKIDREKSAAAFIKNLTNKCTYLLGEDVLPKSSLLYSEFMLLNELNNVRIDGKPLEKVVKEHLIEAVFKQDHKKMTKNRIEQFLKDNGYISETHKHEITGLDGEIKNDLASYRDMVRILGDGFDRSMAEEIITDITIFGESKKMLRETLRKKFASCLDDEAIKKLTKLRYRDWGRLSQKLLNGIEGCDKAGDGTPETIIILMRNFSYNLMELLGDKFSFMERIQEINAKLTEGQIVNPHDIIDDLALSPAVKRAVWQALRIVDEVAHIKKALPARIFVEVTRSNKNEKKKKDSRQKRLSDLYAAIKKDDVLLNGLNNEIFGELKSSLAKYDDAALRSKKLYLYYTQMGRCAYTGEIIELSLLNTDNYDIDHIYPRSLTKDDSFDNLVLCKRTANAQKSDAYPISEEIQKTQKPFWTFLKQQGLISERKYERLTRITPLTADDLSGFIARQLVETNQSVKAATTLLRRLYPGVDVVFVKAENVTDFRHDNNFIKVRSLNHHHHAKDAYLNIVVGNVYHERFTRNFRAFFKKNGANRTYNLAKMFNYDVNCTNAKDGKAWDVKTSMDTVKKMMDSNDVRVTKRLLEQTGALADATIYKATVAGKAKDGAYIGMKTKSSVFADVSKYGGMTKIKNAYSIIVQYTGKKGEVIKEIVPLPIYLTNRNTTDQDLINYVASIIPQAKDISIIYGKLCINQLVKVNGFYYYLGGKTNSKFCIDNAIQVIVSNEWIPYLKVLEKFNNMRKDNKDLKANVVSTRALDNKHTIEVRIVEEKNIEFFDYLVSKLKMPIYQKMKGNKAAELSEKGYGLFKKMSLEEQSIHLIELLNLLTNQKTTFEVKPLGITASRSTVGSKISNQDEFKVINESITGLYSNEVTIVGKRPAATKKAGQAKKKKGSYPYDVPDYAYPYDVPDYAYPYDVPDYA

SEQ ID NO: 10 - protein, 1659 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGSIINFQRRGLMETQASNQLISSHLKGYPIKDYFVGLAIGTSSVGWAVTNKAYELLKFRSHKMWGSRLFDEGESAVARRGFRSMRRRLERRKLRLKLLEELFADAMAQVDPTFFMRLRESKYHYEDKTTGHSSKHILFIDKNYNDQDYFKEYPTVYHLRSELMKSGTDDIRKLFLAVHHILKYRGNFLYEGATFDSNASTLDDVIKQALENITFNCFDCNSAISSIGQILMEAGKTKSDKAKAIEHLVDTYIATDTVDTSSKTQKDQVKEDKKRLKAFANLVLGLNASLIDLFGSVEELEEDLKKLQITGDTYDDKRDELAKAWSDEIYIIDDCKSVYDAIILLSIKEPGLTISESKVKAFNKHKDDLAILKSLLKSDRSIYNTMFKVDEKGLHNYVHYIKQGRTEETSCNREDFYKYTKKIVEGLSDSKDKEYILSQIELQILLPLQRIKDNGVIPYQLHLEELKAILAKCGPKFPFLNEVADGFSVAEKLIKMLEFRIPYYVGPLNTHHNVDNGGFAWAVRKASGRVTPWNFDDKIDREKSAAAFIKNLTNKCTYLLGEDVLPKSSLLYSEFMLLNELNNVRIDGKPLEKVVKEHLIEAVFKQDHKKMTKNRIEQFLKDNGYISETHKHEITGLDGEIKNDLASYRDMVRILGDGFDRSMAEEIITDITIFGESKKMLRETLRKKFASCLDDEAIKKLTKLRYRDWGRLSQKLLNGIEGCDKAGDGTPETIIILMRNFSYNLMELLGDKFSFMERIQEINAKLTEGQIVNPHDIIDDLALSPAVKRAVWQALRIVDEVAHIKKALPARIFVEVTRSNKNEKKKKDSRQKRLSDLYAAIKKDDVLLNGLNNEIFGELKSSLAKYDDAALRSKKLYLYYTQMGRCAYTGEIIELSLLNTDNYDIDHIYPRSLTKDDSFDNLVLCKRTANAQKSDAYPISEEIQKTQKPFWTFLKQQGLISERKYERLTRITPLTADDLSGFIARQLVETNQSVKAATTLLRRLYPGVDVVFVKAENVTDFRHDNNFIKVRSLNHHHHAKDAYLNIVVGNVYHERFTRNFRAFFKKNGANRTYNLAKMFNYDVNCTNAKDGKAWDVKTSMDTVKKMMDSNDVRVTKRLLEQTGALADATIYKATVAGKAKDGAYIGMKTKSSVFADVSKYGGMTKIKNAYSIIVQYTGKKGEVIKEIVPLPIYLTNRNTTDQDLINYVASIIPQAKDISIIYGKLCINQLVKVNGFYYYLGGKTNSKFCIDNAIQVIVSNEWIPYLKVLEKFNNMRKDNKDLKANVVSTRALDNKHTIEVRIVEEKNIEFFDYLVSKLKMPIYQKMKGNKAAELSEKGYGLFKKMSLEEQSIHLIELLNLLTNQKTTFEVKPLGITASRSTVGSKISNQDEFKVINESITGLYSNEVTIVGKRPAATKKAGQAKKKKGSYPYDVPDYAYPYDVPDYAYPYDVPDYA

SEQ ID NO: 11 - protein, 1654 aa, Artificial Sequence
  Description of Artificial Sequence Synthetic polypeptide
MPKKKRKVSIINFQRRGLMETQASNQLISSHLKGYPIKDYFVGLAIGTSSVGWAVTNKAYELLKFRSHKMWGSRLFDEGESAVARRGFRSMRRRLERRKLRLKLLEELFADAMAQVDPTFFMRLRESKYHYEDKTTGHSSKHILFIDKNYNDQDYFKEYPTVYHLRSELMKSGTDDIRKLFLAVHHILKYRGNFLYEGATFDSNASTLDDVIKQALENITFNCFDCNSAISSIGQILMEAGKTKSDKAKAIEHLVDTYIATDTVDTSSKTQKDQVKEDKKRLKAFANLVLGLNASLIDLFGSVEELEEDLKKLQITGDTYDDKRDELAKAWSDEIYIIDDCKSVYDAIILLSIKEPGLTISESKVKAFNKHKDDLAILKSLLKSDRSIYNTMFKVDEKGLHNYVHYIKQGRTEETSCNREDFYKYTKKIVEGLSDSKDKEYILSQIELQILLPLQRIKDNGVIPYQLHLEELKAILAKCGPKFPFLNEVADGFSVAEKLIKMLEFRIPYYVGPLNTHHNVDNGGFAWAVRKASGRVTPWNFDDKIDREKSAAAFIKNLTNKCTYLLGEDVLPKSSLLYSEFMLLNELNNVRIDGKPLEKVVKEHLIEAVFKQDHKKMTKNRIEQFLKDNGYISETHKHEITGLDGEIKNDLASYRDMVRILGDGFDRSMAEEIITDITIFGESKKMLRETLRKKFASCLDDEAIKKLTKLRYRDWGRLSQKLLNGIEGCDKAGDGTPETIIILMRNFSYNLMELLGDKFSFMERIQEINAKLTEGQIVNPHDIIDDLALSPAVKRAVWQALRIVDEVAHIKKALPARIFVEVTRSNKNEKKKKDSRQKRLSDLYAAIKKDDVLLNGLNNEIFGELKSSLAKYDDAALRSKKLYLYYTQMGRCAYTGEIIELSLLNTDNYDIDHIYPRSLTKDDSFDNLVLCKRTANAQKSDAYPISEEIQKTQKPFWTFLKQQGLISERKYERLTRITPLTADDLSGFIARQLVETNQSVKAATTLLRRLYPGVDVVFVKAENVTDFRHDNNFIKVRSLNHHHHAKDAYLNIVVGNVYHERFTRNFRAFFKKNGANRTYNLAKMFNYDVNCTNAKDGKAWDVKTSMDTVKKMMDSNDVRVTKRLLEQTGALADATIYKATVAGKAKDGAYIGMKTKSSVFADVSKYGGMTKIKNAYSIIVQYTGKKGEVIKEIVPLPIYLTNRNTTDQDLINYVASIIPQAKDISIIYGKLCINQLVKVNGFYYYLGGKTNSKFCIDNAIQVIVSNEWIPYLKVLEKFNNMRKDNKDLKANVVSTRALDNKHTIEVRIVEEKNIEFFDYLVSKLKMPIYQKMKGNKAAELSEKGYGLFKKMSLEEQSIHLIELLNLLTNQKTTFEVKPLGITASRSTVGSKISNQDEFKVINESITGLYSNEVTIVKRPAATKKAGQAKKKKSGSETPGTSESATPESSGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDPAAKRVKLDGSYPYDVPDYAYPYDVPDYAYPYDVPDYA

SEQ ID NO: 12 - protein, 1919 aa, Artificial Sequence
  Description of Artificial Sequence Synthetic polypeptide
MPAAKRVKLDTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGSIINFQRRGLMETQASNQLISSHLKGYPIKDYFVGLAIGTSSVGWAVTNKAYELLKFRSHKMWGSRLFDEGESAVARRGFRSMRRRLERRKLRLKLLEELFADAMAQVDPTFFMRLRESKYHYEDKTTGHSSKHILFIDKNYNDQDYFKEYPTVYHLRSELMKSGTDDIRKLFLAVHHILKYRGNFLYEGATFDSNASTLDDVIKQALENITFNCFDCNSAISSIGQILMEAGKTKSDKAKAIEHLVDTYIATDTVDTSSKTQKDQVKEDKKRLKAFANLVLGLNASLIDLFGSVEELEEDLKKLQITGDTYDDKRDELAKAWSDEIYIIDDCKSVYDAIILLSIKEPGLTISESKVKAFNKHKDDLAILKSLLKSDRSIYNTMFKVDEKGLHNYVHYIKQGRTEETSCNREDFYKYTKKIVEGLSDSKDKEYILSQIELQILLPLQRIKDNGVIPYQLHLEELKAILAKCGPKFPFLNEVADGFSVAEKLIKMLEFRIPYYVGPLNTHHNVDNGGFAWAVRKASGRVTPWNFDDKIDREKSAAAFIKNLTNKCTYLLGEDVLPKSSLLYSEFMLLNELNNVRIDGKPLEKVVKEHLIEAVFKQDHKKMTKNRIEQFLKDNGYISETHKHEITGLDGEIKNDLASYRDMVRILGDGFDRSMAEEIITDITIFGESKKMLRETLRKKFASCLDDEAIKKLTKLRYRDWGRLSQKLLNGIEGCDKAGDGTPETIIILMRNFSYNLMELLGDKFSFMERIQEINAKLTEGQIVNPHDIIDDLALSPAVKRAVWQALRIVDEVAHIKKALPARIFVEVTRSNKNEKKKKDSRQKRLSDLYAAIKKDDVLLNGLNNEIFGELKSSLAKYDDAALRSKKLYLYYTQMGRCAYTGEIIELSLLNTDNYDIDHIYPRSLTKDDSFDNLVLCKRTANAQKSDAYPISEEIQKTQKPFWTFLKQQGLISERKYERLTRITPLTADDLSGFIARQLVETNQSVKAATTLLRRLYPGVDVVFVKAENVTDFRHDNNFIKVRSLNHHHHAKDAYLNIVVGNVYHERFTRNFRAFFKKNGANRTYNLAKMFNYDVNCTNAKDGKAWDVKTSMDTVKKMMDSNDVRVTKRLLEQTGALADATIYKATVAGKAKDGAYIGMKTKSSVFADVSKYGGMTKIKNAYSIIVQYTGKKGEVIKEIVPLPIYLTNRNTTDQDLINYVASIIPQAKDISIIYGKLCINQLVKVNGFYYYLGGKTNSKFCIDNAIQVIVSNEWIPYLKVLEKFNNMRKDNKDLKANVVSTRALDNKHTIEVRIVEEKNIEFFDYLVSKLKMPIYQKMKGNKAAELSEKGYGLFKKMSLEEQSIHLIELLNLLTNQKTTFEVKPLGITASRSTVGSKISNQDEFKVINESITGLYSNEVTIVGKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAY

SEQ ID NO: 13 - RNA, 107 nt, Artificial Sequence
  Description of Artificial Sequence Synthetic polynucleotide
guuugagaguagugugaaaacauuacgaguucaaauacaaauuaauuuacaaugccuucgggcugcccgacguagggcaccuacucucaauucuucggaauugaguu

SEQ ID NO: 14 - protein, 1370 aa, Ezakiella peruensis
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 15 - protein, 1424 aa, Artificial Sequence
  Description of Artificial Sequence Synthetic polypeptide
MPKKKRKVGTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGKRPAATKKAGQAKKKKGSYPYDVPDYAYPYDVPDYAYPYDVPDYA

SEQ ID NO: 16 - protein, 1620 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTKVKDYYIGLAIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGKRPAATKKAGQAKKKKGSYPYDVPDYAYPYDVPDYAYPYDVPDYA

SEQ ID NO: 17 - protein, 1615 aa, Artificial Sequence
  Description of Artificial Sequence Synthetic polypeptide
MPKKKRKVTKVKDYYIGLAIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLKRPAATKKAGQAKKKKSGSETPGTSESATPESSGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDPAAKRVKLDGSYPYDVPDYAYPYDVPDYAYPYDVPDYA

SEQ ID NO: 18 - protein, 1880 aa, Artificial Sequence
  Description of Artificial Sequence Synthetic polypeptide
MPAAKRVKLDTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGTKVKDYYIGLAIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAY

SEQ ID NO: 19 - RNA, 112 nt, Artificial Sequence
  Description of Artificial Sequence Synthetic polynucleotide
guuugagaguuauguaauugaaaaauuacaugacgaguucaaauaaaaauuuauucaaaccgccuauuuauaggccgcagauguucugcauuaugcuugcuauugcaagcuu

SEQ ID NO: 20 - protein, 1595 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGGKPYSIGLAIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFDSPVVAYSILVFADVEKGKSKKLRKVQDMVGITIMEKKRFEKNPVDFLEQRGYRNVRLEKIIKLPKYSLFELENKRRRLLASAKELQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPAAFNFFGENIDRKRYTSVTECLNATLIHQSITGLYETRIDLSKLGEDGKRPAATKKAGQAKKKKGSYPYDVPDYAYPYDVPDYAYPYDVPDYA

SEQ ID NO: 21 - protein, 1863 aa, Artificial Sequence
  Description of Artificial Sequence Synthetic polypeptide
MPAAKRVKLDTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGGKPYSIGLAIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFDSPVVAYSILVFADVEKGKSKKLRKVQDMVGITIMEKKRFEKNPVDFLEQRGYRNVRLEKIIKLPKYSLFELENKRRRLLASAKELQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPAAFNFFGENIDRKRYTSVTECLNATLIHQSITGLYETRIDLSKLGEDGKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAYPYDVPDYA

SEQ ID NO: 22 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 23 - protein, 145 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 24 - protein, 198 aa, Homo sapiens [sequence omitted]

SEQ ID NO: 25 - protein, 198 aa, Mus sp.
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 26 - protein, 198 aa, Canis lupus familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 27 - protein, 199 aa, Bos sp.
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 28 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 29 - protein, 198 aa, Canis lupus familiaris; identical to SEQ ID NO: 26

SEQ ID NO: 30 - protein, 199 aa, Bos taurus; identical to SEQ ID NO: 27

SEQ ID NO: 31 - protein, 198 aa, Mus musculus [sequence omitted]

SEQ ID NO: 32 - protein, 229 aa, Rattus norvegicus [sequence omitted]

SEQ ID NO: 33 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 34 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 35 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 36 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 37 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 38 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 39 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 40 - protein, 429 aa, Mus sp.
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 41 - protein, 430 aa, Rattus sp.
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 42 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 43 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 44 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 45 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 46 - protein, 377 aa, Chlorocebus sp.
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 47 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 48 - protein, 373 aa, Homo sapiens; identical to SEQ ID NO: 43

SEQ ID NO: 49 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 50 - protein, 395 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 51 - protein, 227 aa, Bos sp.
MDGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 52 - protein, 490 aa, Pan troglodytes
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 53 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 54 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 55 - protein, 199 aa, Homo sapiens; identical to SEQ ID NO: 42

SEQ ID NO: 56 - protein, 202 aa, Macaca mulatta
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 57 - protein, 185 aa, Bos sp.
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 58 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 59 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 60 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 61 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 62 - protein, 229 aa, Mus sp.
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 63 - protein, 229 aa, Rattus sp. [sequence omitted]

SEQ ID NO: 64 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 65 - protein, 224 aa, Mus sp.
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 66 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 67 - protein, 224 aa, Bos sp.; identical to SEQ ID NO: 38

SEQ ID NO: 68 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 69 - protein, 381 aa, Artificial Sequence
  Description of Artificial Sequence Synthetic polypeptide
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 70 - protein, 182 aa, Homo sapiens
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 71 - protein, 184 aa, Artificial Sequence
  Description of Artificial Sequence Synthetic polypeptide
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 72 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 73 - protein, 198 aa, Mus musculus; identical to SEQ ID NO: 25

SEQ ID NO: 74 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 75 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 76 - protein, 224 aa, Petromyzon marinus
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 77 - protein, 331 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 78 - protein, 209 aa, Petromyzon marinus
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 79 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 80 - protein, 218 aa, Artificial Sequence
  Description of Artificial Sequence Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 81 - protein, 218 aa, Artificial Sequence
  Description of Artificial Sequence Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 82 - protein, 429 aa, Mus sp.; identical to SEQ ID NO: 40

SEQ ID NO: 83 - protein, 167 aa, Artificial Sequence
  Description of Artificial Sequence Synthetic polypeptide
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 84 - protein, 1378 aa, Lactobacillus fermentum
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPAKNQAFIGKLLFPDLLKKNERGYPTLIKMRDELPVEQRAHYPVTNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKEKGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKAGDVASLVMAKTRGVNKPSVEGLSDPKKFNSNLATYLDLKSIVGDKVDDNRYQMDLENIIEWRSVFEDGEIFADKLTEVEWLTDEQRSALVKKRYKGWGRLSKKLLTGIVDENGQRIIDLMWNTDQNFMQIVNQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDDSLDNRVLVSRAENNKKSDRVPAKLYAAKMKPYWNQLLKQGLITQRKFENLTMDVDQTIKYRSLGFVKRQLVETRQVIKLTANILGSMYQEAGTDIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVADYFDWVINLKVMLISNETYEETGKYFDASHESSSLYLKNQNKKSKLVVPLKNKLQPEYYGAYTGITQGYMVILKLLDKKGGFGVYRIPRYAADILNKCHDEVAYRNKIAEIISSDPRAPKSFEVVVPRVLKGTFLVDGEEKFILSSYRYKVNATQLILPVSDIKLIQDNFKALKKLNVEMQTKKLIEIYDNILRQVDKYYKLYDINKFRAKLHDGRSKFVELDDFGQDASKEKVIIKILRGLHFGSDLQNLKEIGFGTTPLGQFQVSEAGIRLSNTAFIIFKSPTGLFNRKLYLKNL

SEQ ID NO: 85 - protein, 1432 aa, Artificial Sequence
  Description of Artificial Sequence Synthetic polypeptide
MPKKKRKVGKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPAKNQAFIGKLLFPDLLKKNERGYPTLIKMRDELPVEQRAHYPVTNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKEKGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKAGDVASLVMAKTRGVNKPSVEGLSDPKKFNSNLATYLDLKSIVGDKVDDNRYQMDLENIIEWRSVFEDGEIFADKLTEVEWLTDEQRSALVKKRYKGWGRLSKKLLTGIVDENGQRIIDLMWNTDQNFMQIVNQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDDSLDNRVLVSRAENNKKSDRVPAKLYAAKMKPYWNQLLKQGLITQRKFENLTMDVDQTIKYRSLGFVKRQLVETRQVIKLTANILGSMYQEAGTDIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVADYFDWVINLKVMLISNETYEETGKYFDASHESSSLYLKNQNKKSKLVVPLKNKLQPEYYGAYTGITQGYMVILKLLDKKGGFGVYRIPRYAADILNKCHDEVAYRNKIAEIISSDPRAPKSFEVVVPRVLKGTFLVDGEEKFILSSYRYKVNATQLILPVSDIKLIQDNFKALKKLNVEMQTKKLIEIYDNILRQVDKYYKLYDINKFRAKLHDGRSKFVELDDFGQDASKEKVIIKILRGLHFGSDLQNLKEIGFGTTPLGQFQVSEAGIRLSNTAFIIFKSPTGLFNRKLYLKNLGKRPAATKKAGQAKKKKGSYPYDVPDYAYPYDVPDYAYPYDVPDYA

SEQ ID NO: 86 - protein, 1359 aa, Peptoniphilus sp.
MEKKTNYTIGLDIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFED

SEQ ID NO: 87 - protein, 1413 aa, Artificial Sequence
  Description of Artificial Sequence Synthetic polypeptide
MPKKKRKVGEKKTNYTIGLDIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFEDGKRPAATKKAGQAKKKKGSYPYDVPDYAYPYDVPDYAYPYDVPDYA

SEQ ID NO: 88 - protein, 1628 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGKEYHIGLAIGTSSIGWAVTDSQFKLMRIKGKTAIGVRLFEEGKTAAERRTFRTTRRRLKRRKWRLHYLDEIFAPHLQEVDENFLRRLKQSNIHPEDPAKNQAFIGKLLFPDLLKKNERGYPTLIKMRDELPVEQRAHYPVTNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKEKGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKAGDVASLVMAKTRGVNKPSVEGLSDPKKFNSNLATYLDLKSIVGDKVDDNRYQMDLENIIEWRSVFEDGEIFADKLTEVEWLTDEQRSALVKKRYKGWGRLSKKLLTGIVDENGQRIIDLMWNTDQNFMQIVNQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDDSLDNRVLVSRAENNKKSDRVPAKLYAAKMKPYWNQLLKQGLITQRKFENLTMDVDQTIKYRSLGFVKRQLVETRQVIKLTANILGSMYQEAGTDIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVADYFDWVINLKVMLISNETYEETGKYFDASHESSSLYLKNQNKKSKLVVPLKNKLQPEYYGAYTGITQGYMVILKLLDKKGGFGVYRIPRYAADILNKCHDEVAYRNKIAEIISSDPRAPKSFEVVVPRVLKGTFLVDGEEKFILSSYRYKVNATQLILPVSDIKLIQDNFKALKKLNVEMQTKKLIEIYDNILRQVDKYYKLYDINKFRAKLHDGRSKFVELDDFGQDASKEKVIIKILRGLHFGSDLQNLKEIGFGTTPLGQFQVSEAGIRLSNTAFIIFKSPTGLFNRKLYLKNLGKRPAATKKAGQAKKKKGSYPYDVPDYAYPYDVPDYAYPYDVPDYA

SEQ ID NO: 89 - protein, 1624 aa, Artificial Sequence
  Description of Artificial Sequence Synthetic polypeptide
MPKKKRKVGKEYHIGLAIGTSSIGWAVTDSQFKLMRIKGKTAIGVRLFEEGKTAAERRTFRTTRRRLKRRKWRLHYLDEIFAPHLQEVDENFLRRLKQSNIHPEDPAKNQAFIGKLLFPDLLKKNERGYPTLIKMRDELPVEQRAHYPVTNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKEKGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKAGDVASLVMAKTRGVNKPSVEGLSDPKKFNSNLATYLDLKSIVGDKVDDNRYQMDLENIIEWRSVFEDGEIFADKLTEVEWLTDEQRSALVKKRYKGWGRLSKKLLTGIVDENGQRIIDLMWNTDQNFMQIVNQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDDSLDNRVLVSRAENNKKSDRVPAKLYAAKMKPYWNQLLKQGLITQRKFENLTMDVDQTIKYRSLGFVKRQLVETRQVIKLTANILGSMYQEAGTDIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVADYFDWVINLKVMLISNETYEETGKYFDASHESSSLYLKNQNKKSKLVVPLKNKLQPEYYGAYTGITQGYMVILKLLDKKGGFGVYRIPRYAADILNKCHDEVAYRNKIAEIISSDPRAPKSFEVVVPRVLKGTFLVDGEEKFILSSYRYKVNATQLILPVSDIKLIQDNFKALKKLNVEMQTKKLIEIYDNILRQVDKYYKLYDINKFRAKLHDGRSKFVELDDFGQDASKEKVIIKILRGLHFGSDLQNLKEIGFGTTPLGQFQVSEAGIRLSNTAFIIFKSPTGLFNRKLYLKNLKRPAATKKAGQAKKKKSGSETPGTSESATPESSGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDPAAKRVKLDGSYPYDVPDYAYPYDVPDYAYPYDVPDYA

SEQ ID NO: 90 - protein, 1888 aa, Artificial Sequence
  Description of Artificial Sequence Synthetic polypeptide
MPAAKRVKLDTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGKEYHIGLAIGTSSIGWAVTDSQFKLMRIKGKTAIGVRLFEEGKTAAERRTFRTTRRRLKRRKWRLHYLDEIFAPHLQEVDENFLRRLKQSNIHPEDPAKNQAFIGKLLFPDLLKKNERGYPTLIKMRDELPVEQRAHYPVTNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKEKGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKAGDVASLVMAKTRGVNKPSVEGLSDPKKFNSNLATYLDLKSIVGDKVDDNRYQMDLENIIEWRSVFEDGEIFADKLTEVEWLTDEQRSALVKKRYKGWGRLSKKLLTGIVDENGQRIIDLMWNTDQNFMQIVNQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDDSLDNRVLVSRAENNKKSDRVPAKLYAAKMKPYWNQLLKQGLITQRKFENLTMDVDQTIKYRSLGFVKRQLVETRQVIKLTANILGSMYQEAGTDIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVADYFDWVINLKVMLISNETYEETGKYFDASHESSSLYLKNQNKKSKLVVPLKNKLQPEYYGAYTGITQGYMVILKLLDKKGGFGVYRIPRYAADILNKCHDEVAYRNKIAEIISSDPRAPKSFEVVVPRVLKGTFLVDGEEKFILSSYRYKVNATQLILPVSDIKLIQDNFKALKKLNVEMQTKKLIEIYDNILRQVDKYYKLYDINKFRAKLHDGRSKFVELDDFGQDASKEKVIIKILRGLHFGSDLQNLKEIGFGTTPLGQFQVSEAGIRLSNTAFIIFKSPTGLFNRKLYLKNLGKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAY

SEQ ID NO: 91 - protein, 1609 aa, Artificial Sequence
  Description of Artificial Sequence Synthetic polypeptide
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGEKKTNYTIGLAIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFEDGKRPAATKKAGQAKKKKGSYPYDVPDYAYPYDVPDYAYPYDVPDYA

SEQ ID NO: 92 - protein, 1604 aa, Artificial Sequence
  Description of Artificial Sequence Synthetic polypeptide
MPKKKRKVEKKTNYTIGLAIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFEDKRPAATKKAGQAKKKKSGSETPGTSESATPESSGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDPAAKRVKLDGSYPYDVPDYAYPYDVPDYAYPYDVPDYA

SEQ ID NO: 93 - protein, 1869 aa, Artificial Sequence
  Description of Artificial Sequence Synthetic polypeptide
MPAAKRVKLDTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGEKKTNYTIGLAIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFEDGKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAY

SEQ ID NO: 94 - protein, 1873 aa, Artificial Sequence
  Description of Artificial Sequence Synthetic polypeptide
MPAAKRVKLDTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSPKKKRKVEKKTNYTIGLAIGTDSVGWAVVKDDLELVKKRMKVLGNTETNYIKKNLWGSLLFESGQTAKDRRLKRVARRRYERRRNRLTELQKIFAPAIDEVDENFFFRLNESFLVPEDKAFSKNPIFGTLGEDKTYYKTYPTIYHLRQHLADSEEKADVRLIYLALAHMIKYRGHFLIEGKLDTEHIAINENLEQFFESYNALFSEEPIELRKEELIAIENILREKNSRTVKEKRITSFLKDIGRANKQSPMMAFITLIVGKKAKFKAAFNLEEEISLNLTDDSYDENLEILLNTIGSDFADLFDHAQRVYNAVELAGILSGDVKNTHAKLSAQMVAMYERHKEQLKEYKSFIKANLPDQYDMTFVAPKDAQKKDLKGYAGYIDGNMSQDSFYKFVKDQLKEVPGSEKFLDSIEKEDFLRKQRSFYNGVIPNQVHLAEMEAILDRQENYYPWLKENREKIISLLTFRIPYYVGPLADGQSEFAWLERKSDEKIKPWNFSDVVDLDRSAEKFIEQLIGRDTYLPDEYVLPKKSLIYQKYMVFNELTKIAYLDERQKRMNLSSVEKKEIFETLFKKRSKVTEKQLVKFFENYLQIDNPTIFGIEDAFNADYSTYVELAKVPGMKSMMDDPDNEDLMEEIVKILTVFEDRKMRRKQLEKYKERLSPEQIKELAKKHYTGWGRLSKKLLVGIRDKETQKTILDYLVEDDNHSGGRQHLNRNLMQLINDDRLSFKKTIAELQMIDPSADLYAQVQEIAGSPAIKKGILLGLKIVDEIIRVMGEKPENIVIEMARENQTTARGKALSKRREAKIKEGLAALGSSLLKENLPGNADLSQRKIYLYYTQNGKDIYLDEPLDFDRLSQYDEDHIIPQSFTVDNSLDNLVLTNSSQNRGNKKDDVPSLEVVNRQLAYWRSLKDAGLMTQRKFDNLTKAMRGGLTDKDRERFIQRQLVETRQITKNVAKLLDMRLNDKKDEAGNKIRETNIVLLKSAMASEFRKMFRLYKVRELNDYHHAHDAYLNAAIAINLLALYPYMADDFVYGEFRYKKKPQAEKATYEKLRQWNLIKRFGEKQLFTPDHEDCWNKERDIKTIKKVMGYRQVNVVKKAEERTGMLFKETINGKTNKGSRIPIKKDLDPSKYGGYIEEKMAYYAVISYEDKKKKPGKTIVGISIMDKKEFEYDSISYLGKLGFSNPVVQIILKNYSLIAYPDGRRRYITGATKTTKGKVELQKANQIAMEQDLVNFIYHLKNYDEISHPESYAFVQSHTDYFDRLFDSIEHYTRRFLDAETNINRLRRIYEEEKKKDPVDIEALVASFIELLKLTSAGAPADFIFMGEAISRRRYNSMTGLFDGQVIYQSLTGLYETRMRFEDKRPAATKKAGQAKKKKGSSGGSSGGSSGSETPGTSESATPESSGGSSGGSTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRYPYDVPDYAYPYDVPDYAYPYDVPDYA

SEQ ID NO: 95 - RNA, 125 nt, Artificial Sequence
  Description of Artificial Sequence Synthetic polynucleotide
gucuuggaugagugugaaaacacucauagucaagaucaaacgagugguuuuccacgaguuauuacuuuugaggucuuauauggcccauacauaaaaaggagucggaauuuccggcuccuuuucuu

SEQ ID NO: 96 - RNA, 88 nt, Artificial Sequence
  Description of Artificial Sequence Synthetic oligonucleotide
guuuuagagccauguagaaauacauugcaaguuaaaauaaggcuuuguccguaaucaacuugaaaaaguggcgcuguuucggcgcuuu

SEQ ID NO: 97 - DNA, 20 bp, Artificial Sequence
  Description of Artificial Sequence Synthetic oligonucleotide
gaaacaatgataacaagacc

SEQ ID NO: 98 - DNA, 20 bp, Artificial Sequence
  Description of Artificial Sequence Synthetic oligonucleotide
gtggcccctgtgcccagccc

SEQ ID NO: 99 - DNA, 20 bp, Artificial Sequence
  Description of Artificial Sequence Synthetic oligonucleotide
gtcccaaatatgtagctgtt

SEQ ID NO: 100 - DNA, 20 bp, Artificial Sequence
  Description of Artificial Sequence Synthetic oligonucleotide
gctcccatcacatcaaccgg

SEQ ID NO: 101 - DNA, 20 bp, Artificial Sequence
  Description of Artificial Sequence Synthetic oligonucleotide
gatgtcacctccaatgacta

SEQ ID NO: 102 - DNA, 20 bp, Artificial Sequence
  Description of Artificial Sequence Synthetic oligonucleotide
gttgaagatgaagcccagag

SEQ ID NO: 103 - DNA, 20 bp, Artificial Sequence
  Description of Artificial Sequence Synthetic oligonucleotide
gccaacaccaaccagaactt

SEQ ID NO: 104 - DNA, 20 bp, Artificial Sequence
  Description of Artificial Sequence Synthetic oligonucleotide
tgctgcacacagcaggcctt

SEQ ID NO: 105 - DNA, 20 bp, Artificial Sequence
  Description of Artificial Sequence Synthetic oligonucleotide
gaacacaaagcatagactgc

SEQ ID NO: 106 - DNA, 20 bp, Artificial Sequence
  Description of Artificial Sequence Synthetic oligonucleotide
gaaacaatgataacaagacc

SEQ ID NO: 107 - DNA, 20 bp, Artificial Sequence
  Description of Artificial Sequence Synthetic oligonucleotide
gtggcccctgtgcccagccc

SEQ ID NO: 108 - DNA, 20 bp, Artificial Sequence
  Description of Artificial Sequence Synthetic oligonucleotide
gtcccaaatatgtagctgtt

SEQ ID NO: 109 - DNA, 20 bp, Artificial Sequence
  Description of Artificial Sequence Synthetic oligonucleotide
agagggacacacagatctat

SEQ ID NO: 110 - DNA, 20 bp, Artificial Sequence
  Description of Artificial Sequence Synthetic oligonucleotide
gctcccatcacatcaaccgg

SEQ ID NO: 111 - DNA, 20 bp, Artificial Sequence
  Description of Artificial Sequence Synthetic oligonucleotide
gatgtcacctccaatgacta

SEQ ID NO: 112 - DNA, 20 bp, Artificial Sequence
  Description of Artificial Sequence Synthetic oligonucleotide
gggcaaccacaaacccacga

SEQ ID NO: 113 - DNA, 20 bp, Artificial Sequence
  Description of Artificial Sequence Synthetic oligonucleotide
gttgaagatgaagcccagag

SEQ ID NO: 114 - DNA, 20 bp, Artificial Sequence
  Description of Artificial Sequence Synthetic oligonucleotide
gccaacaccaaccagaactt

SEQ ID NO: 115 - DNA, 20 bp, Artificial Sequence
  Description of Artificial Sequence Synthetic oligonucleotide
tgctgcacacagcaggcctt

SEQ ID NO: 116 - DNA, 20 bp, Artificial Sequence
  Description of Artificial Sequence Synthetic oligonucleotide
gtgccagaaacaggggtgac

SEQ ID NO: 117 - DNA, 20 bp, Artificial Sequence
  Description of Artificial Sequence Synthetic oligonucleotide
cctgcctcagctgctcactt

SEQ ID NO: 118 - DNA, 20 bp, Artificial Sequence
  Description of Artificial Sequence Synthetic oligonucleotide
aaacggtccccagagggttc

SEQ ID NO: 119 - DNA, 20 bp, Artificial Sequence
  Description of Artificial Sequence Synthetic oligonucleotide
gccaccggttgatgtgatgg

SEQ ID NO: 120 - DNA, 20 bp, Artificial Sequence
  Description of Artificial Sequence Synthetic oligonucleotide
aagtggtcccaggcctcagc

SEQ ID NO: 121 - DNA, 20 bp, Artificial Sequence
  Description of Artificial Sequence Synthetic oligonucleotide
agagaaaatgaaactttcaa

SEQ ID NO: 122 - DNA, 20 bp, Artificial Sequence
  Description of Artificial Sequence Synthetic oligonucleotide
ccaaacccaactccatctac

SEQ ID NO: 123 - DNA, 20 bp, Artificial Sequence
  Description of Artificial Sequence Synthetic oligonucleotide
ggtccttgaattgcagtatc

SEQ ID NO: 124 - DNA, 20 bp, Artificial Sequence
  Description of Artificial Sequence Synthetic oligonucleotide
gcatagactgcggggcgggc

SEQ ID NO: 125 - DNA, 20 bp, Artificial Sequence
  Description of Artificial Sequence Synthetic oligonucleotide
ggaaactggaacacaaagca

SEQ ID NO: 126 - DNA, 20 bp, Artificial Sequence
  Description of Artificial Sequence Synthetic oligonucleotide
gacagcatgtggtaattttc

SEQ ID NO: 127 - DNA, 20 bp, Artificial Sequence
  Description of Artificial Sequence Synthetic oligonucleotide
gcccccggaaactctgtcca

SEQ ID NO: 128 - DNA, 20 bp, Artificial Sequence
  Description of Artificial Sequence Synthetic oligonucleotide
tcgacccccaccaaggttca

SEQ ID NO: 129 - DNA, 20 bp, Artificial Sequence
  Description of Artificial Sequence Synthetic oligonucleotide
tgttaacagctgacccaata

SEQ ID NO: 130 - DNA, 20 bp, Artificial Sequence
  Description of Artificial Sequence Synthetic oligonucleotide
gttactcgcctgtcaagtgg

SEQ ID NO: 131 - DNA, 20 bp, Artificial Sequence
  Description of Artificial Sequence Synthetic oligonucleotide
gggctcccatcacatcaacc

SEQ ID NO: 132 - DNA, 20 bp, Artificial Sequence
  Description of Artificial Sequence Synthetic oligonucleotide
gctttggggaggcctggagt

SEQ ID NO: 133 - DNA, 20 bp, Artificial Sequence
  Description of Artificial Sequence Synthetic oligonucleotide
tagctgccaatgactatagc

SEQ ID NO: 134 - DNA, 20 bp, Artificial Sequence
  Description of Artificial Sequence Synthetic oligonucleotide
ttaaaataggatctacatca

SEQ ID NO: 135 - DNA, 20 bp, Artificial Sequence
  Description of Artificial Sequence Synthetic oligonucleotide
gaatcctgccatacactttg

SEQ ID NO: 136 - DNA, 20 bp, Artificial Sequence
  Description of Artificial Sequence Synthetic oligonucleotide
ctgcggggcgggccagcctg

SEQ ID NO: 137 - DNA, 20 bp, Artificial Sequence
  Description of Artificial Sequence Synthetic oligonucleotide
acattgtcagagggacacac

SEQ ID NO: 138 - DNA, 20 bp, Artificial Sequence
  Description of Artificial Sequence Synthetic oligonucleotide
agcaactccagtcccaaata

SEQ ID NO: 139 - DNA, 20 bp, Artificial Sequence
  Description of Artificial Sequence Synthetic oligonucleotide
gtggtggccgagcgccccct

SEQ ID NO: 140 - DNA, 20 bp, Artificial Sequence
  Description of Artificial Sequence Synthetic oligonucleotide
cattcacccagcttccctgt

SEQ ID NO: 141 - DNA, 20 bp, Artificial Sequence
  Description of Artificial Sequence Synthetic oligonucleotide
gaacacaaagcatagactgc

SEQ ID NO: 142 - DNA, 20 bp, Artificial Sequence
  Description of Artificial Sequence Synthetic oligonucleotide
gaaacaatgataacaagacc

SEQ ID NO: 143 - DNA, 20 bp, Artificial Sequence
  Description of Artificial Sequence Synthetic oligonucleotide
gtggcccctgtgcccagccc

SEQ ID NO: 144 - DNA, 20 bp, Artificial Sequence
  Description of Artificial Sequence Synthetic oligonucleotide
gtcccaaatatgtagctgtt

SEQ ID NO: 145 - DNA, 20 bp, Artificial Sequence
  Description of Artificial Sequence Synthetic oligonucleotide
agagggacacacagatctat

SEQ ID NO: 146 - DNA, 20 bp, Artificial Sequence
  Description of Artificial Sequence Synthetic oligonucleotide
gctcccatcacatcaaccgg

SEQ ID NO: 147 - DNA, 20 bp, Artificial Sequence
  Description of Artificial Sequence Synthetic oligonucleotide
gatgtcacctccaatgacta

SEQ ID NO: 148 - DNA, 20 bp, Artificial Sequence
  Description of Artificial Sequence Synthetic oligonucleotide
gggcaaccacaaacccacga

SEQ ID NO: 149 - DNA, 20 bp, Artificial Sequence
  Description of Artificial Sequence Synthetic oligonucleotide
gttgaagatgaagcccagag

SEQ ID NO: 150 - DNA, 20 bp, Artificial Sequence
  Description of Artificial Sequence Synthetic oligonucleotide
gccaacaccaaccagaactt

SEQ ID NO: 151 - DNA, 20 bp, Artificial Sequence
  Description of Artificial Sequence Synthetic oligonucleotide
tgctgcacacagcaggcctt

SEQ ID NO: 152 - DNA, 20 bp, Artificial Sequence
  Description of Artificial Sequence Synthetic oligonucleotide
gtgccagaaacaggggtgac

SEQ ID NO: 153 - DNA, 20 bp, Artificial Sequence
  Description of Artificial Sequence Synthetic oligonucleotide
tcacggagactgaacactcc

SEQ ID NO: 154 - DNA, 20 bp, Artificial Sequence
  Description of Artificial Sequence Synthetic oligonucleotide
gtaacagacatggaccatca

SEQ ID NO: 155 - DNA, 20 bp, Artificial Sequence
  Description of Artificial Sequence Synthetic oligonucleotide
gggagggaggggcacagatg

SEQ ID NO: 156 - DNA, 20 bp, Artificial Sequence
  Description of Artificial Sequence Synthetic oligonucleotide
tgtggttccagaaccggagg

SEQ ID NO: 157 - DNA, 20 bp, Artificial Sequence
  Description of Artificial Sequence Synthetic oligonucleotide
aatgagagaaaatgaaactt

SEQ ID NO: 158 - DNA, 20 bp, Artificial Sequence
  Description of Artificial Sequence Synthetic oligonucleotide
ggccatcaaggatgcccacg

SEQ ID NO: 159 - DNA, 20 bp, Artificial Sequence
  Description of Artificial Sequence Synthetic oligonucleotide
aaattgtccagccccatctg

SEQ ID NO: 160 - DNA, 20 bp, Artificial Sequence
  Description of Artificial Sequence Synthetic oligonucleotide
cctgtaaaggaaactggaac

SEQ ID NO: 161 - DNA, 20 bp, Artificial Sequence
  Description of Artificial Sequence Synthetic oligonucleotide
tacatgaagcaactccagtc

SEQ ID NO: 162 - DNA, 20 bp, Artificial Sequence
  Description of Artificial Sequence Synthetic oligonucleotide
aaactccccccacccccttt

SEQ ID NO: 163 - DNA, 20 bp, Artificial Sequence
  Description of Artificial Sequence Synthetic oligonucleotide
gagttgggtttggtgctcaa

SEQ ID NO: 164 - DNA, 20 bp, Artificial Sequence
  Description of Artificial Sequence Synthetic oligonucleotide
gcgggccagcctgaatagct

SEQ ID NO: 165 - DNA, 20 bp, Artificial Sequence
  Description of Artificial Sequence Synthetic oligonucleotide
gaaacaatgataacaagacc

SEQ ID NO: 166 - DNA, 20 bp, Artificial Sequence
  Description of Artificial Sequence Synthetic oligonucleotide
gtggcccctgtgcccagccc

SEQ ID NO: 167 - DNA, 20 bp, Artificial Sequence
  Description of Artificial Sequence Synthetic oligonucleotide
gtcccaaatatgtagctgtt

SEQ ID NO: 168 - DNA, 20 bp, Artificial Sequence
  Description of Artificial Sequence Synthetic oligonucleotide
gctcccatcacatcaaccgg

SEQ ID NO: 169 - DNA, 20 bp, Artificial Sequence
  Description of Artificial Sequence Synthetic oligonucleotide
gatgtcacctccaatgacta

SEQ ID NO: 170 - DNA, 20 bp, Artificial Sequence
  Description of Artificial Sequence Synthetic oligonucleotide
gttgaagatgaagcccagag

SEQ ID NO: 171 - DNA, 20 bp, Artificial Sequence
  Description of Artificial Sequence Synthetic oligonucleotide
gccaacaccaaccagaactt

SEQ ID NO: 172 - DNA, 20 bp, Artificial Sequence
  Description of Artificial Sequence Synthetic oligonucleotide
tgctgcacacagcaggcctt

SEQ ID NO: 173 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 174 - RNA, 60 nt, Artificial Sequence
  Description of Artificial Sequence Synthetic oligonucleotide
guuuuagagcugugcuguuuaaacaacacagcaaguuaaaauaaggcuuuguccguacuc

SEQ ID NO: 175 - protein, 1377 aa, Artificial Sequence
  Description of Artificial Sequence Synthetic polypeptide
MPKKKRKVGMGKPYSIGLDIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFMQPVVAYSILVFADVEKGKSKKLRKVQDMVGITIMEKKRFEKNPVDFLEQRGYRNVRLEKIIKLPKYSLFELENKRRRLLASAKFLQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPRAFNFFGENIARKEYRSVTECLNATLIHQSITGLYETRIDLSKLGEDGEGADKRTADGSEFESPKKKRKV

SEQ ID NO: 176 - protein, 1377 aa, Artificial Sequence
  Description of Artificial Sequence Synthetic polypeptide
MPKKKRKVGMGKPYSIGLDIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGIIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERLHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFMQPVVAYSILVFADVEKGKSKKLRKVQDMVGITIMEKKRFEKNPVDFLEQRGYRNVRLEKIIKLPKYSLFELENKRRRLLASAKFLQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPRAFNFFGENIARKEYRSVTECLNATLIHQSITGLYETRIDLSKLGEDGEGADKRTADGSEFESPKKKRKV

SEQ ID NO: 177 - DNA, 20 bp, Artificial Sequence
  Description of Artificial Sequence Synthetic oligonucleotide
atcgacaagaaagggactga

SEQ ID NO: 178 - protein, 1566 aa, Artificial Sequence
  Description of Artificial Sequence Synthetic polypeptide
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSGKPYSIGLAIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGSIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERRHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFMQPVVAYSILVFADVEKGKSKKLRKVQDMVGITIMEKKRFEKNPVDFLEQRGYRNVRLEKIIKLPKYSLFELENKRRRLLASAKFLQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPRAFNFFGENIARKEYRSVTECLNATLIHQSITGLYETRIDLSKLGEDGEGADKRTADGSEFESPKKKRKV

SEQ ID NO: 179 - protein, 1566 aa, Artificial Sequence
  Description of Artificial Sequence Synthetic polypeptide
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSGKPYSIGLAIGTNSVGWAVVTDDYKVPAKKMKVLGNTDKQSIKKNLLGALLFDSGETAEATRLKRTARRRYTRRKNRLRYLQEIFTGEMNKVDENFFQRLDDSFLVDEDKRGEHHPIFGNIAAEVKYHDDFPTIYHLRRHLADTSKKADLRLVYLALAHMIKFRGHFLYEGDLKAENTDVQALFKDFVEEYDKTIEESHLSEITVDALSILTEKVSKSSRLENLIAHYPTEKKNTLFGNLIALSLDLHPNFKTNFQLSEDAKLQFSKDTYEEDLEGFLGEVGDEYADLFASAKNLYDAILLSGILTVDDNSTKAPLSASMVKRYEEHQKDLKKLKDFIKVNAPDQYNAIFKDKNKKGYASYIESGVKQDEFYKYLKGILLKINGSGDFLDKIDREDFLRKQRTFDNGIIPHQIHLQEMHAILRRQGEHYPFLKENQDKIEKILTFRIPYYVGPLARKGSRFAWAEYKADEKITPWNFDDILDKEKSAEKFITRMTLNDLYLPEEKVLPKHSPLYEAFTVYNELTKVKYVNEQGEAKFFDTNMKQEIFDHVFKENRKVTKDKLLNYLNKEFEEFRIVNLTGLDKENKAFNSSLGTYHDLRKILDKSFLDDKANEKTIEDIIQTLTLFEDREMIRQRLQKYSDIFTKAQLKKLERLHYTGWGRLSYKLINGIRNKENKKTILDYLIDDGYANRNFMQLINDDALSFKEEIARAQIIDDVDDIANVVHDLPGSPAIKKGILQSVKIVDELVKVMGHNPANIIIEMARENQTTDKGRRNSQQRLKLLQDSLKNLDNPVNIKNVENQQLQNDRLFLYYIQNGKDMYTGETLDINNLSQYDIDHIIPQAFIKDNSLDNRVLTRSDKNRGKSDDVPSIEVVHEMKSFWSKLLSVKLITQRKFDNLTKAERGGLTEEDKAGFIKRQLVETRQITKHVAQILDERFNTEFDGNKRRIRNVKIITLKSNLVSNFRKEFELYKVREINDYHHAHDAYLNAVVGNALLLKYPQLEPEFVYGEYPKYNSYRSRKSATEKFLFYSNILRFFKKEDIQTNEDGEIAWNKEKHIKILRKVLSYPQVNIVKKTEEQTGGFSKESILPKGESDKLIPRKTKNSYWDPKKYGGFMQPVVAYSILVFADVEKGKSKKLRKVQDMVGITIMEKKRFEKNPVDFLEQRGYRNVRLEKIIKLPKYSLFELENKRRRLLASAKFLQKGNELVIPQRFTTLLYHSYRIEKDYEPEHREYVEKHKDEFKELLEYISVFSRKYVLADNNLTKIEMLFSKNKDAEVSSLAKSFISLLTFTAFGAPRAFNFFGENIARKEYRSVTECLNATLIHQSITGLYETRIDLSKLGEDGEGADKRTADGSEFESPKKKRKV

SEQ ID NO: 180 - RNA, 98 nt, Artificial Sequence
  Description of Artificial Sequence Synthetic oligonucleotide
guuuuagagcugugcuguuuaaacaacacagcaaguuaaaauaaggcuuuguccguacucaagcuugcaaaagcgugcaccgauucggugcuuuuuuu

SEQ ID NO: 181 - DNA, 107 bp, Artificial Sequence
  Description of Artificial Sequence Synthetic oligonucleotide
  Description of Combined DNA/RNA Molecule Synthetic oligonucleotide
guuugagaguagugugaaaacauuacgaguucaaauacaaauuaauuuacaaugccuucgggcugcccgacguagggcaccuacucucaauucuucggaauugagtt

SEQ ID NO: 182 - RNA, 128 nt, Artificial Sequence
  Description of Artificial Sequence Synthetic polynucleotide
gucuuggaugagugugaaaacacucauagucaagaucaaacgagugguuuuccacgaguuauuacuuuugaggucuuauauggcccauacauaaaaaggagucggaauuuccggcuccuuuucuuuuu